(19) United States
(12) Patent Application Publication
(10) Pub. No.: US 2009/0099179 A1
(43) Pub. Date: Apr. 16, 2009

(54) COMPOUNDS FOR DISEASES AND DISORDERS

(75) Inventors: Christine Klein, Salt Lake City, UT (US); Andrew D. Gassman, Salt Lake City, UT (US); Leena Bhoite, Salt Lake City, UT (US); John Manfredi, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: MYRIAD GENETICS, INCORPORATED, SALT LAKE CITY, UT (US)

(21) Appl. No.: 12/295,922

(22) PCT Filed: Apr. 4, 2007
(86) PCT No.: PCT/US07/65969

§ 371 Date: Oct. 3, 2008

Related U.S. Application Data
(60) Provisional application No. 60/789,524, filed on Apr. 4, 2006.

(51) Int. Cl. A61K 31/5377; A61K 31/404; A61K 31/403; A61K 31/4439; A61K 31/501; A61K 31/506; A61P 25/00; A61K 31/497; A61K 31/5025; A61K 31/519; A61K 31/4985

(52) U.S. Cl. 514/235.2; 514/415; 514/411; 514/339; 514/252.06; 514/256; 514/255.05; 514/248; 514/267; 514/250

(57) ABSTRACT

The invention provides novel compounds useful for the treatment of disorders associated with a defect in vesicular transport (e.g., axonal transport). The compounds have a substituents chosen from -L-C(═O)OH, -L-CH═CHC(═O)OH, -L-C(═O)NH₂, -L-C(═O)NH(C1-3 alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, where L is a linker.

### CROSS-REFERENCE TO RELATED APPLICATIONS

This application claims priority under 35 U.S.C. § 119(e) to U.S. provisional application Ser. No. 60/789,524, filed Apr. 4, 2007, which is hereby incorporated by reference in its entirety.

### TECHNICAL FIELD OF THE INVENTION

The invention provides a method for the therapeutic treatment of disorders associated with axonal transport defects.

### BACKGROUND OF THE INVENTION

### Summary of the Invention

In general, the invention relates to compounds of Formulae I-XIV, pharmaceutically acceptable salts thereof, and pharmaceutical compositions containing the compounds and salts. The compounds of the invention can be used for the treatment and prophylaxis of disorders associated with a defect in vesicular transport (e.g., axonal transport).

In a first aspect, the invention provides compounds of Formula I and II, pharmaceutically acceptable salts thereof, and pharmaceutical compositions having such compounds for use in treating and/or preventing disorders associated with vesicular transport defects.

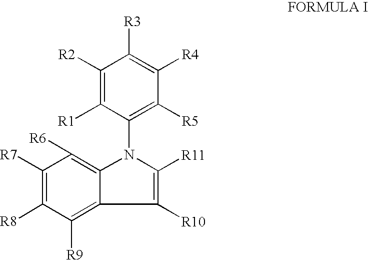

According to the first aspect of the invention, compounds of Formula I have one or more of R1-R5 independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R11 is an optionally substituted phenyl group;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, and wherein each carbon can be optionally substituted with one or more C₁₋₃ alkyl or C₃₋₆ cycloalkyl.

The first aspect of the invention also includes compounds of Formula II.

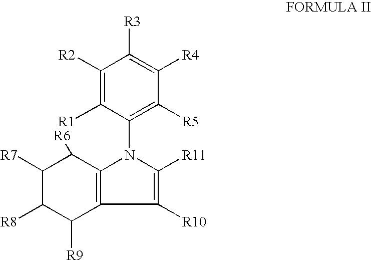

In the first aspect of the invention, compounds of Formula II are provided having one or more of R1-R5 independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -LC(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; with the provision when R2 is —C(═O)OH, then R3 is not hydroxyl (or —O—C(═O)CH₃), —SH, —Cl, —NH₂, methoxy, and —NHC(═O)CH₃;

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, -(N-methyl)-piperazinyl, —OCH₂-phenyl, -pyridinyl, methylenedioxy, ethylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R11 is an optionally substituted phenyl group; and

Rₒ is chosen from haloalkyl and alkyl.

According to one embodiment of the first aspect of the invention, R8 and R9 in the compounds of Formula I are taken together to form a 6 member aryl ring as in Formula III.

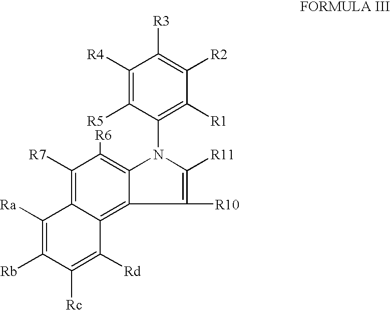

According to one embodiment of the first aspect of the invention, compounds of Formula III are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the first aspect of the invention.

According to one embodiment of the first aspect of the invention, R8 and R9 in the compounds of Formula II are taken together to form a 6 member aryl ring as in Formula IV.

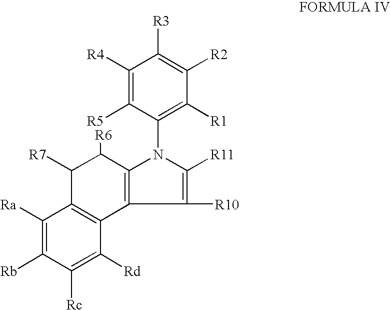

According to one embodiment of the first aspect of the invention, compounds of Formula IV are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the first aspect of the invention.

In a second aspect, the invention provides compounds of Formula I and II for use in treating and/or preventing disorders associated with axonal transport defects, wherein R1-R5 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

one or more of R6-R9 are chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; or two adjacent of R6-R9 can be taken together to form a 4-7 member substituted aryl or cycloalkyl ring wherein the substituent is chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; and the others of R6-R9, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R10 is chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₁₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

R11 is an optionally substituted phenyl group; and

L is as defined above.

In a third aspect, the invention provides compounds of Formula I and II for use in treating and/or preventing disorders associated with axonal transport defects,

wherein R1-R9 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R10 is chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L is as defined above.

In a fourth aspect, the invention provides compounds of Formula I and II for use in treating and/or preventing disorders associated with axonal transport defects, wherein R1-R10 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R11 is a phenyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, —L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl and the others are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl; and

L is as defined above.

In a fifth aspect, the invention provides compounds of Formula I and II for use in treating and/or preventing disorders associated with axonal transport defects, wherein R1-R9 and R11 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R10 is a phenyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L is as defined above.

In a sixth aspect, the invention provides compounds of Formula I and II for use in treating and/or preventing disorders associated with axonal transport defects, wherein R1-R9 and R11 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R10 is -L-R12 wherein L is as defined above; and

R12 is a phenyl ring substituted with one or more substituents independently chosen from of -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)Rₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L is as defined above.

In a seventh embodiment, the invention provides compounds of Formula I and II for use in treating and/or preventing disorders associated with axonal transport defects, wherein R1-R10 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R11 is -L-R12 wherein L is as defined above; and

R12 is a phenyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl; and

L is as defined above.

In an eighth embodiment, the invention provides compounds of Formula I and II for use in treating and/or preventing disorders associated with axonal transport defects, wherein R1-R9 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl ring;

R10 and R11 are independently chosen from hydro, hydroxyl, halo, alkyl, alkoxy, haloalkyl, haloalkoxy, —N(C₁₋₃ alkyl)₂, —NH(C₁₋₃ alkyl), —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, and -L-R12; and

R12 is a phenyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L is as defined above.

In a ninth aspect, the invention provides compounds of Formula V and VI for use in treating and/or preventing disorders associated with axonal transport defects,

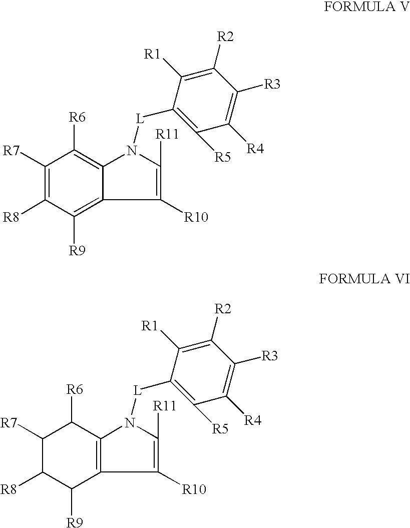

wherein one or more of R1-R5 is independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

L is as defined above;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring; and

R11 is an optionally substituted phenyl group.

In one embodiment of the ninth aspect of the invention, R8 and R9 in the compound of Formula V are taken together to form a 6 member aryl ring as in Formula VII.

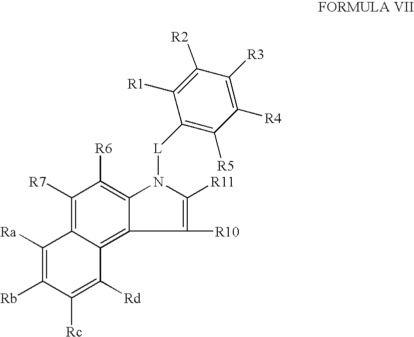

According to one embodiment of the ninth aspect of the invention, compounds of Formula VII are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the ninth aspect of the invention.

In one embodiment of the ninth aspect of the invention, R8 and R9 in the compounds of Formula VI are taken together to form a 6 member aryl ring as in Formula VIII.

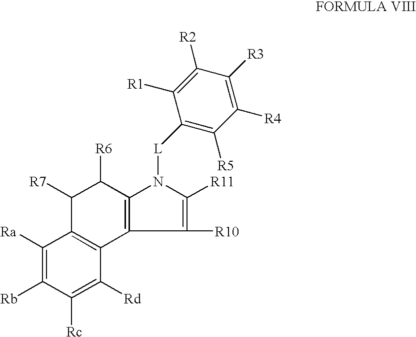

According to one embodiment of the ninth aspect of the invention, compounds of Formula VIII are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the ninth aspect of the invention. (10)

In a tenth aspect, the invention provides compounds of Formula IX and X for use in treating and/or preventing disorders associated with axonal transport defects:

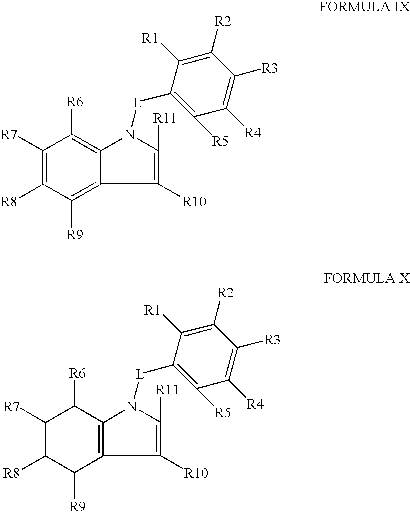

wherein one or more of R1-R11 are chosen from -L-R12, -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; wherein R12 is a phenyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃ alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

L is as defined above; and the others of R1-R11 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; and two adjacent of R6-R9 can be taken together to form a 4-7 member optionally substituted aryl or cycloalkyl ring.

In one embodiment of the tenth aspect of the invention, R8 and R9 in the compounds of Formula IX are taken together to form a 6 member aryl ring as in Formula XI

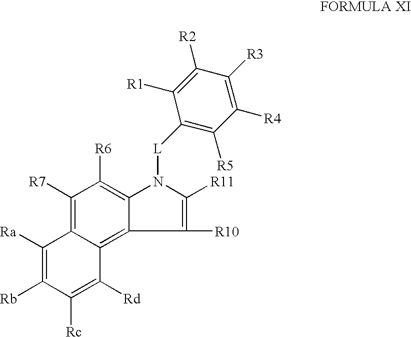

According to one embodiment of the tenth aspect of the invention, compounds of Formula XI are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the tenth aspect of the invention.

In one embodiment of the tenth aspect of the invention, R8 and R9 in the compounds of Formula X are taken together to form a 6 member aryl ring as in Formula XII.

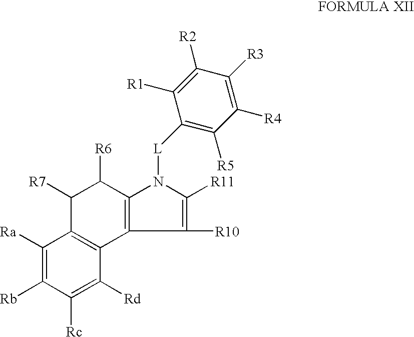

According to one embodiment of the tenth aspect of the invention, compounds of Formula XII are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the tenth aspect of the invention.

In an eleventh aspect, the invention provides compounds of Formula XIII and XIV for use in treating and/or preventing disorders associated with axonal transport defects:

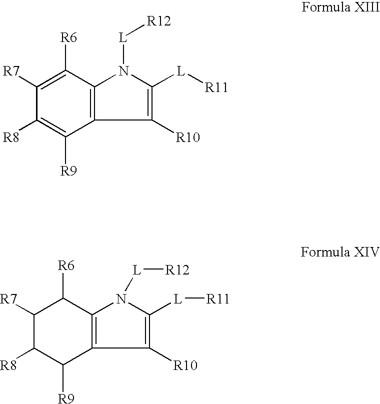

wherein L is as defined above or is selected from an optionally substituted, saturated or partially saturated cyclopropyl, cyclobutyl, cyclopentyl, cyclohexyl, cycloheptyl, and C₁₋₁₂alkyl;
R1-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R11 is chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

R12 is chosen from optionally substituted C₁₋₁₂ alkyl, phenyl, and C₃₋₇ cycloalkyl.

In one embodiment of the eleventh aspect of the invention, R8 and R9 in the compounds of Formula XI are taken together to form a 6 member aryl ring as in Formula XIII.

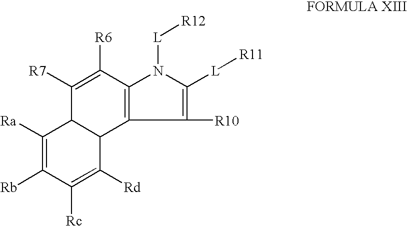

According to one embodiment of the eleventh aspect of the invention, compounds of Formula XIII are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the eleventh aspect of the invention.

In one embodiment of the eleventh aspect of the invention, R8 and R9 in the compounds of Formula XII are taken together to form a 6 member aryl ring as in Formula XIV.

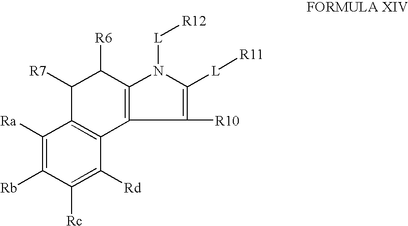

According to one embodiment of the eleventh aspect of the invention, compounds of Formula XIV are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the eleventh aspect of the invention.

In a twelfth aspect, the invention provides compounds of Formula I and II, wherein one or more of R1-R5 is chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others of R1-R5, independent of one another, are chosen hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R11 is an optionally substituted heterocyclic group; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In a thirteenth aspect, the invention provides compounds of Formula I and II for use in treating and/or preventing disorders associated with axonal transport defects, wherein R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂NH₂, —S(═O)₂(C₁₋₃alkyl), —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

one or more of R6-R9 is independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; or two adjacent of R6-R9 can be taken together to form an optionally substituted 4-7 member aryl, heterocyclic, or cycloalkyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; and the others of R6-R9, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

R11 is an optionally substituted heterocyclic group; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl.

In a fourteenth aspect, the invention provides compounds of Formula I and II for use in treating and/or preventing disorders associated with axonal transport defects, wherein R1-R9 are independently chosen hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form an optionally substituted C₄₋₇ member aryl, heterocyclic, or cycloalkyl ring;

R10 is chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl;

R11 is an optionally substituted heterocyclic group; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In a fifteenth aspect, the invention provides compounds of Formula I and II for use in treating and/or preventing disorders associated with axonal transport defects, wherein R1-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R11 is a heterocyclic group with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In a sixteenth aspect, the invention provides compounds of Formula I and II for use in treating and/or preventing disorders associated with axonal transport defects, wherein R1-R9 and R11 independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R10 is a heterocyclic group with one or more substituents independently chosen -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In a seventeenth aspect, the invention provides compounds of Formula I and II for use in treating and/or preventing disorders associated with axonal transport defects, wherein R1-R9 and R11 independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

### R10 is -L-R12;

R12 is a heterocyclic group with one or more substituents chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In an eighteenth embodiment, the invention provides compounds of Formula I and II for use in treating and/or preventing disorders associated with axonal transport defects, wherein R1-R9 and R11 independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R10 is a heterocyclic group with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In a nineteenth aspect, the invention provides compounds of Formula I and II for use in treating and/or preventing disorders associated with axonal transport defects, wherein R1-R9, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R10 and R11 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂NH₂, —S(═O)₂(C₁₋₃alkyl), —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, and -L-R12;

R12 is a heterocyclic group with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In a twentieth aspect, the invention provides compounds of Formula V and VI for use in treating and/or preventing disorders associated with axonal transport defects,

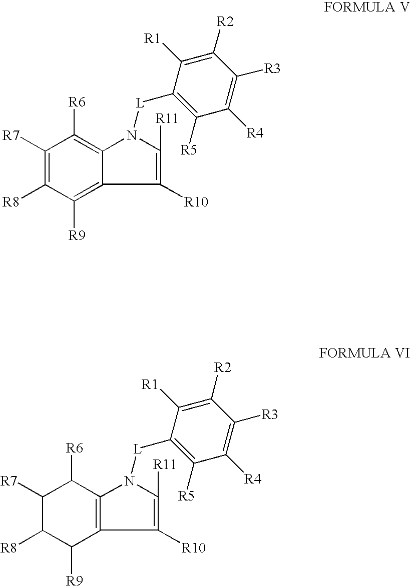

wherein one or more of R1-R5 is independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R11 is an optionally substituted heterocyclic group; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In a twenty-first aspect, the invention provides compounds of Formula V and VI for use in treating and/or preventing disorders associated with axonal transport defects,

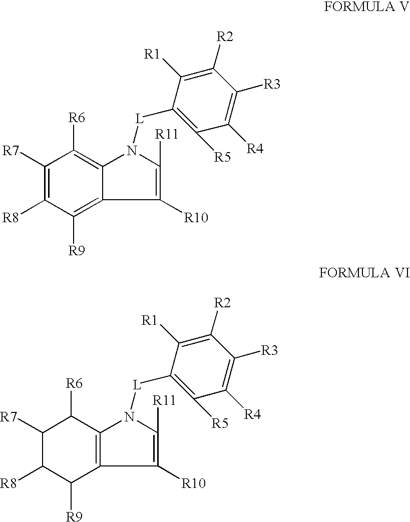

wherein R1-R11, independent of one another, are chosen from -L-R12, -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl;

R12 is a heterocyclic group with one or more substituents independently chosen -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; and

the others of R1-R11 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In one embodiment of this twenty-first aspect, the invention includes analogs where the ring to which R1-R5 are attached is a 4-7 member heterocyclic ring instead a phenyl ring.

In another aspect of the invention, one or more of the carbon atoms of the indole core are replaced by a heteroatom independently chosen from —N—, —O—, and —S—.

In some embodiments of the invention, Rₒ is independently chosen from methyl or ethyl.

Optionally substituted, when used herein without reference to further definition, refers to a substituent independently chosen from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

Furthermore, the invention provides derivatives or analog of the compounds defined in first through twenty-first aspects of the invention, where the derivative or analog is chosen from an ester (e.g., methyl or ethyl ester), an amide, a carbamate, a urea, an amadine, or a combination thereof. Methods for generating an ester, an amide, a carbamate, a urea, an amadine, or a combination thereof, of the compounds of the first aspect through the twenty-first aspects are known to an ordinary artisan skilled in organic chemical synthesis.

As the skilled artisan readily recognizes, in some of the embodiments of the first twenty-one aspects of the invention, some of the compounds can have more than one -L-group, each of which is independent chosen.

In a twenty-second aspect, the invention provides a method of treating and/or preventing a disorder characterized by an axonal transport defect, by identifying a patient in need of such treatment, and administering to the patient a therapeutically effective amount of a pharmaceutical composition having one or more compounds of Formulae I-XVI. Administration of a compound of Formulae I-XVI for at least 4 weeks, preferably at least 4 months, and more desirably at least 8 months, can provide an improvement or lessening in function as characterized by appropriate tests, biochemical disease marker progression, and/or pathology. Desirably, the oral dose is provided in capsule or tablet form. The pharmaceutical composition for use in the invention is formulated with one or more pharmaceutically acceptable excipients, salts, or carriers. The pharmaceutical composition for use in the invention is delivered orally, preferably in a tablet or capsule dosage form.

In a twenty-third aspect, the invention provides a method for prophylaxis against a neurodegenerative disorder characterized by a defect in axonal transport (and/or vesicular transport), by identifying a patient in need of or desiring such treatment, and administering to the patient a prophylactically effective amount of a pharmaceutical composition having one or more compounds of Formulae I-XVI. Administration of a compound of Formulae I-XVI for at least 4 weeks, preferably at least 4 months, and more desirably at least 8 months, can delay the onset of the neurodegenerative disorder or slow the rate of onset of symptoms of the disorder. Patients having a predisposition to a disorder or suspected of needing prophylaxis can be identified by any method known to the skilled artisan for diagnosis such disorders.

In a twenty-fourth aspect, the invention provides a method of treating a disease characterized by abnormal axonal (and/or vesicular) transport by (1) identifying a patient in need of such treatment, and (2) administering to the patient a therapeutically effective amount of a pharmaceutical composition having one or more compounds of Formulae I-XVI. Oral administration of the pharmaceutical composition for use in the method of this aspect the invention for at least 4 weeks, preferably at least 4 months, and more desirably at least 8 months, provides an improvement or lessening in decline of e function as characterized by cognition tests, biochemical disease marker progression, and/or pathology Desirably, the composition is provided as an oral dose, preferably in capsule or tablet form.

In a twenty-fifth aspect, the invention provides a method of prophylaxis or delaying the onset of a disease (or one or more symptoms thereof) characterized by abnormal axonal transport (and/or vesicular transport), by identifying a patient in need of such treatment and administering to the patient a prophylactically effective amount of a pharmaceutical composition having one or more compounds of Formulae I-XVI. Oral administration of the pharmaceutical composition for use in the method of this aspect the invention for at least 4 weeks, preferably at least 4 months, and more desirably at least 8 months, prevents or delays the onset of the disease (or symptoms thereof).

In a twenty-sixth aspect, the invention provides a method of treating a disease chosen from amyotrophic lateral sclerosis (ALS), Charcot-Marie-Tooth Disease 2 (CMT2), spinal muscular atrophy (SPA), spinal muscular atrophy (SMA), Parkinson's Disease (PD), hereditary sensory motor neuropathy, Optic neuropathies (e.g., Leber's hereditary optic neuropathy (LHON) and Cuban epidemic of optic neuropathy (CEON)), Niemann-Pick type C disease (NPC), Down syndrome, Dementia with Lewy Bodies (DLB), Parkinson's disease, Tauopathies (e.g., progressive supranuclear palsy, corticobasal degeneration, Pick's disease, argyrophilic grain disease, and frontotemporal dementia and parkinsonism linked to chromosome 17 (FTDP-17)), miscellaneous motor neuron disorders (e.g., Primary lateral sclerosis (PLS)), Hereditary spastic paraplegia, spinal muscular atrophy, multiple sclerosis, Guillain-Barré syndrome, traumatic brain injury, spinal cord injury, and polyQ diseases (e.g., Huntington disease, spinobulbar muscular atrophy, dentatorubral-pallidoluysian atrophy, Kennedy's disease (also called spinobulbar muscular atrophy [SBMA]), spinocerebellar ataxia 1, spinocerebellar ataxia 2, spinocerebellar ataxia 3, spinocerebellar ataxia 6, spinocerebellar ataxia 7, and spinocerebellar ataxia 17) comprising administering to a patient in need of such treatment, a pharmaceutical composition having one or more compounds of Formulae I-XVI. Oral administration of the pharmaceutical composition for use in the method of this aspect of the invention for at least 4 weeks, preferably at least 4 months, and more desirably at least 8 months, provides an improvement or lessening in decline of function, biochemical disease marker progression, and/or pathology. Desirably, the oral dose is provided in capsule or tablet form. According to this aspect of the invention, a patient in need of treatment is administered disease treating (and/or preventing) effective amount of a pharmaceutical composition having one or more compounds of Formulae I-XVI and one or more pharmaceutically acceptable salts, excipients and carriers. The method of this aspect of the invention involves identifying an individual having a particular disorder characterized (at-least in part) as being associated with a defect in axonal or vesicular transport. An individual having a particular disease can be diagnosed by any method available to the ordinary artisan skilled in such diagnoses. For example, diagnosis can be according to DSM IV (TR) and/or meets clinically accepted criteria for having the disease. According to this aspect of the invention, individuals with the disease take an oral dose of a pharmaceutical composition for a specified period of time. Individuals undergoing such treatment are likely to see an improvement or lessening in decline of function, an improvement or lessening in decline in biochemical disease marker progression, and/or an improvement or lessening decline in pathology. A lessening in decline in function can be assessed using a clinically appropriate test of function.

In a twenty-seventh aspect, the invention provides a method of preventing the onset of a disease associated with a defect in vesicular transport comprising administering to a patient in need of or desiring such treatment, a pharmaceutical composition having one or more compounds of Formulae I-XVI. Oral administration of the pharmaceutical composition for use in the method of this aspect of the invention for at least 4 weeks, preferably at least 4 months, and more desirably at least 8 months, delays the onset of decline of cognitive function, biochemical disease marker progression, and/or plaque pathology. According to this embodiment, an individual desiring or needing preventative treatment against the onset of AD is administered a pharmaceutical composition having one or more compounds of Formulae I-XVI. Desirably, the oral dose is provided in capsule or tablet form. The preventive treatment is preferably maintained as long as the individual continues to desire or need the treatment. Individuals needing or desiring preventative treatment against AD can be those having risk factors for developing AD. For example, risk factors for developing AD can be genetic factors or environmental factors. In one embodiment, the risk factor is age. Genetic risk factors can be assessed in a variety of ways, such as ascertaining the family medical history of the individual, or performing a genetic test to identify genes that confer a predisposition for developing AD. Additionally, risk factors can be assessed by monitoring genetic and biochemical markers.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

N/A

### DETAILED DESCRIPTION OF THE INVENTION

In general, the invention relates to the use of pharmaceutical compositions having one or more compounds of Formulae I-XVI as the active ingredient, for treating and/or preventing disorders characterized by abnormal vesicular transport (e.g., axonal transport). When the pharmaceutical composition is administered, according to the treatment regimens of the invention, to an individual desiring or needing such treatment, it provides an improvement or lessening in decline of cognitive function, biochemical disease marker progression, and/or pathology associated with the disorder. The composition of the invention is formulated with one or more pharmaceutically acceptable excipients, salts, or carriers. The pharmaceutical composition of the invention is delivered orally, preferably in a tablet or capsule dosage form. The pharmaceutical compositions can be used in methods for treating, preventing, and prophylaxis against the disorders characterized by defects in vesicular transport (e.g., axonal transport).

The invention therefore provides compounds of Formulae I-XVI as described in the Summary of the Invention (and in more detail below) and pharmaceutical composition having such compounds. In one specific use, the compounds can be used for the treatment and/or prophylaxis of disorders characterized by defects in axonal and/or vesicular transport. The inventors have found that compounds of Formulae I-XVI as described in the summary can amerliorate disease models of vesicular transport associated diseases (e.g., axonal transport).

Some of the compounds of Formulae I-XVI, for use in the invention may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the compounds that are optically active are used in optically pure form. Furthermore, some of the compound for use in the invention can exist as cis and trans geometric isomers all such isomers and mixtures thereof are intended to be within the scope of the present invention.

Additionally, the formulas are intended to cover solvated as well as unsolvated forms of the identified structures. For example, Formulae I-XVI includes compounds of the indicated structure in both hydrated and non-hydrated forms. Other examples of solvates include the structures in combination with isopropanol, ethanol, methanol, DMSO, ethyl acetate, acetic acid, or ethanolamine.

In addition to compounds of Formulae I-XVI, the invention includes pharmaceutically acceptable prodrugs, pharmaceutically active metabolites, and pharmaceutically acceptable salts of such compounds.

Prodrugs and active metabolites of compound may be identified using routine techniques known in the art. See, e.g., Bertolini, G et al., J. Med. Chem., 40, 2011-2016 (1997); Shan, D. et al., J. Pharm. Sci., 86(7), 756-767; Bagshawe K., Drug Dev. Res., 34, 220-230 (1995); Bodor N; Advance in Drug Res., 13, 224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991).

### Compounds of the Invention

In general, the invention relates to compounds of Formulae I-XIV, pharmaceutically acceptable salts thereof, and pharmaceutical compositions containing the compounds and salts. The compounds of the invention can be used for the treatment and prophylaxis of disorders characterized by a defect in axonal transport (and/or vesicular transport).

In a first aspect, the invention provides for the use of compounds of Formula I and II, pharmaceutically acceptable salts thereof, and pharmaceutical compositions having such compounds to treat (and/or prevent) diseases characterized by a defect in vesicular transport (e.g., axonal transport).

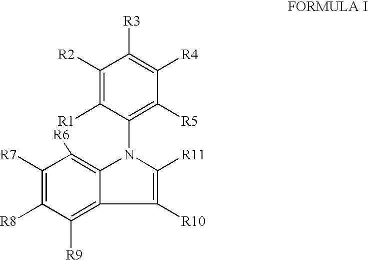

According to the first aspect of the invention, compounds of Formula I have one or more of R1-R5 independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂, with the provision that R3 is not hydroxyl;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R11 is an optionally substituted phenyl group;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, and wherein each carbon can be optionally substituted with one or more C₁₋₃ alkyl or C₃₋₆ cycloalkyl.

In one sub-embodiment, with the compound is not 1-[4-(methylsulfonyl)phenyl]-2-phenyl-1H-Indole.

According to one embodiment of the first aspect of the invention, one or more of R1-R5 in the compounds of Formula I, are independently chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₁₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, —S(═O)₂N(C₁₋₃alkyl)₂, and the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two adjacent of R6-R9 can be taken together to form a 4-7 member optionally substituted aryl or cycloalkyl ring;

L is as defined above; and

R11 is an optionally substituted phenyl group.

In one sub-embodiment R3 is not hydroxyl.

According to another embodiment of this first aspect of the invention, in the compounds of Formula I, one of R1-R5 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others of R1-R5 independently are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two of R6-R9 can be taken together to form an optionally substituted C₄₋₇ aryl or cycloalkyl ring; and

R11 is an optionally substituted phenyl.

According to one embodiment of the first aspect of the invention, in the compounds of Formula I, R1 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

According to one embodiment of the first aspect of the invention, in the compounds of Formula I, R1 is chosen from —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₁₃alkyl), —C(═O)N(C₁₋₁₃alkyl)₂, —S(═O)₂NH₂, and —S(═O)₂N(C₁₋₃alkyl)₂.

According to another embodiment of the first aspect of the invention, in the compounds of Formula I, R1 is chosen from -L-C(═O)OH, -L-CH═CHC(═O)OH, -L-CH₂CH₂C(═O)OH, -L-CH₂CH₂CH₂C(═O)OH, -L-C(CH₂CH₂)C(═O)OH, -L-CH(CH₃)C(═O)OH, -L-CH(CH₂CH₃)C(═O)OH, -L-C(CH₃)(CH₂CH₃)C(═O)OH, -L-CH═C(CH₃)C(═O)OH, -L-C(CH₂CH₃)₂C(═O)OH, -LCH₂C(═O)OH, -L-C(CH₃)₂C(═O)OH, -L-C(═O)NH₂, -L-C(═O)NHCH₃, -L-C(═O)N(CH₃)₂, -L-S(═O)₂(C₁₋₃alkyl), -L-S(═O)₂NH₂, -L-S(═O)₂NHCH₃, -L-S(═O)₂N(CH₃)₂, -L-C(═O)NH(C₁₋₃alkyl), -L-C(═O)N(C₁₋₃alkyl)₂, -L-S(═O)₂NH₂, and -L-S(═O)₂N(C₁₋₃alkyl)₂, with the provision that if R1 is —COOH , or an ester thereof, then R10 is not —COOH, or an ester thereof.

According to one embodiment of the first aspect of the invention, in the compounds of Formula I, R2 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

According to yet another embodiment of the first aspect of the invention, in the compounds of Formula I, R2 is chosen from —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

According to another embodiment of the first aspect of the invention, in the compounds of Formula I, R2 is chosen from -L-C(═O)OH, -L-CH═CHC(═O)OH, -L-CH₂CH₂C(═O)OH, -L-CH₂CH₂CH₂C(═O)OH, -L-C(CH₂CH₂)C(═O)OH, -L-CH(CH₃)C(═O)OH, -L-CH(CH₂CH₃)C(═O)OH, -L-C(CH₃)(CH₂CH₃)C(═O)OH, -L-CH═C(CH₃)C(═O)OH, -L-C(CH₂CH₃)₂C(═O)OH, -LCH₂C(═O)OH, -L-C(CH₃)₂C(═O)OH, -L-C(═O)NH₂, -L-C(═O)NHCH₃, -L-C(═O)N(CH₃)₂, -L-S(═O)₂(C₁₋₃alkyl), -L-S(═O)₂NH₂, -L-S(═O)₂NHCH₃, -L-S(═O)₂N(CH₃)₂, -L-C(═O)NH(C₁₋₃alkyl), -L-C(═O)N(C₁₋₃alkyl)₂, -L-S(═O)₂NH₂, and -L-S(═O)₂N(C₁₋₃alkyl)₂, with the provision that when R2 is —C(═O)OH, R3 is not —OH or —OC(═O)CH₃.

According to another embodiment of the first aspect of the invention, in the compounds of Formula I, R3 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

According to still another embodiment of the first aspect of the invention, in the compounds of Formula I, R3 is chosen from -L-C(═O)OH, -L-CH═CHC(═O)OH, -L-CH₂CH₂C(═O)OH, -L-CH₂CH₂CH₂C(═O)OH, -L-C(CH₂CH₂)C(═O)OH, -L-CH(CH₃)C(═O)OH, -L-CH(CH₂CH₃)C(═O)OH, -L-C(CH₃)(CH₂CH₃)C(═O)OH, -L-CH═C(CH₃)C(═O)OH, -L-C(CH₂CH₃)₂C(═O)OH, -LCH₂C(═O)OH, -L-C(CH₃)₂C(═O)OH, -L-C(═O)NH₂, -L-C(═O)NHCH₃, -L-C(═O)N(CH₃)₂, -L-S(═O)₂(C₁₋₃alkyl), -L-S(═O)₂NH₂, -L-S(═O)₂NHCH₃, -L-S(═O)₂N(CH₃)₂, -L-C(═O)NH(C₁₋₃alkyl), -L-C(═O)N(C₁₋₃alkyl)₂, -L-S(═O)₂NH₂, and -L-S(═O)₂N(C₁₋₃alkyl)₂.

The first aspect of the invention also includes the use of compounds of Formula II.

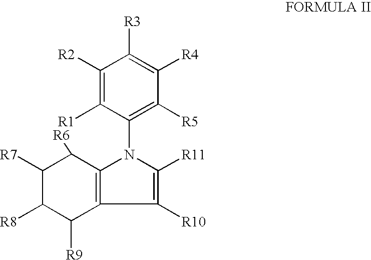

In the first aspect of the invention, compounds of Formula II are provided having one or more of R1-R5 independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -LC(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R11 is an optionally substituted phenyl group; and

Rₒ is chosen from haloalkyl and alkyl.

In one sub-embodiment, when R2 is —C(═O)OH, then R3 is not hydroxyl (or —O—C(═O)CH₃), —SH, —Cl, —NH₂, methoxy, and —NHC(═O)CH₃;

In one sub-embodiment, the compound is not
4-(4,5-dihydro-2-phenyl-3H-benz[e]indol-3-yl)-2-hydroxy-benzoic acid,
4-(4,5-dihydro-2-phenyl-3H-benz[e]indol-3-yl)-benzoic acid,
4-(7-chloro-4,5-dihydro-2-phenyl-3H-benz[e]indol-3-yl)-2-hydroxy-benzoic acid,
2-hydroxy-4-(4,5,6,7-tetrahydro-2-phenyl-1H-indol-1-yl)-benzoic acid,
4-(4,5,6,7-tetrahydro-2-phenyl-1H-indol-1-yl)-benzoic acid,
3-(4,5-dihydro-2-phenyl-3H-benz[e]indol-3-yl)-benzamide,
4-(4,5-dihydro-2-phenyl-3H-benz[e]indol-3-yl)-benzamide,
3-(4,5-dihydro-2-phenyl-1H-benz[g]indol-1-yl)-benzoic acid,
2-(4,5-dihydro-2-phenyl-1H-benz[g]indol-1-yl)-benzoic acid, or
3-[2-(4-bromophenyl)-4,5,6,7-tetrahydro-1H-indol-1-yl]-benzoic acid.

In one embodiment of the first aspect of the invention, one of R1-R5 in the compounds of Formula II is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂, and the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two adjacent of R6-R9 can be taken together to form a 4-7 member optionally substituted aryl or cycloalkyl ring;

L is as defined above; and

R11 is an optionally substituted phenyl.

According to another embodiment of this first aspect of the invention, in the compounds of Formula II, one of R1-R5 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others of R1-R5 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two adjacent of R6-R9 can be taken together to form a 4-7 member optionally substituted aryl or cycloalkyl ring; and

R11 is an optionally substituted phenyl.

According to another embodiment of the first aspect of the invention, in the compounds of Formula II, R1 is chosen from -L-C(═O)OH, -L-CH═CHC(═O)OH, -L-CH₂CH₂C(═O)OH, -L-CH₂CH₂CH₂C(═O)OH, -L-C(CH₂CH₂)C(═O)OH, -L-CH(CH₃)C(═O)OH, -L-CH(CH₂CH₃)C(═O)OH, -L-C(CH₃)(CH₂CH₃)C(═O)OH, -L-CH═C(CH₃)C(═O)OH, -L-C(CH₂CH₃)₂C(═O)OH, -LCH₂C(═O)OH, -L-C(CH₃)₂C(═O)OH, -L-C(═O)NH₂, -L-C(═O)NHCH₃, -L-C(═O)N(CH₃)₂, -L-S(═O)₂(C₁₋₃alkyl), -L-S(═O)₂NH₂, -L-S(═O)₂NHCH₃, -L-S(═O)₂N(CH₃)₂, -L-C(═O)NH(C₁₋₃alkyl), -L-C(═O)N(C₁₋₃alkyl)₂, -L-S(═O)₂NH₂, and -L-S(═O)₂N(C₁₋₃alkyl)₂. In one sub-embodiment, the compound is not 2-(4,5-dihydro-2-phenyl-1H-benz[g]indol-1-yl)benzoic acid (CAS No. 54670-19-8).

According to yet another embodiment of the first aspect of the invention, in the compounds of Formula II, R1 is chosen from —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

According to still another embodiment of the first aspect of the invention, in the compounds of Formula II, R2 is chosen from -L-C(═O)OH, -L-CH═CHC(═O)OH, -L-CH₂CH₂C(═O)OH, -L-CH₂CH₂CH₂C(═O)OH, -L-C(CH₂CH₂)C(═O)OH, -L-CH(CH₃)C(═O)OH, -L-CH(CH₂CH₃)C(═O)OH, -L-C(CH₃)(CH₂CH₃)C(═O)OH, -L-CH═C(CH₃)C(═O)OH, -L-C(CH₂CH₃)₂C(═O)OH, -LCH₂C(═O)OH, -L-C(CH₃)₂C(═O)OH, -L-C(═O)NH₂, -L-C(═O)NHCH₃, -L-C(═O)N(CH₃)₂, -L-S(═O)₂(C₁₋₃-alkyl), -L-S(═O)₂NH₂, -L-S(═O)₂NHCH₃, -L-S(═O)₂N(CH₃)₂, -L-C(═O)NH(C₁₋₃alkyl), -L-C(═O)N(C₁₋₃alkyl)₂, -L-S(═O)₂NH₂, and -L-S(═O)₂N(C₁₋₃alkyl)₂. In one sub-embodiment, (1) if R2 is —C(═O)NH₂, —C(═O)NH(CH₂CH₃), —C(═O)N(CH₂CH₃)₂, then R3 is not —OH or if R3 is —OH then one or more R1 and R4-R9 has a substituent which is not hydro or a carbon, (2), if R2 is —C(═O)OH, then R3 is not —OH, —SH, —Cl, —NH₂, —OCH₃, —NHC(═O)CH₃, (3) R6 and R7 cannot be taken together to form a 6 member unsubstituted aryl ring, (4) R8 and R9 cannot be taken together to form a 6 member unsubstituted aryl ring, and/or (5) R11 is not para-bromo substituted phenyl.

According to another embodiment of the first aspect of the invention, in the compounds of Formula II, R2 is chosen from —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

According to still another embodiment of the first aspect of the invention, in the compounds of Formula II, R3 is chosen from -L-C(═O)OH, -L-CH═CHC(═O)OH, -L-CH₂CH₂C(═O)OH, -L-CH₂CH₂CH₂C(═O)OH, -L-C(CH₂CH₂)C(═O)OH, -L-CH(CH₃)C(═O)OH, -L-CH(CH₂CH₃)C(═O)OH, -L-C(CH₃)(CH₂CH₃)C(═O)OH, -L-CH═C(CH₃)C(═O)OH, -L-C(CH₂CH₃)₂C(═O)OH, -L-CH₂C(═O)OH, -L-C(CH₃)₂C(═O)OH, -L-C(═O)NH₂, -L-C(═O)NHCH₃, -L-C(═O)N(CH₃)₂, -L-S(═O)₂(C₁₋₃alkyl), -L-S(═O)₂NH₂, -L-S(═O)₂NHCH₃, -L-S(═O)₂N(CH₃)₂, -L-C(═O)NH(C₁₋₃alkyl), -L-C(═O)N(C₁₋₃alkyl)₂, -L-S(═O)₂NH₂, and -L-S(═O)₂N(C₁₋₃alkyl)₂. In one sub-embodiment, if R3 is —C(═O)OH then R2 is not hydroxyl or if R3 is —C(═O)NH₂ or —C(═O)OH, then one or more of a 4-7 member aryl or cycloalkyl formed from two adjacent of R6-R9, R2, R3, R4, R5, R6, R7, R8, R9, R10 and R11, is substituted with one or more non-hydrogen substituents excluding R6-R9 attachments to form another ring system.

According to another embodiment of the first aspect of the invention, in the compounds of Formula II, R3 is chosen from —CH═CHC(═O)OH, —CH₂CH₂C(═O)OH, —CH(CH₂CH₂)C(═O)OH, —C(C₃(CH₂CH₂) C(═O)O H, —CH═(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂5-S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

According to another embodiment of the first aspect of the invention, in the compounds of Formula II, R4 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂5 S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

According to yet another embodiment of the first aspect of the invention, in the compounds of Formula II, R5 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂5 S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

According to one embodiment of the first aspect of the invention, R8 and R9 in the compounds of Formula I are taken together to form a 6 member aryl ring as in Formula III.

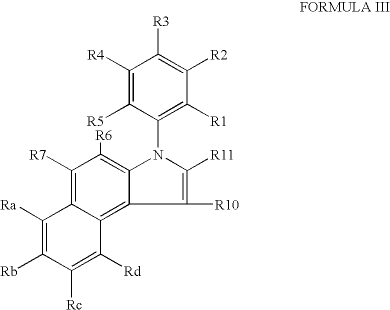

According to one embodiment of the first aspect of the invention, compounds of Formula III are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the first aspect of the invention.

According to one embodiment of the first aspect of the invention, R8 and R9 in the compounds of Formula II are taken together to form a 6 member aryl ring as in Formula IV.

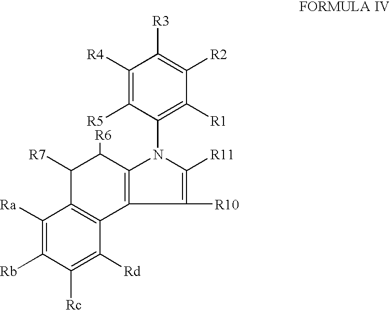

According to one embodiment of the first aspect of the invention, compounds of Formula IV are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the first aspect of the invention.

In a second aspect, the invention provides for the use compounds of Formula I and II for the treatment (and/or prevention) of diseases characterized by a defect in vesicular transport (e.g., axonal transport):

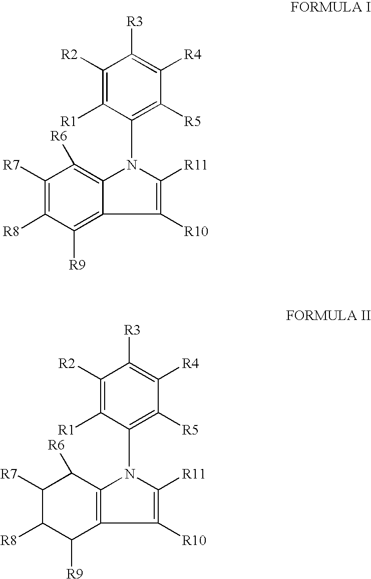

wherein R1-R5 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

one or more of R6-R9 are chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂5-L-NH(C═O)N(Rₒ)₂, -L-sulfo, -L-(2,6 difluorophenol), -L-phosphono, and -L-tetrazolyl; or two adjacent of R6-R9 can be taken together to form a 4-7 member substituted aryl or cycloalkyl ring wherein the substituent is chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; and the others of R6-R9, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₁₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R10 is chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

R11 is an optionally substituted phenyl group; and

L is as defined above.

In one sub-embodiment, the compound is not, 1,2-diphenyl-indole-4-acetic acid.

According to one embodiment of the second aspect of the invention, one of R6-R9 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₁₃alkyl), —C(═O)N(C₁₋₁₃alkyl)₂, —S(═O)₂NH₂, and —S(═O)₂N(C₁₋₃alkyl)₂.

In another embodiment of this second aspect of the invention, one of R6-R9 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; or two adjacent of R6-R9 can be taken together to form a 4-7 member aryl or cycloalkyl ring substituted with one or more substituents chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others of R6-R9 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₁₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R1-R5 and R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; and

R11 is an optionally substituted phenyl.

In one embodiment of the second aspect of the invention, R6 is chosen from -L-C(═O)OH, -L-CH═CHC(═O)OH, -L-CH₂CH₂C(═O)OH, -L-CH₂CH₂CH₂C(═O)OH, -L-C(CH₂CH₂)C(═O)OH, -L-CH(CH₃)C(═O)OH, -L-CH(CH₂CH₃)C(═O)OH, -L-C(CH₃)(CH₂CH₃)C(═O)OH, -L-CH═C(CH₃)C(═O)OH, -L-C(CH₂CH₃)₂C(═O)OH, -LCH₂C(═O)OH, -L-C(CH₃)₂C(═O)OH, -L-C(═O)NH₂, -L-C(═O)NHCH₃, -L-C(═O)N(CH₃)₂, -L-S(═O)₂(C₁₋₃alkyl), -L-S(═O)₂NH₂, -L-S(═O)₂NHCH₃, -L-S(═O)₂N(CH₃)₂, -L-C(═O)NH(C₁₋₃alkyl), -L-C(═O)N(C₁₋₃alkyl)₂, -L-S(═O)₂NH₂, and -L-S(═O)₂N(C₁₋₃alkyl)₂.

In one embodiment of the second aspect of the invention, R6 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In one embodiment of the second aspect of the invention, R7 is chosen from -L-C(═O)OH, -L-CH═CHC(═O)OH, -L-CH₂CH₂C(═O)OH, -L-CH₂CH₂CH₂C(═O)OH, -L-C(CH₂CH₂)C(═O)OH, -L-CH(CH₃)C(═O)OH, -L-CH(CH₂CH₃)C(═O)OH, -L-C(CH₃)(CH₂CH₃)C(═O)OH, -L-CH═C(CH₃)C(═O)OH, -L-C(CH₂CH₃)₂C(═O)OH, -LCH₂C(═O)OH, -L-C(CH₃)₂C(═O)OH, -L-C(═O)NH₂, -L-C(═O)NHCH₃, -L-C(═O)N(CH₃)₂, -L-S(═O)₂(C₁₋₃alkyl), -L-S(═O)₂NH₂, -L-S(═O)₂NHCH₃, -L-S(═O)₂N(CH₃)₂, -L-C(═O)NH(C₁₋₃alkyl), -L-C(═O)N(C₁₋₃alkyl)₂, -L-S(═O)₂NH₂, and -L-S(═O)₂N(C₁₋₃alkyl)₂.

In one embodiment of the second aspect of the invention, R7 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In one embodiment of the second aspect of the invention, R8 is chosen from -L-C(═O)OH, -L-CH═CHC(═O)OH, -L-CH₂CH₂C(═O)OH, -L-CH₂CH₂CH₂C(═O)OH, -L-C(CH₂CH₂)C(═O)OH, -L-CH(CH₃)C(═O)OH, -L-CH(CH₂CH₃)C(═O)OH, -L-C(CH₃)(CH₂CH₃)C(═O)OH, -L-CH═C(CH₃)C(═O)OH, -L-C(CH₂CH₃)₂C(═O)OH, -LCH₂C(═O)OH, -L-C(CH₃)₂C(═O)OH, -L-C(═O)NH₂, -L-C(═O)NHCH₃, -L-C(═O)N(CH₃)₂, -L-S(═O)₂(C₁₋₃alkyl), -L-S(═O)₂NH₂, -L-S(═O)₂NHCH₃, -L-S(═O)₂N(CH₃)₂, -L-C(═O)NH(C₁₋₃alkyl), -L-C(═O)N(C₁₋₃alkyl)₂, -L-S(═O)₂NH₂, and -L-S(═O)₂N(C₁₋₃alkyl)₂.

In one embodiment of the second aspect of the invention, R8 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In one embodiment of the second aspect of the invention, R9 is chosen from -L-C(═O)OH, -L-CH═CHC(═O)OH, -L-CH₂CH₂C(═O)OH, -L-CH₂CH₂CH₂C(═O)OH, -L-C(CH₂CH₂)C(═O)OH, -L-CH(CH₃)C(═O)OH, -L-CH(CH₂CH₃)C(═O)OH, -L-C(CH₃)(CH₂CH₃)C(═O)OH, -L-CH═C(CH₃)C(═O)OH, -L-C(CH₂CH₃)₂C(═O)OH, -LCH₂C(═O)OH, -L-C(CH₃)₂C(═O)OH, -L-C(═O)NH₂, -L-C(═O)NHCH₃, -L-C(═O)N(CH₃)₂, -L-S(═O)₂(C₁₋₃alkyl), -L-S(═O)₂NH₂, -L-S(═O)₂NHCH₃, -L-S(═O)₂N(CH₃)₂, -L-C(═O)NH(C₁₋₃alkyl), -L-C(═O)N(C₁₋₃alkyl)₂, -L-S(═O)₂NH₂, and -L-S(═O)₂N(C₁₋₃alkyl)₂.

In one embodiment of the second aspect of the invention, R9 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In one embodiment of the second aspect of the invention, R8 and R9 in the compounds of Formula I are taken together to form a 6 member aryl ring as in Formula III.

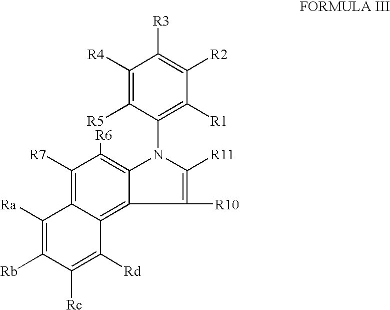

According to one embodiment of the second aspect of the invention, compounds of Formula III are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the second aspect of the invention.

In one embodiment of the second aspect of the invention, R8 and R9 in the compounds of Formula II are taken together to form a 6 member aryl ring as in Formula IV.

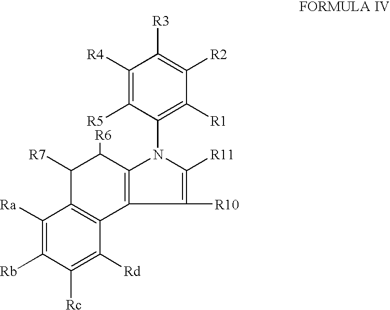

According to one embodiment of the second aspect of the invention, compounds of Formula IV are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the second aspect of the invention.

In a third aspect, the invention provides for the use of compounds of Formula I and II for the treatment (and/or prevention) of diseases associated with a defect in vesicular transport (e.g., axonal transport):

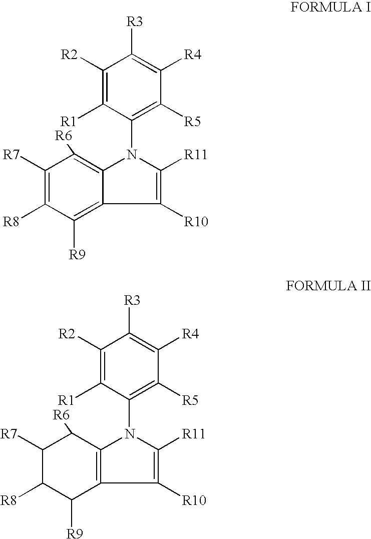

wherein R1-R9 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R10 is chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L is as defined above.

In one sub-embodiment, the compound is not 1-(O-carboxyphenyl)-2-phenyl-indole-3-carboxylic acid, or the methyl or ethyl ester thereof.

According to one embodiment of this aspect of the invention, R10 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In another embodiment of this third aspect of the invention, R10 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; R1-R9 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two adjacent of R6-R9 can be taken together to form a 4-7 member optionally substituted aryl or cycloalkyl ring; and

R11 is an optionally substituted phenyl.

In one embodiment of the third aspect of the invention, R8 and R9 in the compounds of Formula I are taken together to form a 6 member aryl ring as in Formula III.

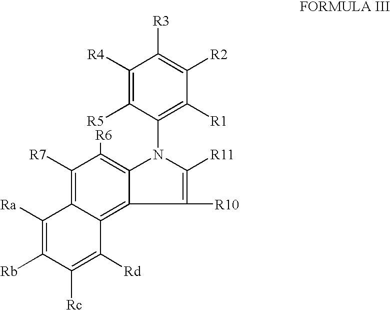

According to one embodiment of the third aspect of the invention, compounds of Formula III are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in the other embodiments of the third aspect of the invention.

In one embodiment of the third aspect of the invention, R8 and R9 in the compounds of Formula II are taken together to form a 6 member aryl ring as in Formula IV:

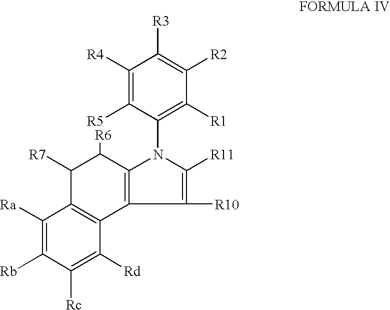

According to one embodiment of the third aspect of the invention, compounds of Formula IV are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the third aspect of the invention.

In a fourth aspect, the invention provides for the use of compounds of Formula I and II for treating (and/or preventing) disorders characterized by a defect in vesicular transport (e.g., vesicular transport):

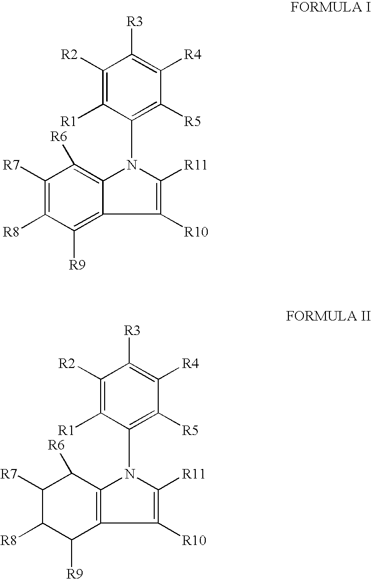

wherein R1-R10 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R11 is a phenyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl and the others are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl; and

L is as defined above.

In one sub-embodiment, the compound is not 5-(4,5-dihydro-3-phenyl-3H-benz[e]indol-2-yl)-2-hydroxy-benzoic acid or 2-hydroxy-5-(4,5,6,7-tetrahydro-1-phenyl-1H-indol-2-yl)-benzoic acid.

According to one embodiment of the fourth aspect of the invention, one substituent on the phenyl of R11 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In another embodiment of fourth aspect of the invention, R11 is a phenyl ring substituted with a substituent chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the other substituents on the phenyl are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R1-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; and two adjacent of R6-R9 can be taken together to form an optionally substituted 4-7 member aryl or cycloalkyl ring.

In one embodiment of the fourth aspect of the invention, R8 and R9 in the compounds of Formula I are taken together to form a 6 member aryl ring as in Formula III.

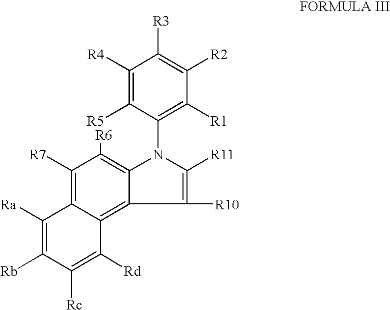

According to one embodiment of the fourth aspect of the invention, compounds of Formula III are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the fourth aspect of the invention.

In one embodiment of the fourth aspect of the invention, R8 and R9 in the compounds of Formula II are taken together to form a 6 member aryl ring as in Formula IV.

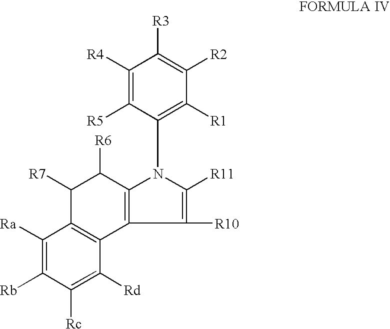

According to one embodiment of the fourth aspect of the invention, compounds of Formula IV are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in the other embodiments of the fourth aspect of the invention.

In a fifth aspect, the invention provides for the use of compounds of Formula I and II for the treatment (and/or prevention) of disorders characterized by a defect in vesicular transport (e.g., axonal transport):

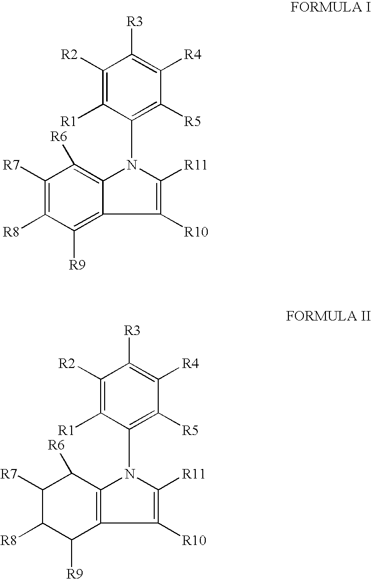

wherein R1-R9 and R11 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R10 is a phenyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L is as defined above.

According to one embodiment of this fifth aspect of the invention, one substituent on the phenyl of R10 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂(C₁₋₃alkyl), —S(═O)₂NH₂, —S(═O)₂NH(C₁₋₃alkyl), and —S(═O)₂N(C₁₋₃alkyl)₂, and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

In another embodiment of this fifth aspect of the invention, the phenyl group of R10 has a substituent chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the other substituents are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R1-R9, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two adjacent of R6-R9 can be taken together to form an optionally substituted 4-7 member aryl or cycloalkyl ring; and

R11 is an optionally substituted phenyl.

In one embodiment of the fifth aspect of the invention, R8 and R9 in the compounds of Formula I are taken together to form a 6 member aryl ring as in Formula III.

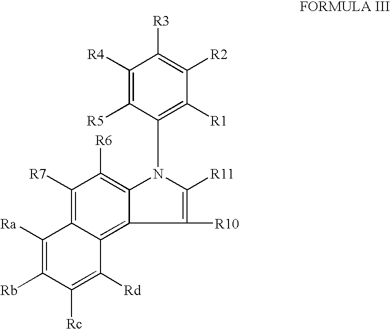

According to one embodiment of the fifth aspect of the invention, compounds of Formula III are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the fifth aspect of the invention.

In one embodiment of the fifth aspect of the invention, R8 and R9 in the compounds of Formula II are taken together to form a 6 member aryl ring as in Formula IV.

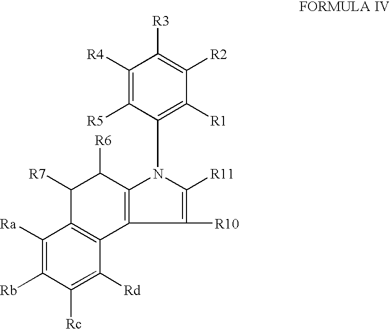

According to one embodiment of the fifth aspect of the invention, compounds of Formula IV are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the fifth aspect of the invention.

In a sixth aspect, the invention provides for the use compounds of Formula I and II for the treatment (and/or prevention) of disorders characterized by a defect in vesicular transport (e.g., axonal transport):

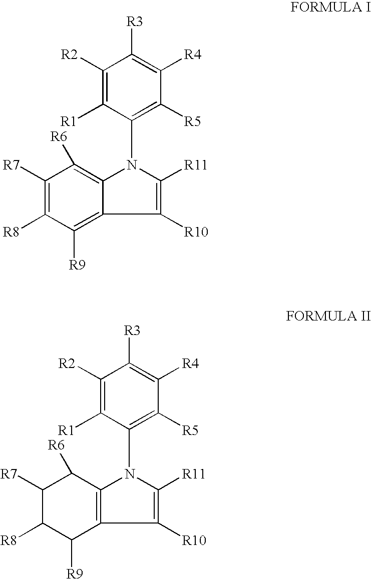

wherein R1-R9 and R11 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R10 is -L-R12 wherein L is as defined above; and

R12 is a phenyl ring substituted with one or more substituents independently chosen from of -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L is as defined above.

According to one embodiment of the sixth aspect of the invention, one substituent on the phenyl of R12 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂(C₁₋₃alkyl), —S(═O)₂NH₂, —S(═O)₂N(C₁₋₃alkyl), and —S(═O)₂N(C₁₋₃alkyl)₂, and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

In another embodiment of this sixth aspect of the invention, one of the substituents of R12 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R1-R9, and R11, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; and two adjacent of R6-R9 can be taken together to form an optionally substituted 4-7 member aryl or cycloalkyl ring.

In one embodiment of the sixth aspect of the invention, R8 and R9 in the compounds of Formula I are taken together to form a 6 member aryl ring as in Formula III.

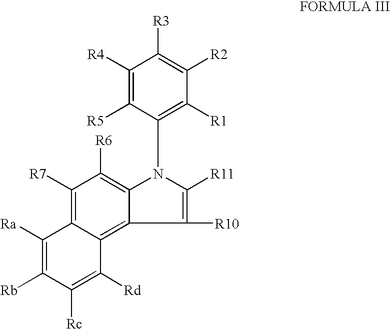

According to one embodiment of the sixth aspect of the invention, compounds of Formula III are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the sixth aspect of the invention.

In one embodiment of the sixth aspect of the invention, R8 and R9 in the compounds of Formula II are taken together to form a 6 member aryl ring as in Formula IV.

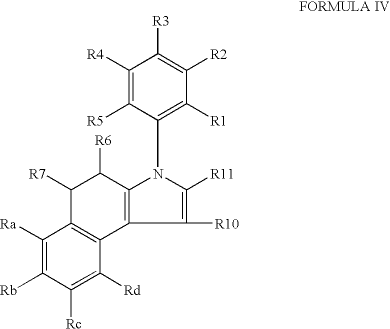

According to one embodiment of the sixth aspect of the invention, compounds of Formula IV are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the sixth aspect of the invention.

In a seventh embodiment, the invention provides for the use compounds of Formula I and II for the treatment (and/or prevention) of disorders characterized by a defect in vesicular transport (e.g., axonal transport):

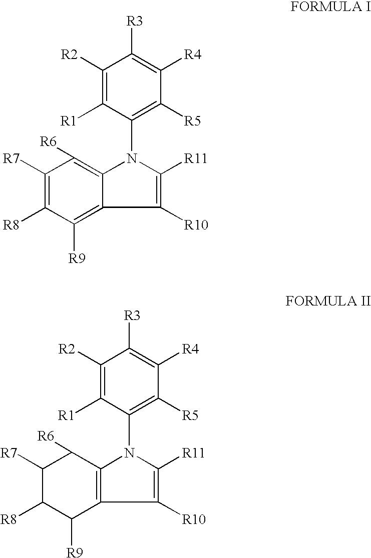

wherein R1-R10 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R11 is -L-R12 wherein L is as defined above; and

R12 is a phenyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl; and

L is as defined above.

According to one embodiment of this seventh aspect of the invention, one substituent on the phenyl of R12 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, -and —S(═O)₂N(C₁₋₃alkyl)₂, and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

In another embodiment of this seventh aspect of the invention, one substituent on the phenyl of R12 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₁₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R1-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two adjacent of R6-R9 can be taken together to form an optionally substituted 4-7 member aryl or cycloalkyl ring; and

R11 is an optionally substituted phenyl.

In one embodiment of the seventh aspect of the invention, R8 and R9 in the compounds of Formula I are taken together to form a 6 member aryl ring as in Formula III

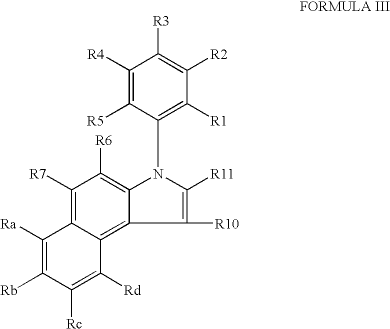

According to one embodiment of the seventh aspect of the invention, compounds of Formula III are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the seventh aspect of the invention.

In one embodiment of the seventh aspect of the invention, R8 and R9 in the compounds of Formula II are taken together to form a 6 member aryl ring as in Formula IV.

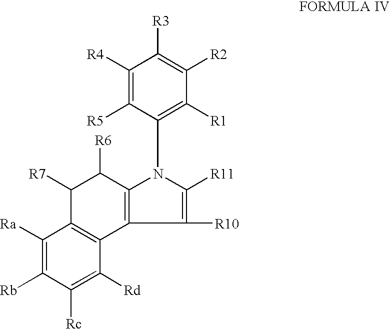

According to one embodiment of the seventh aspect of the invention, compounds of Formula IV are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the seventh aspect of the invention.

In an eighth embodiment, the invention provides for the use of compounds of Formula I and II for the treatment (and/or prevention) of disorders associated with a defect in vesicular transport (e.g., axonal transport):

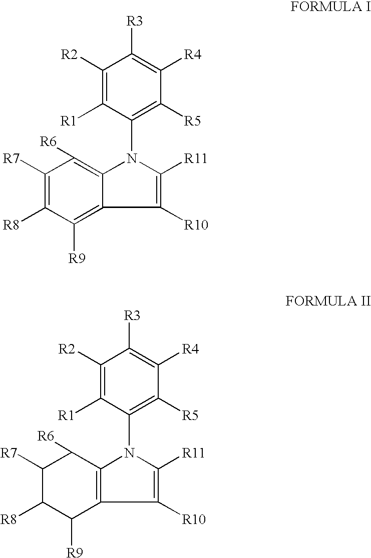

wherein R1-R9 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl ring;

R10 and R11 are independently chosen from hydro, hydroxyl, halo, alkyl, alkoxy, haloalkyl, haloalkoxy, —N(C₁₋₃ alkyl)₂, —NH(C₁₋₃ alkyl), —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, and -L-R12; and

R12 is a phenyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L is as defined above.

According to one embodiment of the eighth of the invention, R12 is present and one substituent on the phenyl of R12 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂(C₁₋₃alkyl), —S(═O)₂NH₂, —S(═O)₂NH(C₁₋₃alkyl), and —S(═O)₂N(C₁₋₃alkyl)₂, and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

In another embodiment of the eighth aspect of the invention, R12 is present and one substituent on the phenyl of R12 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₁₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R1-R9, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two adjacent of R6-R9 can be taken together to form an optionally substituted 4-7 member aryl or cycloalkyl ring.

In one embodiment of the eighth aspect of the invention, R8 and R9 in the compounds of Formula I are taken together to form a 6 member aryl ring as in Formula III.

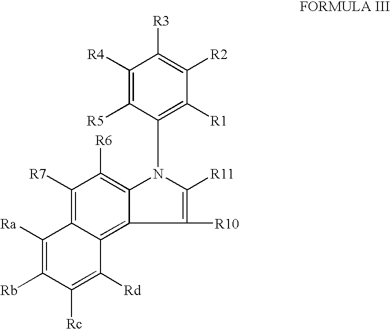

According to one embodiment of the eighth aspect of the invention, compounds of Formula III are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the eighth aspect of the invention.

In one embodiment of the eighth aspect of the invention, R8 and R9 in the compounds of Formula II are taken together to form a 6 member aryl ring as in Formula IV.

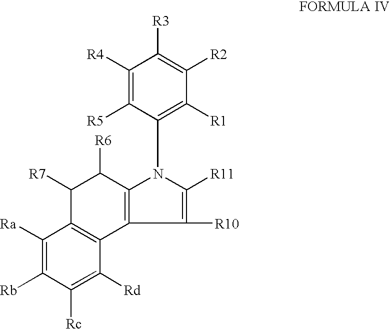

According to one embodiment of the eighth aspect of the invention, compounds of Formula IV are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the eighth aspect of the invention.

In a ninth aspect, the invention provides for the use of compounds of Formula V and VI for the treating (and/or preventing) disorders associated with a defect in vesicular transport (e.g., axonal transport):

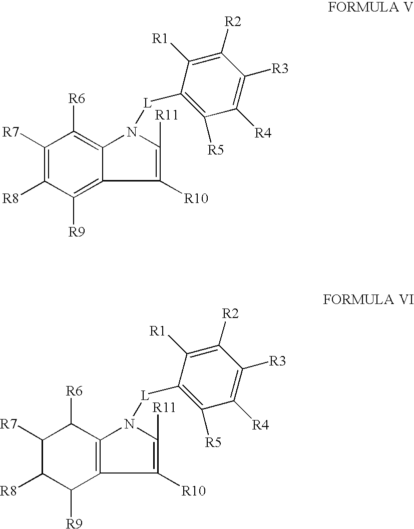

wherein one or more of R1-R5 is independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

L is as defined above;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring; and

R11 is an optionally substituted phenyl group.

In one sub-embodiment, R3 is not hydroxyl

According to one embodiment of this ninth aspect of the invention, one of R1-R5 is chosen from —C(═O)OH, —CH₂CH₂C(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, —S(═O)₂NH(C₁₋₃alkyl), and —S(═O)₂N(C₁₋₃alkyl)₂, and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

In another embodiment of this ninth aspect of the invention, L is a bond, one of R1-R5 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R6-R9, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; or two adjacent of R6-R9 can be taken together to form an optionally substituted 4-7 member aryl or cycloalkyl ring.

In one embodiment of the ninth aspect of the invention, R8 and R9 in the compound of Formula V are taken together to form a 6 member aryl ring as in Formula VII.

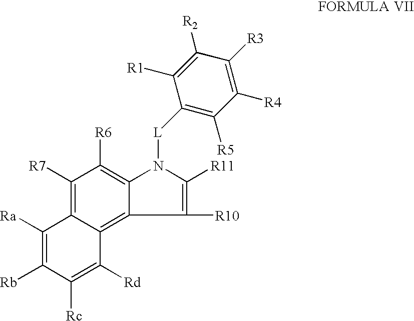

According to one embodiment of the ninth aspect of the invention, compounds of Formula VII are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the ninth aspect of the invention.

In one embodiment of the ninth aspect of the invention, R8 and R9 in the compounds of Formula VI are taken together to form a 6 member aryl ring as in Formula VIII.

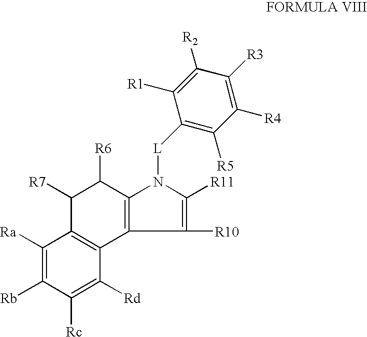

According to one embodiment of the ninth aspect of the invention, compounds of Formula VIII are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the ninth aspect of the invention.

In a tenth aspect, the invention provides for the use of compounds of Formula IX and X for treating (and/or preventing) disorders associated with a defect in vesicular transport (e.g., axonal transport):

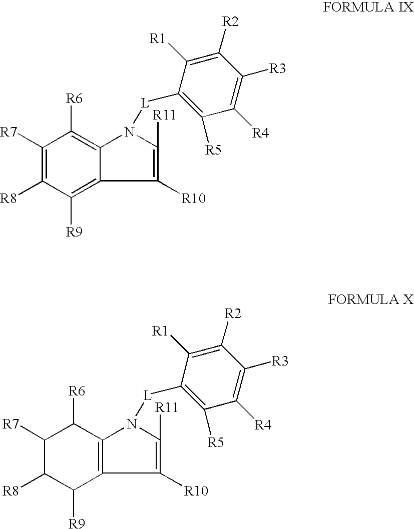

wherein one or more of R1-R11 are chosen from -L-R12, -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; wherein R12 is a phenyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

L is as defined above; and the others of R1-R11 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; and two adjacent of R6-R9 can be taken together to form a 4-7 member optionally substituted aryl or cycloalkyl ring.

In another embodiment of this tenth aspect of the invention, L is a bond, R12 is present and one substituents on the phenyl of R12 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R1-R9, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; and two adjacent of R6-R9 can be taken together to form an optionally substituted 4-7 member aryl or cycloalkyl ring.

In one embodiment of the tenth aspect of the invention, R8 and R9 in the compounds of Formula IX are taken together to form a 6 member aryl ring as in Formula XI

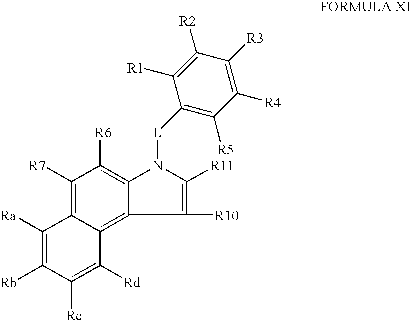

According to one embodiment of the tenth aspect of the invention, compounds of Formula XI are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the tenth aspect of the invention.

In one embodiment of the tenth aspect of the invention, R8 and R9 in the compounds of Formula X are taken together to form a 6 member aryl ring as in Formula XII.

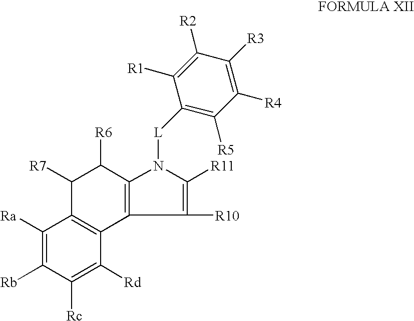

According to one embodiment of the tenth aspect of the invention, compounds of Formula XII are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the tenth aspect of the invention.

In an eleventh aspect, the invention provides for the use of compounds of Formula XIII and XIV for treating (and/or preventing) disorders associated with a defect in vesicular transport (e.g., axonal transport):

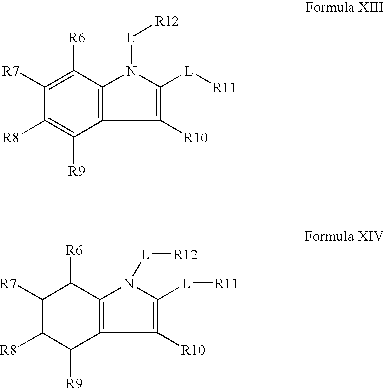

wherein L is as defined above or is selected from an optionally substituted, saturated or partially saturated cyclopropyl, cyclobutyl, cyclopentyl, cyclohexyl, cycloheptyl, and C₁₋₁₂ alkyl;
R1-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;

R11 is chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

R12 is chosen from optionally substituted C₁₋₁₂ alkyl, phenyl, and C₃₋₇ cycloalkyl.

In one embodiment of the eleventh aspect of the invention, R8 and R9 in the compounds of Formula XIII are taken together to form a 6 member aryl ring as in Formula XV.

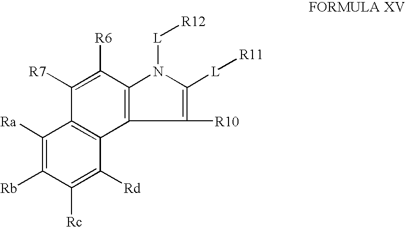

According to one embodiment of the eleventh aspect of the invention, compounds of Formula XV are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the eleventh aspect of the invention.

In one embodiment of the eleventh aspect of the invention, R8 and R9 in the compounds of Formula XIV are taken together to form a 6 member aryl ring as in Formula XVI.

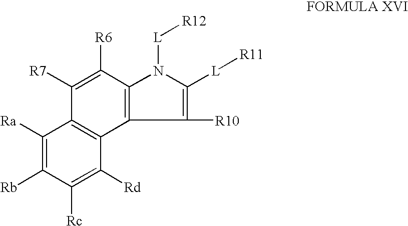

According to one embodiment of the eleventh aspect of the invention, compounds of Formula XVI are provided wherein Ra, Rb, Rc, and Rd are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); and the other variables can be defined as in one of the other embodiments of the eleventh aspect of the invention.

In a twelfth aspect, the invention provides for the use of compounds of Formula I and II pharmaceutically acceptable salts thereof, and pharmaceutical compositions having such compounds for treating (and/or preventing) a disorder associated with a defect in vesicular transport:

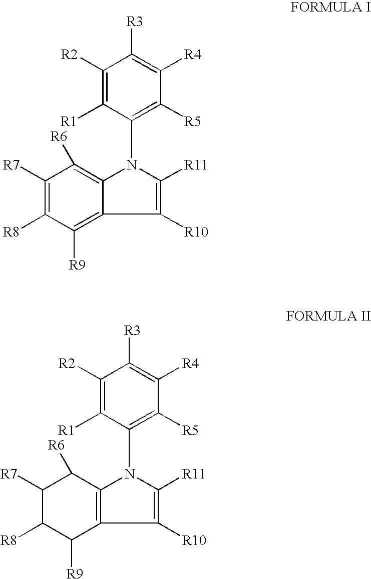

wherein one or more of R1-R5 is chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂5-L-sulfo, -L-(2,6 difluorophenol), -L-phosphono, and -L-tetrazolyl, and the others of R1-R5, independent of one another, are chosen hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R11 is an optionally substituted heterocyclic group; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In one embodiment of the twelfth aspect of the invention, one of R1-R5 in the compounds of Formulae I and II, is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₁₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, —S(═O)₂N(C₁₋₃alkyl)₂, and the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R6-R10, independent of one another, are chosen hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two adjacent of R6-R9 can be taken together to form a 4-7 member optionally substituted aryl or cycloalkyl ring;

L is —(CH₂)ₙ—(CH₂)ₙ—, with n independently 0, 1, 2, or 3; and

R11 is an optionally substituted heterocyclic group.

In another embodiment of this twelfth aspect of the invention, one of R1-R5 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others of R1-R5 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R6-R10 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two of R6-R9 can be taken together to form an optionally substituted C₄₋₇ member aryl, heterocyclic, or cycloalkyl ring; and

R11 is an optionally substituted heterocyclic group.

In one embodiment of this aspect of the invention the heterocyclic group is chosen from thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, 2H-pyrrolyl, imidazolyl, pyrazolyl, pyridyl (pyridinyl), 2-pyridyl, 3-pyridyl, 4-pyridyl, pyrazinyl, pyrimidinyl, pyridazinyl, tetrahydrofuranyl, pyranyl, piperidinyl, piperazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, quinuclidinyl, morpholinyl, cinnolinyl, pteridinyl, carbazolyl, beta-carbolinyl, phenanthridinyl, acrindinyl, perimidinyl, phenanthrolinyl, phenazinyl, isothiazolyl, phenothiazinyl, oxazolyl, isoxazolyl, furazanyl, phenoxazinyl, 1,4-dihydroquinoxaline-2,3-dione, 7 aminoisocoumarin, pyrido[1,2-a]pyrimidin-4-one, pyrazolo[1,5-a]pyrimidinyl, pyrazolo[1,5-a]pyrimidin-3-yl, 1,2-benzoisoxazol-3-yl, benzimidazolyl, 2-oxindolyl and 2 oxobenzimidazolyl. In one sub-embodiment of this embodiment, the heterocyclic group is chosen from pyridinyl, isoxazolyl, furanyl, thiazolyl, pyrimidinyl, pyrrolyl, thiophenyl, triazolyl, benzo[1,3]dioxolyl, and benzofuranyl.

In a thirteenth aspect, the invention provides for the use compounds of Formula I and II for treating (and/or preventing) a disorder associated with a defect in vesicular transport (e.g., axonal transport):

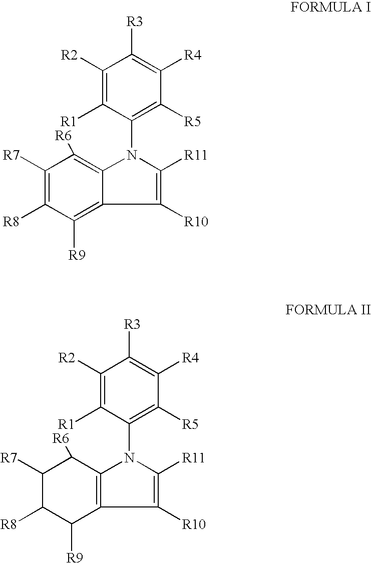

wherein R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂NH₂, —S(═O)₂(C₁₋₃alkyl), —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;
one or more of R6-R9 is independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; or two adjacent of R6-R9 can be taken together to form an optionally substituted 4-7 member aryl, heterocyclic, or cycloalkyl ring substituted with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; and the others of R6-R9, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₁₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

R11 is an optionally substituted heterocyclic group; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently selected from 0, 1, 2, 3, 4, 5, 6, 7, and 8, wherein each carbon can be optionally substituted with one or more C₁₋₃ alkyl or C₃₋₆ cycloalkyl.

In one embodiment of the thirteenth aspect of the invention, one of R6-R9 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂; or two adjacent of R6-R9 can be taken together to form an optionally substituted 4-7 member aryl, heterocyclic, or cycloalkyl ring substituted with one or more substituents chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂; and the others of R6-R9, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₁₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R1-R5, and R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R11 is an optionally substituted heterocyclic group.

In another embodiment of this thirteenth aspect of the invention, one of R6-R9 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; or two adjacent of R6-R9 can be taken together to form an optionally substituted 4-7 member aryl, heterocyclic, or cycloalkyl ring substituted with one or more substituents chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others of R6-R9 independently are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R1-R5, and R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; and

R11 is an optionally substituted heterocyclic group.

In one embodiment of this aspect of the invention the heterocyclic group is chosen from thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, 2H-pyrrolyl, imidazolyl, pyrazolyl, pyridyl (pyridinyl), 2-pyridyl, 3-pyridyl, 4-pyridyl, pyrazinyl, pyrimidinyl, pyridazinyl, tetrahydrofuranyl, pyranyl, piperidinyl, piperazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, quinuclidinyl, morpholinyl, cinnolinyl, pteridinyl, carbazolyl, beta-carbolinyl, phenanthridinyl, acrindinyl, perimidinyl, phenanthrolinyl, phenazinyl, isothiazolyl, phenothiazinyl, oxazolyl, isoxazolyl, furazanyl, phenoxazinyl, 1,4-dihydroquinoxaline-2,3-dione, 7 aminoisocoumarin, pyrido[1,2-a]pyrimidin-4-one, pyrazolo[1,5-a]pyrimidinyl, pyrazolo[1,5-a]pyrimidin-3-yl, 1,2-benzoisoxazol-3-yl, benzimidazolyl, 2-oxindolyl and 2 oxobenzimidazolyl. In one sub-embodiment of this embodiment, the heterocyclic group is chosen from pyridinyl, isoxazolyl, furanyl, thiazolyl, pyrimidinyl, pyrrolyl, thiophenyl, triazolyl, benzo[1,3]dioxolyl, and benzofuranyl.

In a fourteenth aspect, the invention provides for the use of compounds of Formula I and II for treating (and/or preventing) disorders associated with a defect in vesicular transport (e.g., axonal transport):

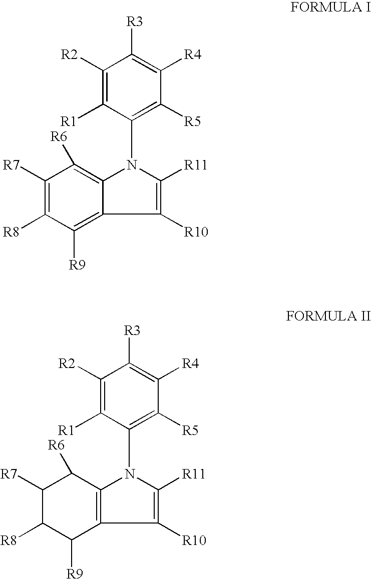

wherein R1-R9 are independently chosen hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form an optionally substituted C₄₋₇ member aryl, heterocyclic, or cycloalkyl ring;

R10 is chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl;

R11 is an optionally substituted heterocyclic group; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl;

In one embodiment of the fourteenth aspect of the invention, R10 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂; and

R11 is an optionally substituted heterocyclic group.

In another embodiment of this third aspect of the invention, R10 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and

R11 is an optionally substituted heterocyclic group.

In one embodiment of this aspect of the invention the heterocyclic group is chosen from thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, 2H-pyrrolyl, imidazolyl, pyrazolyl, pyridyl (pyridinyl), 2-pyridyl, 3-pyridyl, 4-pyridyl, pyrazinyl, pyrimidinyl, pyridazinyl, tetrahydrofuranyl, pyranyl, piperidinyl, piperazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, quinuclidinyl, morpholinyl, cinnolinyl, pteridinyl, carbazolyl, beta-carbolinyl, phenanthridinyl, acrindinyl, perimidinyl, phenanthrolinyl, phenazinyl, isothiazolyl, phenothiazinyl, oxazolyl, isoxazolyl, furazanyl, phenoxazinyl, 1,4-dihydroquinoxaline-2,3-dione, 7 aminoisocoumarin, pyrido[1,2-a]pyrimidin-4-one, pyrazolo[1,5-a]pyrimidinyl, pyrazolo[1,5-a]pyrimidin-3-yl, 1,2-benzoisoxazol-3-yl, benzimidazolyl, 2-oxindolyl and 2 oxobenzimidazolyl. In one sub-embodiment of this embodiment, the heterocyclic group is chosen from pyridinyl, isoxazolyl, furanyl, thiazolyl, pyrimidinyl, pyrrolyl, thiophenyl, triazolyl, benzo[1,3]dioxolyl, and benzofuranyl.

In a fifteenth aspect, the invention provides for the use of compounds of Formula I and II for treating (and/or preventing) a disorder associated with a defect in vesicular transport (e.g., axonal transport):

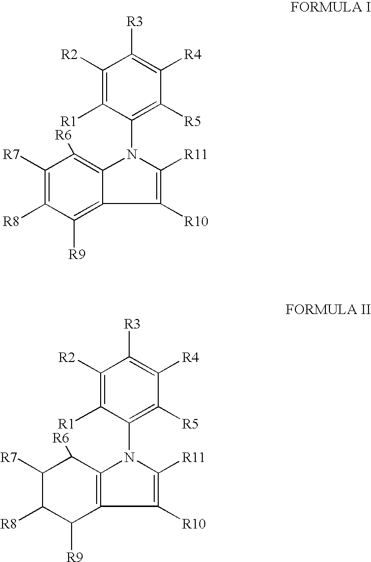

wherein R1-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R11 is a heterocyclic group with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In one embodiment of the fifteenth aspect of the invention, one substituent on the heterocyclic group of R11 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₁₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In another embodiment of this fifteenth aspect of the invention, one of the substituents on the heterocyclic group of R11 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH.

In one embodiment of this aspect of the invention the heterocyclic group is chosen from thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, 2H-pyrrolyl, imidazolyl, pyrazolyl, pyridyl (pyridinyl), 2-pyridyl, 3-pyridyl, 4-pyridyl, pyrazinyl, pyrimidinyl, pyridazinyl, tetrahydrofuranyl, pyranyl, piperidinyl, piperazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, quinuclidinyl, morpholinyl, cinnolinyl, pteridinyl, carbazolyl, beta-carbolinyl, phenanthridinyl, acrindinyl, perimidinyl, phenanthrolinyl, phenazinyl, isothiazolyl, phenothiazinyl, oxazolyl, isoxazolyl, furazanyl, phenoxazinyl, 1,4-dihydroquinoxaline-2,3-dione, 7 aminoisocoumarin, pyrido[1,2-a]pyrimidin-4-one, pyrazolo[1,5-a]pyrimidinyl, pyrazolo[1,5-a]pyrimidin-3-yl, 1,2-benzoisoxazol-3-yl, benzimidazolyl, 2-oxindolyl and 2 oxobenzimidazolyl. In one sub-embodiment of this embodiment, the heterocyclic group is chosen from pyridinyl, isoxazolyl, furanyl, thiazolyl, pyrimidinyl, pyrrolyl, thiophenyl, triazolyl, benzo[1,3]dioxolyl, and benzofuranyl.

In a sixteenth aspect, the invention provides for the use of compounds of Formula I and II for treating (and/or preventing) a disorder associated with a defect in vesicular transport (e.g., axonal transport):

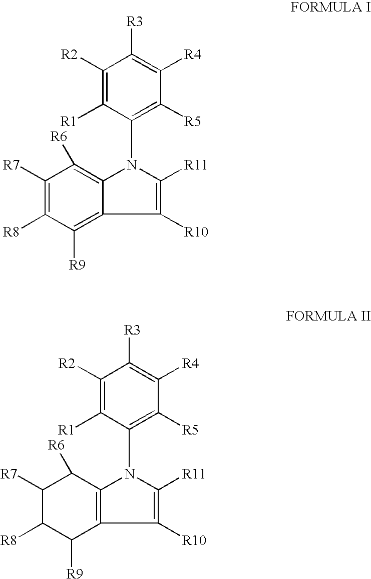

wherein R1-R9 and R11 independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R10 is a heterocyclic group with one or more substituents independently chosen -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)Rₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In one embodiment of the sixteenth aspect of the invention, one substituent on the heterocyclic group of R10 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In another embodiment of this sixteenth aspect of the invention, one substituent on the heterocyclic group of R10 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH.

In one embodiment of this aspect of the invention the heterocyclic group is chosen from thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, 2H-pyrrolyl, imidazolyl, pyrazolyl, pyridyl (pyridinyl), 2-pyridyl, 3-pyridyl, 4-pyridyl, pyrazinyl, pyrimidinyl, pyridazinyl, tetrahydrofuranyl, pyranyl, piperidinyl, piperazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, quinuclidinyl, morpholinyl, cinnolinyl, pteridinyl, carbazolyl, beta-carbolinyl, phenanthridinyl, acrindinyl, perimidinyl, phenanthrolinyl, phenazinyl, isothiazolyl, phenothiazinyl, oxazolyl, isoxazolyl, furazanyl, phenoxazinyl, 1,4-dihydroquinoxaline-2,3-dione, 7 aminoisocoumarin, pyrido[1,2-a]pyrimidin-4-one, pyrazolo[1,5-a]pyrimidinyl, pyrazolo[1,5-a]pyrimidin-3-yl, 1,2-benzoisoxazol-3-yl, benzimidazolyl, 2-oxindolyl and 2 oxobenzimidazolyl. In one sub-embodiment of this embodiment, the heterocyclic group is chosen from pyridinyl, isoxazolyl, furanyl, thiazolyl, pyrimidinyl, pyrrolyl, thiophenyl, triazolyl, benzo[1,3]dioxolyl, and benzofuranyl.

In a seventeenth aspect, the invention provides for the use of compounds of Formula I and II for treating (and/or preventing) a disorder associated with a defect in vesicular transport (e.g., axonal transport):

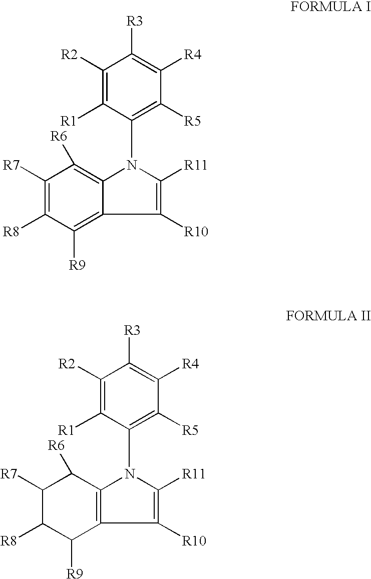

wherein R1-R9 and R11 independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

### R10 is -L-R12;

R12 is a heterocyclic group with one or more substituents chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)Rₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In one embodiment of the seventeenth aspect of the invention, one substituent on the heterocyclic group of R12 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In another embodiment of this seventeenth aspect of the invention, one of the substituent on the heterocyclic group of R12 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH.

In one embodiment of this aspect of the invention the heterocyclic group is chosen from thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, 2H-pyrrolyl, imidazolyl, pyrazolyl, pyridyl (pyridinyl), 2-pyridyl, 3-pyridyl, 4-pyridyl, pyrazinyl, pyrimidinyl, pyridazinyl, tetrahydrofuranyl, pyranyl, piperidinyl, piperazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, quinuclidinyl, morpholinyl, cinnolinyl, pteridinyl, carbazolyl, beta-carbolinyl, phenanthridinyl, acrindinyl, perimidinyl, phenanthrolinyl, phenazinyl, isothiazolyl, phenothiazinyl, oxazolyl, isoxazolyl, furazanyl, phenoxazinyl, 1,4-dihydroquinoxaline-2,3-dione, 7 aminoisocoumarin, pyrido[1,2-a]pyrimidin-4-one, pyrazolo[1,5-a]pyrimidinyl, pyrazolo[1,5-a]pyrimidin-3-yl, 1,2-benzoisoxazol-3-yl, benzimidazolyl, 2-oxindolyl and 2 oxobenzimidazolyl. In one sub-embodiment of this embodiment, the heterocyclic group is chosen from pyridinyl, isoxazolyl, furanyl, thiazolyl, pyrimidinyl, pyrrolyl, thiophenyl, triazolyl, benzo[1,3]dioxolyl, and benzofuranyl.

In an eighteenth embodiment, the invention provides for the use of compounds of Formula I and II for the treatment (and/or prevention) of a disorder associated with a defect in vesicular transport (e.g., axonal transport):

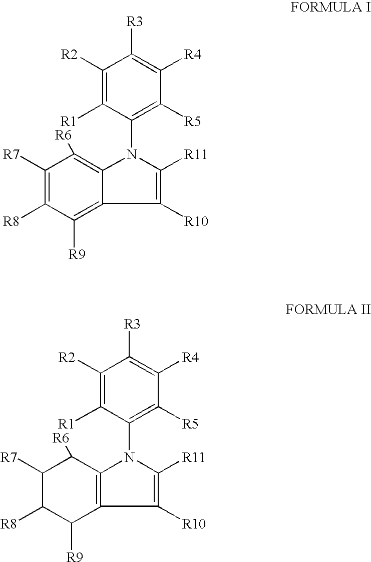

wherein R1-R9 and R11 independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R10 is a heterocyclic group with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In one embodiment of the eighteenth aspect of the invention, one substituent on the heterocyclic group of R10 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In another embodiment of the eighteenth aspect of the invention, one substituent on the heterocyclic group of R10 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH.

In one embodiment of this aspect of the invention the heterocyclic group is chosen from thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, 2H-pyrrolyl, imidazolyl, pyrazolyl, pyridyl (pyridinyl), 2-pyridyl, 3-pyridyl, 4-pyridyl, pyrazinyl, pyrimidinyl, pyridazinyl, tetrahydrofuranyl, pyranyl, piperidinyl, piperazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, quinuclidinyl, morpholinyl, cinnolinyl, pteridinyl, carbazolyl, beta-carbolinyl, phenanthridinyl, acrindinyl, perimidinyl, phenanthrolinyl, phenazinyl, isothiazolyl, phenothiazinyl, oxazolyl, isoxazolyl, furazanyl, phenoxazinyl, 1,4-dihydroquinoxaline-2,3-dione, 7 aminoisocoumarin, pyrido[1,2-a]pyrimidin-4-one, pyrazolo[1,5-a]pyrimidinyl, pyrazolo[1,5-a]pyrimidin-3-yl, 1,2-benzoisoxazol-3-yl, benzimidazolyl, 2-oxindolyl and 2 oxobenzimidazolyl. In one sub-embodiment of this embodiment, the heterocyclic group is chosen from pyridinyl, isoxazolyl, furanyl, thiazolyl, pyrimidinyl, pyrrolyl, thiophenyl, triazolyl, benzo[1,3]dioxolyl, and benzofuranyl.

In a nineteenth aspect, the invention provides for the use of compounds of Formula I and II for treating (and/or preventing) disorders associated with a defect in vesicular transport (e.g., axonal transport):

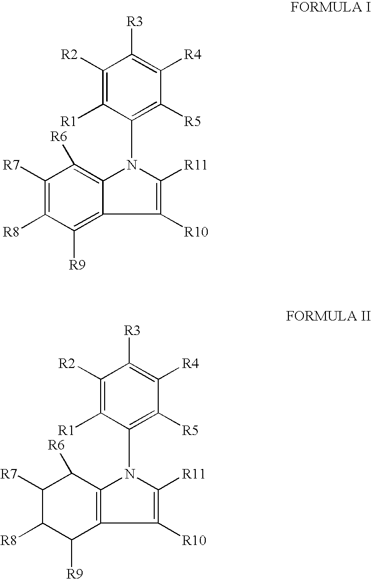

wherein R1-R9, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R10 and R11 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂NH₂, —S(═O)₂(C₁₋₃alkyl), —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, and -L-R12;

R12 is a heterocyclic group with one or more substituents independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl;

Rₒ is chosen from alkyl and haloalkyl; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In one embodiment of the nineteenth aspect of the invention, R12 is present and has one or more substituents independently chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In another embodiment of this nineteenth aspect of the invention, R12 is present and has one substituent chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH.

In one embodiment of this aspect of the invention the heterocyclic group is chosen from thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, 2H-pyrrolyl, imidazolyl, pyrazolyl, pyridyl (pyridinyl), 2-pyridyl, 3-pyridyl, 4-pyridyl, pyrazinyl, pyrimidinyl, pyridazinyl, tetrahydrofuranyl, pyranyl, piperidinyl, piperazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, quinuclidinyl, morpholinyl, cinnolinyl, pteridinyl, carbazolyl, beta-carbolinyl, phenanthridinyl, acrindinyl, perimidinyl, phenanthrolinyl, phenazinyl, isothiazolyl, phenothiazinyl, oxazolyl, isoxazolyl, furazanyl, phenoxazinyl, 1,4-dihydroquinoxaline-2,3-dione, 7 aminoisocoumarin, pyrido[1,2-a]pyrimidin-4-one, pyrazolo[1,5-a]pyrimidinyl, pyrazolo[1,5-a]pyrimidin-3-yl, 1,2-benzoisoxazol-3-yl, benzimidazolyl, 2-oxindolyl and 2 oxobenzimidazolyl. In one sub-embodiment of this embodiment, the heterocyclic group is chosen from pyridinyl, isoxazolyl, furanyl, thiazolyl, pyrimidinyl, pyrrolyl, thiophenyl, triazolyl, benzo[1,3]dioxolyl, and benzofuranyl.

In a twentieth aspect, the invention provides for the use of compounds of Formula V and VI for treating (and/or preventing) a disorder associated with a defect in vesicular transport (e.g., axonal transport):

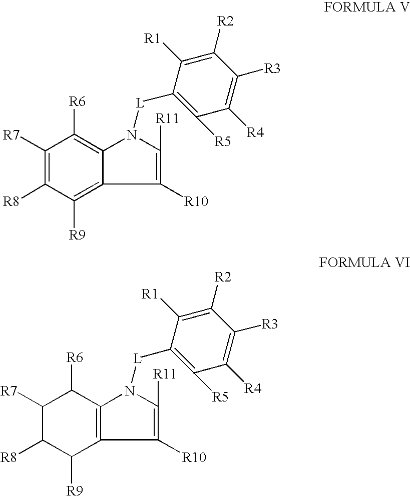

wherein one or more of R1-R5 is independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₁₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

Rₒ is chosen from alkyl and haloalkyl;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring;

R11 is an optionally substituted heterocyclic group; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In one embodiment of the twentieth aspect of the invention, one of R1-R5 in the compounds of Formulae I and II, is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂, and the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₁₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two adjacent of R6-R9 can be taken together to form a 4-7 member optionally substituted aryl or cycloalkyl ring;

L is —(CH₂)ₙ—(CH₂)ₙ—, with n independently 0, 1, 2, or 3; and

R11 is an optionally substituted heterocyclic group.

In another embodiment of this twentieth of the invention, L is a bond, one of R1-R5 is chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH; and the others of R1-R5 independently are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;

R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; two of R6-R9 can be taken together to form an optionally substituted 4-7 member aryl, heterocyclic, or cycloalkyl ring; and

R11 is an optionally substituted heterocyclic group.

In one embodiment of this aspect of the invention the heterocyclic group is chosen from thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, 2H-pyrrolyl, imidazolyl, pyrazolyl, pyridyl (pyridinyl), 2-pyridyl, 3-pyridyl, 4-pyridyl, pyrazinyl, pyrimidinyl, pyridazinyl, tetrahydrofuranyl, pyranyl, piperidinyl, piperazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, quinuclidinyl, morpholinyl, cinnolinyl, pteridinyl, carbazolyl, beta-carbolinyl, phenanthridinyl, acrindinyl, perimidinyl, phenanthrolinyl, phenazinyl, isothiazolyl, phenothiazinyl, oxazolyl, isoxazolyl, furazanyl, phenoxazinyl, 1,4-dihydroquinoxaline-2,3-dione, 7 aminoisocoumarin, pyrido[1,2-a]pyrimidin-4-one, pyrazolo[1,5-a]pyrimidinyl, pyrazolo[1,5-a]pyrimidin-3-yl, 1,2-benzoisoxazol-3-yl, benzimidazolyl, 2-oxindolyl and 2 oxobenzimidazolyl. In one sub-embodiment of this embodiment, the heterocyclic group is chosen from pyridinyl, isoxazolyl, furanyl, thiazolyl, pyrimidinyl, pyrrolyl, thiophenyl, triazolyl, benzo[1,3]dioxolyl, and benzofuranyl.

In a twenty-first aspect, the invention provides for the use of compounds of Formula V and VI for treating (and or preventing) a disorder associated with a defect in vesicular transport (e.g., axonal transport):

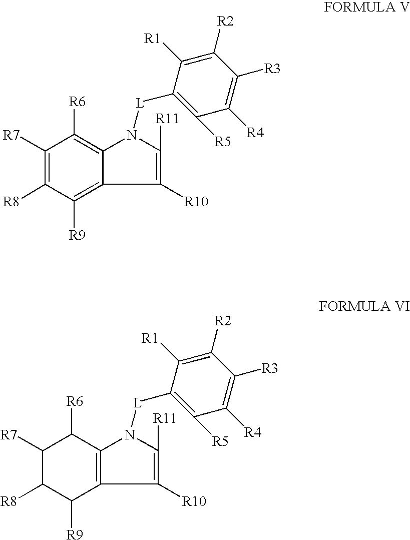

wherein R1-R11, independent of one another, are chosen from -L-R12, -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂5-L-NH(C═O)N(Rₒ)₂, -L-sulfo, -L-(2,6 difluorophenol), -L-phosphono, and -L-tetrazolyl;

Rₒ is chosen from alkyl and haloalkyl;

R12 is a heterocyclic group with one or more substituents independently chosen -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₁₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl; and

the others of R1-R11 are independently chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl, heterocyclic, or cycloalkyl ring; and

L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, wherein each carbon can be optionally substituted with one or more C₁₋₃ alkyl or C₃₋₆ cycloalkyl.

In one embodiment of the twenty-first aspect of the invention, R12 is present and has one or more substituents independently chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, —C(CH₃)₂C(═O)OH, —C(═O)NH₂, —C(═O)NHCH₃, —C(═O)N(CH₃)₂, —S(═O)₂(C₁₋₃alkyl), —S(═O)₂NH₂, —S(═O)₂NHCH₃, —S(═O)₂N(CH₃)₂, —C(═O)NH(C₁₋₃alkyl), —C(═O)N(C₁₋₃alkyl)₂, —S(═O)₂NH₂, and —S(═O)₂N(C₁₋₃alkyl)₂.

In another embodiment of this twenty-first aspect of the invention, L is a bond, R12 is present and has one substituent chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH.

In one embodiment of this twenty-first aspect, the invention includes analogs where the ring to which R1-R5 are attached is a 4-7 member heterocyclic ring instead a phenyl ring.

In one embodiment of this aspect of the invention the heterocyclic group is chosen from thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, 2H-pyrrolyl, imidazolyl, pyrazolyl, pyridyl (pyridinyl), 2-pyridyl, 3-pyridyl, 4-pyridyl, pyrazinyl, pyrimidinyl, pyridazinyl, tetrahydrofuranyl, pyranyl, piperidinyl, piperazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, quinuclidinyl, morpholinyl, cinnolinyl, pteridinyl, carbazolyl, beta-carbolinyl, phenanthridinyl, acrindinyl, perimidinyl, phenanthrolinyl, phenazinyl, isothiazolyl, phenothiazinyl, oxazolyl, isoxazolyl, furazanyl, phenoxazinyl, 1,4-dihydroquinoxaline-2,3-dione, 7 aminoisocoumarin, pyrido[1,2-a]pyrimidin-4-one, pyrazolo[1,5-a]pyrimidinyl, pyrazolo[1,5-a]pyrimidin-3-yl, 1,2-benzoisoxazol-3-yl, benzimidazolyl, 2-oxindolyl and 2 oxobenzimidazolyl. In one sub-embodiment of this embodiment, the heterocyclic group is chosen from pyridinyl, isoxazolyl, furanyl, thiazolyl, pyrimidinyl, pyrrolyl, thiophenyl, triazolyl, benzo[1,3]dioxolyl, and benzofuranyl.

In another aspect of the invention, one or more of the carbon atoms of the indole core are replaced by a heteroatom independently chosen from —N—, —O—, and —S—. In one embodiment, the substituents are as in any one of the other aspects and/or sub-embodiments of the invention.

In another aspect of the invention, the core indole group is replace with a group chosen from 5,7-Dihydro-6H-pyrrolo[2,3-h]cinnoline; 5,7-Dihydro-6H-pyrrolo[2,3-h]quinazoline; 4,5-Dihydro-3H-3,6,7-triaza-cyclopenta[a]naphthalene; 5,7-Dihydro-6H-pyrrolo[3,2-f]quinoxaline; 5,7-Dihydro-6H-pyrrolo[3,2-f]phthalazine; 5,7-Dihydro-6H-pyrrolo[2,3-h]quinoline; 5,7-Dihydro-6H-pyrrolo[3,2-f]quinazoline; 4,5-Dihydro-3H-pyrrolo[3,2-f]isoquinoline; 4,5-Dihydro-3H-pyrrolo[3,2-f]quinoline; and 5,7-Dihydro-6H-pyrrolo[2,3-h]isoquinoline. In one embodiment, the substituents are as in any one of the other aspects and/or sub-embodiments of the invention.

In some aspects of the invention, L is substituted with one or more substituents independently chosen from —C(═O)OH, —CH═CHC(═O)OH, —CH₂CH₂C(═O)OH, —CH₂CH₂CH₂C(═O)OH, —C(CH₂CH₂)C(═O)OH, —CH(CH₃)C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH, —CH₂C(═O)OH, and —C(CH₃)₂C(═O)OH, in lieu of having one of said substituents elsewhere in the compounds of Formulae I-XVI.

In some embodiments, of the first through twenty-first aspects of the invention, if a position in Formulae I-XVI is not specified then it can be specified as in one of the other embodiments of that aspect of the invention. Alternatively, the position can be substituted with one or more substituents independently chosen from the list of optional substituents below.

Optionally substituted, when used herein without reference to further definition, refers to a substituent independently chosen from the group consisting of hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

Furthermore, the invention provides derivatives or analog of the compounds defined in first through twenty-first aspects of the invention, where the derivative or analog is chosen from an ester (e.g., methyl or ethyl ester), an amide, a carbamate, a urea, an amadine, or a combination thereof. Methods for generating an ester, an amide, a carbamate, a urea, an amadine, or a combination thereof, of the compounds of the first aspect through the twenty-first aspects are known to an ordinary artisan skilled in organic chemical synthesis.

As the skilled artisan readily recognizes, in some of the embodiments of the first twenty-one aspects of the invention, some of the compounds can have more than one -L-group, each of which is independent chosen.

### Methods of Prevention and Treatment

In one embodiment of the invention, a method for treating (and/or preventing) a disorder associated with a defect in vesicular transport (e.g., axonal transport), in an individual in need of such treatment, is provided that includes the step of administering an effective amount of a compound of Formulae I-XVI as described above.

While not wishing to be bound by theory, it is believed that the compound of Formulae I-XVI acts in vivo to treat and/or prevent certain by modulating a biochemical pathway associated with a vesicular transport pathway (e.g., axonal transport). Such disease include, but are not limited to, amyotrophic lateral sclerosis (ALS), Charcot-Marie-Tooth Disease 2 (CMT2), spinal muscular atrophy (SPA), spinal muscular atrophy (SMA), Parkinson's Disease (PD), and hereditary sensory motor neuropathy, Optic neuropathies (e.g., Leber's hereditary optic neuropathy (LHON) and Cuban epidemic of optic neuropathy (CEON)), Niemann-Pick type C disease (NPC), Down syndrome, Dementia with Lewy Bodies (DLB), Parkinson's disease, Tauopathies (E.G., progressive supranuclear palsy, corticobasal degeneration, Pick's disease, argyrophilic grain disease, and frontotemporal dementia and parkinsonism linked to chromosome 17 (FTDP-17)), Miscellaneous motor neuron disorders (e.g., Primary lateral sclerosis (PLS)), Hereditary spastic paraplegia, spinal muscular atrophy, multiple sclerosis, Guillain-Barré syndrome, traumatic brain, spinal cord injury, and polyQ diseases (e.g., Huntington disease, spinobulbar muscular atrophy, dentatorubral-pallidoluysian atrophy, Kennedy's disease (also called spinobulbar muscular atrophy [SBMA]), spinocerebellar ataxia 1, spinocerebellar ataxia 2, spinocerebellar ataxia 3, spinocerebellar ataxia 6, spinocerebellar ataxia 7, and spinocerebellar ataxia 17).

The following section providers a brief description of disorders associated with a defect in vesicular transport.

PolyQ disease. The expansion of CAG repeats encoding glutamine is known to cause several late-onset progressive neurodegenerative disorders: Huntington disease, spinobulbar muscular atrophy, dentatorubral-pallidoluysian atrophy, Kennedy's disease (also called spinobulbar muscular atrophy [SBMA]), spinocerebellar ataxia 1, spinocerebellar ataxia 2, spinocerebellar ataxia 3, spinocerebellar ataxia 6, spinocerebellar ataxia 7, and spinocerebellar ataxia 17. These polyQ disorders commonly exhibit defects in axonal transport (Neuron. 40:1, 2003; Neuron 40:25, 2003; Neuron 40:41, 2003). Indeed, evidence suggests that perturbations in transport pathways are an early event in polyQ disease (Arch Neurol. 62:46, 2005).

Traumatic brain and spinal cord injury. Traumatic brain injury (TBI) is marked by rapid and long-term accumulation of proteins, including beta-amyloid precursor protein. TBI is also an epigenetic risk factor for developing neurodegenerative disorders, such as Alzheimer's disease and Parkinson's disease (Neuromolecular Med. 4:59, 2003).

Hereditary spastic paraplegia and spinal muscular atrophy. These motor neuron diseases exhibit clear cytoskeletal abnormalities that suggest the involvement of axonal transport in the pathogenesis of the diseases (Trends Neurosci. 25:532, 2002).

Multiple sclerosis. Inflammation is the cause of much neural damage in multiple sclerosis, resulting in disruption of axonal transport (Curr Opin Neurol. 16:267, 2003). These observations admit the possibility that the neurodegeneration experienced by MS patients may be attenuated by agents that enhance axonal transport. In a similar vein, diseases such as Guillain-Barré syndrome, an inflammatory disorder of the peripheral nerves, may be amenable to therapeutic intervention with agents that enhance axonal transport.

Miscellaneous motor neuron disorders. Primary lateral sclerosis (PLS) is a rare degenerative disorder of the upper motor neuron, whose classification is controversial (J Neurol Sci. 170:5, 1999). In fact, a recent study has concluded that PLS is not a discrete nosological entity but represents one end of a continuous spectrum of motor neuron disease (Brain 124:1989, 2001). A therapeutic that successfully treats one motor neuron dysfunction is therefore a candidate for treatment of other motor neuron disorders.

Tauopathies. Aberrant functions of the microtubule-associated proteins collectively called tau can lead to neurodegenerative disorders like progressive supranuclear palsy, corticobasal degeneration, Pick's disease, argyrophilic grain disease, and frontotemporal dementia and parkinsonism linked to chromosome 17 (FTDP-17) (Biochim Biophys Acta. 1739:240, 2005; Brain Res Brain Res Rev. 33:95, 2000). One feature of tauopathies is their disruption of axonal transport that accompanies them.

Dementia with Lewy Bodies. Dementia with Lewy Bodies (DLB) is characterized by the presence of cytoplasmic inclusions of alpha-synuclein in the cerebral cortex and in the nuclei of the brain stem Arch Gerontol Geriatr 39:1, 2004). Protein aggregates, whether they are aggregates of tau, Aβ, prions or other proteins, apparently disrupt vesicle transport. A therapy that treats dysfunctional vesicle transport is a candidate regimen for treatment of DLB.

Down syndrome. Nearly all individuals with Down syndrome develop amyloid plaques and the attendant neuropathologic lesions by the age of 45 (Arch Neurol 46:849, 1989). This admits the possibility that Ab42-lowering compounds such as certain fendosal derivatives may moderate or delay the onset of the dementia of Down syndrome.

Niemann-Pick type C disease (NPC). The primary lesion of NPC appears to be impaired cholesterol trafficking and excessive glycosphingolipid storage. One consequence of this impairment is abnormal vesicle trafficking in neural tissue, which likely contributes to the neurodegeneration characteristic of the disease (Neurobiol Aging 26:373, 2005). A recent study indicates that the abnormal vesicle trafficking contributes to increased deposition of Aβ42 in brain tissue of NPC patients (Am J Pathol. 164:975, 2004), which suggests that Aβ peptides may participate in the neurodegeneration.

Optic neuropathies. Histological evidence suggests impaired axonal transport of mitochondria in Leber's hereditary optic neuropathy (LHON) and in Cuban epidemic of optic neuropathy (CEON). Since mitochondria are transported along microtubules by mechanisms similar to microtubule-directed transport of vesicles.

Parkinson's disease (Acta. Neuropathol.(Berl) 98:157-164, 1999).

Amyotrophic lateral sclerosis (J. Neurol. Sci. 63:241-250, 1984; Acta. Neuropathol. (Berl) 94:294-299, 1997).

In another embodiment, the invention provides a method of treating a disorder associated with a defect in axonal transport, by identifying a patient in need of such treatment, and administering to the patient a therapeutically effective amount of a pharmaceutical composition having one or more compounds of Formulae I-XVI. Administration of a compound of Formulae I-XVI for at least 4 weeks, preferably at least 4 months, and more desirably at least 8 months, can provide an improvement or lessening in decline of cognitive function as characterized by clinically acceptable tests, biochemical disease marker progression, and/or pathology. The pharmaceutical composition for use in the invention is formulated with one or more pharmaceutically acceptable excipients, salts, or carriers. The pharmaceutical composition for use in the invention is delivered orally, preferably in a tablet or capsule dosage form.

In yet another embodiment, the invention provides a method for prophylaxis against a disorder associated with a defect in axonal transport, by identifying a patient in need of or desiring such treatment, and administering to the patient a prophylactically effective amount of a pharmaceutical composition having one or more compounds of Formulae I-XVI. Preferred compounds for use in this embodiment of the invention include those in Tables 1-6. Administration of a compound of Formulae I-XVI for at least 4 weeks, preferably at least 4 months, and more desirably at least 8 months, can delay the onset of the disorder or slow the rate of onset of symptoms of the disorder.

The skilled artisan readily recognizes that the invention includes the use of compounds of Formulae I-XVI, pharmaceutically acceptable salts, metabolites and prodrugs thereof in each of the described embodiments.

### DEFINITIONS

As used herein, the term “alkyl” refers to a saturated aliphatic hydrocarbon including straight chain and branched chain groups. Preferably, the alkyl group has 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc. up to and including 20 carbon atoms). More preferably, it is a medium size alkyl having 1 to 10 carbon atoms. Even more preferably, it is a lower alkyl having 1 to 6 carbon atoms, and even more preferably 1 to 4 carbon atoms. The alkyl group may be substituted or unsubstituted. When substituted, the substituent group(s) is preferably one or more individually selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, C-carboxy, O-carboxy, cyanato, isocyanato, thiocyanato, isothiocyanato, nitro, silyl, and amino.

As used herein, the term “halo” refers to chloro, fluoro, bromo, and iodo.

As used herein, the term “hydro” refers to a hydrogen atom (—H group).

As used herein, the term “hydroxy” refers to an —OH group.

As used herein, the term “alkoxy” refers to both an —O-alkyl and an —O-cycloalkyl group, as defined herein. Lower alkoxy refers to —O-lower alkyl groups.

As used herein, the term “aryloxy” refers to both an —O-aryl and an —O-heteroaryl group, as defined herein.

As used herein, the term “mercapto” group refers to an —SH group.

As used herein, the term “alkylthio” group refers to both an S-alkyl and an —S-cycloalkyl group, as defined herein.

As used herein, the term “arylthio” group refers to both an —S-aryl and an —S-heteroaryl group, as defined herein.

As used herein, the term “carbonyl” group refers to a —C(═O)R″ group, where R″ is selected from the group consisting of hydro, alkyl, cycloalkyl, aryl, heteroaryl (bonded through a ring carbon) and heterocyclic (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has a “═O” bonded to it; i.e. one of the ring carbon atoms is a —C(═O)-group.

As used herein, the term “thiocarbonyl” group refers to a —C(═S)R″ group, with R″ as defined herein.

As used herein, the term “O-carboxy” group refers to a R″C(═O)O-group, with R″ as defined herein.

As used herein, the term “C-carboxy” group refers to a —C(═O)OR″ groups with R″ as defined herein.

As used herein, the term “ester” is a C-carboxy group, as defined herein, wherein R″ is any of the listed groups other than hydro.

As used herein, the term “C-carboxy salt” refers to a —C(═O)O⁻M⁺ group wherein M⁺ is selected from the group consisting of lithium, sodium, magnesium, calcium, potassium, barium, iron, zinc and quaternary ammonium.

As used herein, the term “acetyl” group refers to a —C(═O)CH₃ group.

As used herein, the term “carboxyalkyl” refers to —(CH₂)ᵣC(═O)OR″ wherein r is 1-6 and R″ is as defined above.

As used herein, the term “carboxyalkyl salt” refers to a —(CH₂)ᵣC(═O)O⁻M⁺ wherein M⁺ is selected from the group consisting of lithium, sodium, potassium, calcium, magnesium, barium, iron, zinc and quaternary ammonium.

As used herein, the term “carboxylic acid” refers to a C-carboxy group in which R″ is hydro.

As used herein, the term “haloalkyl” refers to an alkyl group substituted with 1 to 6 halo groups, preferably haloalkyl is a —CX₃ group wherein X is a halo group. The halo groups can be independently selected.

As used herein, the term “trihalomethanesulfonyl” refers to a X₃ CS(═O)₂— group with X as defined above.

As used herein, the term “cyano” refers to a —C≡N group.

As used herein, the term “cyanato” refers to a —CNO group.

As used herein, the term “isocyanato” refers to a —NCO group.

As used herein, the term “thiocyanato” refers to a —CNS group.

As used herein, the term “isothiocyanato” refers to a —NCS group.

As used herein, the term “sulfinyl” refers to a —S(═O)R″ group, with R″ as defined herein.

As used herein, the term “sulfonyl” refers to a —S(═O)₂R″ group, with R″ as defined herein.

As used herein, the term “sulfonamido” refers to a —S(═O)₂ NR¹⁷R¹⁸, with R¹⁷ and

R¹⁸ as defined herein.

As used herein, the term “trihalomethanesulfonamido” refers to a X₃CS(═O)₂NR¹⁷-group with X and R¹⁷ as defined herein.

As used herein, the term “O-carbamyl” refers to a —OC(═O)NR¹⁷R¹⁸ group with R¹⁷ and R¹⁸ as defined herein.

As used herein, the term “N-carbamyl” refers to a R¹⁸ OC(═O)NR¹⁷— group, with R¹⁷ and R¹⁸ as defined herein.

As used herein, the term “0-thiocarbamyl” refers to a —OC(═S)NR¹⁷R¹⁸ group with R¹⁷ and R¹⁸ as defined herein.

As used herein, the term “N-thiocarbamyl” refers to a R¹⁷OC(═S)NR¹⁸— group, with R¹⁷ and R¹⁸ as defined herein.

As used herein, the term “amino” refers to an —NR¹⁷R¹⁸ group, with R¹⁷ and R¹⁸ both being hydro.

As used herein, the term “C-amido” refers to a —C(═O)NR¹⁷R¹⁸ group with R¹⁷ and R¹⁸ as defined herein. An “N-amido” refers to a R¹⁷C(═O)NR¹⁸— group with R¹⁷ and R¹⁸ as defined herein.

As used herein, the term “nitro” refers to a —NO₂ group.

As used herein, the term “quaternary ammonium” refers to a -+NR¹⁷R¹⁸R¹⁹ group wherein R¹⁷, R¹⁸, and R¹⁹ are independently selected from the group consisting of hydro and unsubstituted lower alkyl.

As used herein, the term “methylenedioxy, ethylenedioxy” refers to a —OCH₂O-group wherein the oxygen atoms are bonded to adjacent ring carbon atoms.

As used herein, the term “ethylenedioxy” refers to a —OCH₂CH₂O— group wherein the oxygen atoms are bonded to adjacent ring carbon atoms.

As used herein, the term “cycloalkyl” refers to an all-carbon monocyclic or fused ring (i.e., rings which share an adjacent pair of carbon atoms) group wherein one or more of the rings does not have a completely conjugated pi-electron system. Examples, without limitation, of cycloalkyl groups are cyclopropane, cyclobutane, cyclopentane, cyclopentene, cyclohexane, adamantane, cyclohexadiene, cycloheptane and, cycloheptatriene. A cycloalkyl group may be substituted or unsubstituted. When substituted, the substituent group(s) is preferably one or more individually selected from alkyl, aryl, heteroaryl, heterocyclic, hydroxy, alkoxy, aryloxy, mercapto, alkylthio, arylthio, cyano, halo, carbonyl, thiocarbonyl, carboxy, O-carbamyl, N-carbamyl, C-amido, N-amido, nitro, and amino.

As used herein, the term “heterocycle” or heterocyclic” refers to a saturated or partially saturated 3-7 membered monocyclic, or 7-10 membered bicyclic ring system, which consists of carbon atoms and from one to four heteroatoms independently selected from the group consisting of O, N, and S, wherein the nitrogen and sulfur heteroatoms can be optionally oxidized, the nitrogen can be optionally quaternized, and including any bicyclic group in which any of the above-defined heterocyclic rings is fused to a benzene ring, and wherein the heterocyclic ring can be substituted on carbon or on a nitrogen atom if the resulting compound is stable. Non-limiting saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups. Example of “heterocycles” or “heterocyclic” rings also include, but are not limited to, morpholino, piperidyl, piperazinyl, pyrrolidinyl, thiomorpholino, homopiperazinyl, imidazolyl, imidazolidinyl, pyrazolidinyl, dioxanyl and dioxolanyl. “Heterocycle” can include heteroaryls when the pi-electron system of a heterocycle is completely conjugated.

As used herein, the term “aryl” refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituted group(s) is preferably one or more selected from halo, trihalomethyl, alkyl, hydroxy, alkoxy, aryloxy, mercapto, alkylthio, arylthio, cyano, nitro, carbonyl, thiocarbonyl, C-carboxy, O-carboxy, O-carbamyl, N-carbamyl, O-thiocarbamyl, N-thiocarbamyl, C-amido, N-amido, sulfinyl, sulfonyl, S-sulfonamido, N-sulfonamido, trihalo-methanesulfonamido, and amino.

As used herein, the term “heteroaryl” refers to groups having 5 to 14 ring atoms; 6, 10 or 14 pi electrons shared in a cyclic array; and containing carbon atoms and 1, 2 or 3 oxygen, nitrogen or sulfur heteroatoms. Non-limiting heteroaryl groups include thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, beta-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7 aminoisocoumarin, pyrido[1,2-a]pyrimidin-4-one, pyrazolo[1,5-a]pyrimidinyl, including without limitation pyrazolo[1,5-a]pyrimidin-3-yl, 1,2-benzoisoxazol-3-yl, benzimidazolyl, 2-oxindolyl and 2 oxobenzimidazolyl. Where the heteroaryl group contains a nitrogen atom in a ring, such nitrogen atom may be in the form of an N-oxide, e.g., a pyridyl N oxide, pyrazinyl N-oxide and pyrimidinyl N-oxide. When substituted, the substituted group(s) is preferably one or more selected from alkyl, cycloalkyl, halo, trihalomethyl, hydroxy, alkoxy, aryloxy, mercapto, alkylthio, arylthio, cyano, nitro, carbonyl, thiocarbonyl, sulfonamido, carboxy, sulfinyl, sulfonyl, O-carbamyl, N-carbamyl, O-thiocarbamyl, N-thiocarbamyl, C-amido, N-amido, and amino.

As used herein, the term “preventing an increase in a symptom” refers to both not allowing a symptom to increase or worsen, as well as reducing the rate of increase in the symptom. For example, a symptom can be measured as the amount of particular disease marker, i.e. a protein. In another example the symptom can be cognitive decline. Preventing an increase, according to the definition provided herein, means that the amount of symptom (e.g., protein or cognitive decline) does not increase or that the rate at which it increases is reduced.

As used herein, the term “treating a disease or disorder” refers to a slowing of or a reversal of the progress of the disease. Treating a disease or disorder includes treating a symptom and/or reducing the symptoms of the disease.

As used herein, the term “preventing a disease or disorder” refers to a slowing of the disease or of the onset of the disease or the symptoms thereof. Preventing a disease or disorder can include stopping the onset of the disease or symptoms thereof.

As used herein, the term “unit dosage form” refers to a physically discrete unit, such as a capsule or tablet suitable as a unitary dosage for a human patient. Each unit contains a predetermined quantity of a compound of Formulae I-XVI, which was discovered or believed to produce the desired pharmacokinetic profile which yields the desired therapeutic effect. The dosage unit is composed of a compound of Formulae I-XVI in association with at least one pharmaceutically acceptable carrier, salt, excipient, or combination thereof.

As used herein, the term “dose” or “dosage” refers the amount of active ingredient that an individual takes or is administered at one time. For example, an 800 mg dose of a compound of Formulae I-XVI refers to, in the case of a twice-daily dosage regimen, a situation where the individual takes 800 mg of a compound of Formulae I-XVI twice a day, e.g., 800 mg in the morning and 800 mg in the evening. The 800 mg of a compound of Formulae I-XVI dose can be divided into two or more dosage units, e.g., two 400 mg dosage units of a compound of Formulae I-XVI in tablet form or two 400 mg dosage units of a compound of Formulae I-XVI in capsule form.

“A pharmaceutically acceptable prodrug” is a compound that may be converted under physiological conditions or by solvolysis to the specified compound or to a pharmaceutically acceptable salt of such compound.

“A pharmaceutically active metabolite” is intended to mean a pharmacologically active product produced through metabolism in the body of a specified compound or salt thereof. Metabolites of a compound may be identified using routine techniques known in the art and their activities determined using tests such as those described herein.

“A pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrophosphates, dihydrophosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates, propanesulfonates, naphthalene-1-sulfonates, naphthalene-2-sulfonates, and mandelates.

### Preparation of the Compounds of the Invention

Representative synthetic schemes and experimental descriptions for the compounds of Formulae I-XVI for use in the methods of the invention are given in the Examples below.

### Dosages, Formulations, and Route of Administration

The active compounds of this invention are typically administered in combination with a pharmaceutically acceptable carrier through any appropriate routes such as parenteral, oral, or topical administration, in a therapeutically (or prophylactically) effective amount according to the methods set forth above. A preferred route of administration for use in the invention is oral administration.

Generally, the toxicity profile and therapeutic efficacy of the therapeutic agents can be determined by standard pharmaceutical procedures in suitable cell models or animal models. As is known in the art, the LD50 represents the dose lethal to about 50% of a tested population. The ED50 is a parameter indicating the dose therapeutically effective in about 50% of a tested population. Both LD50 and ED50 can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED50 and/or IC₅₀, but remains significantly below the LD50 dosage level, as determined from cell or animal models.

Typically, the compounds and compositions for use in the invention can be effective at an amount of from about 0.05 mg to about 4000 mg per day, preferably from about 0.1 mg to about 2000 mg per day. However, the amount can vary with the body weight of the patient treated and the state of disease conditions. The active ingredient may be administered at once, or may be divided into a number of smaller doses to be administered at predetermined intervals of time.

In the case of combination therapy, a therapeutically effective amount of another therapeutic compound can be administered in a separate pharmaceutical composition, or alternatively included in the pharmaceutical composition according to the present invention. The pharmacology and toxicology of other therapeutic compositions are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in the art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can also be adjusted as the various factors change over time.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetate, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al, J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network that swells in water to form a gel like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci. 73:1718-1720 (1984).

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

Soft gelatin capsules can be prepared in which capsules contain a mixture of the active ingredient and vegetable oil or non-aqueous, water miscible materials such as, for example, polyethylene glycol and the like. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, or gelatin.

Tablets for oral use are typically prepared in the following manner, although other techniques may be employed. The solid substances are ground or sieved to a desired particle size, and the binding agent is homogenized and suspended in a suitable solvent. The active ingredient and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the machine may be selected by the skilled artisan.

If the compound for use in the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound for use in the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium. These substituents may optionally be further substituted with a substituent selected from such groups.

### EXAMPLES

### Example 1

### Tablets

| Ingredient | Amount | | Preferred Ranges |
|---|---|---|---|
| Compound of Formulae I-XVI | 400 | mg | +50% to −50% |
| Microcrystalline Cellulose | 392 | mg | +50% to −50% |
| Colloidal Silicon Dioxide | 4 | mg | +50% to −50% |
| Magnesium Stearate | 4 | mg | +50% to −50% |

The tablets are prepared using art known procedures.

### Example 2

### Coated Tablets

| Ingredient | Amount | | Preferred Ranges |
|---|---|---|---|
| Compound of Formulae I-XVI | 400 | mg | +50% to −50% |
| Microcrystalline Cellulose | 392 | mg | +50% to −50% |
| Colloidal Silicon Dioxide | 4 | mg | +50% to −50% |
| Magnesium Stearate | 4 | mg | +50% to −50% |
| Coated with | | | |
| Lactose monohydrate | | | |
| Hydroxyl propyl methyl | | | |
| cellulose | | | |
| Titanium dioxide | | | |
| Tracetin/glycerol triacetate | | | |
| Iron oxide | | | |

The coated tablets are produced using art known procedures.

### Example 3

### Capsules

| Ingredient | Amount | | Preferred Ranges |
|---|---|---|---|
| Compound of Formulae I-XVI | 400 | mg | +50% to −50% |
| Microcrystalline Cellulose | 392 | mg | +50% to −50% |
| Colloidal Silicon Dioxide | 4 | mg | +50% to −50% |
| Magnesium Stearate | 4 | mg | +50% to −50% |
| Encapsulated in gelatin | | | |

The capsules are produced using art known procedures.

### Example 4

### Tablets

| Ingredient | Amount | | Preferred Ranges |
|---|---|---|---|
| Compound of Formulae I-XVI | 200 | mg | +50% to −50% |
| Microcrystalline Cellulose | 196 | mg | +50% to −50% |
| Colloidal Silicon Dioxide | 2 | mg | +50% to −50% |
| Magnesium Stearate | 2 | mg | +50% to −50% |

### Example 5

We generated a stock of Drosophila that is heterozygous for both KHC and KLC, which encodes proteins that associate to form functional kinesin-1, also called conventional kinesin. As a result of the approximately 50% reduction in the level of kinesin-1, these khc/+; klc/+ larvae exhibit a motor defect termed “tail-flipping”. Specifically, the mutant larvae exhibit loss of motor activity in the ventral posterior segments that causes an imbalance in body wall contractions; as a result, the larvae rhythmically flip their tails upward during locomotion. In preliminary studies we found that the penetrance of the tail-flipping phenotype was less than 100%; that is, not all khc/+; klc/+ larvae show the phenotype. We identified a number of factors that contribute to this incomplete penetrance:

1. The flipper phenotype of a given animal appears to be suppressed by the number of larvae that precede the animal in development. That is, if a larva is among the first to develop in a vial of eggs, it is more likely to show the flipper phenotype than if it is one of the last emerging larvae.
2. The flipper phenotype appears to be less robust on hard than on soft media.
3. The phenotype is diminished by physically disturbing the larvae.
4. The clearest expression of the flipper phenotype is restricted to that phase of the 3rd instar stage of development that follows appearance of spiracles.
We attempted to accommodate these observations in order to optimize penetrance of the phenotype. Specifically:
1. Virgin females and males were confined to a single vial for only 2 days; the flies were then transferred to fresh vials for an additional 2 days; and this process was repeated to minimize the number of larvae that would emerge in each vial.
2. Efforts were taken to minimize handling of the larvae.
3. We attempted to score the phenotype late in the 3rd instar stage of development.

After optimization, the penetrance of the phenotype appeared to be consistent with literature values (Mol Cel Bio 10:3717 (1999)).

### Example 6

In a blinded experiment we tested the compound below for its ability to suppress the flipper phenotype of khc/+; klc/+ Drosophila larvae (as described in Example 5). When results are expressed in terms of the number of flies exhibiting no observable motor dysfunction (Non-Flipper) relative to the number with some degree of dysfunction (Flipper), the compound is seen to suppress the flipper phenotype, in a statistical significant manner as compared to flys treated with vehicle alone.

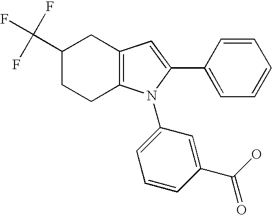

The flipper phenotype of khc/+; klc/+ Drosophila larvae is considered to be a model of some human motor neuropathies (e.g., disease associated with a defect in vesicular transport), including certain forms of amyotrophic lateral sclerosis (ALS) (Genetics 144:1075, 1996). Indeed, the relevance of the Drosophila model to ALS is supported by a recent report using the SOD1G93A mouse model of ALS (J Cell Biol 169:561, 2005). This report showed amelioration of disease when the ALS-prone mice were made mutant for the dynein heavy chain. This result, which is paradoxical on several grounds, was anticipated by dynein mutations in Drosophila models of ALS (Neuron 32:389, 2001). In view of the predictive power of Drosophila for interventions that ameliorate ALS, we anticipate the use of the compounds of the invention for treating ALS, and other disorders. Thus it is believed that the compounds of the invention can be used to modulate vesicular transport and treat disease associated with defects in vesicular transport

### Example 7

### Synthesis of Compounds

General: Chemicals were purchased from standard commercial vendors and used as received unless otherwise noted. “Degassed” means reduced pressure then nitrogen gas for three cycles. Abbreviations are consistent with those in the ACS Style Guide, plus: satd (saturated), DCM (dichloromethane), pRPLC (preparative HPLC), “dry” glassware means oven/desiccator dried. Solvents were ACS grade unless otherwise noted. Analytical TLC plates (Silica Gel 60 F254, EM Science, Gibbstown, N.J., or Merck # 5715) were used to follow the course of reactions, and the MPLC system used for purifications was from Isco (Foxy Jr fraction collector, UA-6 detector), using Isco silica gel flash columns (10 or 40 g). ¹H NMR spectra in CDCl₃, CD₃OD, and/or d6-DMSO were recorded on either a Varian Mercury 400 MHz or Brucker ARX-300 MHz instrument and chemical shifts are expressed in parts per million (ppm, 6) relative to TMS as the internal standard. Mass spectra were obtained on a Thermo Finnigan LCQ-Deca (injection volume 5 uL, XTerra MS-C₁₈ 3.5 μm 2.1×50 mm column, XTerra MS-C₁₈ 5 μm 2.1×20 mm guard column), ESI source, analytical HPLC was performed on an HP1050 (injection volume 5 μl, XTerra RP-C₁₈ 5 μm 4.6×250 mm column, with an XTerra MS-C₁₈ 5 μm 2.1×20 mm guard column), and preparative HPLC was performed on an Agilent 1100 Prep-LC with various columns and conditions depending on the compound. GCMS was performed on either an Agilent Technology 6890N or Shimadzu QP5000/17A instrument. Yields are unoptimized.

### 1-(2-Oxo-2-phenyl-ethyl)-3,4-dihydro-1H-naphthalen-2-one (3)

A solution of phenacylbromide (5.21 g, 26.1 mmol) in toluene (16 mL) was added over 15 minutes to a boiling, stirred solution of 1-(3,4-dihydro-2-naphthyl)pyrrolidine (5.21 g, 26.2 mmol) in toluene (17 mL). The reaction was refluxed 3 hours, diluted with water (15 mL) and refluxed for 4 hours then cooled. The layers were separated and the aqueous phase was extracted with toluene and dried over MgSO₄ and concentrated. The material was purified by MPLC using a gradient from 0 to 20% ethyl acetate/hexanes to afford 4.85 g (70% yield) title product as a yellow oil.

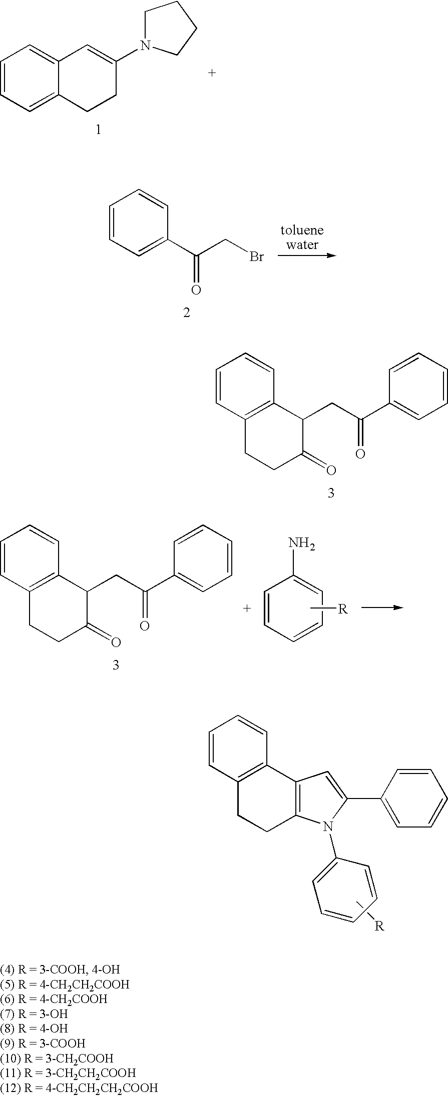

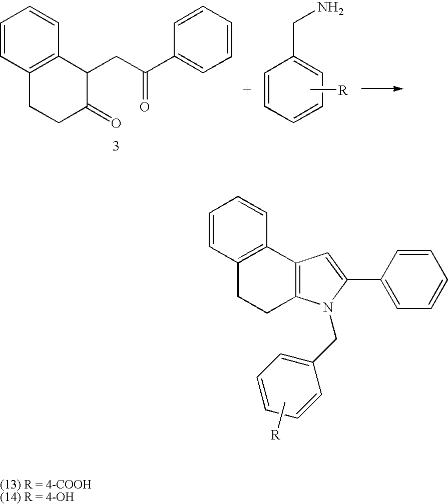

Compounds 4-14 were prepared in the same way. Compound 4 is given as an example.

### [2-Hydroxy-5-(2-phenyl-4,5-dihydro-benzo[e]indol-3-yl)-phenyl]-methanediol (4)

A mixture of 3 (2.41 g, 9.1 mmol), 5-aminosalicylic acid (1.40 g, 9.1 mmol) and glacial acetic acid (9 mL) was heated under reflux for 2 hours. After cooling, the precipitate was filtered and washed with acetic acid and water. The solid was recrystallized from acetic acid to afford 1.75 g (50% yield) title product as a yellow solid; MS m/z 380 (M-1-H) 9.92 min; ¹H NMR (DMSO-d₆) δ 2.63 (t, 2H), 2.94 (t, 2H), 4.89 (s, 1H), 7.16 (m, 13H).

### 3-[4-(2-Phenyl-4,5-dihydro-benzo[e]indol-3-yl)-phenyl]-propionic acid (5)

MS m/z 392 (M-1-H) 6.99 min; ¹H NMR (CDCl₃) δ 2.7 (d, 8H), 7.18 (m, 15H).

### [4-(2-Phenyl-4,5-dihydro-benzo[e]indol-3-yl)-phenyl]-acetic acid (6)

MS m/z 380 (M⁺+H) 6.90 min; ¹H NMR (CDCl₃) δ 2.75 (d, 2H), 3.74 (d, 2H), 7.40 (m, 17H).

### 3-(2-Phenyl-4,5-dihydro-benzo[e]indol-3-yl)-phenol (7)

MS m/z 336 (M-1-H), 6.97 min, 338 (M⁺+H) 6.95 min; ¹H NMR (CDCl₃) δ 2.75 (d, 4H), 7.08 (m, 15H).

### 4-(2-Phenyl-4,5-dihydro-benzo[e]indol-3-yl)-phenol (8)

MS m/z 336 (M-1-H) 6.85 min, 338 (M⁺+H) 6.86 min; ¹H NMR (CDCl₃) δ 2.60 (s, 2H), 2.87 (s, 2H), 3.89 (s, 2H), 6.91 (m, 13H).

### 3-(2-Phenyl-4,5-dihydro-benzo[e]indol-3-yl)-benzoic acid (9)

MS m/z 364 (M-1-H) 6.97 min, 366 (M⁺+H) 6.97 min; ¹H NMR (CDCl₃) δ 2.66 (t, 2H), 2.94 (t, 2H), 7.12 (m, 15H).

### [3-(2-Phenyl-4,5-dihydro-benzo[e]indol-3-yl)-phenyl]-acetic acid (10)

MS m/z 378 (M-1-H) 6.92 min; ¹H NMR (DMSO-d₆) δ 2.50 (s, 1H), 3.29 (s, 4H), 3.68 (s, 2H), 7.35 (m, 14H).

### 3-[3-(2-Phenyl-4,5-dihydro-benzo[e]indol-3-yl)-phenyl]-propionic acid (11)

MS m/z 392 (M-1-H) 7.33 min; ¹H NMR (CDCl₃) δ 2.12 (t, 3H), 2.47 (t, 4H) 2.80 (t, 2H), 7.08 (m, 14H).

### 4-[4-(2-Phenyl-4,5-dihydro-benzo[e]indol-3-yl)-phenyl]-butyric acid (12)

MS m/z 406 (M-1-H) 8.22 min; ¹H NMR(C₆D₆) δ 1.99 (m, 10H), 7.07 (m, 15H).

### 4-(2-Phenyl-4,5-dihydro-benzo[e]indol-3-ylmethyl)-benzoic acid (13)

MS m/z 378 (M-1-H) 6.81 min, 380 (M⁺+H) 6.81 min; 6 2.66 (t, 2H), 2.98 (t, 2H), 6.61 (s, 2H), 7.22 (m, 15H).

### 4-(2-Phenyl-4,5-dihydro-benzo[e]indol-3-ylmethyl)-phenol (14)

MS m/z 352 (M⁺+H) 6.83 min; ¹H NMR (CDCl₃) δ 2.68 (t, 2H), 2.97 (t, 2H), 5.09 (s, 2H), 7.21 (m, 15H).

### 3-[3-(2-Phenyl-benzo[e]indol-3-yl)-phenyl]-propionic acid (15)

MS m/z 390 (M-1-H) 7.45 min; ¹H NMR (CDCl₃) δ 2.15 (m, 4H), 7.07 (m, 15H).

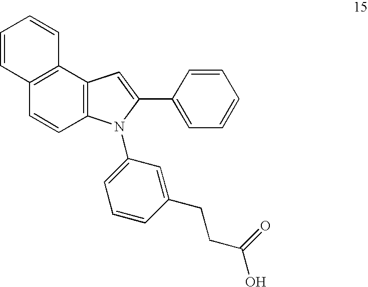

### Example 8

The following synthetic routes can be employed to make the compounds of Formulae I-XVI (e.g., those in the Tables below).

Route A: Allen, et al, J. Med. Chem. 1976, 19(2), 318-325.

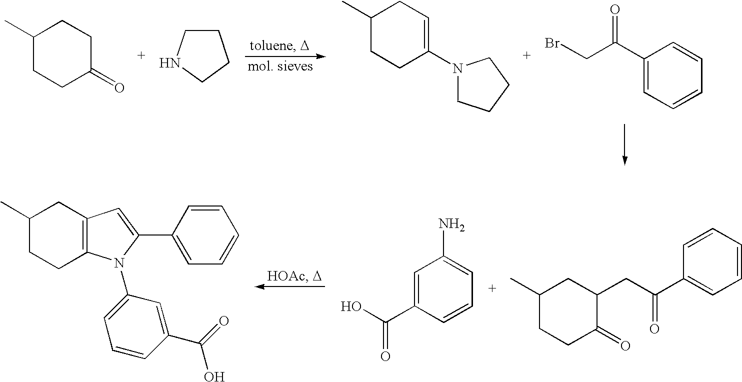

Route B: Murakami, et al, Chem. Pharm. Bull. 1995, 43(8), 1281-1286.

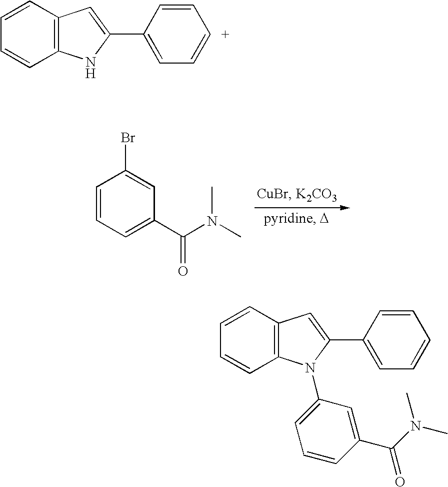

Route C: Allen, et al, J. Med. Chem. 1976, 19(2), 318-325.

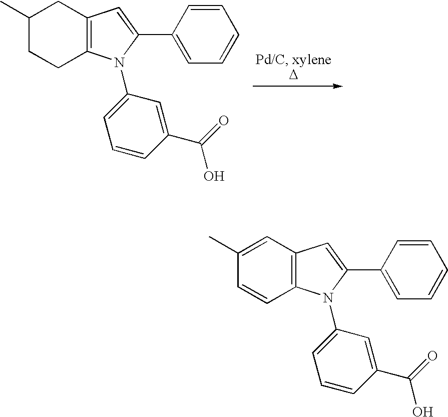

Compounds 16-90 below in Table 1, can be prepared in a similar manner as described for Compounds 4-14.

TABLE 1

| | product structure | SM ketone |
|---|---|---|
| 16 | 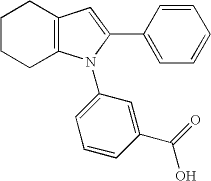 | 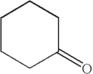 |
| 17 | 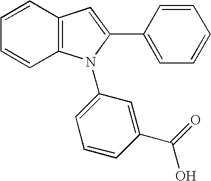 | 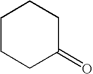 |
| 18 | 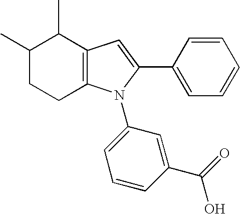 | 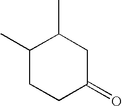 |
| 19 | 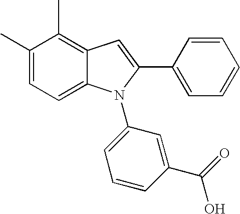 | 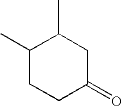 |
| 20 | 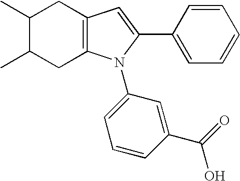 | 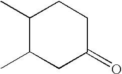 |
| 21 | 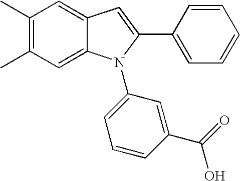 | 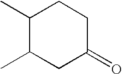 |
| 22 | 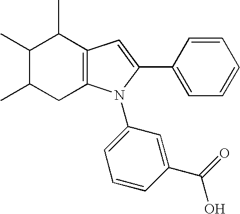 | 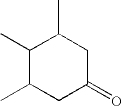 |
| 23 | 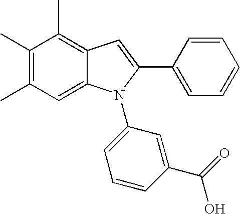 | 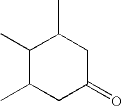 |
| 24 | 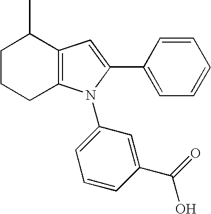 | 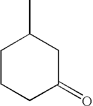 |
| 25 | 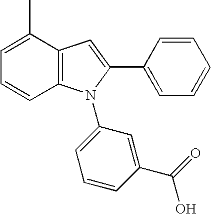 | 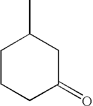 |
| 26 | 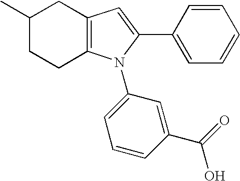 | 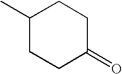 |
| 27 | 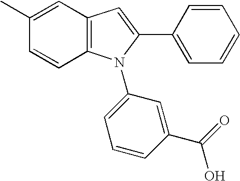 | 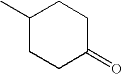 |
| 28 | 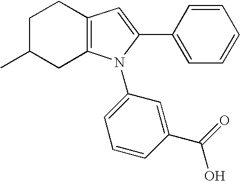 | 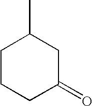 |
| 29 | 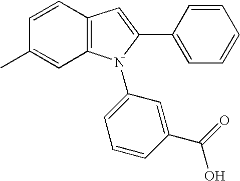 | 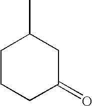 |
| 30 | 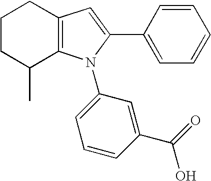 | 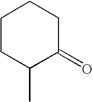 |
| 31 | 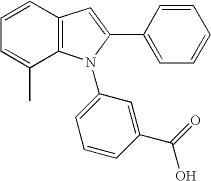 | 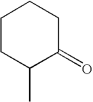 |
| 32 | 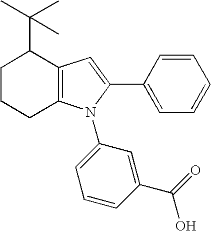 | 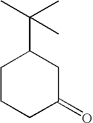 |
| 33 | 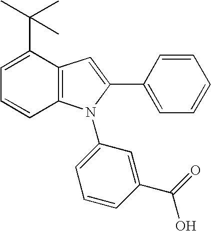 | 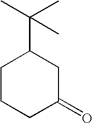 |
| 34 | 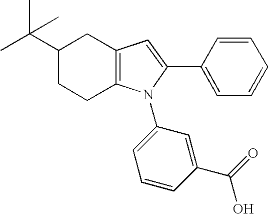 | 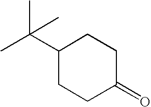 |
| 35 | 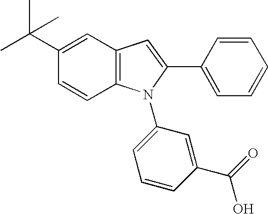 | 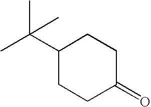 |
| 36 | 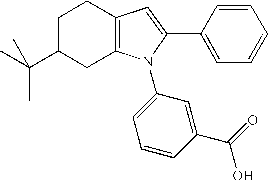 | 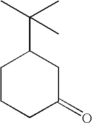 |
| 37 | 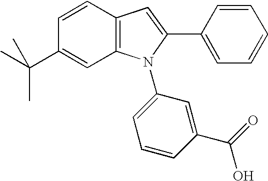 | 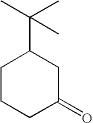 |
| 38 | 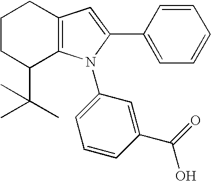 | 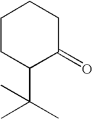 |
| 39 | 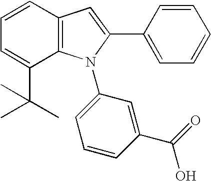 | 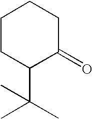 |
| 40 | 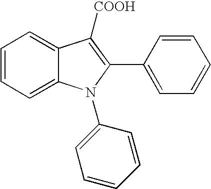 | 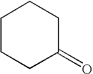 |
| 41 | 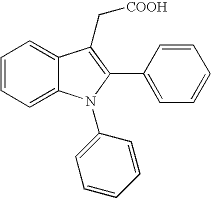 | 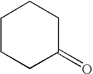 |
| 42 | 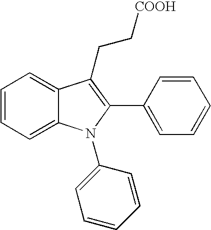 | 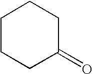 |
| 43 | 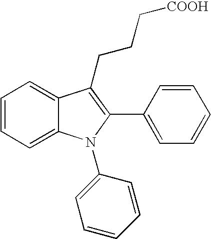 | 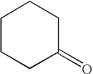 |
| 44 | 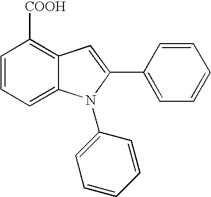 | 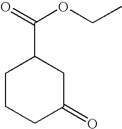 |
| 46 | 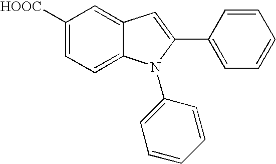 | 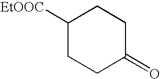 |
| 47 | 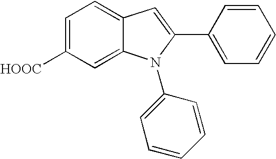 | 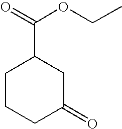 |
| 48 | 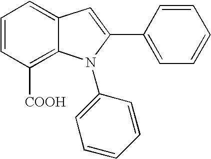 | 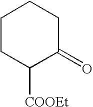 |
| 49 | 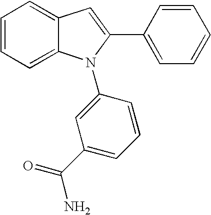 | 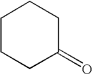 |
| 50 | 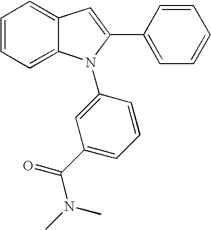 | 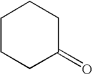 |
| 51 | 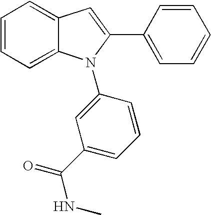 | 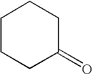 |
| 52 | 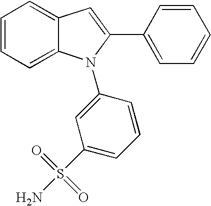 | 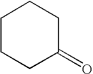 |
| 53 | 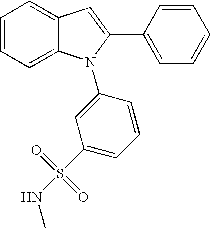 | 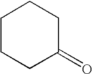 |
| 54 | 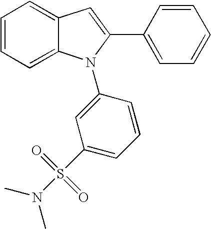 | 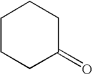 |
| 55 | 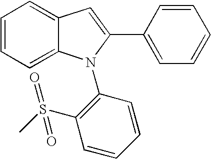 | 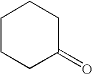 |
| 56 | 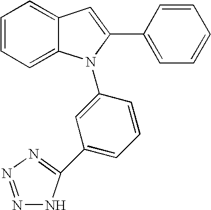 | 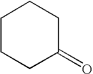 |
| | product structure | SM ketone/enamine |
| 57 | 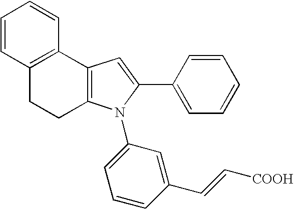 | 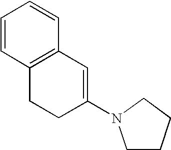 |
| 58 | 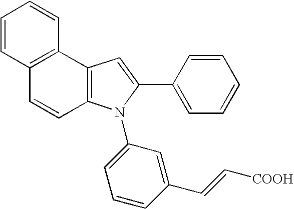 | 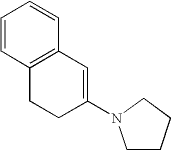 |
| 59 | 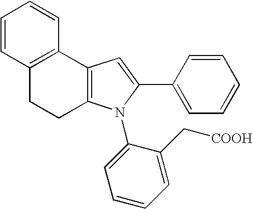 | 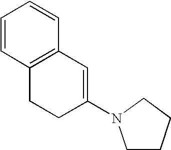 |
| 60 | 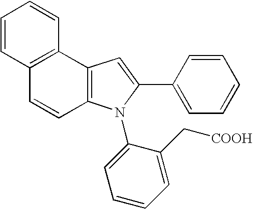 | 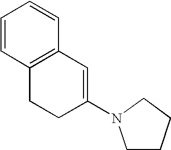 |
| 61 | 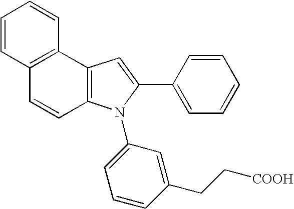 | 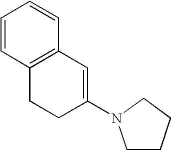 |
| 62 | 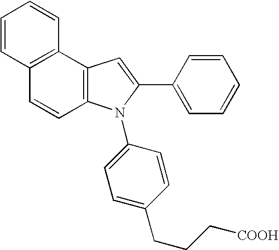 | 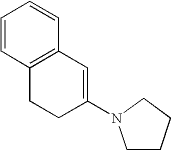 |
| 63 | 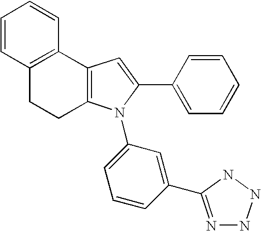 | 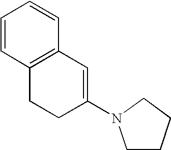 |
| 64 | 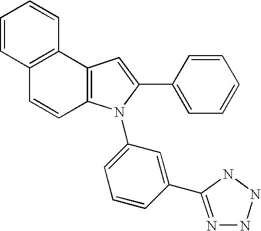 | 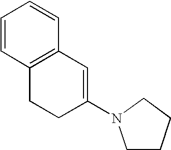 |
| 65 | 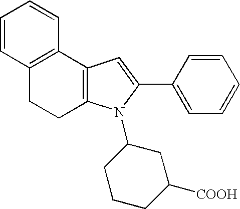 | 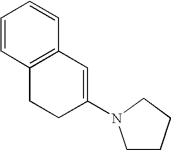 |
| 66 | 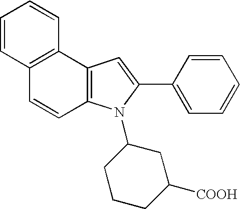 | 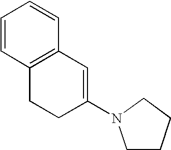 |
| 67 | 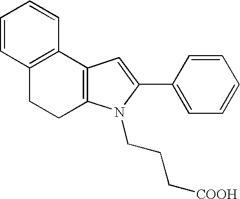 | 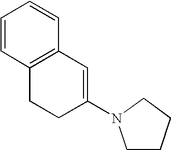 |
| 68 | 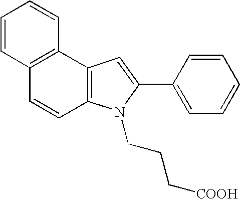 | 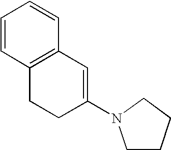 |
| 69 | 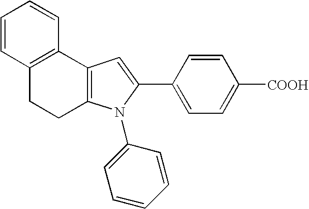 | 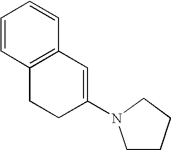 |
| 70 | 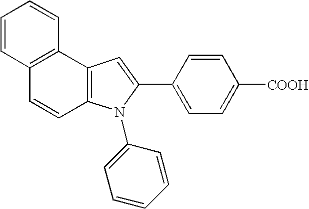 | 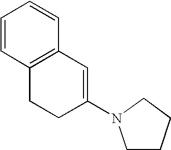 |
| 71 | 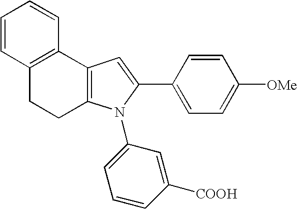 | 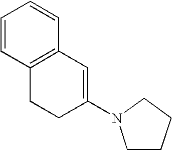 |
| 72 | 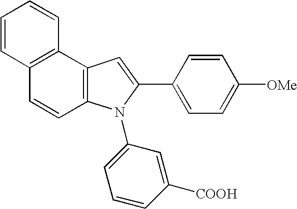 | 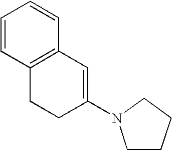 |
| 19 | 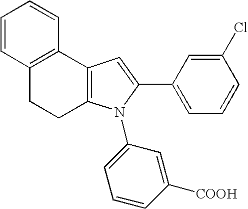 | 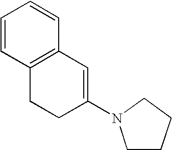 |
| 73 | 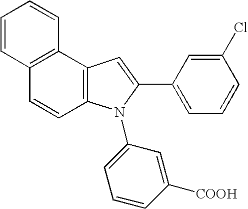 | 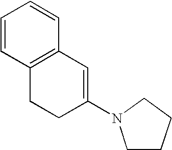 |
| 74 | 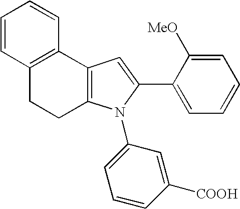 | 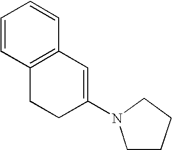 |
| 75 | 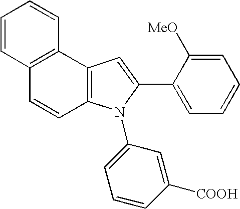 | 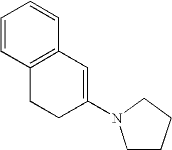 |
| 76 | 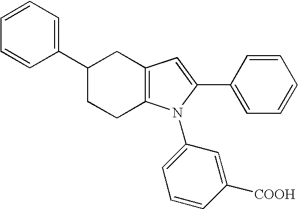 | 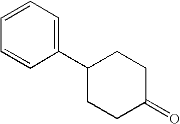 |
| 77 | 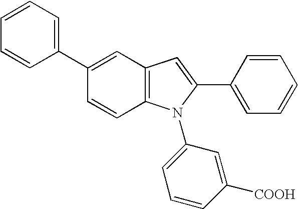 | 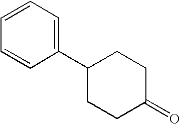 |
| 78 | 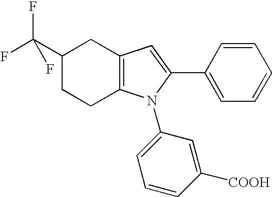 | 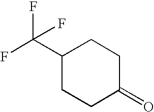 |
| 79 | 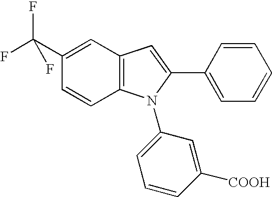 | 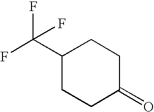 |
| 80 | 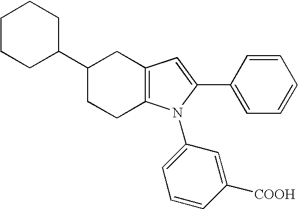 | 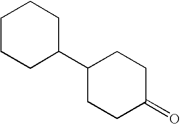 |
| 81 | 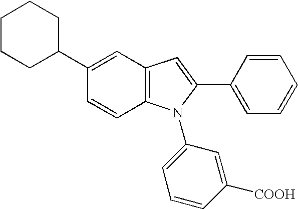 | 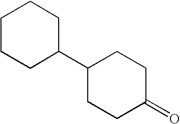 |
| 82 | 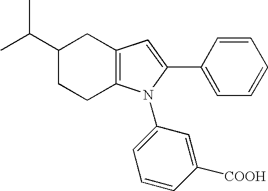 | 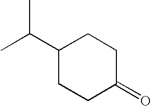 |
| 83 | 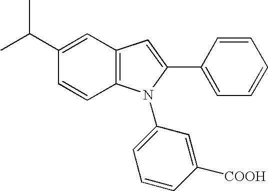 | 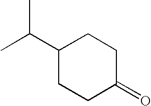 |

| | | | synthetic |
|---|---|---|---|
| | alpha-bromo ketone | aniline | route |
| 16 | 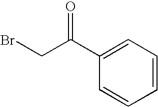 | 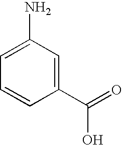 | A |
| 17 | 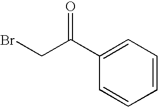 | 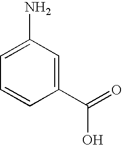 | A, C |
| 18 | 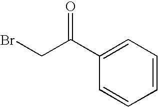 | 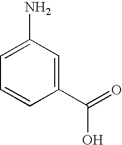 | A |
| 19 | 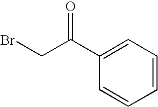 | 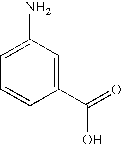 | A, C |
| 20 | 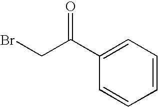 | 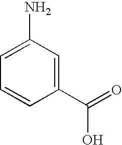 | A |
| 21 | 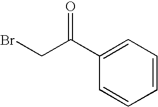 | 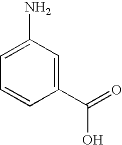 | A, C |
| 22 | 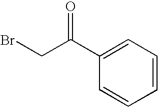 | 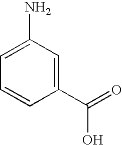 | A |
| 23 | 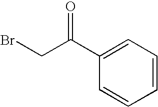 | 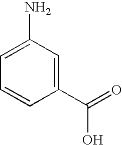 | A, C |
| 24 | 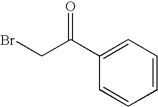 | 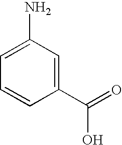 | A |
| 25 | 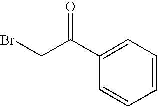 | 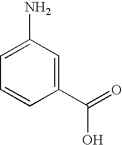 | A, C |
| 26 | 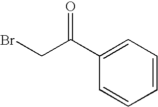 | 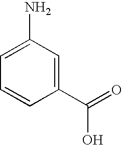 | A |
| 27 | 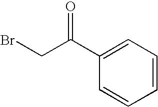 | 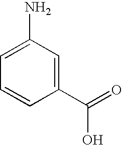 | A, C |
| 28 | 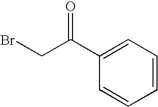 | 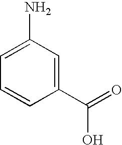 | A |
| 29 | 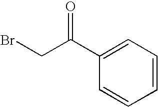 | 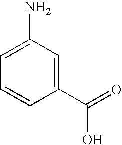 | A, C |
| 30 | 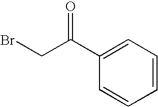 | 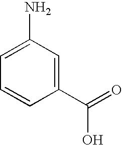 | A |
| 31 | 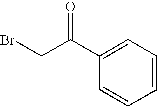 | 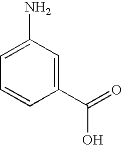 | A, C |
| 32 | 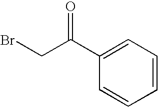 | 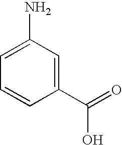 | A |
| 33 | 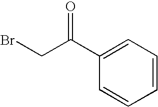 | 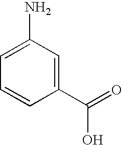 | A, C |
| 34 | 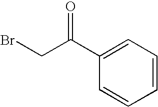 | 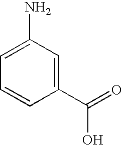 | A |
| 35 | 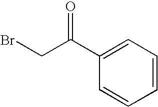 | 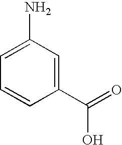 | A, C |
| 36 | 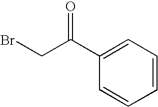 | 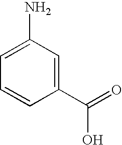 | A |
| 37 | 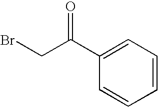 | 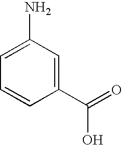 | A, C |
| 38 | 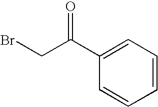 | 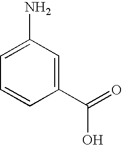 | A |
| 39 | 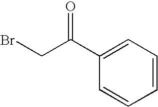 | 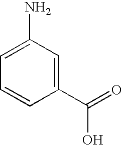 | A, C |
| 40 | 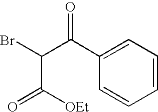 | 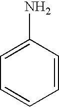 | A, C, B |
| 41 | 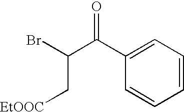 | 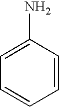 | A, C, B |
| 42 | 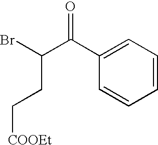 | 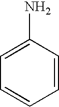 | A, C, B |
| 43 | 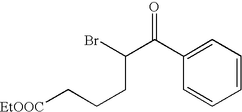 | 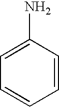 | A, C, B |
| 44 | 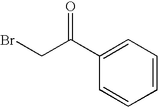 | 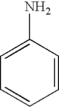 | A, C, B |
| 46 | 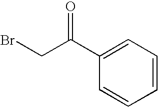 | 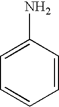 | A, C, B |
| 47 | 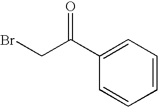 | 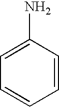 | A, C, B |
| 48 | 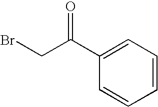 | 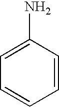 | A, C, B |
| 49 | 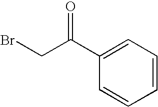 | 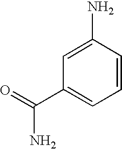 | A, C |
| 50 | 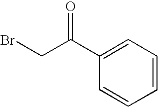 | 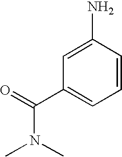 | A, C |
| 51 | 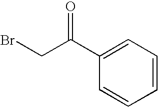 | 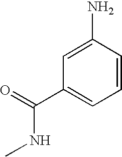 | A, C |
| 52 | 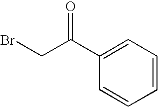 | 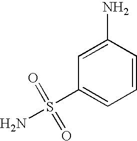 | A, C |
| 53 | 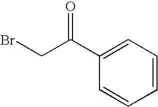 | 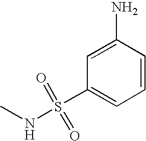 | A, C |
| 54 | 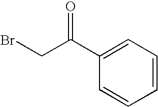 | 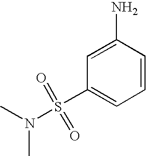 | A, C |
| 55 | 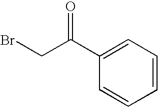 | 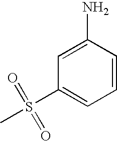 | A, C |
| 56 | 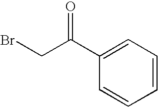 | 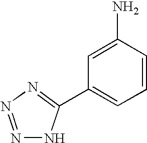 | A, C |
| 57 | 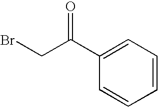 | 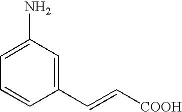 | A |
| 58 | 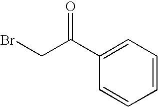 | 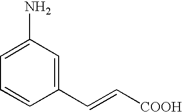 | A, C |
| 59 | 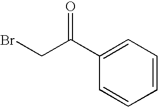 | 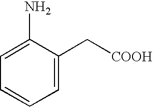 | A |
| 60 | 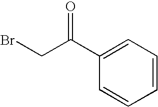 | 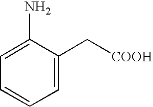 | A, C |
| 61 | 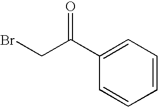 | 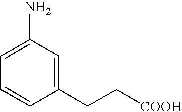 | A, C |
| 62 | 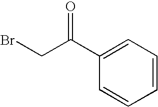 | 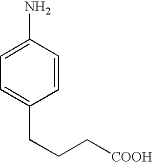 | A, C |
| 63 | 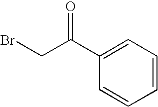 | 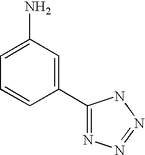 | A |
| 64 | 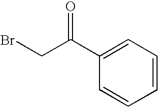 | 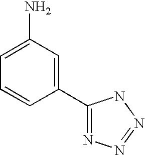 | A, C |
| 65 | 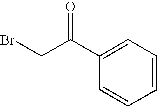 | 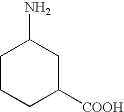 | A |
| 66 | 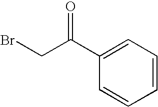 | 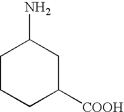 | A, C |
| 67 | 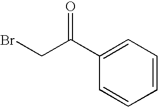 | 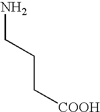 | A |
| 68 | 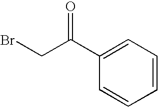 | 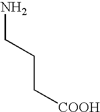 | A, C |
| 69 | 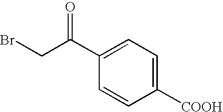 | 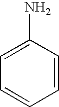 | A |
| 70 | 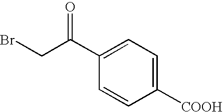 | 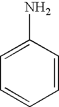 | A, C |
| 71 | 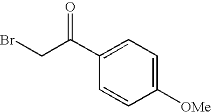 | 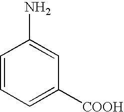 | A |
| 72 | 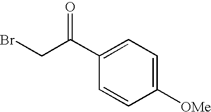 | 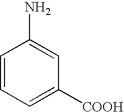 | A, C |
| 19 | 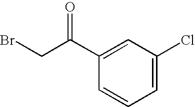 | 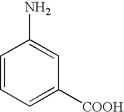 | A |
| 73 | 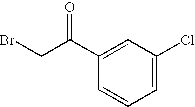 | 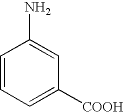 | A, C |
| 74 | 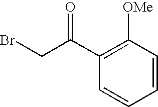 | 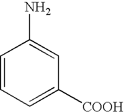 | A |
| 75 | 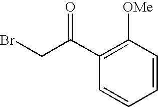 | 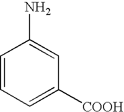 | A, C |
| 76 | 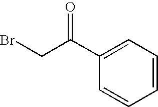 | 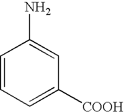 | A |
| 77 | 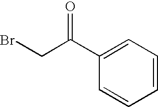 | 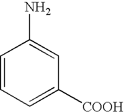 | A, C |
| 78 | 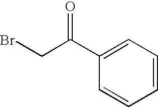 | 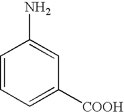 | A |
| 79 | 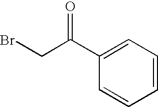 | 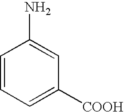 | A, C |
| 80 | 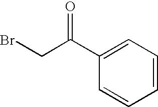 | 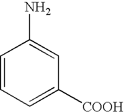 | A |
| 81 | 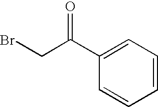 | 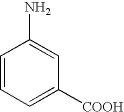 | A, C |
| 82 | 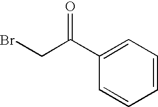 | 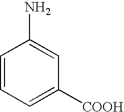 | A |
| 83 | 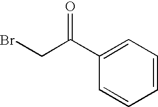 | 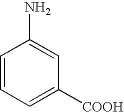 | A, C |

### Example 9

### Synthesis of Compound 34

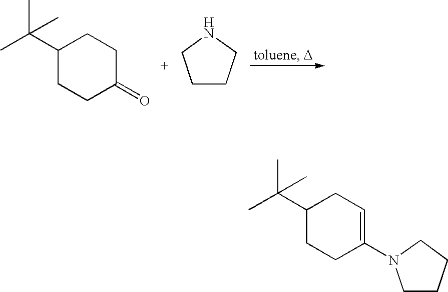

1-(4-tert-butylcyclohex-1-enyl)pyrrolidine: A 50 mL round-bottomed flask containing 4-tert-butylcyclohexanone (6.01 gm) in anhydrous toluene (20 mL) was fitted with a Dean-Stark trap containing 3 A molecular sieves, reflux condenser and a heating mantle. Pyrrolidine (6.00 mL) was added, and the solution heated to reflux for 18 hr. The solvent was evaporated and the crude product was used directly for the next reaction.

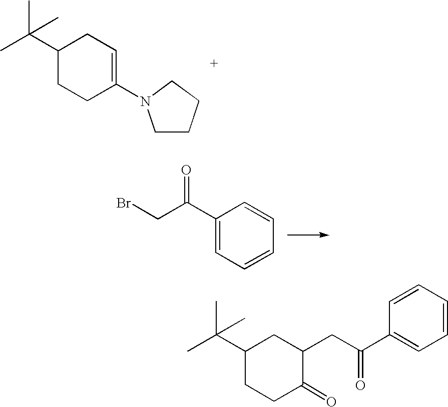

4-tert-butyl-2-(2-oxo-2-phenylethyl)-cyclohexanone: To a 250-mL round-bottomed flask containing 3.3 mL of 1-(4-tert-butylcyclohex-1-enyl)pyrrolidine was added 100 mL anhydrous DMF, under nitrogen. The flask was fitted with an addition funnel containing 2-bromoacetophenone (4.12 gm) in 35 mL anhydrous DMF, which was dripped into the enamine solution over 60 min. This solution was stirred at ambient temperature for 10 hr, then 90 mL water was added to the solution and it was stirred for another 11 hr, under nitrogen. The solution was then extracted twice with ethyl acetate and water, the organic layers combined and further washed with water (3×), dried over sodium sulfate, filtered and rotovapped down to give a yellow oil. The oil was purified by MPLC using 10% ethyl acetate/hexanes.

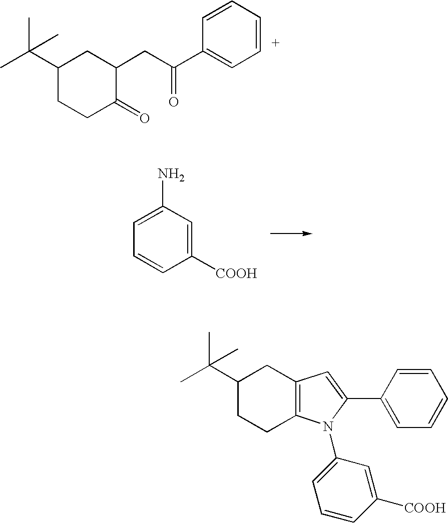

3-(5-tert-butyl-2-phenyl-4,5,6,7-tetrahydroindol-1-yl)benzoic acid (Compound 34): A solution of 4-tert-butyl-2-(2-oxo-2-phenylethyl)-cyclohexanone (0.219 gm) in glacial acetic acid (3.0 mL) in a 25-mL round-bottomed flask, under nitrogen, was fitted with a heating mantle and reflux condenser. To this solution was added 3-aminobenzoic acid (0.138 gm), which was then heated at 110 C for 3 hr. The solution was cooled to ambient temperature, 8 mL water was added, and the suspension was stirred 18 hr under nitrogen. The solid was filtered, washed with water, and recrystallized in acetonitrile to provide 0.123 gm of the pure product.

### Example 10

Analytical data for compounds of Formulae I and II. These compounds were synthesized via the indicated synthetic route. Ab42 IC50 (uM) refers to IC₅₀ value for Ab42 lowering in e.g., the assay described in Example 6.

TABLE 2

| | | | | | Syn. |
|---|---|---|---|---|---|
| Compound | product | 1H NMR, | | | route |
| Number | structure | δ | MS | name | used |
| 17 | 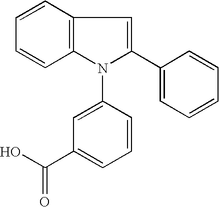 | CDCl3; 8.1 (m, 2 H);7.7 (m, 1 H); 7.5 (t,1 H); 7.4 (m, 1 H);7.2-7.3 (m, 8 H, ArH);6.8 (s, 1 H). | pos. mode 314 (M + H);neg. mode 312 (M − H) | 3-(2-phenylindol-1-yl)benzoic acid | A, C |
| 34 | 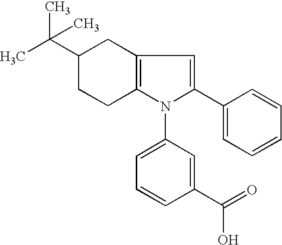 | CDCl3/d3-MeOD; 8.0(m, 2 H); 7.4 (t, 1 H);7.2 (m, 1 H); 7.0-7.2(m, 5 H, ArH); 6.2 (s,1 H); 2.7 (m, 1 H);2.5 (s, 1 H); 2.4 (m,2 H); 2.0 (m, 1 H);1.5 (m, 1 H); 1.4 (m,1 H); 0.9 (s, 9 H). | pos. mode 374 (M + H);neg. mode 372 (M − H) | 3-(5-tert-Butyl-2-phenyl-4,5,6,7-tetra-hydroindol-1-yl)benzoic acid | A |
| 85 | 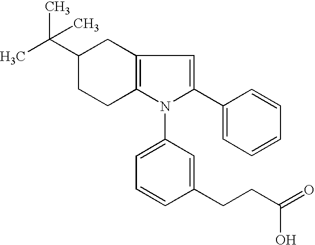 | CDCl3; 7.2 (m, 1 H);6.9-7.1 (m, 8 H, ArH);6.2 (s, 1 H); 2.9 (t,2 H); 2.7 (m, 1 H); 2.5(m, 3 H); 2.4 (m, 2 H);2.0 (m, 1 H); 1.5 (m,1 H); 1.4 (m, 1 H); 0.9(s, 9 H). | pos. mode 402 (M + H);neg. mode 400 (M − H) | 3-[3-(5-tert-Butyl-2-phenyl-4,5,6,7-tetra-hydroindol-1-yl)-phenyl] propionic acid | A |
| 63 | 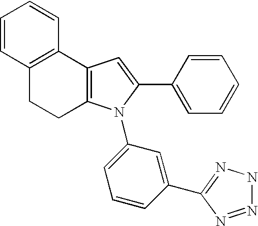 | DMSO-d6; 7.0-8.4(13 H, ArH); 6.9(1 H), 2.9 (2 H, CH2),2.5 (2 H, CH2). | pos. mode 342 (M + H);neg. mode 340 (M − H) | 2-phenyl-3-[3-(2H-tetrazol-5-yl)-phenyl]-4,5-dihydro-3H-benzo[e]indole | A |
| 69 | 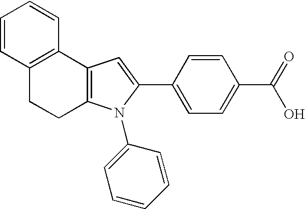 | DMSO-d6; 6.8-7.9(14 H, ArH); 3.0 (2 H,CH2) 2.7 (2 H, CH2). | neg. mode 364 (M − 1) | 4-(3-phenyl-4,5-dihydro-3H-benzo[e]indol-2-yl) benzoicacid | A |
| 86 | 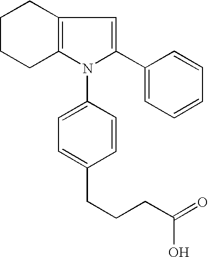 | CDCl3; 7.0-7.2 (m,9 H, ArH); 6.2 (s,1 H); 2.6 (m, 4 H);2.4 (m, 4 H); 2.0 (m,3 H); 1.8 (s, 3 H). | neg. mode 358 (M − H) | 4-[4-(2-phenyl-4,5,6,7-tetrahydroindol-1-yl)-phenyl] butyric acid | A |
| 87 | 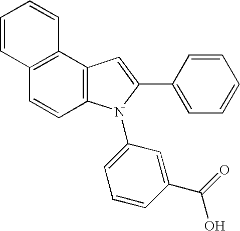 | DMSO-d6; 7.2-8.4(16 H, ArH). | pos. mode 364 (M + 1);neg. mode 362 (M − 1) | 3-(2-phenylbenzo[e]indol-3-yl) benzoicacid | A, C |
| 88 | 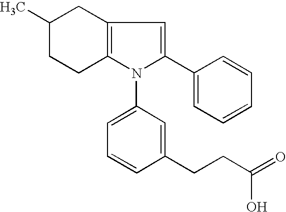 | CDCl3; 7.3 (t, 1 H);6.9-7.1 (m, 8 H, ArH);6.2 (s, 1 H); 2.9 (t,2 H); 2.7 (m, 1 H); 2.5(m, 3 H); 2.4 (m, 1 H);2.2 (m, 1 H); 1.9 (m,2 H); 1.4 (m, 1 H); 1.0(d, 3 H). | pos. mode 360 (M + H);neg. mode 358 (M − H) | 3-[3-(5-methyl-2-phenyl-4,5,6,7-tetra-hydroindol-1-yl)-phenyl] propionic acid | A |
| 89 | 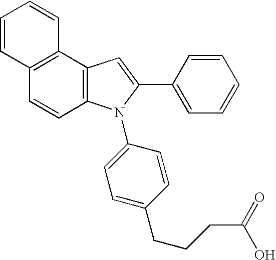 | DMSO-d6; 7.2-8.4(16 H, ArH); 2.7 (2 H,CH2); 2.3 (2 H, CH2);1.9 (2 H, CH2). | pos. mode 406 (M + 1);neg. mode 404 (M − 1) | 4-[4-(2-phenyl-benzo[e]indol-3-yl)-phenyl]butyric acid | A, C |
| 90 | 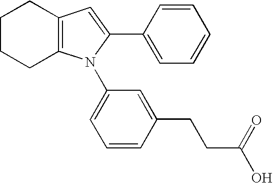 | CDCl3; 7.3 (t, 1 H);6.9-7.2 (m, 8 H, ArH);6.2 (s, 1 H); 2.9 (t,2 H); 2.6 (br. s, 2 H);2.5 (t, 2 H); 2.4 (br. s,2 H); 1.8 (br. s, 4 H). | pos. mode 346 (M + H) | 3-[3-(2-phenyl-4,5,6,7-tetrahydroindol-1-yl)-phenyl] propionic acid | A |
| 66 | 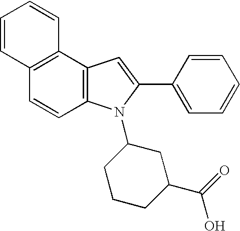 | CDCl3; 7.1-8.4 (11 H,ArH), 6.4 (1 H, ArH),4.4 (1 H, CH) 1.4-2.7(9 H, CH2). | pos. mode 372 (M + 1) | 3-(2-phenylbenzo[e]indol-3-yl) cyclo-hexane carboxylic acid | A, C |
| 67 | 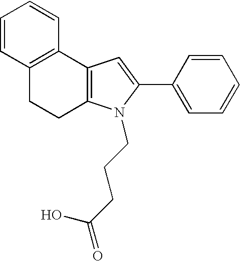 | CD3OD-d4; 7.1-8.2(10 H, ArH), 4.0 (2 H,CH2), 3.0 (2 H, CH2),2.9 (2 H, CH2), 2.1(2 H, CH2), 1.9 (2 H,CH2). | pos. mode 332 (M + 1) | 4-(2-phenyl-4,5-di-hydrobenzo[e]indol-3-yl) butyric acid | A |
| 68 | 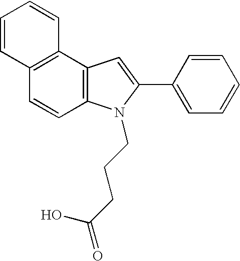 | CD3OD-d4; 7.1-8.2(12 H, ArH) 4.4 (2 H,CH2) 2.1 (2 H, CH2)1.9 (2 H, CH2). | pos. mode 330 (M + 1) | 4-(2-phenyl-benzo[e]indol-3-yl) butyric acid | A, C |
| 71 | 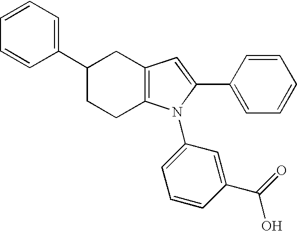 | DMSO-d6; 7.0-7.9(14 H, ArH), 6.3 (1 H,ArH), 3.0 (1 H, CH),2.8 (1 H, CH2), 2.7(2 H, CH2), 2.4 (1 H,CH2), 1.9 (2 H, CH2). | pos. mode 394 (M + 1) | 3-(2,5-diphenyl-4,5,6,7-tetrahydro-indol-1-yl) benzoicacid | A, C |
| 91 | 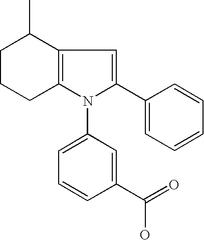 | CDCl3; 8.0 (m, 1 H);7.9 (m, 1 H); 7.4 (t,1 H); 7.0-7.3 (m, 6 H,ArH); 6.2 (s, 1 H); 2.6(m, 1 H); 2.5 (br. s,1 H); 2.4 (m, 1 H); 2.1(m, 1 H); 1.9 (m, 2 H);1.4 (m, 1 H); 1.0 (d,3 H). | pos. mode 332 (M + H) | 3-(4-methyl-2-phenyl-4,5,6,7-tetrahydro-indol-1-yl) benzoicacid | A |
| 92 | 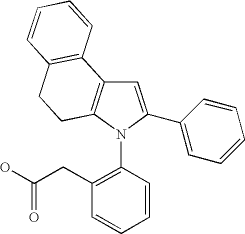 | acetone-d6; 7.5 (m,5 H); 7.2 (m, 7 H);7.0 (t, 1 H); 6.8 (s,1 H); 3.2 (s, 2 H,CH2); 2.9 (m, 2 H);2.6 (m, 1 H); 2.4 (m,1 H). | pos. mode 380 (M + H) | [2-(2-phenyl-4,5-di-hydrobenzo[e]indol-3-yl)-phenyl] acetic acid | A |

### Example 11

The following synthetic routes can be used to make the compounds of Formulae I-XVI.

### Synthetic Routes for Heteroaromatics

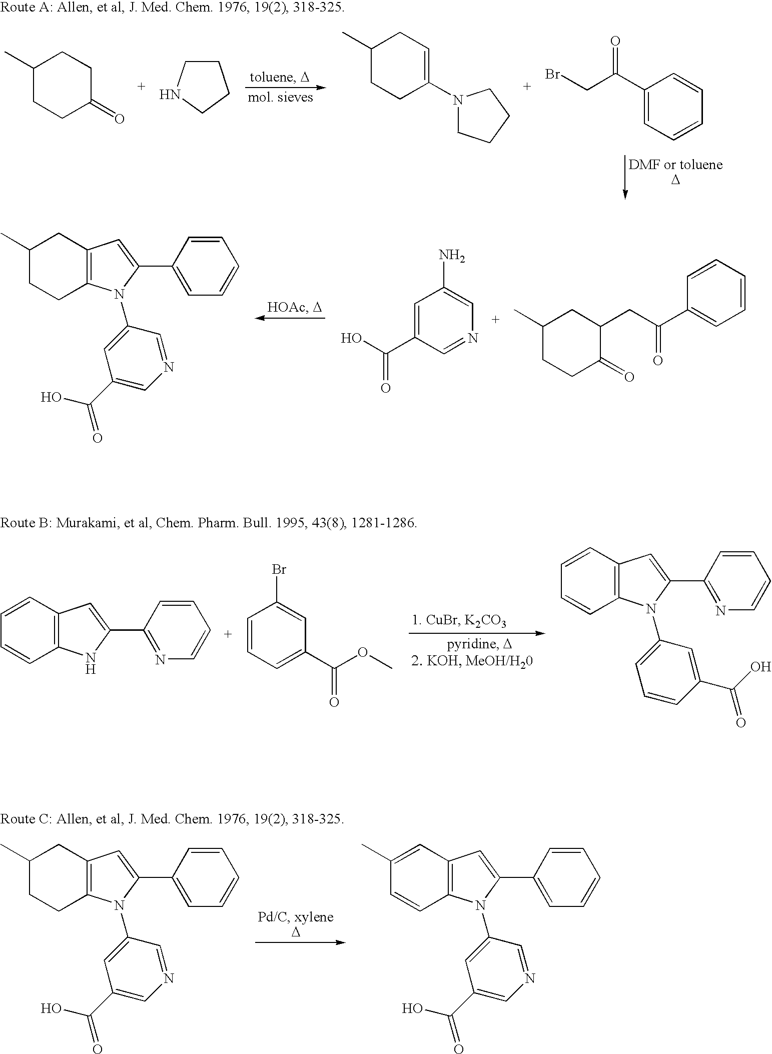

### Compounds for Heteroaromatics

### Heteroaromatic N-Alkylated Analogs:

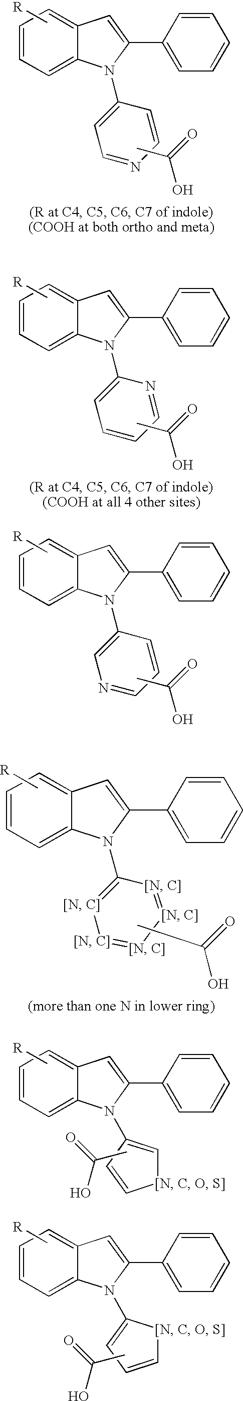

also, all of the above with a partially saturated ring (4,5,6,7-tetrahydroindoles):

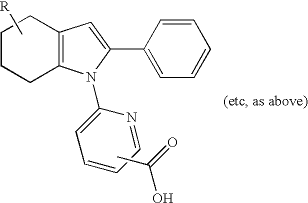

rearranging the acid group placement:

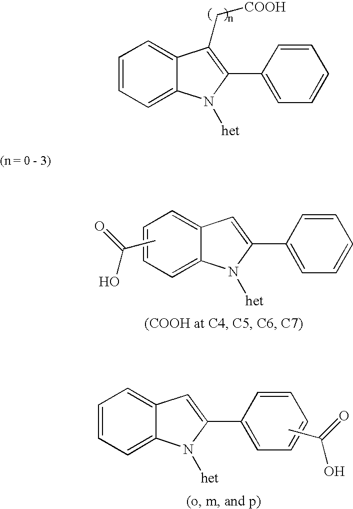

placing the heterocycle at the indole C-1 or C-2 position:

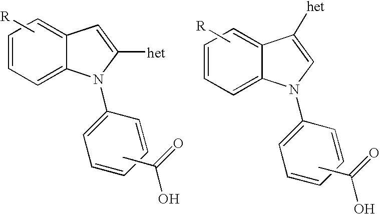

changing the acid group moiety:

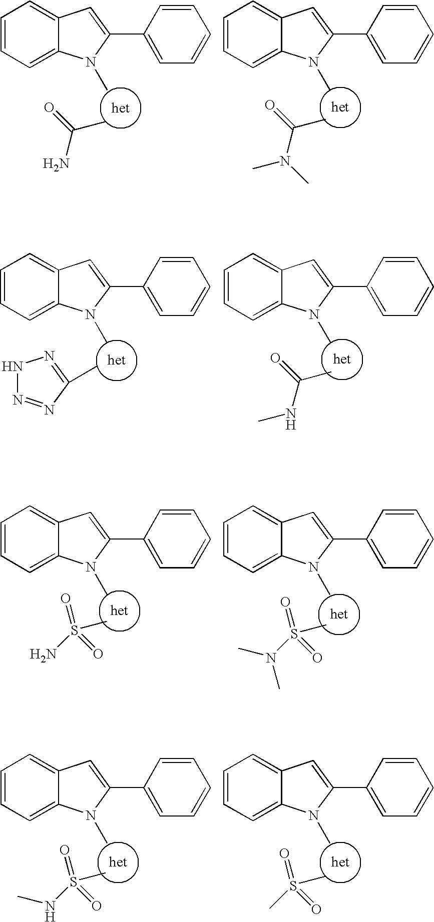

Compounds of Formulae I-XVI include, but are not limited to:

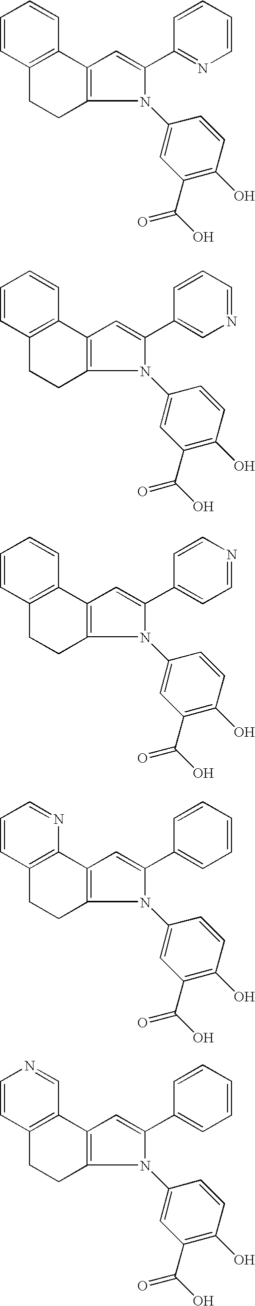

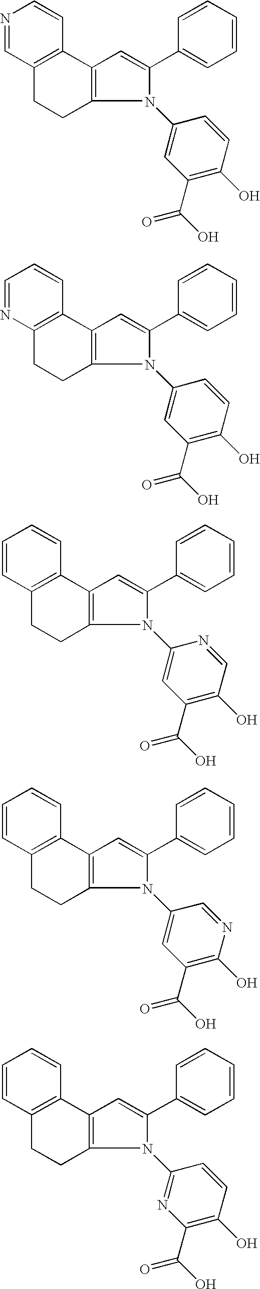

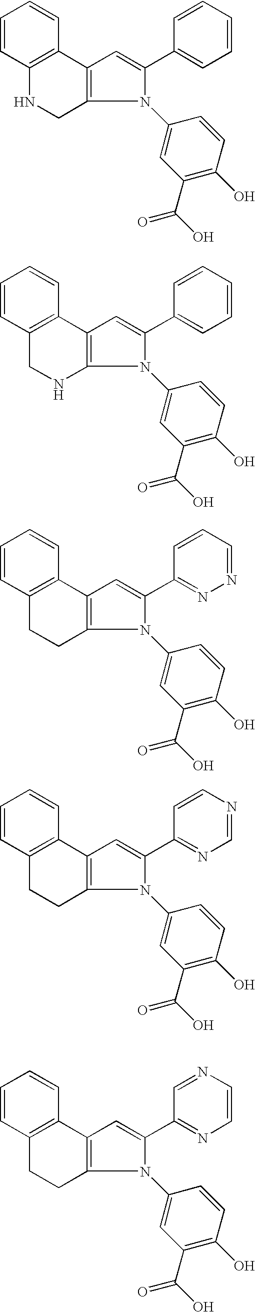

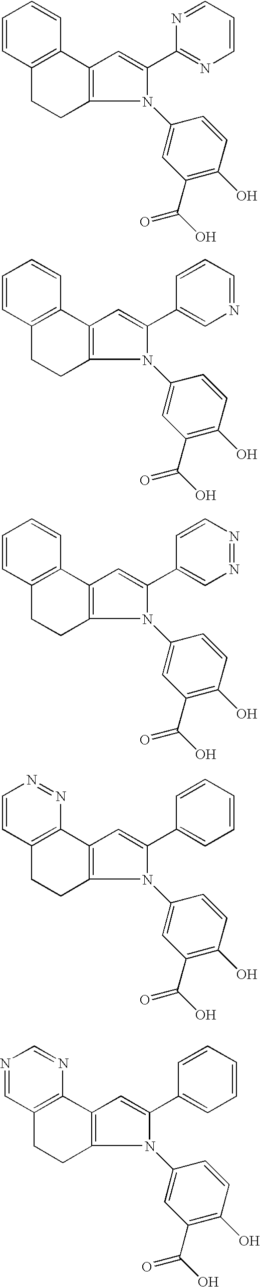

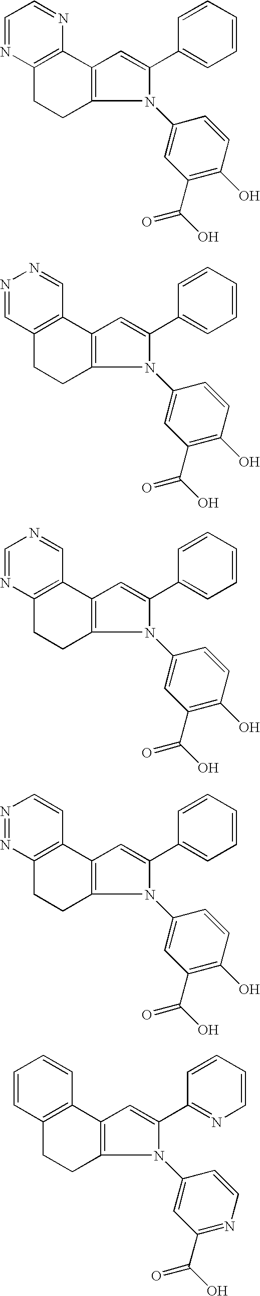

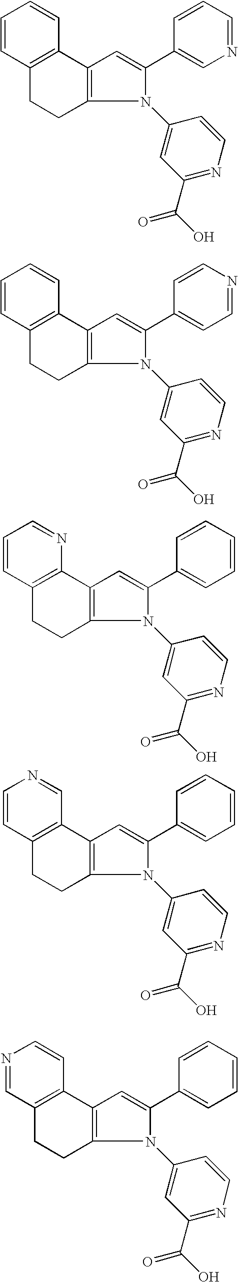

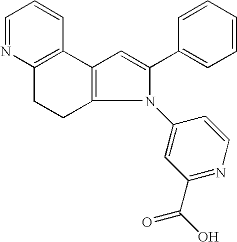

TABLE 3
Exemplary Compounds of the Invention

| | | | alpha- | | syn- |
|---|---|---|---|---|---|
| | product | SM | bromo | | thetic |
| | structure | ketone | ketone | aniline | route |
| 93 | 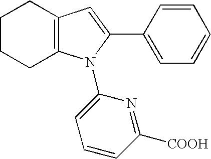 | 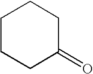 | 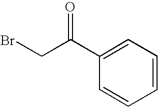 | 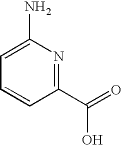 | A |
| 94 | 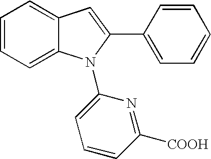 | 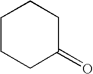 | 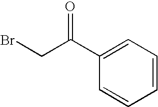 | 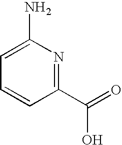 | A, C |
| 95 | 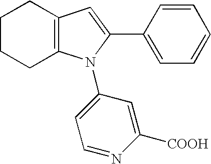 | 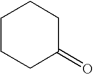 | 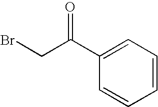 | 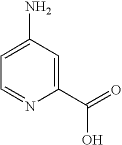 | A |
| 96 | 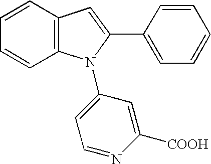 | 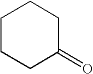 | 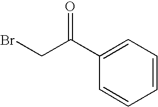 | 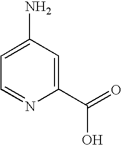 | A, C |
| 97 | 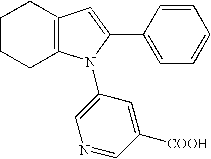 | 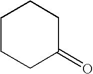 | 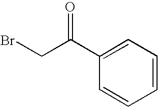 | 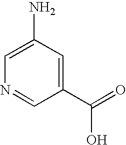 | A |
| 98 | 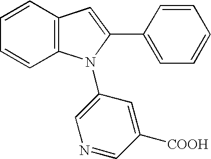 | 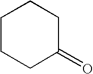 | 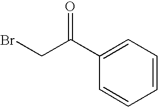 | 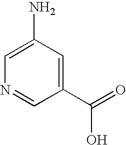 | A, C |
| 99 | 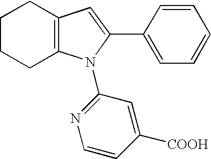 | 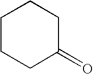 | 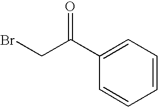 | 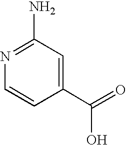 | A |
| 100 | 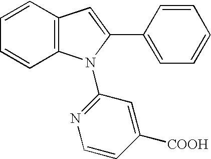 | 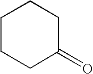 | 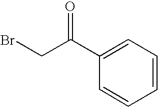 | 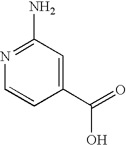 | A, C |
| 101 | 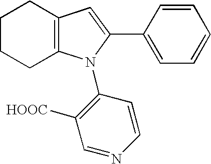 | 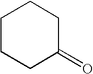 | 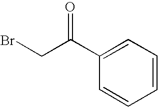 | 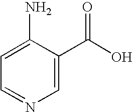 | A |
| 102 | 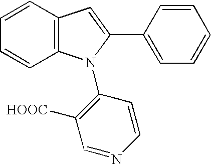 | 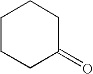 | 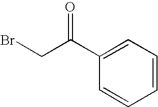 | 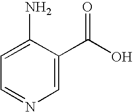 | A, C |
| 103 | 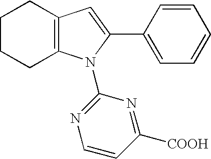 | 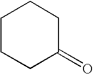 | 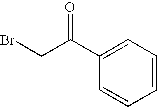 | 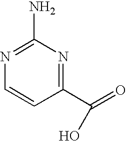 | A |
| 104 | 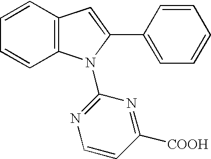 | 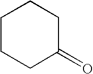 | 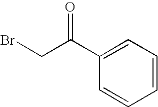 | 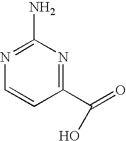 | A, C |
| 105 | 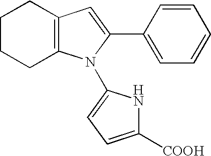 | 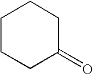 | 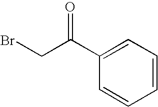 | 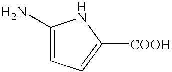 | A |
| 106 | 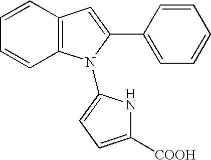 | 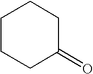 | 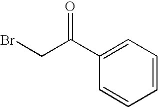 | 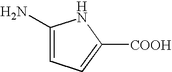 | A, C |
| 107 | 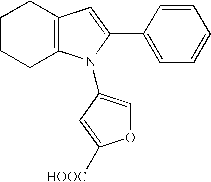 | 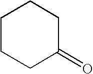 | 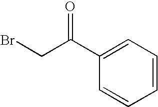 | 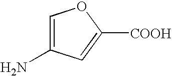 | A |
| 108 | 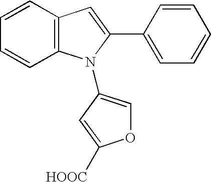 | 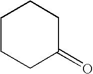 | 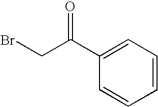 | 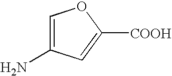 | A, C |
| 109 | 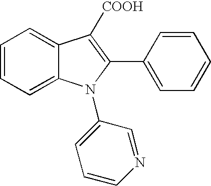 | 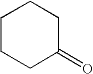 | 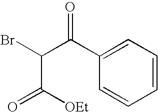 | 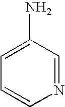 | A, C |
| 110 | 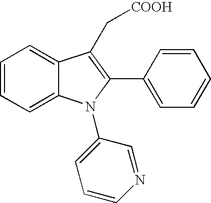 | 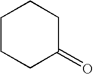 | 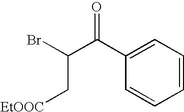 | 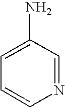 | A, C |
| 111 | 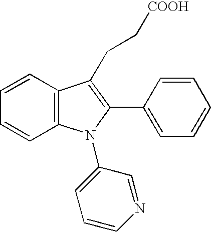 | 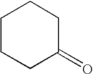 | 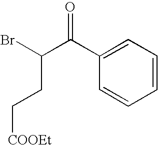 | 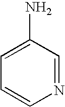 | A, C |
| 112 | 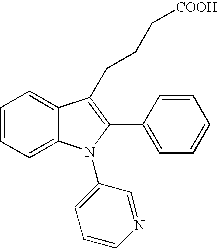 | 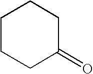 | 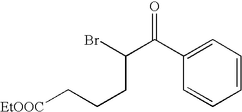 | 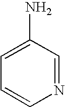 | A, C |
| 113 | 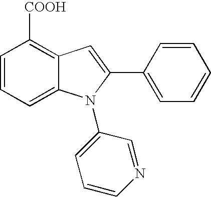 | 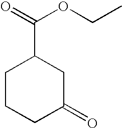 | 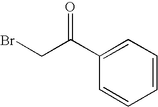 | 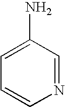 | A, C |
| 114 | 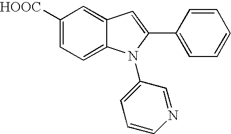 | 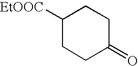 | 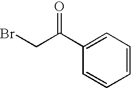 | 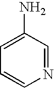 | A, C |
| 115 | 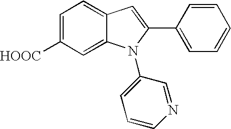 | 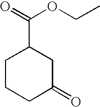 | 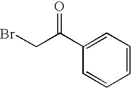 | 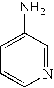 | A, C |
| 116 | 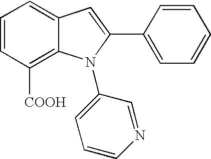 | 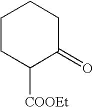 | 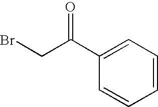 | 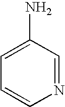 | A, C |
| 117 | 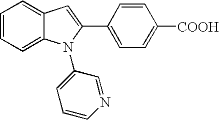 | 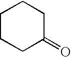 | 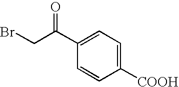 | 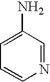 | A, C |
| 118 | 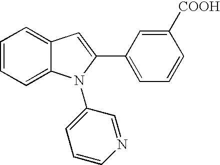 | 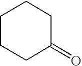 | 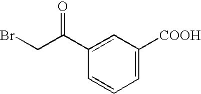 | 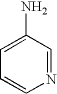 | A, C |
| 119 | 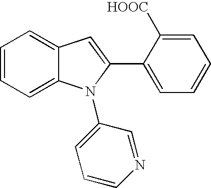 | 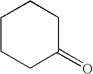 | 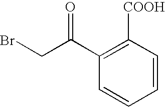 | 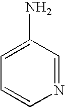 | A, C |
| 120 | 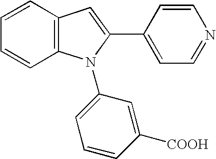 | 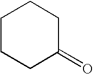 | 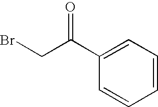 | 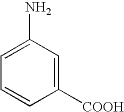 | A, C,B |
| 121 | 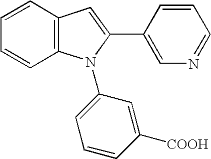 | 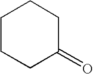 | 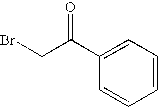 | 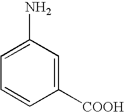 | A, C,B |
| 122 | 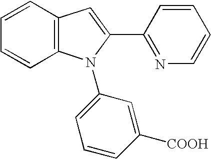 | 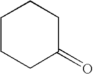 | 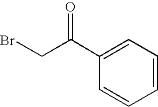 | 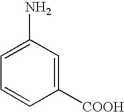 | A, C,B |
| 123 | 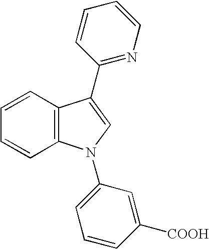 | 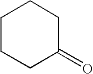 | 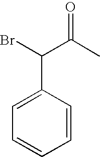 | 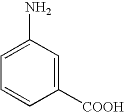 | A, C |
| 124 | 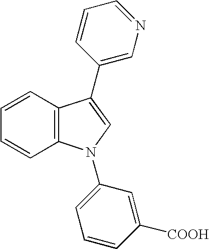 | 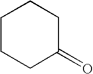 | 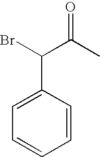 | 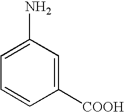 | A, C |
| 125 | 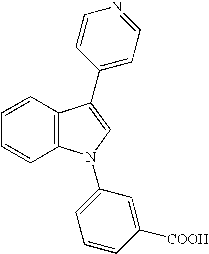 | 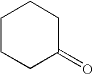 | 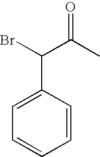 | 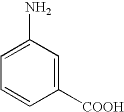 | A, C |
| 126 | 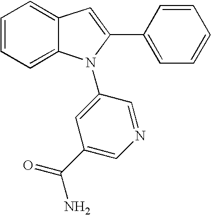 | 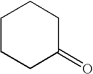 | 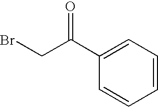 | 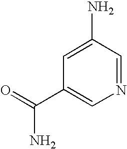 | A, C |
| 127 | 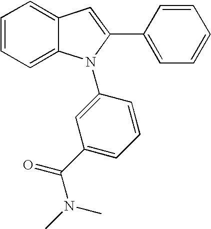 | 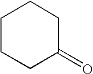 | 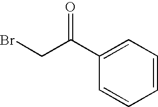 | 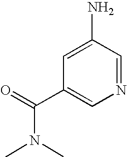 | A, C |
| 128 | 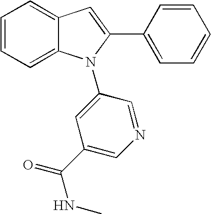 | 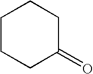 | 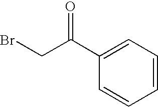 | 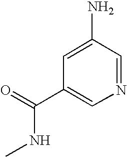 | A, C |
| 129 | 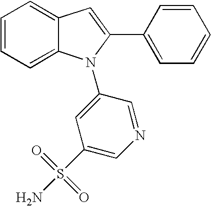 | 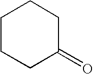 | 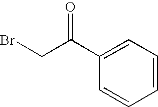 | 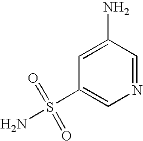 | A, C |
| 130 | 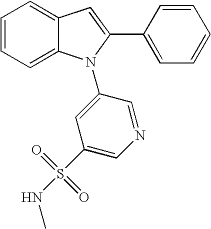 | 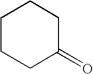 | 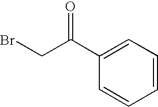 | 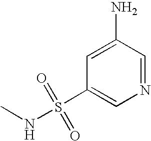 | A, C |
| 131 | 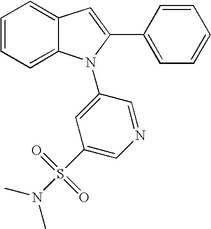 | 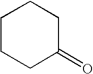 | 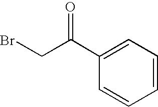 | 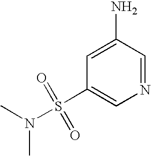 | A, C |
| 132 | 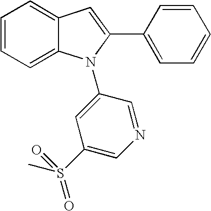 | 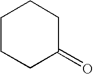 | 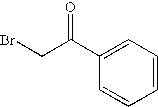 | 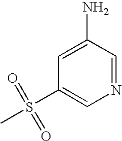 | A, C |

TABLE 4
Exemplary Compounds of the Invention

| Com- | | ketone/ | a-bromo | |
|---|---|---|---|---|
| pound | product | diketone | ketone | |
| Number | structure | SM | SM | aniline |
| 133 | 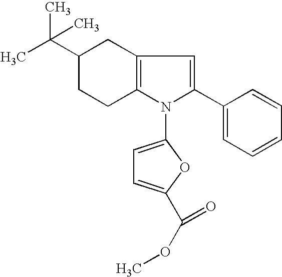 | 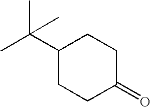 | 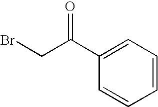 | 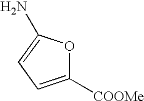 |
| 134 | 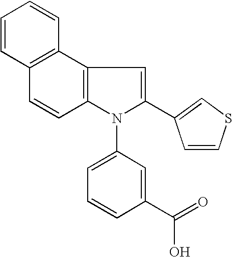 | 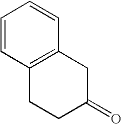 | 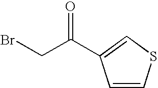 | 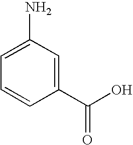 |
| 135 | 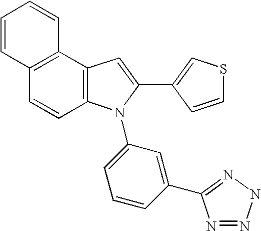 | 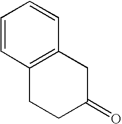 | 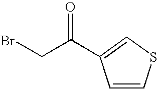 | 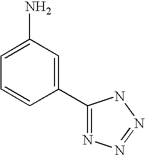 |
| 136 | 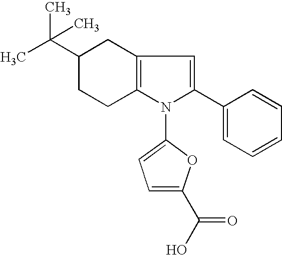 | 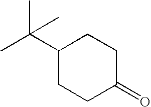 | 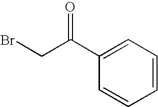 | 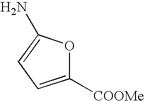 |
| 137 | 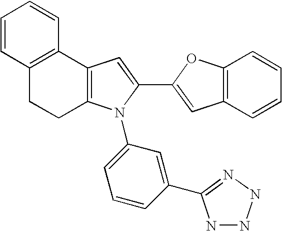 | 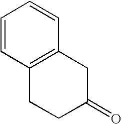 | 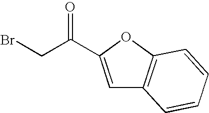 | 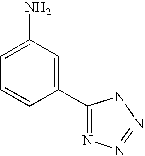 |
| 138 | 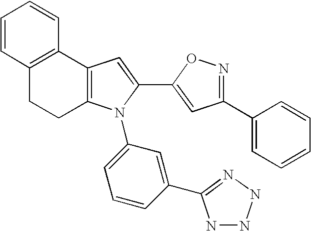 | 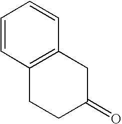 | 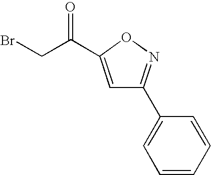 | 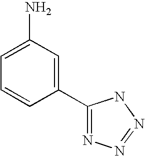 |
| 139 | 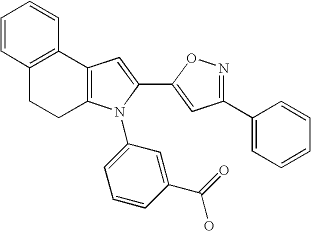 | 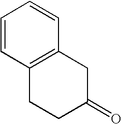 | 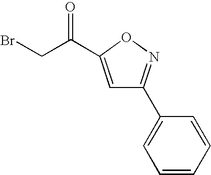 | 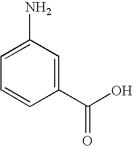 |
| 140 | 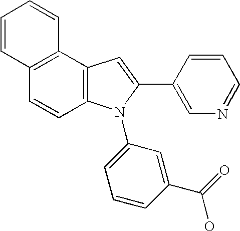 | 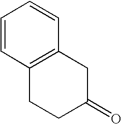 | 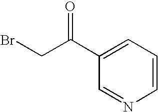 | 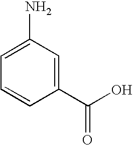 |
| 141 | 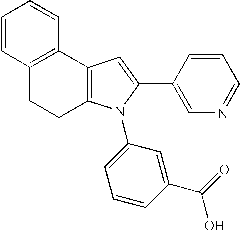 | 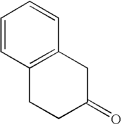 | 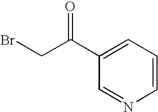 | 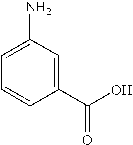 |
| 142 | 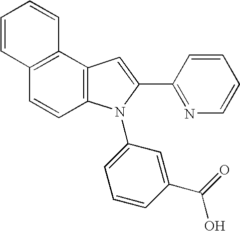 | 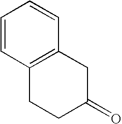 | 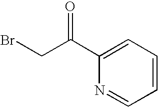 | 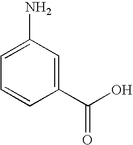 |
| 143 | 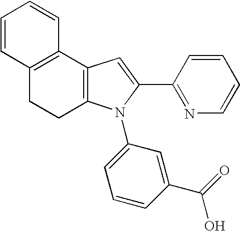 | 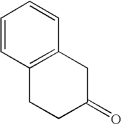 | 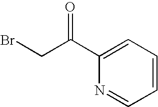 | 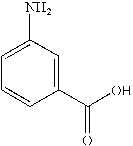 |
| 144 | 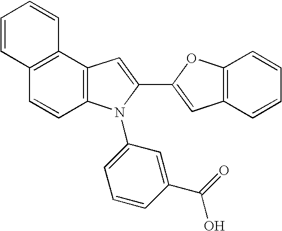 | 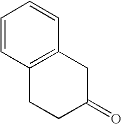 | 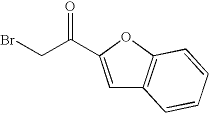 | 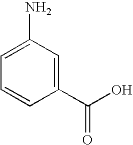 |
| 145 | 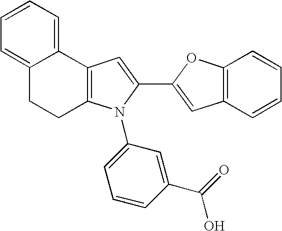 | 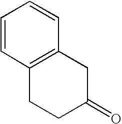 | 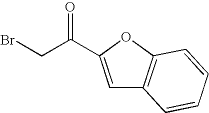 | 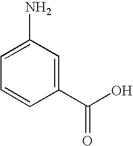 |
| 146 | 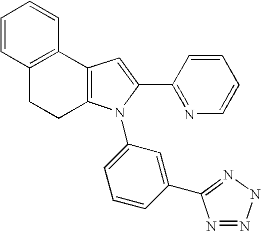 | 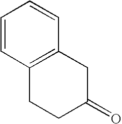 | 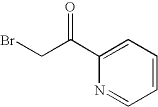 | 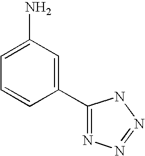 |
| 147 | 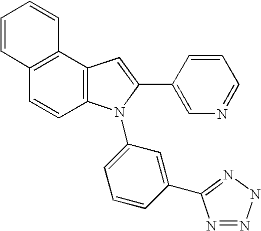 | 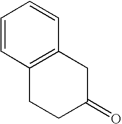 | 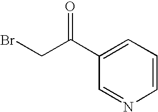 | 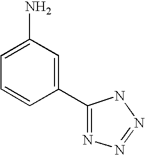 |
| 148 | 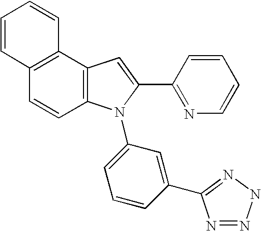 | 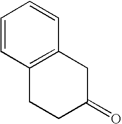 | 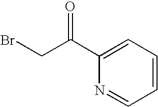 | 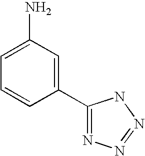 |

TABLE 5
Analytical data for the Compounds in Table 4

| Compound | | | | Synthetic |
|---|---|---|---|---|
| Number | 1H NMR, δ | MS | Name | route used |
| 133 | CDCl3; 7.1-7.3 | pos. mode | 5-(5-tertButyl-2-phenyl- | A |
| | (m, 6H, ArH); 6.2 | 378 (M + H). | 4,5,6,7-tetrahydroindol-1- | |
| | (s, 1H); 6.0 (d, | | yl) furan-2-carboxylic acid | |
| | 1H); 3.9 (s, 3H); | | methyl ester | |
| | 2.6 (m, 3H); 2.3 | | | |
| | (m, 1H); 2.o (m, | | | |
| | 1H); 1.3-1.5 (m, | | | |
| | 2H); 1.0 (s, 9H). | | | |
| 134 | DMSO - d6; 6.6- | pos. mode | 3-(2-thiophen-3-yl- | A, C |
| | 8.4(14H, ArH), | 370 (M + 1) | benzo[e]indol-3-yl) | |
| | | | benzoic acid | |
| 135 | DMSO - d6; 6.6- | pos. mode | 3-[3-(2H-tetrazol-5-yl)- | A, C |
| | 8.4(13H, ArH), | 396 (M + 1); | phenyl]-2-thiophen-3-yl- | |
| | 2.9(2H, CH2), | 394 (M − 1). | 3H benzo[e]indole | |
| | 2.6(2H,CH2) | | | |
| 136 | CDCl3; 7.1-7.3 | pos. mode | 5-(5-tertButyl-2-phenyl- | A |
| | (m, 6H, ArH); 6.2 | 364 (M + H); | 4,5,6,7-tetrahydroindol-1- | |
| | (s, 1H); 6.0 (d, | neg. mode | yl) furan-2-carboxylic acid | |
| | 1H); 2.6 (m, 2H); | 362 (M − H) | | |
| | 2.4-2.5 (m, 2H); | | | |
| | 2.0 (m, 1H); 1.5 | | | |
| | (m, 2H); 1.0 (s, | | | |
| | 9H). | | | |
| 137 | DMSO - d6; 7.1- | pos. mode | 2-benzofuran-2-yl-3-[3- | A |
| | 8.5 (14H, | 430 (M + 1); | (2H-tetrazol-5-yl)-phenyl]- | |
| | ArH/NH); 5.8 (1H); | neg. mode | 4,5-dihydro-3H- | |
| | 2.9 (2H, CH2); 2.6 | 429 (M − 1). | benzo[e]indole | |
| | (2H, CH2). | | | |
| 138 | DMSO - d6; 7.0- | pos. mode | 2-(3-phenylisoxazol-5-yl)- | A |
| | 8.2 (15H, | 457 (M + 1); | 3-[3-(2H-tetrazol-5-yl)- | |
| | ArH/NH); 6.3 (1H); | neg mode | phenyl]-4,5-dihydro-3H- | |
| | 2.9 (2H, CH2); 2.6 | 455 (M − 1). | benzo[e]indole | |
| | (2H, CH2). | | | |
| 139 | DMSO d6; 7.0-8.1 | pos. mode | 3-(2-phenylisoxazol-5-yl)- | A |
| | (14H, ArH); 6.2 | 433 (M + 1); | 4,5-dihydrobenzo[e]indol- | |
| | (1H); 2.9 (2H, | heg. mode | 3-yl] benzoic acid | |
| | CH2); 2.6 (2H, | 431 (M − 1). | | |
| | CH2). | | | |
| 140 | DMSO - d6; 7.3- | pos. mode | 3-(2-pyridin-3-yl- | A, C |
| | 8.8 (15H, ArH). | 365 (M + 1). | benzo[e]indol-3-yl) | |
| | | | benzoic acid | |
| 141 | DMSO - d6; 7.0- | pos. mode | 3-(2-pyridin-3-yl-4,5- | A |
| | 8.6 (13H, ArH), 2.9 | 367 (M + 1). | dihydrobenzo[e]indol-3- | |
| | (2H, CH2), 2.6 | | yl) benzoic acid | |
| | (2H, CH2). | | | |
| 142 | DMSO - d6; 7.2- | pos. mode | 3-(2-pyridin-2-yl- | A, C |
| | 8.5(15H, ArH). | 365 (M + 1). | benzo[e]indol-3-yl) | |
| | | | benzoic acid | |
| 143 | DMSO - d6; 6.9- | pos. mode | 3-(2-pyridin-2-yl-4,5- | A |
| | 8.5 (13H, ArH), 2.9 | 367 (M + 1); | dihydrobenzo[e]indol-3- | |
| | (2H, CH2), 2.6 | 365 (M + 1). | yl) benzoic acid | |
| | (2H, CH2). | | | |
| 144 | DMSO - d6; 7.1- | pos. mode | 3-(2-benzofuran-2-yl- | A, C |
| | 8.2 (16H, ArH). | 404 (M + 1). | benzo[e]indol-3-yl) | |
| | | | benzoic acid | |
| 145 | DMSO - d6; 7.0- | pos. mode | 3-(2-benzofuran-2-yl-4,5- | A |
| | 8.1 (14H, ArH), 2.9 | 406 (M + 1). | dihydrobenzo[e]indol-3- | |
| | (2H, CH2), 2.6 | | yl) benzoic acid | |
| | (2H, CH2). | | | |
| 146 | DMSO - d6; 7.0- | pos. mode | 2-pyridin-2-yl-3-[3-(2H- | A |
| | 8.6 (14H, ArH), 2.9 | 391 (M + 1). | tetrazol-5-yl)-phenyl]-4,5- | |
| | (2H, CH2), 2.6 | | dihydro-3H- | |
| | (2H, CH2). | | benzo[e]indole | |
| 147 | DMSO - d6; 7.4- | pos. mode | 2-pyridin-3-yl-3-[3-(2H- | A, C |
| | 8.6(16H, ArH). | 389 (M + 1). | tetrazol-5-yl)-phenyl]-3H- | |
| | | | benzo[e]indole | |
| 148 | DMSO - d6; 7.2- | pos. mode | 2-pyridin-2-yl-3-[3-(2H- | A, C |
| | 8.4 (16H, ArH). | 389 (M + 1). | tetrazol-5-yl)-phenyl]-3H- | |
| | | | benzo[e]indole | |

Compounds of Formulae I and II, e.g., those disclosed in Table 4 and 5, are capable of modulating APP processing and lower Ab42 in the cell based assay described in Example 6. Compounds 138 and 139 have an Ab42 lowering IC50 of 10 μM and 2 μM, respectively.

### Example 12

### More Compounds of the Invention

Additional compounds of the invention, synthesized according to the above described routes are given below along with relevant characterization data. These compounds exemplify the compounds of the invention including those of aspects 1-21 of the invention.

TABLE 6
Compounds of the Invention and Starting Materials

| | | a-bromo | |
|---|---|---|---|
| product | ketone | ketone | aniline/ |
| structure | SM | SM | amine |
| 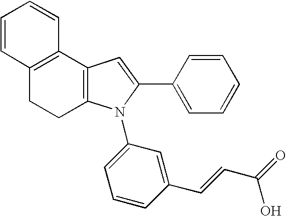 | 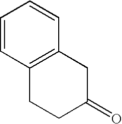 | 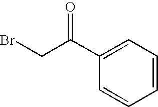 | 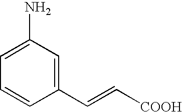 |
| 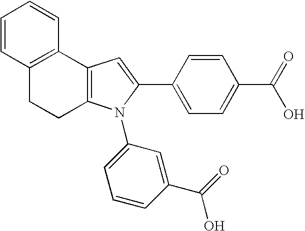 | 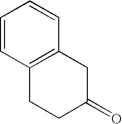 | 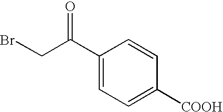 | 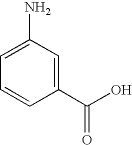 |
| 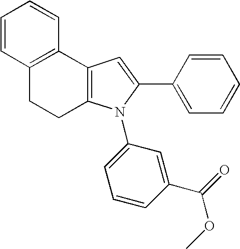 | 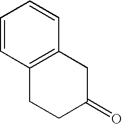 | 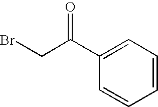 | 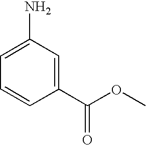 |
| 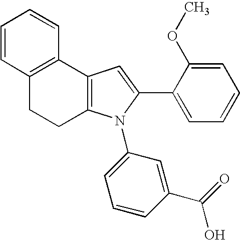 | 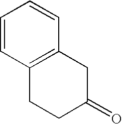 | 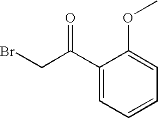 | 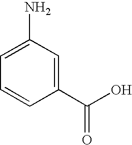 |
| 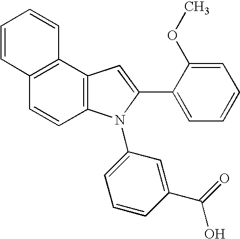 | 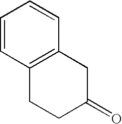 | 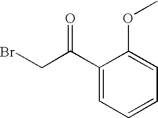 | 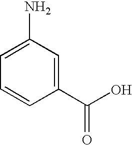 |
| 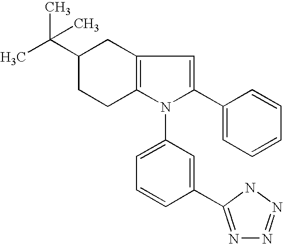 | 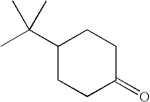 | 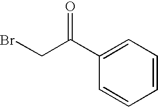 | 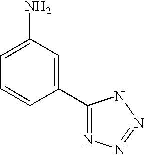 |
| 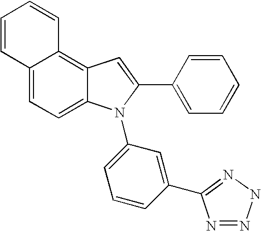 | 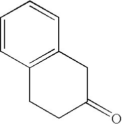 | 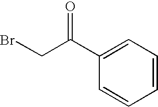 | 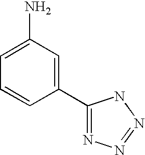 |
| 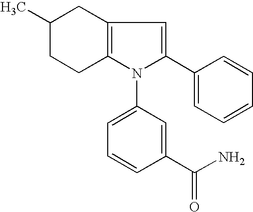 | 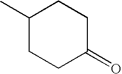 | 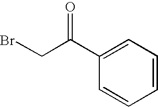 | 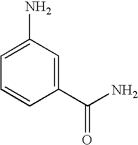 |
| 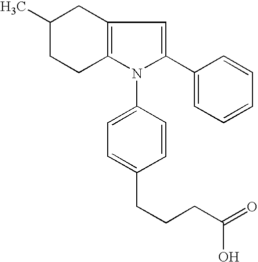 | 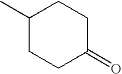 | 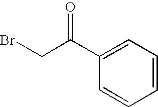 | 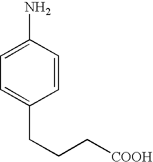 |
| 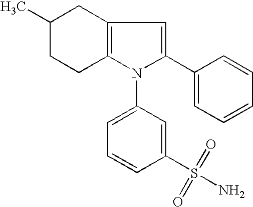 | 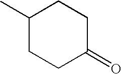 | 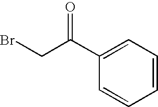 | 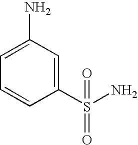 |
| 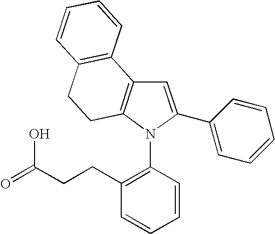 | 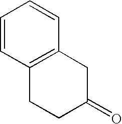 | 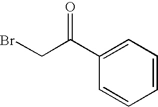 | 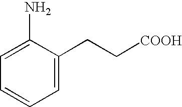 |
| 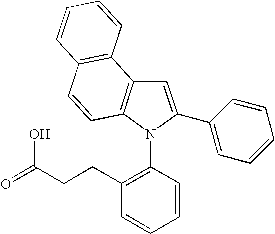 | 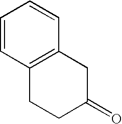 | 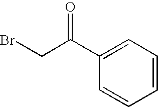 | 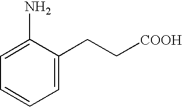 |
| 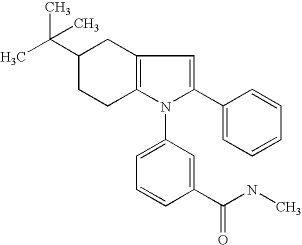 | 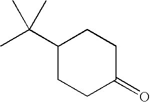 | 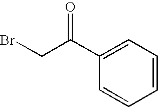 | 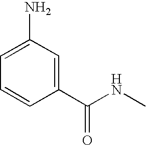 |
| 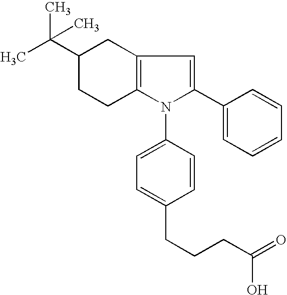 | 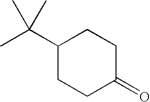 | 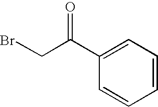 | 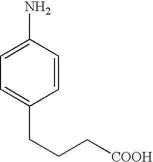 |
| 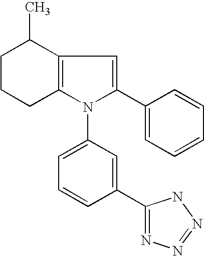 | 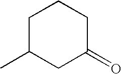 | 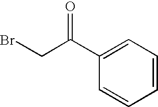 | 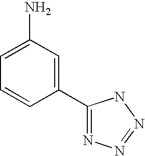 |
| 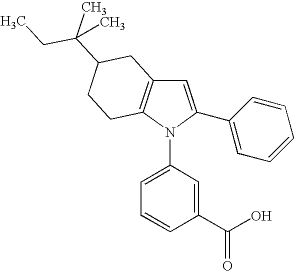 | 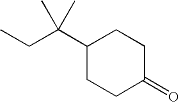 | 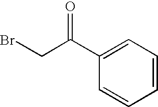 | 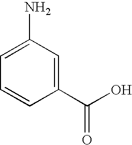 |
| 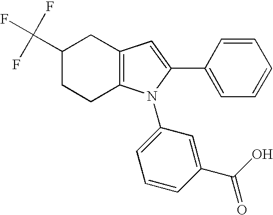 | 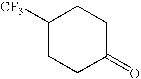 | 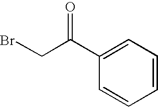 | 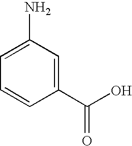 |
| 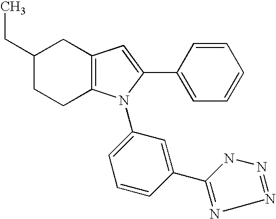 | 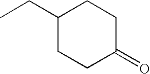 | 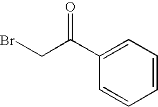 | 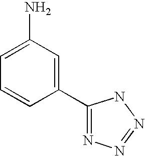 |
| 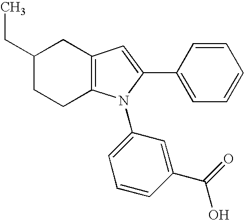 | 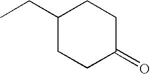 | 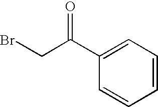 | 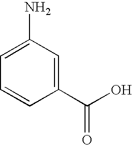 |
| 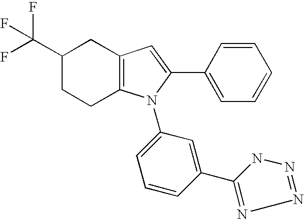 | 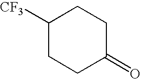 | 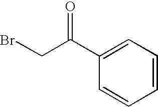 | 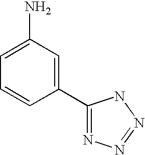 |
| 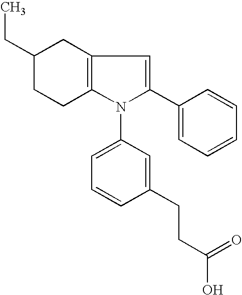 | 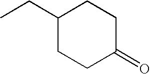 | 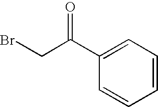 | 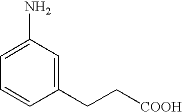 |
| 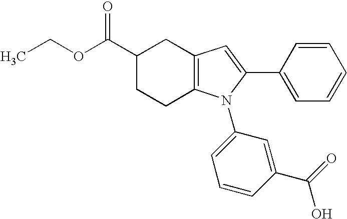 | 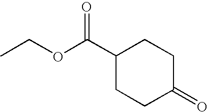 | 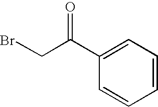 | 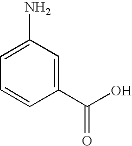 |
| 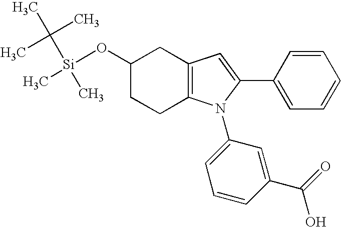 | 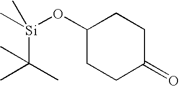 | 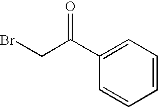 | 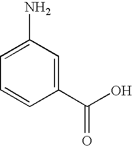 |
| 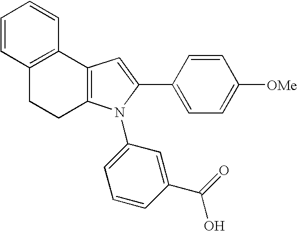 | 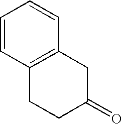 | 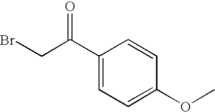 | 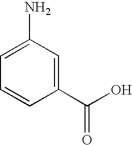 |
| 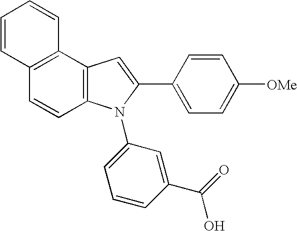 | 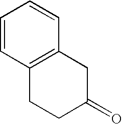 | 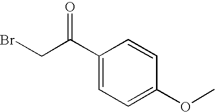 | 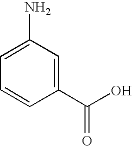 |
| 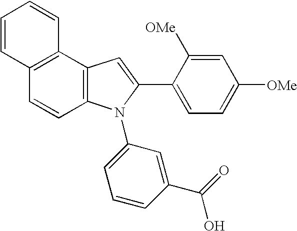 | 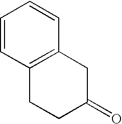 | 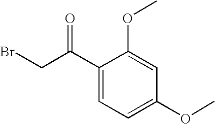 | 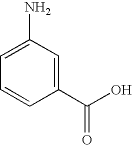 |
| 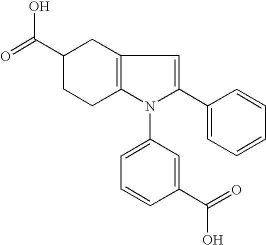 | 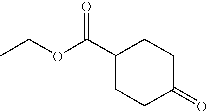 | 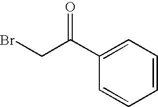 | 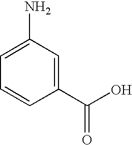 |
| 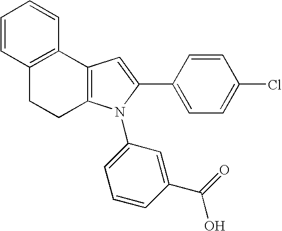 | 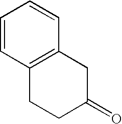 | 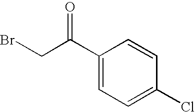 | 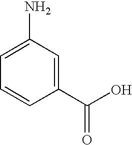 |
| 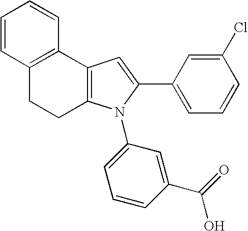 | 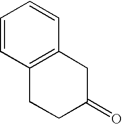 | 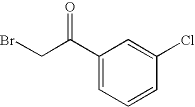 | 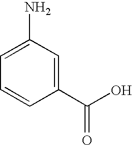 |
| 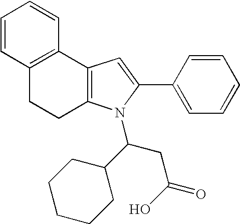 | 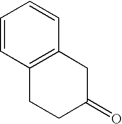 | 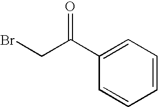 | 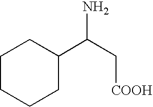 |
| 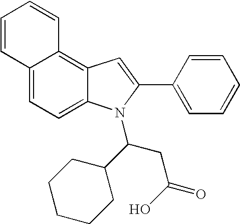 | 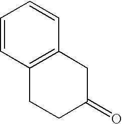 | 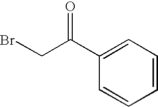 | 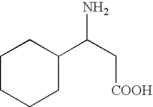 |
| 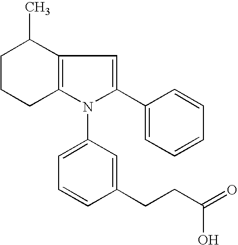 | 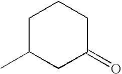 | 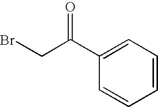 | 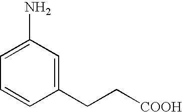 |
| 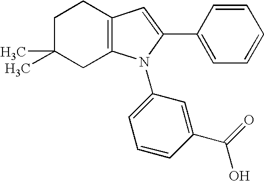 | 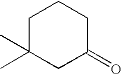 | 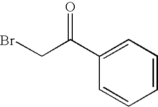 | 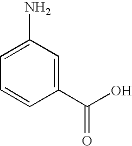 |
| 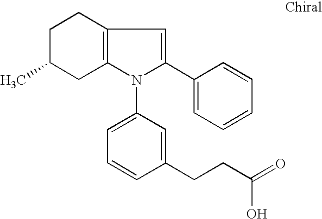 | 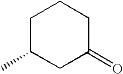 | 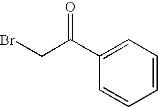 | 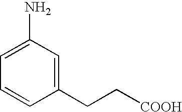 |
| 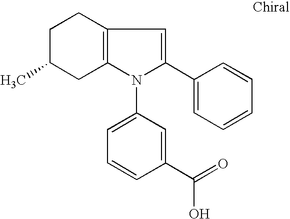 | 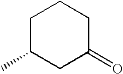 | 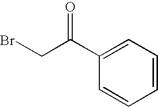 | 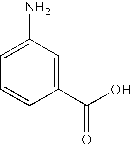 |
| 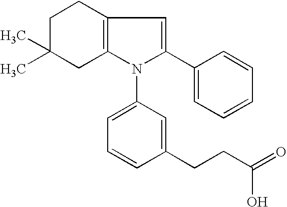 | 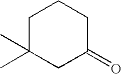 | 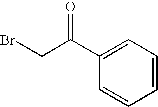 | 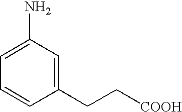 |
| 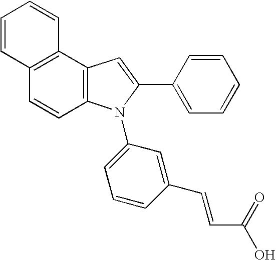 | 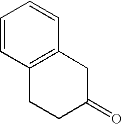 | 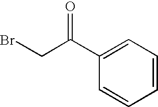 | 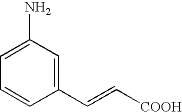 |
| 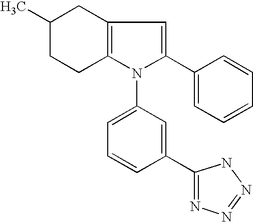 | 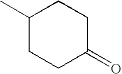 | 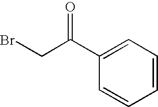 | 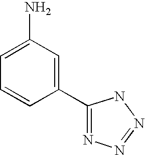 |
| 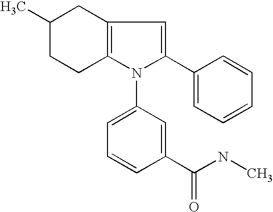 | 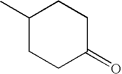 | 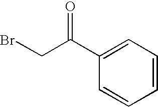 | 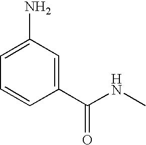 |
| 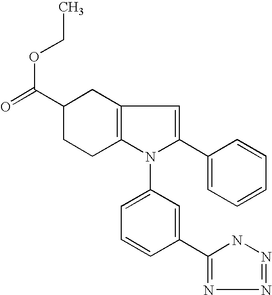 | 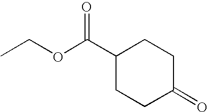 | 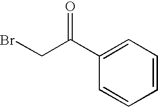 | 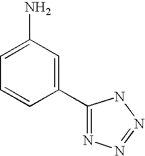 |
| 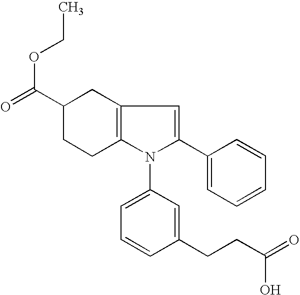 | 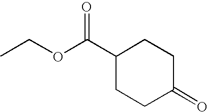 | 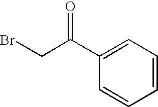 | 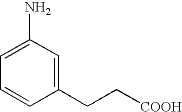 |
| 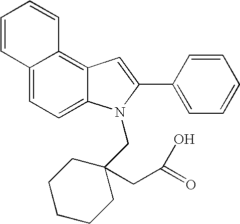 | 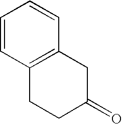 | 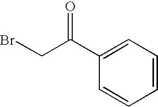 | 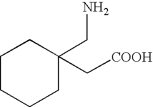 |
| 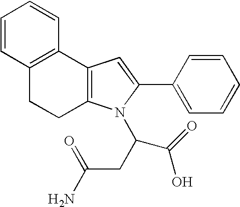 | 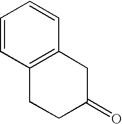 | 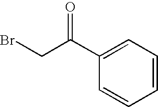 | 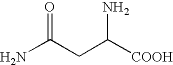 |
| 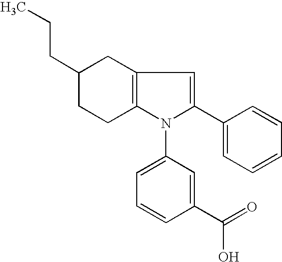 | 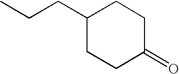 | 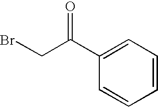 | 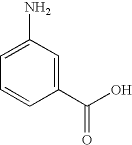 |
| 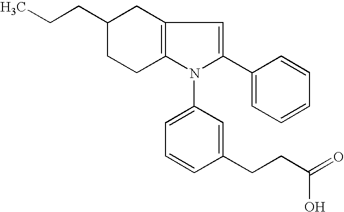 | 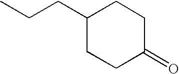 | 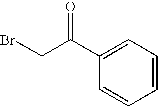 | 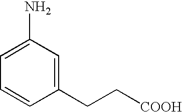 |
| 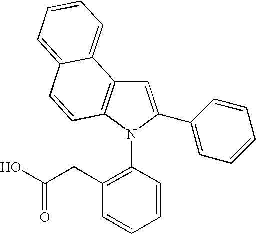 | 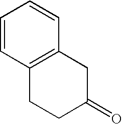 | 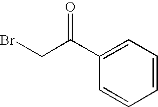 | 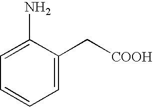 |
| 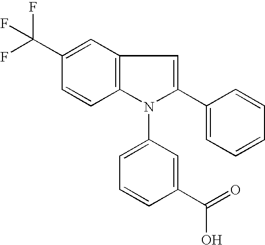 | 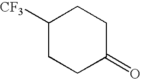 | 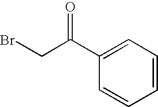 | 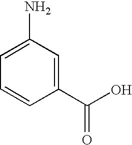 |
| 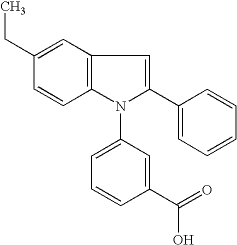 | 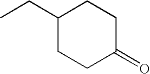 | 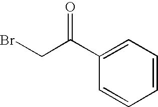 | 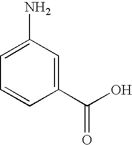 |
| 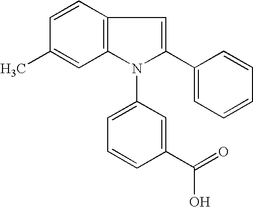 | 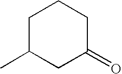 | 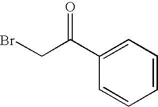 | 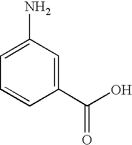 |
| 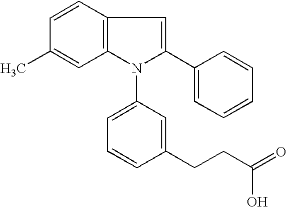 | 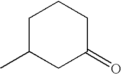 | 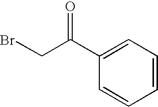 | 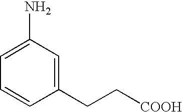 |
| 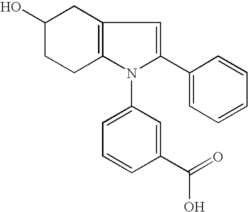 | 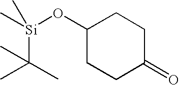 | 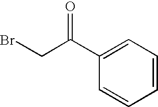 | 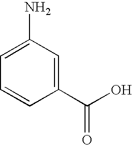 |
| 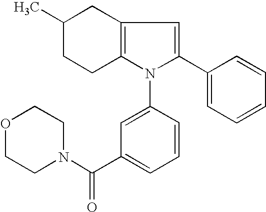 | 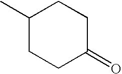 | 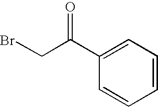 | 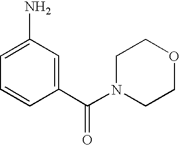 |
| 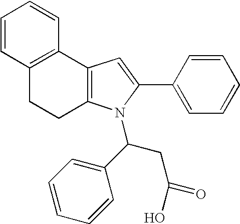 | 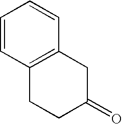 | 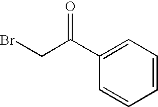 | 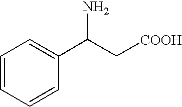 |
| 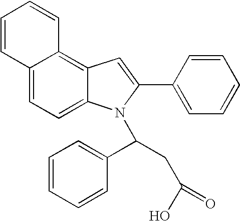 | 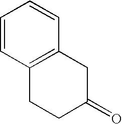 | 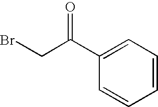 | 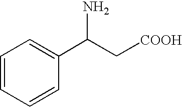 |
| 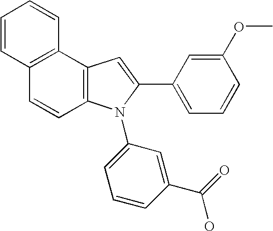 | 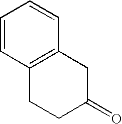 | 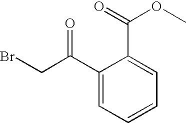 | 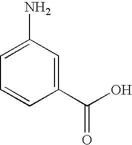 |
| 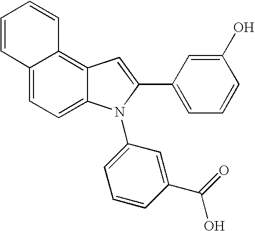 | 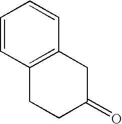 | 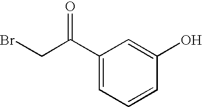 | 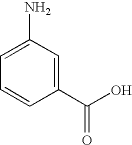 |
| 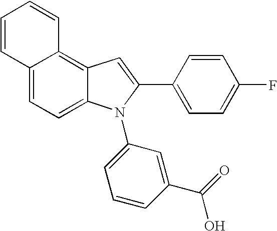 | 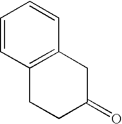 | 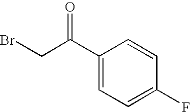 | 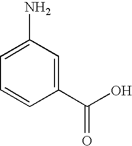 |
| 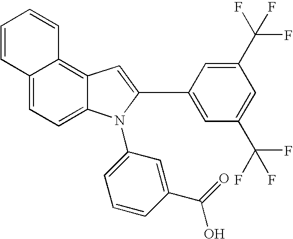 | 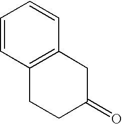 | 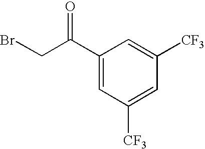 | 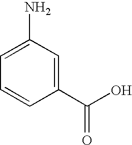 |
| 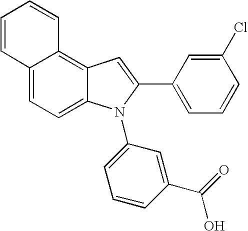 | 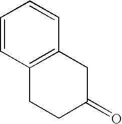 | 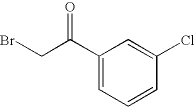 | 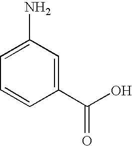 |
| 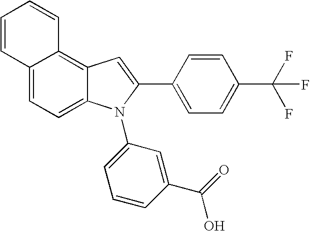 | 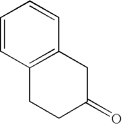 | 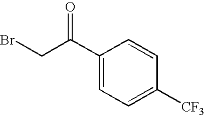 | 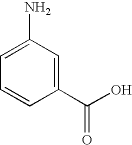 |
| 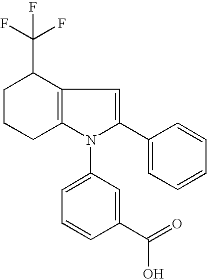 | 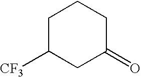 | 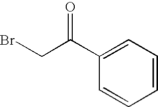 | 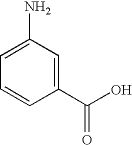 |
| 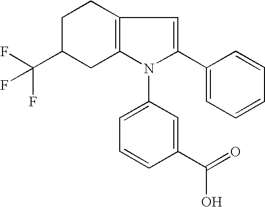 | 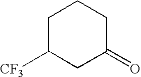 | 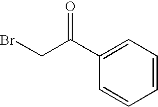 | 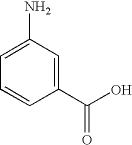 |
| 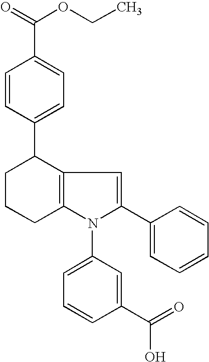 | 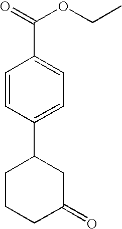 | 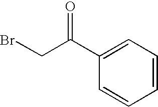 | 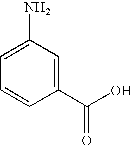 |
| 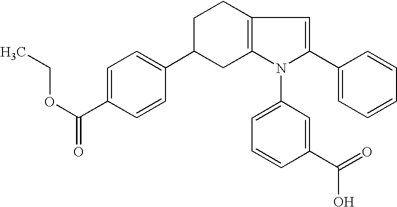 | 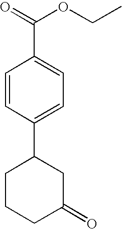 | 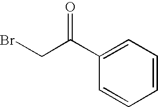 | 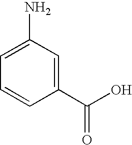 |
| 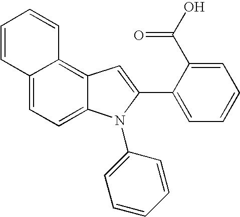 | 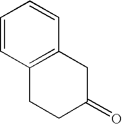 | 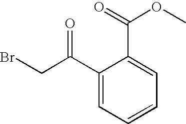 | 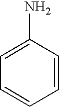 |
| 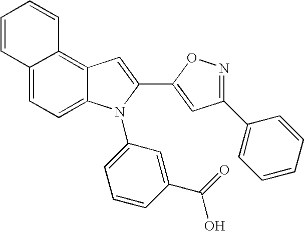 | 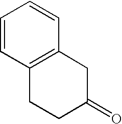 | 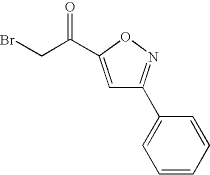 | 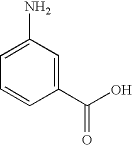 |
| 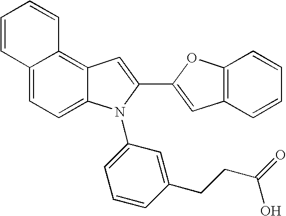 | 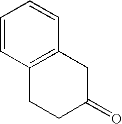 | 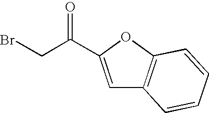 | 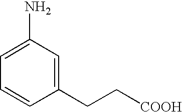 |
| 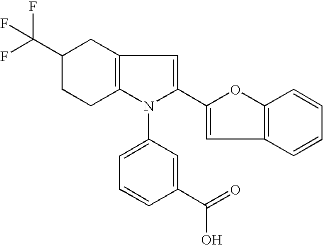 | 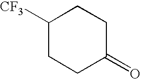 | 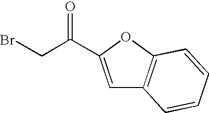 | 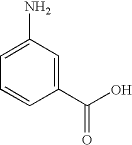 |
| 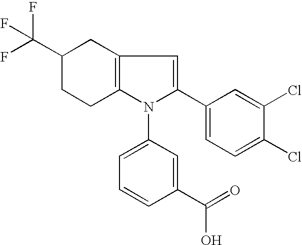 | 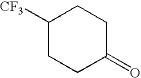 | 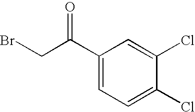 | 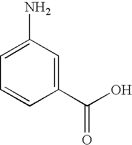 |
| 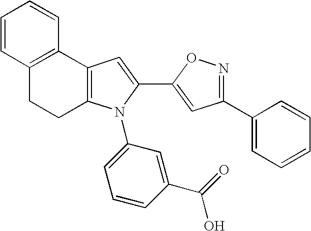 | 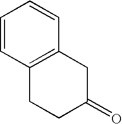 | 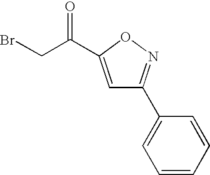 | 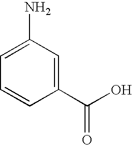 |
| 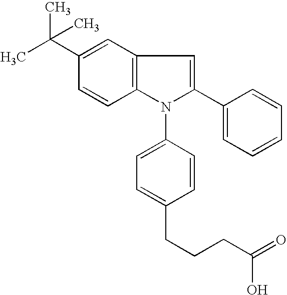 | 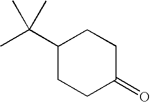 | 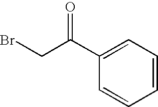 | 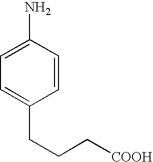 |
| 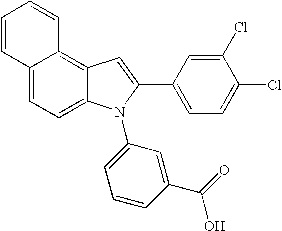 | 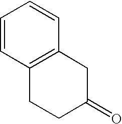 | 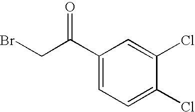 | 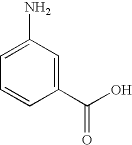 |
| 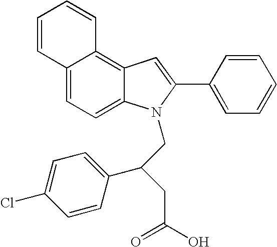 | 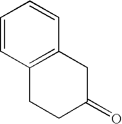 | 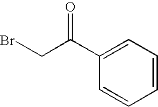 | 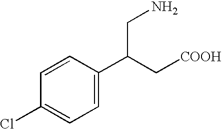 |
| 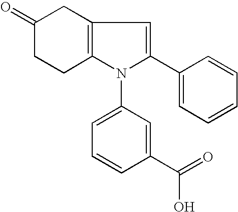 | 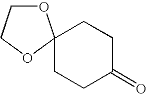 | 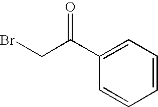 | 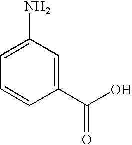 |
| 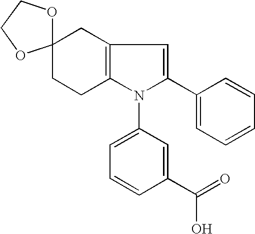 | 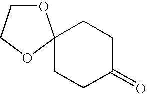 | 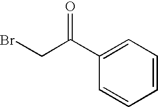 | 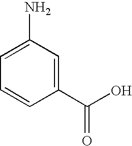 |
| 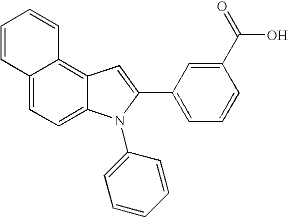 | 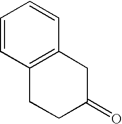 | 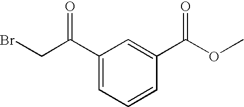 | 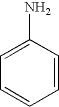 |
| 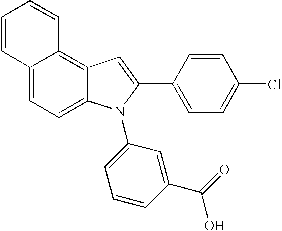 | 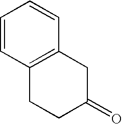 | 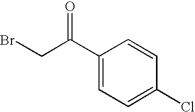 | 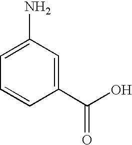 |
| 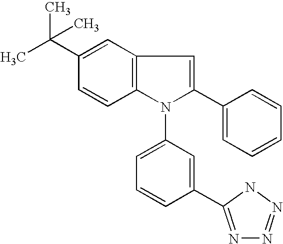 | 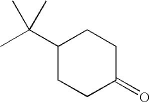 | 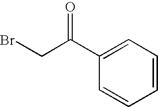 | 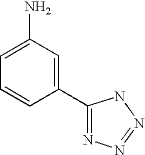 |
| 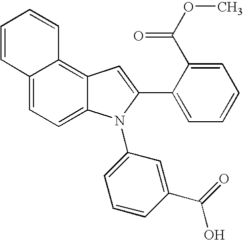 | 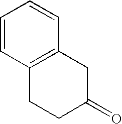 | 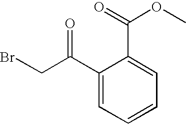 | 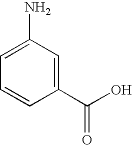 |
| 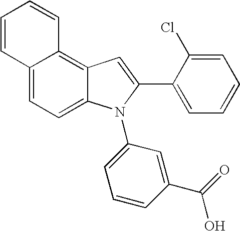 | 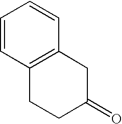 | 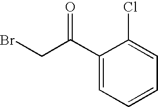 | 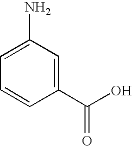 |
| 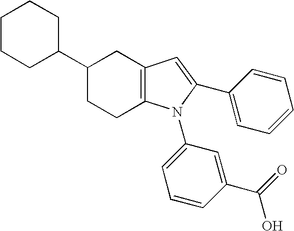 | 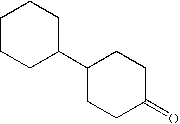 | 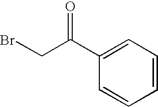 | 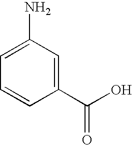 |
| 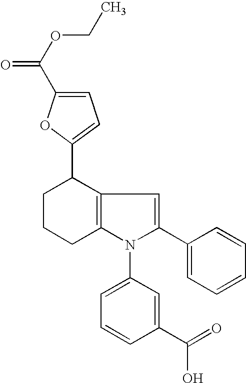 | 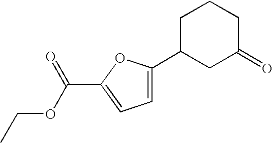 | 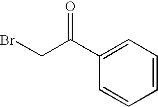 | 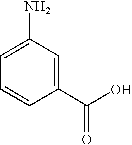 |
| 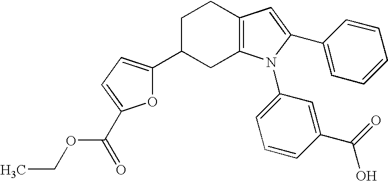 | 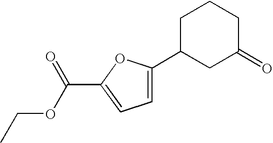 | 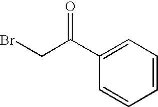 | 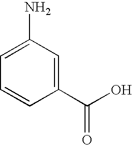 |
| 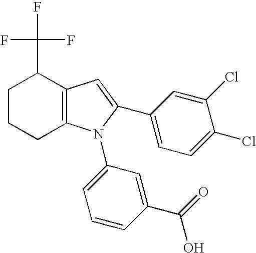 | 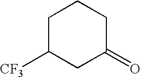 | 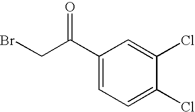 | 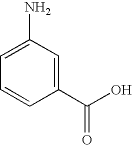 |
| 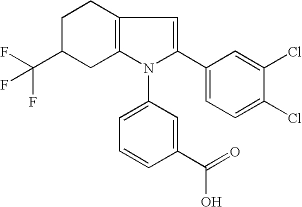 | 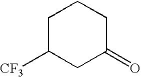 | 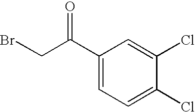 | 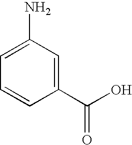 |
| 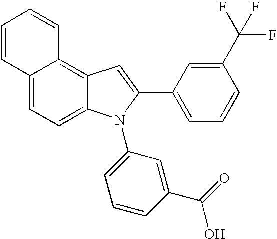 | 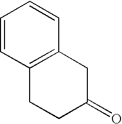 | 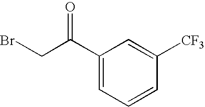 | 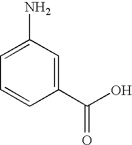 |
| 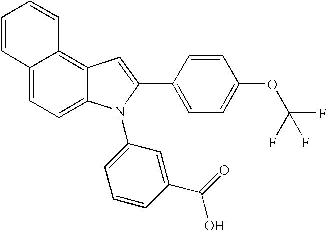 | 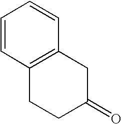 | 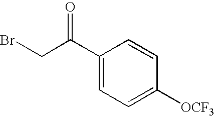 | 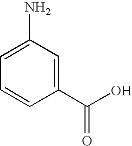 |
| 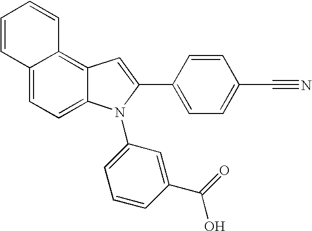 | 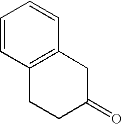 | 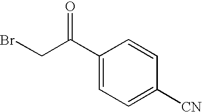 | 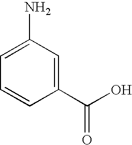 |
| 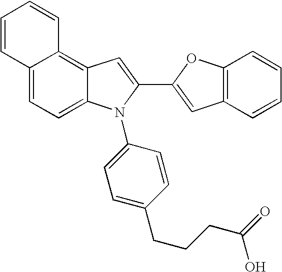 | 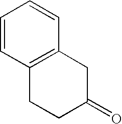 | 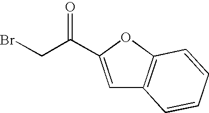 | 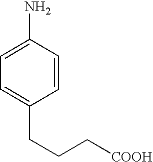 |
| 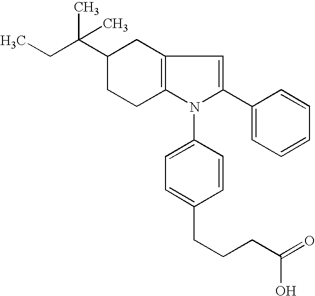 | 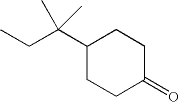 | 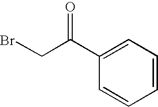 | 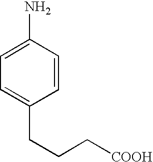 |
| 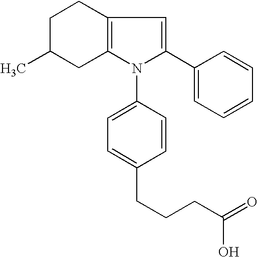 | 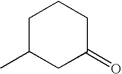 | 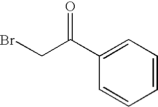 | 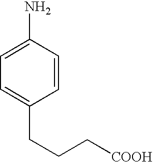 |
| 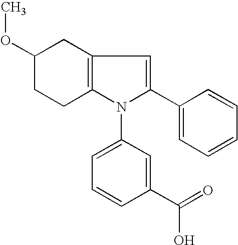 | 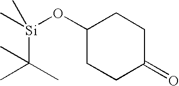 | 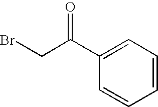 | 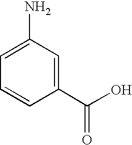 |
| 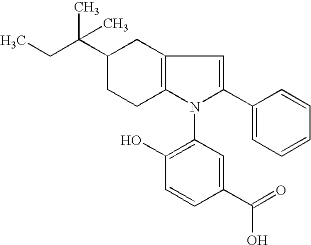 | 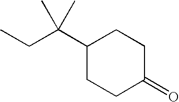 | 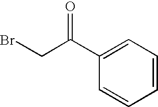 | 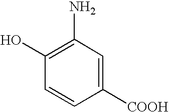 |
| 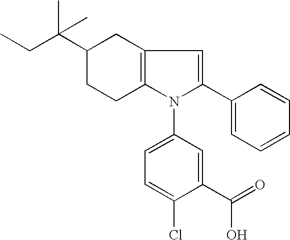 | 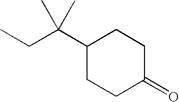 | 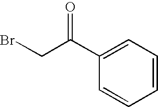 | 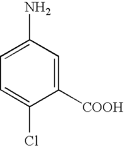 |
| 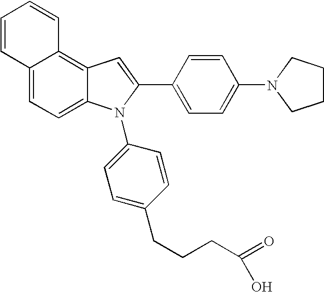 | 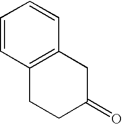 | 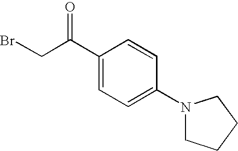 | 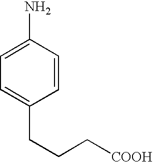 |
| 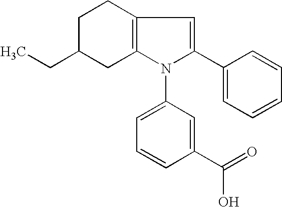 | 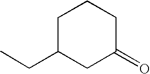 | 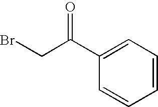 | 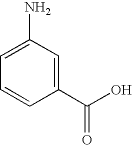 |
| 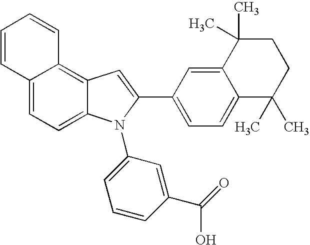 | 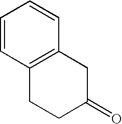 | 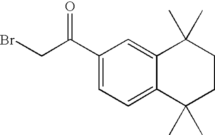 | 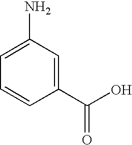 |
| 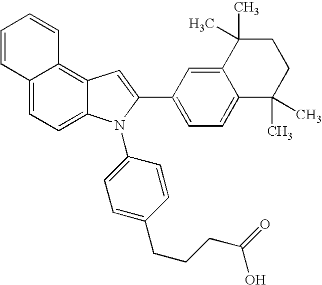 | 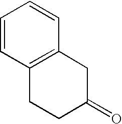 | 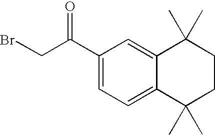 | 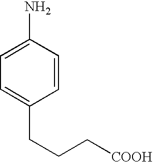 |
| 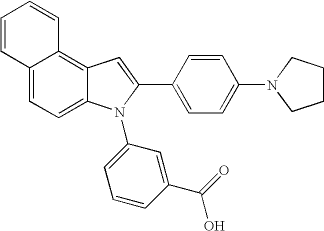 | 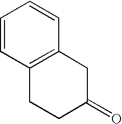 | 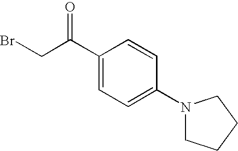 | 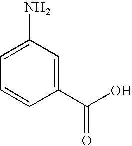 |
| 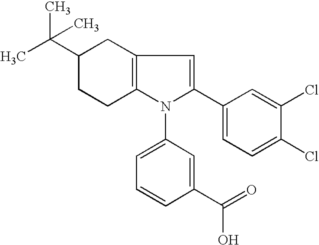 | 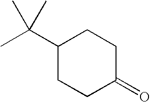 | 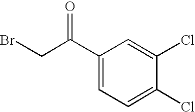 | 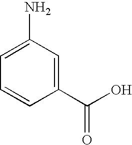 |
| 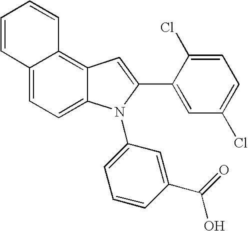 | 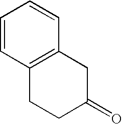 | 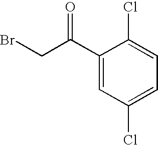 | 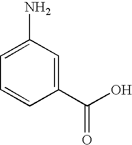 |
| 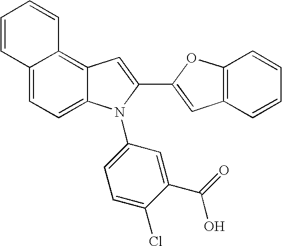 | 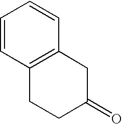 | 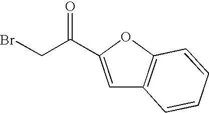 | 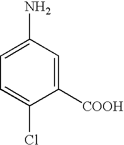 |
| 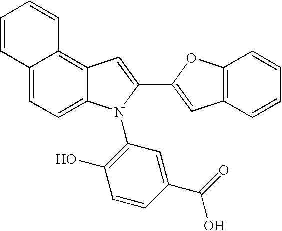 | 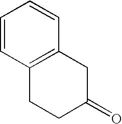 | 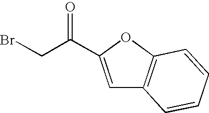 | 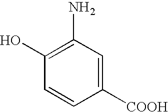 |
| 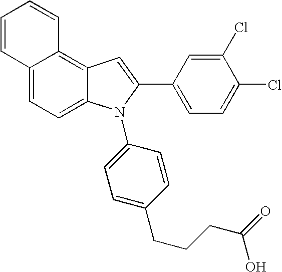 | 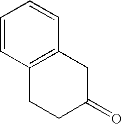 | 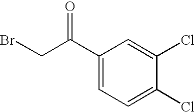 | 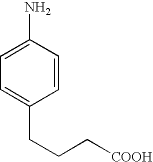 |
| 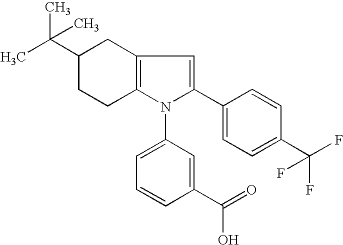 | 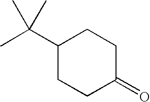 | 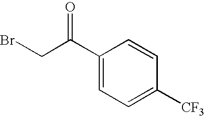 | 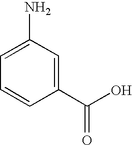 |
| 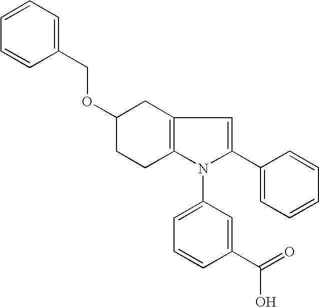 | 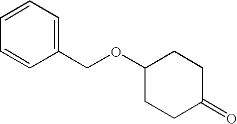 | 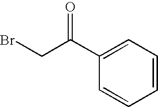 | 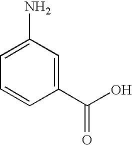 |
| 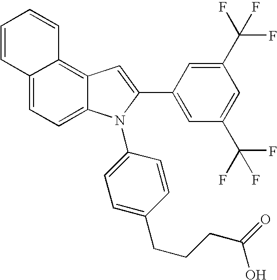 | 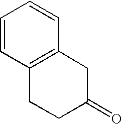 | 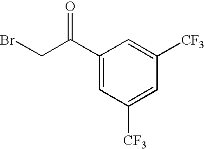 | 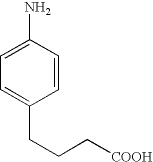 |
| 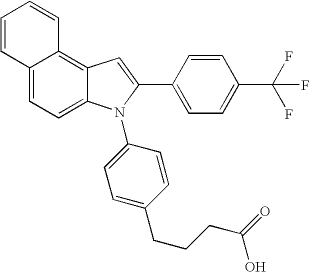 | 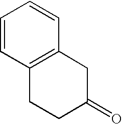 | 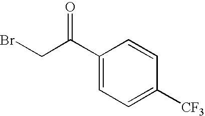 | 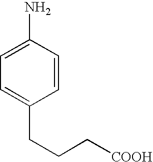 |
| 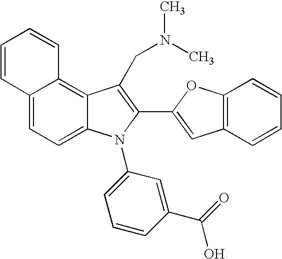 | 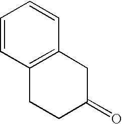 | 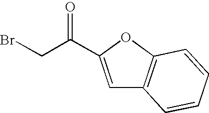 | 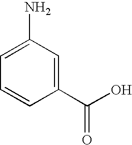 |
| 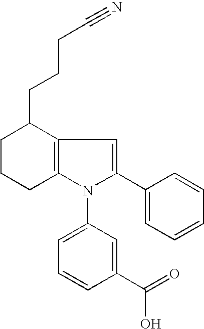 | 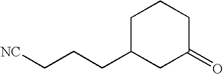 | 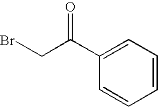 | 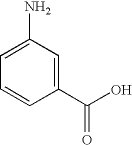 |
| 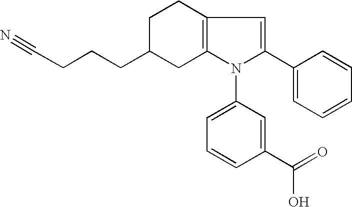 | 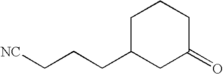 | 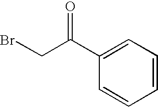 | 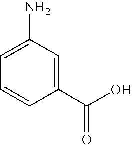 |
| 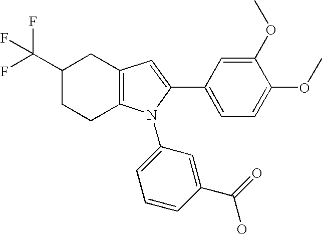 | 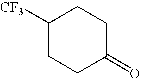 | 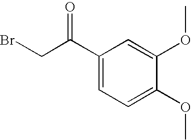 | 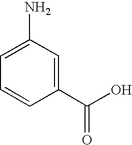 |
| 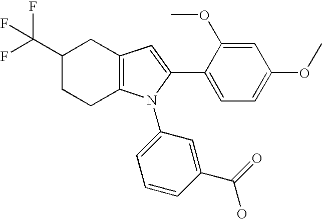 | 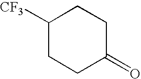 | 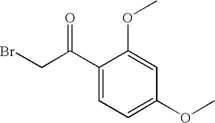 | 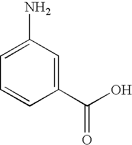 |
| 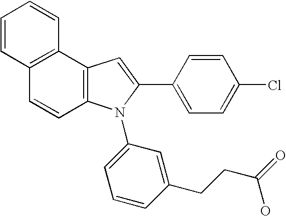 | 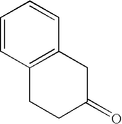 | 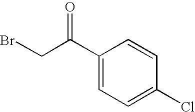 | 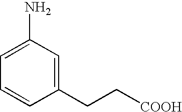 |
| 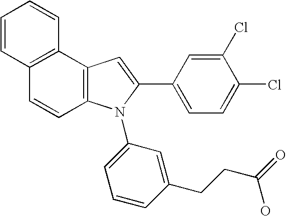 | 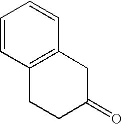 | 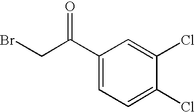 | 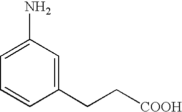 |
| 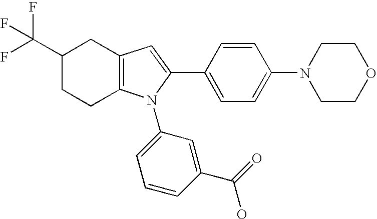 | 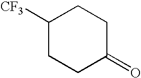 | 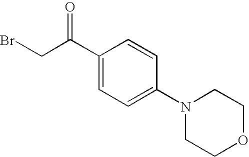 | 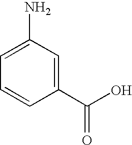 |
| 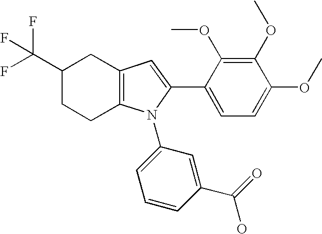 | 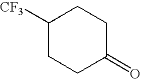 | 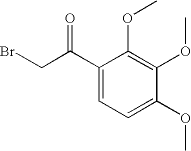 | 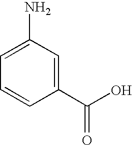 |
| 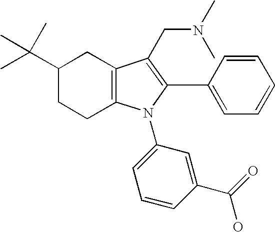 | 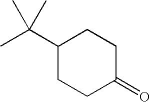 | 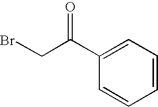 | 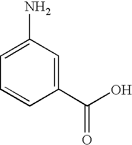 |
| 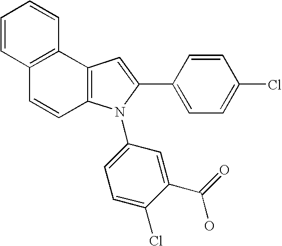 | 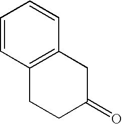 | 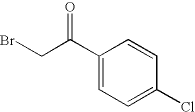 | 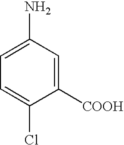 |
| 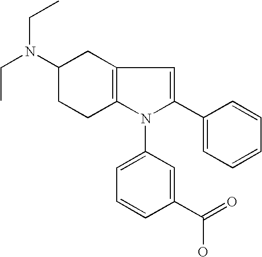 | 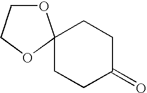 | 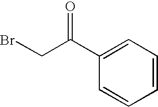 | 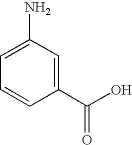 |
| 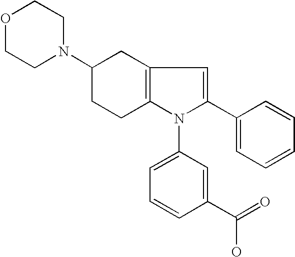 | 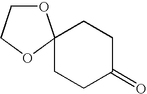 | 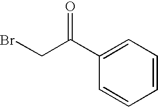 | 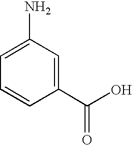 |
| 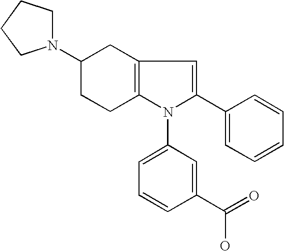 | 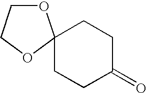 | 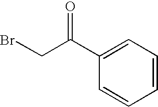 | 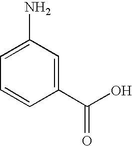 |
| 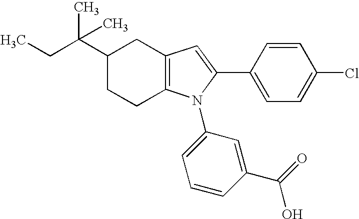 | 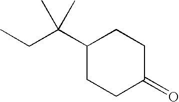 | 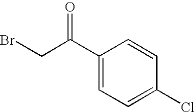 | 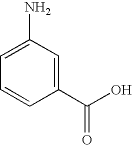 |
| 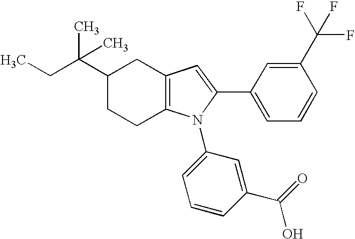 | 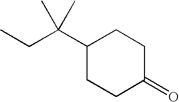 | 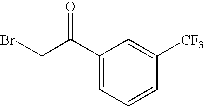 | 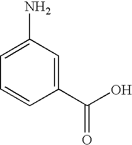 |
| 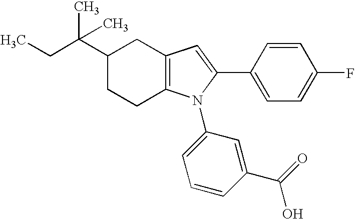 | 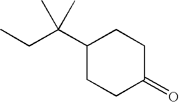 | 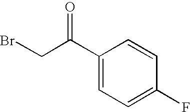 | 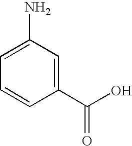 |
| 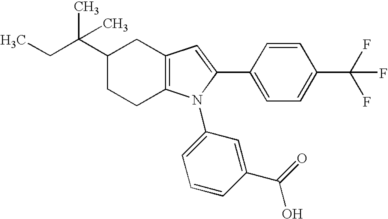 | 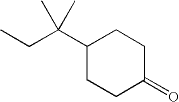 | 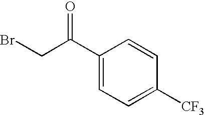 | 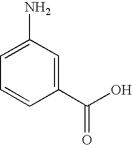 |
| 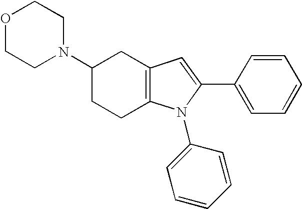 | 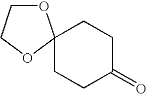 | 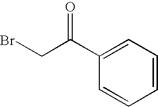 | 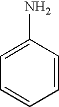 |
| 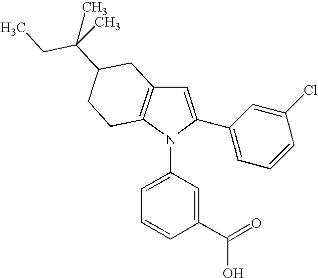 | 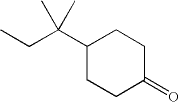 | 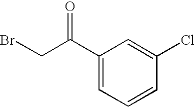 | 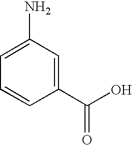 |
| 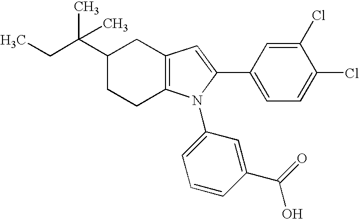 | 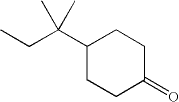 | 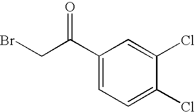 | 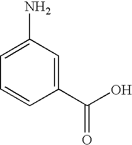 |
| 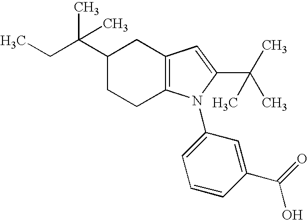 | 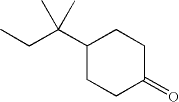 | 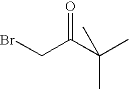 | 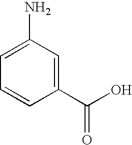 |
| | 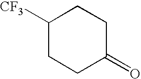 | 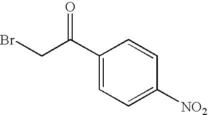 | 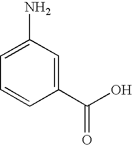 |
| 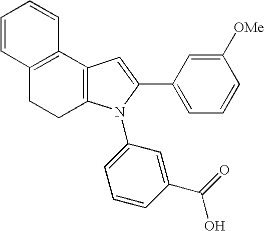 | 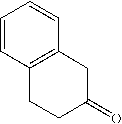 | 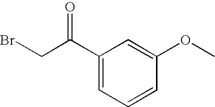 | 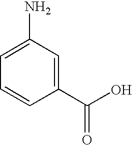 |
| 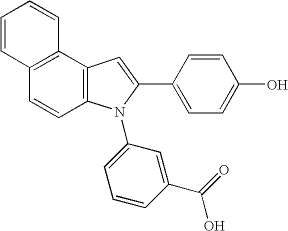 | 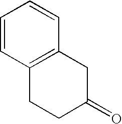 | 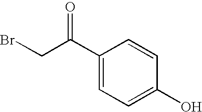 | 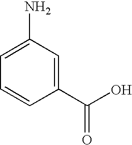 |
| 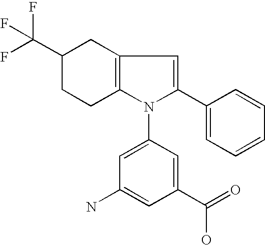 | 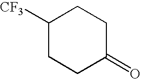 | 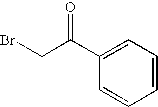 | 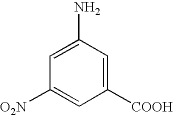 |
| 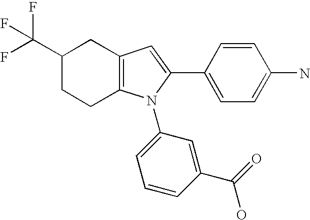 | 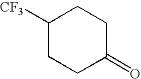 | 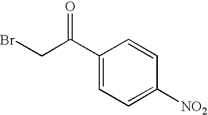 | 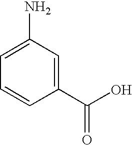 |
| 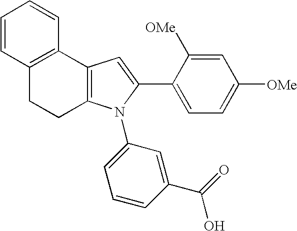 | 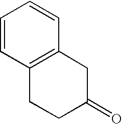 | 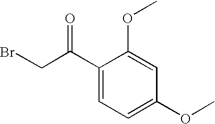 | 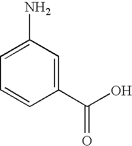 |
| 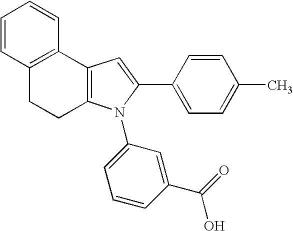 | 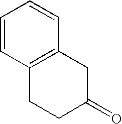 | 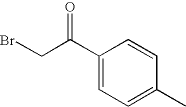 | 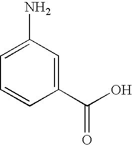 |
| 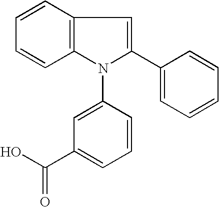 | 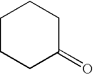 | 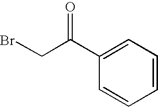 | 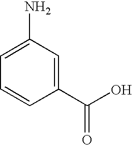 |
| 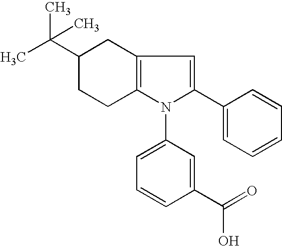 | 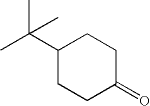 | 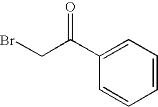 | 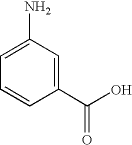 |
| 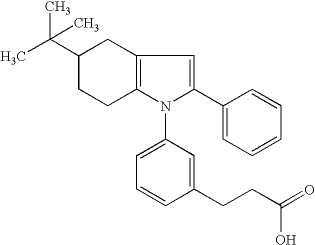 | 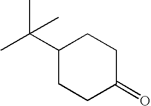 | 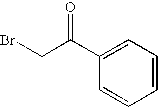 | 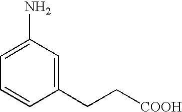 |
| 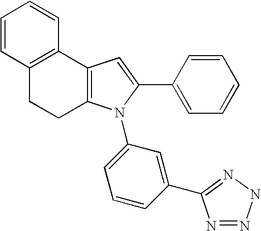 | 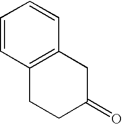 | 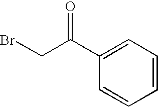 | 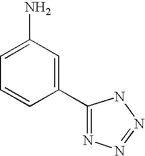 |
| 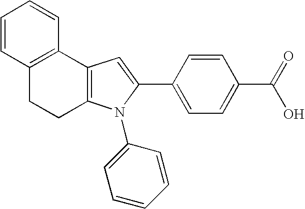 | 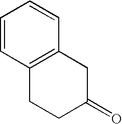 | 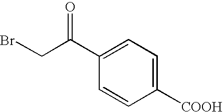 | 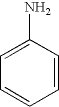 |
| 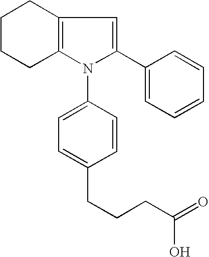 | 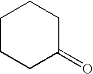 | 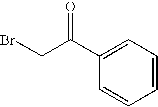 | 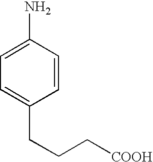 |
| 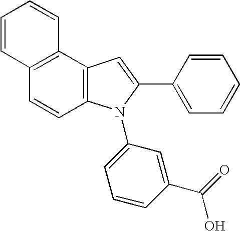 | 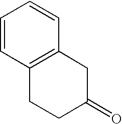 | 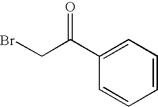 | 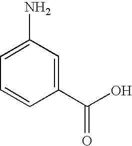 |
| 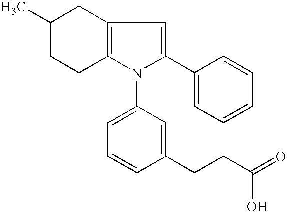 | 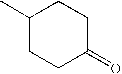 | 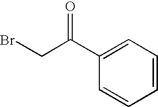 | 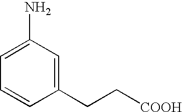 |
| 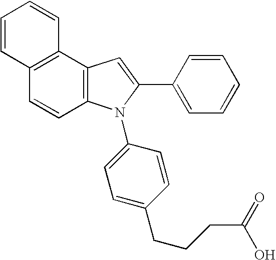 | 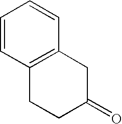 | 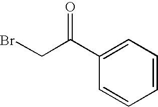 | 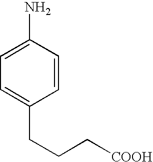 |
| 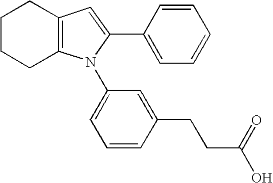 | 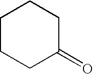 | 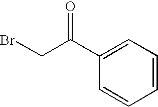 | 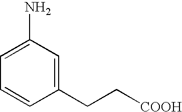 |
| 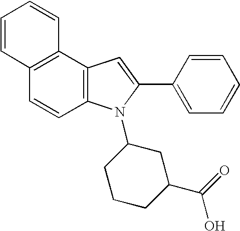 | 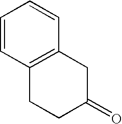 | 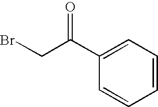 | 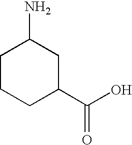 |
| 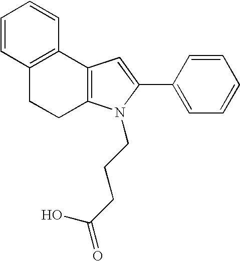 | 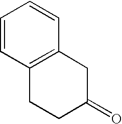 | 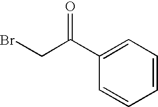 | 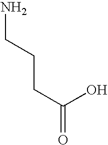 |
| 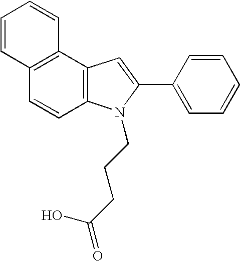 | 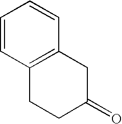 | 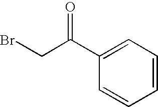 | 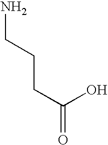 |
| 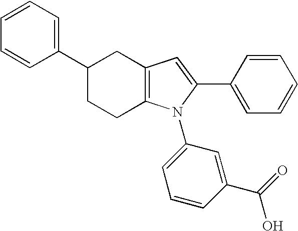 | 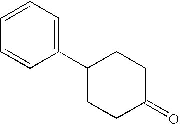 | 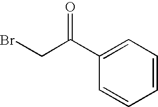 | 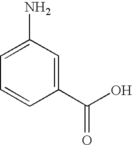 |
| 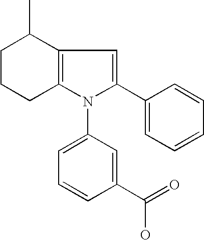 | 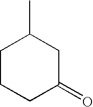 | 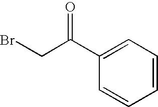 | 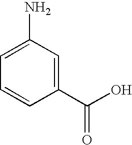 |
| 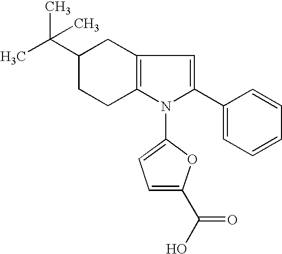 | 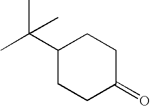 | 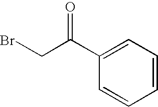 | 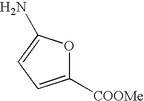 |
| 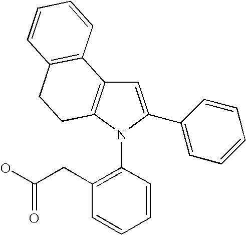 | 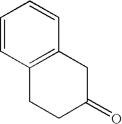 | 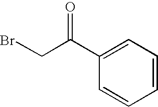 | 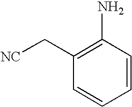 |
| 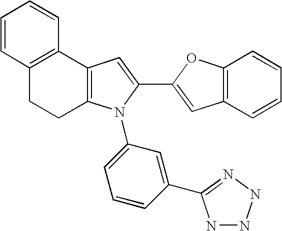 | 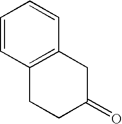 | 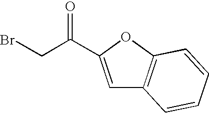 | 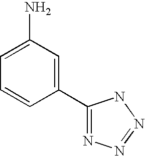 |
| 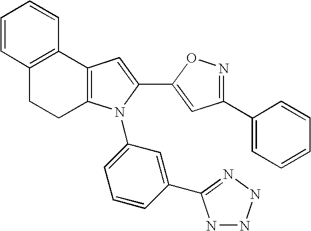 | 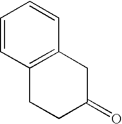 | 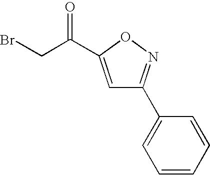 | 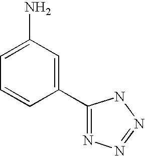 |
| 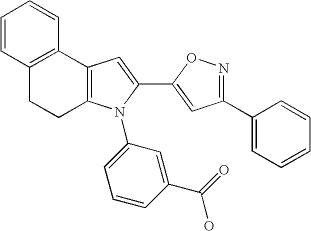 | 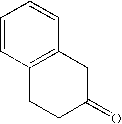 | 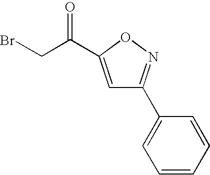 | 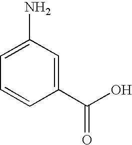 |

TABLE 7
Compounds from Table 6 and Characterization Data

| product structure | 1H NMR, d | MS | name |
|---|---|---|---|
| 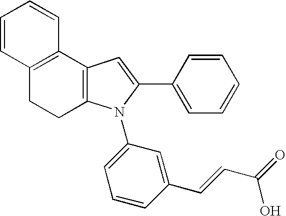 | DMSO-d6; 7.1-7.3 (m,16 H); 6.8 (s, 1 H); 2.9 (t,2 H); 2.6 (t, 2 H). | pos. mode392 (M + H),neg. mode390 (M − 1). | 3-[3-(2-phenyl-4,5-dihydrobenzo[e]indol-3-yl)-phenyl] acrylic acid |
| 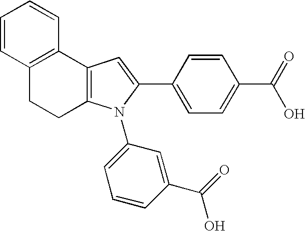 | DMSO-d6; 8.03 (d, 1 H),7.82-7.73 (m, 3 H), 7.68-7.52 (m, 3 H), 7.26-7.19 (m, 4 H), 7.12-7.07(m, 2 H), 2.98 (t, 2 H), 2.70(t, 2 H). | pos. mode410 (M + H);neg. mode408 (M − H). | 3-[2-(4-carboxyphenyl)-4,5-dihydrobenzo[e]indol-3-yl] benzoic acid |
| 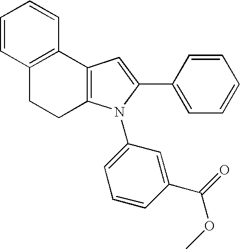 | CDCl3; 8.02 (tt, 1 H), 7.93(t, 1 H); 7.46 (d, 1 H), 7.42(t, 1 H), 7.28 (d, 1 H); 7.24(m, 1 H), 7.21-7.05 (m,7 H), 6.74 (s, 1 H), 3.92 (s,3 H), 3.0 (t, 2 H), 2.72 (t,2 H). | pos. mode380 (M + H). | methyl 3-(2-phenyl-4,5-dihydrobenzo[e]indol-3-yl)benzoate |
| 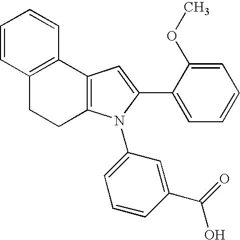 | MeOH-d4; 7.75 (t, 1 H),7.42-7.36 (m, 3 H), 7.32-7.14 (m, 5 H), 7.0 (t, 1 H),6.91 (t, 1 H), 6.73 (d, 1 H),6.5 (s, 1 H), 3.33 (s, 3 H)2.96 (t, 2 H), 2.71 (t, 2 H). | pos. mode396 (M + H). | 3-[2-(2-methoxyphenyl)-4,5-dihydrobenzo[e]indol-3-yl] benzoic acid |
| 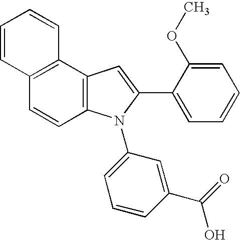 | MeOH-d4; 8.3 (d, 1 H);7.99-7.94 (m, 2 H); 7.88(d, 1 H); 7.59-7.38 (m,7 H); 7.33-7.28 (m, 1 H);7.21 (s, 1 H); 6.99 (t, 1 H);6.81 (s, 1 H); 3.38 (s, 3 H). | pos. mode394 (M + H). | 3-[2-(2-methoxyphenyl)-benzo[e]indol-3-yl]benzoic acid |
| 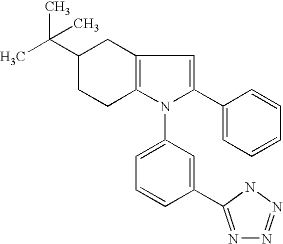 | CDCl3: 8.0 (d, 1 H); 7.9(br. s, 1 H); 7.5 (t, 1 H); 7.2(m, 1 H); 7.1 (m, 5 H); 6.3(s, 1 H); 2.7 (m, 1 H); 2.5(m, 1 H); 2.3-2.4 (m, 2 H);2.0 (m, 1 H); 1.5 (m, 1 H);1.4 (m, 1 H); 1.0 (s, 9 H). | pos. mode398 (M + H). | 5-tButyl-2-phenyl-1-[3-(1H-tetrazol-5-yl) phenyl]-4,5,6,7-tetrahydro-1H-indole |
| 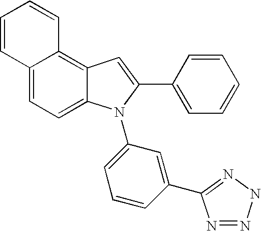 | DMSO-d6; 7.2-8.4 (m,16 H). | pos. mode388 (M + H). | 2-phenyl-3-[3-(2H-tetrazol-5-yl)-phenyl] 3H-benzo[e]indole |
| 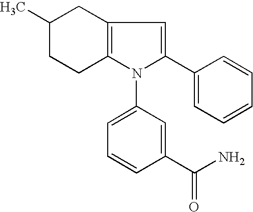 | CDCl3: 7.7 (dm, 1 H); 7.5(br. s, 1 H); 7.4 (t, 1 H); 7.3(m, 1 H); 7.0-7.2 (m, 5 H);6.2 (s, 1 H); 2.7 (m, 1 H);2.6 (m, 1 H); 2.4 (m, 1 H);2.2 (m, 1 H); 1.9 (m, 2 H);1.4 (m, 1 H); 1.1 (d, 3 H). | pos. mode331 (M + H). | 3-(5-methyl-2-phenyl-4,5,6,7-tetrahydroindol-1-yl) benzamide |
| 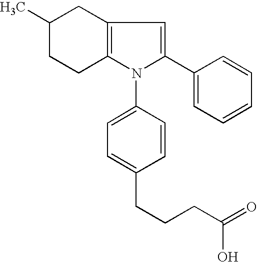 | CDCl3: 7.0-7.2 (m, 9 H);6.2 (s, 1 H); 2.7 (m, 3 H);2.5 (m, 1 H); 2.4 (m, 3 H);2.2 (m, 1 H); 2.0 (m, 2 H);1.9 (m, 2 H); 1.4 (m, 1 H);1.0 (d, 3 H). | pos. mode374 (M + H). | 4-[4-(5-methyl-2-phenyl-4,5,6,7-tetrahydroindol-1-yl) phenyl] butyric acid |
| 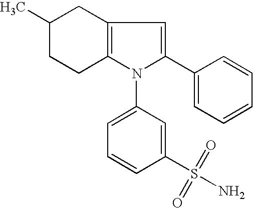 | CDCl3: 7.8 (dm, 1 H); 7.7(br. s, 1 H); 7.5 (t, 1 H); 7.3(m, 1 H); 7.0-7.2 (m, 5 H);6.2 (s, 1 H); 2.7 (m, 1 H);2.6 (m, 1 H); 2.4 (m, 1 H);2.2 (t, 1 H); 1.9 (m, 2 H);1.4 (m, 1 H); 1.1 (d, 3 H). | pos. mode367 (M + H). | 3-(5-methyl-2-phenyl-4,5,6,7-tetrahydroindol-1-yl) benzenesulfonamide |
| 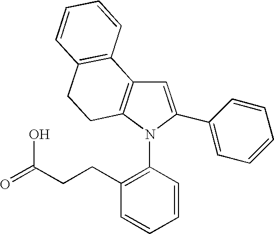 | CD3OD: 7.44 (m, 1 H),7.41-7.36 (m, 3 H), 7.30(m, 1 H), 7.20-7.08 (m,7 H), 7.00 (m, 1 H), 6.77(s, 1 H), 2.97-2.92 (m,2 H), 2.67-2.48 (m, 2 H),2.44-2.34 (m, 2 H), 2.25(m, 1 H), 2.04 (m, 1 H). | neg. mode392(M − H) | 3-[2-(2-phenyl-4,5-dihydro-benzo[e]indol-3-yl)-phenyl] propionic acid |
| 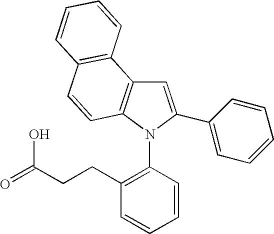 | CD3OD: 8.34 (m, 1 H),7.88 (m, 1 H), 7.58-7.52(m, 2 H), 7.50-7.34 (m,8 H), 7.26-7.20 (m, 3 H),7.04 (m, 1 H), 2.44 (m,1 H), 2.37 (m, 1 H), 2.09(m, 1 H), 1.98 (m, 1 H). | neg. mode390 (M − H) | 3-[2-(2-phenyl-benzo[e]indol-3-yl)-phenyl] propionic acid |
| 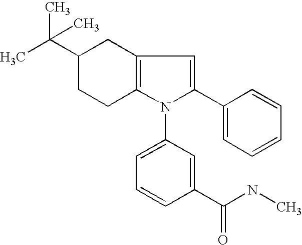 | CDCl3: 7.7 (dm, 1 H); 7.5(br. s, 1 H); 7.4 (t, 1 H); 7.2(m, 1 H); 7.0-7.1 (m, 5 H);6.2 (s, 1 H); 5.9 (br. s,1 H); 3.0 (d, 3 H); 2.7 (d,1 H); 2.6 (m, 1 H); 2.4 (m,2 H); 2.0 (m, 1 H); 1.5 (m,1 H); 1.4 (m, 1 H); 1.0 (s,9 H). | pos. mode387 (M + H). | 3-(5-tButyl-2-phenyl-4,5,6,7-tetrahydroindol-1-yl) N-methyl benzamide |
| 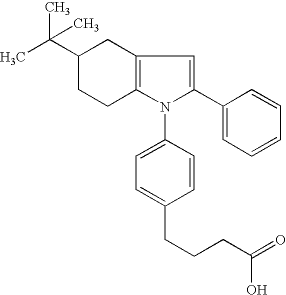 | CDCl3: 7.0-7.2 (m, 9 H);6.2 (s, 1 H); 2.5 (m, 3 H);2.4-2.5 (m, 4 H); 2.0 (m,3 H); 1.5 (m, 2 H); 1.4 (m,1 H); 0.9 (s, 9 H). | pos. mode416 (M + H). | 4-[4-(5-tButyl-2-phenyl-4,5,6,7-tetrahydroindol-1-yl) phenyl] butyric acid |
| 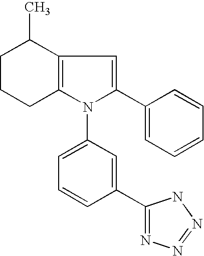 | CDCl3: 8.0 (d, 1 H); 7.9(br. s, 1 H); 7.4 (m, 2 H);7.0-7.2 (m, 6 H); 6.2 (s,1 H); 2.6 (m, 1 H); 2.5 (m,1 H); 2.2 (m, 2 H); 1.9 (m,1 H); 1.4 (m, 2 H); 1.0 (d,3 H). | pos. mode356 (M + H);neg. mode354 (M − H). | 4-methyl-2-phneyl-1-[3-(1H-tetrazol-5-yl) phenyl]-4,5,6,7-tetrahydro-1H-indole |
| 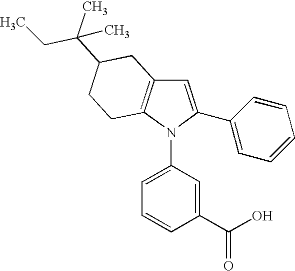 | CDCl3; 8.00 (dt, 1 H),7.94 (br s, 1 H), 7.39 (t,1 H), 7.26-7.28 (m, 1 H),7.05-7.18 (m, 5 H), 6.27(s, 1 H), 2.50-2.70 (m,2 H), 2.30-2.45 (m, 2 H),1.95-2.05 (m, 1 H), 1.55-1.70 (m, 1 H), 1.30-1.45 (m, 3 H), 0.90 (s,3 H), 0.89 (s, 3 H), 0.85 (t,3 H). | pos. mode388 (M + H). | 3-[5-(1,1-dimethylpropyl)-2-phenyl-4,5,6,7-tetrahydroindol-1-yl]benzoic acid |
| 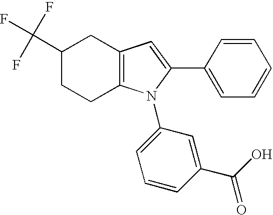 | CDCl3; 8.03 (dt, 1 H),7.93 (br s, 1 H), 7.43 (t,1 H), 7.26-7.30 (m, 1 H),7.08-7.20 (m, 3 H), 7.04-7.06 (m, 2 H), 6.28 (s,1 H), 2.91 (dd, 1 H), 2.55-2.74 (m, 2 H), 2.45-2.55(m, 2 H), 2.15-2.25 (m,1 H), 1.75 (qd, 1 H). | pos. mode386 (M + H). | 3-(2-phenyl-5-trifluoromethyl-4,5,6,7-tetrahydroindol-1-yl)benzoic acid |
| 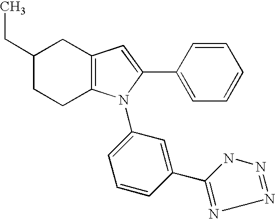 | CDCl3; 8.02 (d, 1 H),7.84 (br s, 1 H), 7.50 (t,1 H), 7.05-7.20 (m, 6 H),6.28 (s, 1 H), 2.76 (dd,1 H), 2.52-2.64 (m, 1 H),2.40-2.50 (m, 1 H), 2.17-2.27 (m, 1 H), 1.88-1.98 (m, 1 H), 1.67 (br s,1 H), 1.32-1.50 (m, 3 H),0.99 (t, 3 H). | pos. mode370 (M + H). | 5-ethyl-2-phenyl-1-[3-(1H-tetrazol-5-yl)-phenyl]-4,5,6,7-tetrahydro-1H-indole |
| 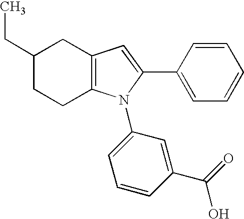 | CDCl3; 8.01 (dt, 1 H),7.97 (br s, 1 H), 7.40 (t,1 H), 7.28 (br d, 1 H), 7.03-7.19 (m, 5 H), 6.26 (s,1 H), 3.51 (dd, 1 H), 2.54(br s, 1 H), 2.41 (br d, 1 H),2.22 (dd, 1 H), 1.93 (br d,1 H), 1.67 (br s, 1 H), 1.33-1.49 (m, 3 H), 0.99 (t,3 H). | pos. mode346 (M + H). | 3-(5-ethyl-2-phenyl-4,5,6,7-tetrahydroindol-1-yl) benzoic acid |
| 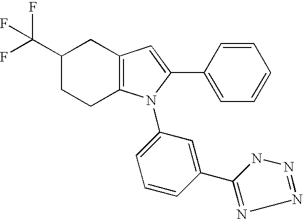 | CDCl3; 8.06 (dt, 1 H),7.90 (br s, 1 H), 7.50 (t,1 H), 7.10-7.20 (m, 6 H),6.29 (s, 1 H), 2.76 (dd,1 H), 2.60-2.75 (m, 2 H),2.53 (br dd, 2 H), 2.21 (brd, 1 H), 1.74 (qd, 1 H). | pos. mode410 (M + H). | 2-phenyl-1-[3-(1H-tetrazol-5-yl)-phenyl]-5-trifluoromethyl-4,5,6,7-tetrahydro-1H-indole |
| 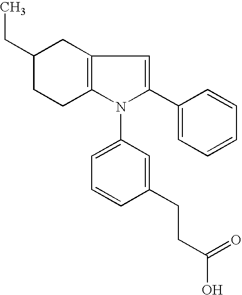 | CDCl3; 7.26 (t, 1 H), 6.95-7.20 (m, 7 H), 6.93 (br s,1 H), 6.23 (s, 1 H), 2.88 (t,2 H), 2.75 (dd, 1 H), 2.48-2.59 (m, 3 H), 2.41 (br d,1 H), 2.22 (dd, 1 H), 1.91(br d, 1 H), 1.65 (br s, 1 H),1.35-1.50 (m, 3 H), 0.98(t, 3 H). | pos. mode374 (M + H). | 3-[3-(5-ethyl-2-phenyl-4,5,6,7-tetrahydroindol-1-yl)-phenyl] propionic acid |
| 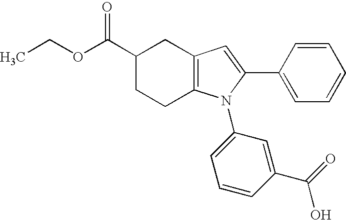 | CDCl3; 8.03 (d, 1 H),7.95 (br s, 1 H), 7.42 (t,1 H), 7.28 (d, 1 H), 7.05-7.20 (m, 5 H), 6.28 (s,1 H), 4.19 (q, 2 H), 2.94(dd, 1 H), 2.85 (d, 1 H),2.72-2.81 (m, 1 H), 2.59(br s, 1 H), 2.48 (br d, 1 H),2.23 (br d, 1 H), 1.82-1.90 (m, 1 H), 1.30 (t, 3 H). | pos. mode390 (M + H). | 3-(5-ethoxycarbonyl-2-phenyl-4,5,6,7-tetrahydroindol-1-yl)benzoic acid |
| 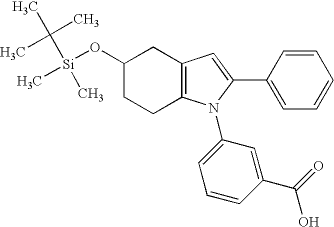 | CDCl3; 8.01 (dt, 1 H),7.97 (br s, 1 H), 7.41 (t,1 H), 7.25-7.30 (m, 1 H),7.08-7.18 (m, 3 H), 7.02-7.06 (m, 2 H), 6.23 (s,1 H), 2.87 (dd, 1 H), 2.46-2.64 (m, 3 H), 1.92-2.02(m, 1 H), 1.78-1.88 (m,2 H), 0.93 (s, 9 H), 0.12 (s,3 H), 0.11 (s, 3 H). | pos. mode448 (M + H);neg. mode446 (M − H). | 3-[5-(tButyldimethylsilyl)-2-phenyl-4,5,6,7-tetrahydroindol-1-yl]benzoic acid |
| 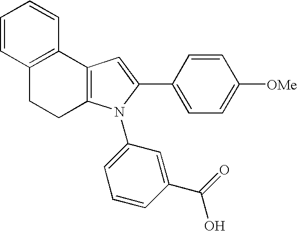 | MeOH-d4; 7.95 (t, 1 H),7.67 (t, 1 H), 7.58 (t, 1 H),7.51-7.44 (m, 2 H), 7.18(d, 2 H), 7.01 (d, 3 H), 6.8(d, 2 H), 6.74 (s, 1 H), 3.35(s, 3 H), 2.92 (t, 2 H), 2.62(t, 2 H). | pos. mode396 (M + H). | 3-[2-(4-methoxyphenyl)-4,5-dihydrobenzo[e]indol-3-yl] benzoic acid |
| 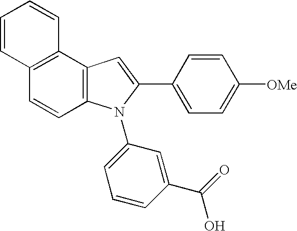 | MeOH-d4; 8.4 (d, 1 H),8.03 (tt, 1 H), 7.95 (d, 1 H),7.84 (t, 1 H), 7.7-7.58(m, 4 H), 7.49-7.43 (m,2 H), 7.32-7.24 (m, 3 H),6.9 (m, 2 H), 3.74 (s, 3 H). | pos. mode394 (M + H). | 3-[2-(4-methoxyphenyl)-benzo[e]indol-3-yl]benzoic acid |
| 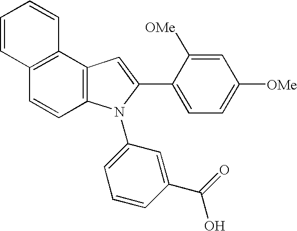 | CDCl3; 8.35 (d, 1 H), 7.96-7.92 (m, 2 H), 7.79 (t,1 H), 7.63-7.56 (m, 3 H),7.50-7.35 (m, 4 H), 7.27(s, 1 H), 6.59 (dd, 1 H),6.43 (d, 1 H), 3.76 (s, 3 H),3.34 (s, 3 H). | pos. mode424 (M + H).neg. mode422(M − H). | 3-[2-(2,4-dimethoxyphenyl)-benzo[e]indol-3-yl]benzoic acid |
| 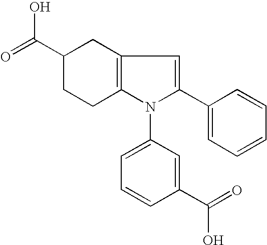 | MeOH-d4; 7.96 (dt, 1 H),7.75 (br s, 1 H), 7.46 (t,1 H), 7.27-7.33 (m, 1 H),7.09-7.15 (m, 2 H), 7.01-7.09 (m, 3 H), 6.22 (s,1 H), 2.80-2.90 (m, 1 H),2.70-2.80 (m, 2 H), 2.40-2.60 (m, 2 H), 2.15-2.25 (m, 1 H), 1.80-1.90(m, 1 H). | pos. mode362 (M + H);neg. mode360 (M − H). | 3-(5-carboxy-2-phenyl-4,5,6,7-tetrahydroindol-1-yl) benzoic acid |
| 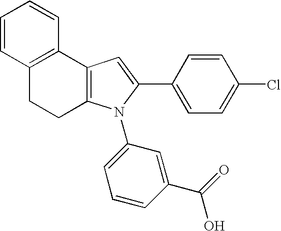 | DMSO-d6; 7.99 (tt, 1 H),7.70 (t, 1 H), 7.61 (t, 1 H),7.54-7.47 (m, 2 H), 7.29(d, 2 H), 7.19 (d, 2 H), 7.11-7.05 (m, 3 H), 6.92 (s,1 H), 2.94 (t, 2 H), 2.64 (t,2 H). | pos. mode400 (M + H). | 3-[2-(4-chlorophenyl)-4,5-dihydrobenzo[e]indol-3-yl]benzoic acid |
| 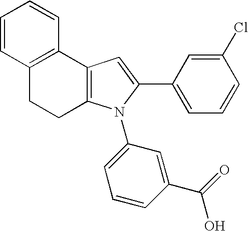 | DMSO-d6; 7.71-7.48(m, 5 H), 7.22-7.18 (m,5 H), 7.07-6.95 (m, 3 H),2.94 (t, 2 H), 2.64 (t, 2 H). | pos. mode400 (M + H). | 3-[2-(3-chlorophenyl)-4,5-dihydrobenzo[e]indol-3-yl]benzoic acid |
| 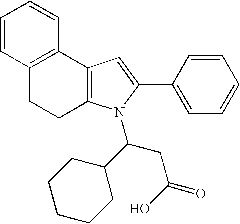 | DMSO-d6; 6.9-7.4 (m,9 H), 6.3 (s, 1 H), 4.4 (t,1 H), 2.9 (t, 2 H), 2.6 (t,2 H), 3.3 (t, 2 H), 1.8-0.4(m, 10 H). | pos. mode400 (M + H);neg. mode.398 (M − 1). | 3-cyclohexyl-3-(2-phenyl-4,5-dihydrobenzo[e]indol-3-yl) propionic acid |
| 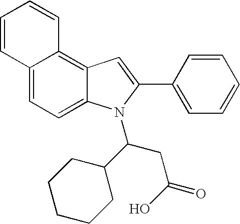 | DMSO-d6; 7.4-8.2 (m,11 H), 7.0 (s, 1 H), 4.6 (s,1 H), 3.3 (t, 2 H), 2.1-0.3(m, 10 H). | pos. mode398 (M + H),neg. mode.396 (M − 1). | 3-cyclohexyl-3-(2-phenylbenzo[e]indol-3-yl)propionic acid |
| 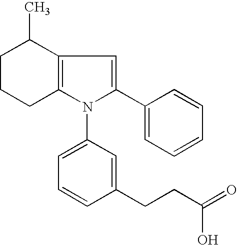 | CDCl3: 7.3 (m, 1 H); 7.0-7.2 (m, 7 H); 6.9 (br. s,1 H); 6.2 (s, 1 H); 2.9 (t,2 H); 2.6 (m, 2 H); 2.5 (m,2 H); 2.4 (m, 1 H); 2.1 (m,1 H); 1.9 (m, 2 H); 1.4 (m,1 H); 1.0 (d, 3 H). | pos. mode360 (M + H). | 3-[3-(4-methyl-2-phenyl-4,5,6,7-tetrahydroindol-1-yl) phenyl] propionic acid |
| 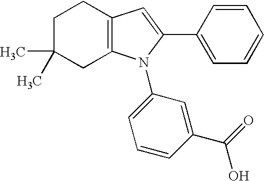 | CDCl3; 8.03 (d, 1 H),7.93 (br s, 1 H), 7.42 (t,1 H), 7.29 (d, 1 H), 7.00-7.17 (m, 5 H), 6.27 (s,1 H), 2.61 (br s, 2 H), 2.21(br s, 2 H), 1.56 (t, 2 H),1.00 (s, 6 H). | pos. mode346 (M + H);neg. mode344(M − H). | 3-(6,6-dimethyl-2-phenyl-4,5,6,7-tetrahydroindol-1-yl) benzoic acid |
| 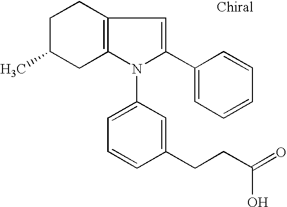 | CDCl3; 7.27 (t, 1 H), 7.10-7.16 (m, 3 H), 7.02-7.10 (m, 4 H), 6.92 (br s,1 H), 6.25 (s, 1 H), 2.88 (t,2 H), 2.60-2.70 (m, 2 H),2.53 (t, 2 H), 2.43 (dd,1 H), 2.05-2.20 (m, 1 H), 1.80-1.90 (m, 2 H), 1.35-1.50 (m, 1 H), 1.04 (d,3 H). | pos. mode360 (M + H);neg. mode358(M − H). | 3-[3-((R)-6-methyl-2-phenyl-4,5,6,7-tetrahydroindol-1-yl)phenyl] propionic acid |
| 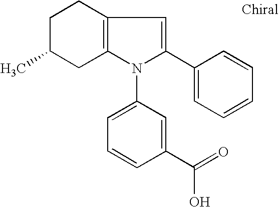 | CDCl3; 8.03 (d, 1 H),7.96 (br s, 1 H), 7.42 (t,1 H), 7.29 (br d, 1 H), 7.00-7.20 (m, 5 H), 6.27 (s,1 H), 2.60-2.70 (m, 2 H),2.42 (dd, 1 H), 2.14 (t,1 H), 1.88 (br d, 2 H), 1.40-1.50 (m, 1 H), 1.05 (d,3 H). | pos. mode332 (M + H);neg. mode330(M − H). | 3-((R)-6-methyl-2-phenyl-4,5,6,7-tetrahydroindol-1-yl) benzoic acid |
| 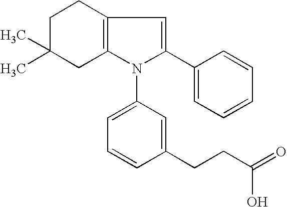 | CDCl3; 7.28 (t, 1 H), 7.10-7.16 (m, 3 H), 6.99-7.09 (m, 4 H), 6.90 (t, 1 H),6.25 (s, 1 H), 2.88 (t, 2 H),2.60 (t, 2 H), 2.52 (t, 2 H),2.22 (s, 2 H), 1.55 (t, 2 H),0.99 (s, 6 H). | pos. mode374 (M + H);neg. mode372(M − H). | 3-[3-(6,6-dimethyl-2-phenyl-4,5,6,7-tetrahydroindol-1-yl)phenyl] propionic acid |
| 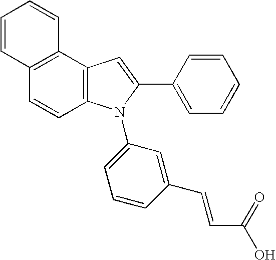 | DMSO-d6; 7.2-8.2 (m,17 H), 6.6 (s, 1 H). | pos. mode390 (M + H),neg. mode.388(M − 1). | 3-[3-(2-phenylbenzo[e]indol-3-yl)-phenyl] acrylic acid |
| 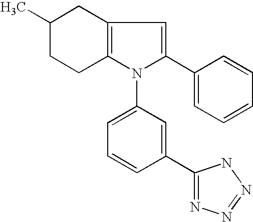 | CDCl3: 8.0 (d, 1 H); 7.9(br. s, 1 H); 7.4 (t, 2 H); 7.0-7.2 (m, 6 H); 6.2 (s, 1 H);2.7 (m, 1 H); 2.5 (m, 1 H);2.4 (m, 1 H); 2.2 (m, 1 H);1.8 (m, 2 H); 1.4 (m, 1 H);1.0 (d, 3 H). | pos. mode356 (M + H);neg. mode354(M − H). | 5-methyl-2-phenyl-1-[3-(1H-tetrazol-5-yl) phenyl]-4,5,6,7-tetrahydro-1H-indole |
| 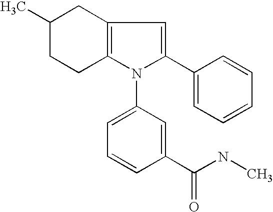 | CDCl3: 7.7 (dm, 1 H); 7.5(br. s, 1 H); 7.4 (t, 1 H); 7.2(m, 1 H); 7.0-7.1 (m, 5 H);6.2 (s, 1 H); 5.9 (br. s,1 H); 3.0 (d, 3 H); 2.7 (dd,1 H); 2.5 (m, 1 H); 2.4 (m,1 H); 2.2 (m, 1 H); 1.9 (m,2 H); 1.4 (m, 1 H); 1.1 (d,3 H). | pos. mode345 (M + H). | N-methyl-3-(5-methyl-2-phenyl-4,5,6,7-tetrahydroindol-1-yl)benzamide |
| 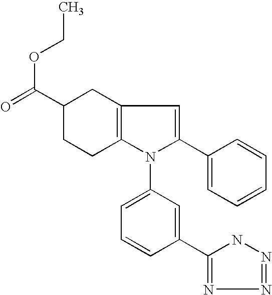 | CDCl3; 8.07 (d, 1 H),7.82 (br s, 1 H), 7.50 (t,1 H), 7.05-7.18 (m, 6 H),6.25 (s, 1 H), 4.20 (q, 2 H),2.75-2.95 (m, 3 H), 2.45-2.65 (m, 2 H), 2.15-2.25 (m, 1 H), 1.85-1.95(m, 1 H), 1.30 (t, 3 H). | neg. mode412 (M − H). | 3-[3-(5-ethoxycarbonyl-2-phenyl-4,5,6,7-tetrahydroindol-1-yl)-phenyl] propionic acid |
| 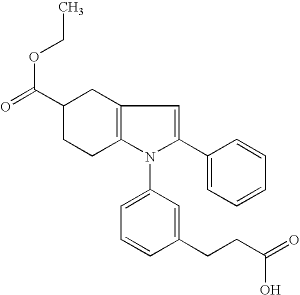 | CDCl3; 7.24-7.30 (m,1 H), 7.00-7.20 (m, 7 H),6.93 (br s, 1 H), 6.26 (s,1 H), 4.19 (q, 2 H), 2.79-3.00 (m, 4 H), 2.68-2.78(m, 1 H), 2.20-2.45 (m,5 H), 2.15-2.25 (m, 1 H),1.29 (t, 3 H). | pos. mode418 (M + H);neg. mode416 (M − H). | 1-[3-(2-carboxy-ethyl)-phenyl]-2-phenyl-4,5,6,7-tetrahydro-1H-indole-5-carboxylic acid ethyl ester |
| 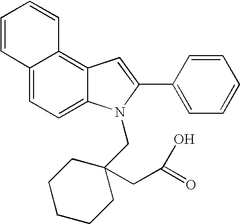 | DMSO-d6; 7.2-8.2 (m,11 H), 6.6. (s, 1 H), 4.6 (s,2 H), 2.2 (s, 2 H), 1.2-1.1(m, 10 H). | EM 397. | [1-(2-phenylbenzo[e]indol-3-ylmethyl)-cyclohexyl]acetic acid |
| 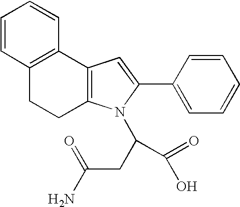 | DMSO-d6; 7.2-8.3 (m,10 H), 5.5 (s, 1 H), 2.9 (t,2 H), 2.6 (t, 2 H), 2.5 (br. s,2 H). | pos. mode361 (M + 1),neg. mode359 (M − 1). | 2-(2-phenyl-4,5-dihydrobenzo[e]indol-3-yl)succinamic acid |
| 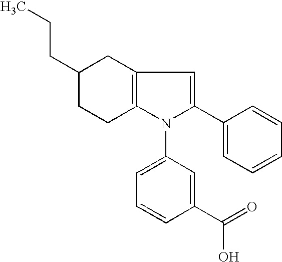 | CDCl3; 8.01 (dt, 1 H),7.96 (br s, 1 H), 7.40 (t,1 H), 7.28 (br d, 1 H), 7.05-7.20 (m, 5 H), 6.25 (s,1 H), 2.74 (dd, 1 H), 2.50-2.60 (m, 1 H), 2.41 (br d,1 H), 2.22 (dd, 1 H), 1.92(br d, 1 H), 1.77 (br s, 1 H),1.30-1.50 (m, 5 H), 0.94(t, 3 H). | neg. mode358 (M − H). | 3-(2-phenyl-5-propyl-4,5,6,7-tetrahydroindol-1-yl) benzoic acid |
| 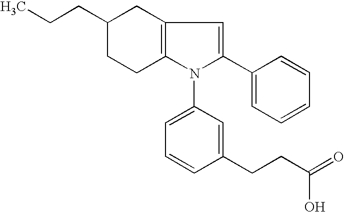 | CDCl3; 7.26 (t, 1 H), 6.98-7.18 (m, 7 H), 6.93 (br s,1 H), 6.23 (s, 1 H), 2.88 (t,2 H), 2.74 (dd, 1 H), 2.45-2.60 (m, 3 H), 2.41 (br d,1 H), 2.21 (dd, 1 H), 1.91(br d, 1 H), 1.72 (br s, 1 H),1.30-1.50 (m, 5 H), 0.93(t, 3 H). | pos. mode388 (M + H);neg. mode386 (M − H). | 3-[3-(2-phenyl-5-propyl-4,5,6,7-tetrahydroindol-1-yl) phenyl] propionic acid |
| 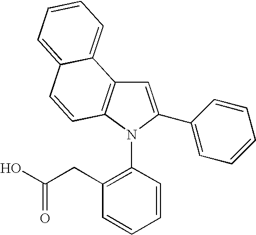 | acetone-d6: 8.41 (m, 1 H),7.94 (m, 1 H), 7.62-7.37(m, 10 H), 7.31-7.22 (m,3 H), 7.11 (m, 1H), 3.33(s, 2 H). | pos. mode378 (M + H) | [2-(2-phenyl-benzo[e]indol-3-yl)-phenyl] acetic acid |
| 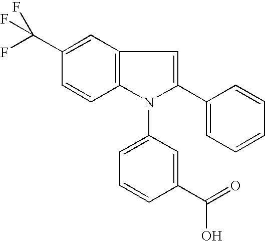 | CDCl3; 8.14 (dt, 1 H),8.09 (t, 1 H), 8.00 (t, 1 H),7.54 (t, 1 H), 7.37-7.46(m, 2 H), 7.33 (d, 1 H),7.20-7.32 (m, 5 H), 6.89(d, 1 H). | neg. mode380 (M − H). | 3-(2-phenyl-5-trifluoromethyl-indol-1-yl)benzoic acid |
| 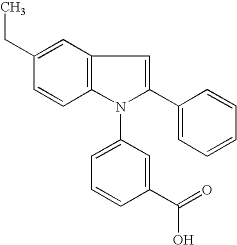 | CDCl3; 8.10 (s, 1 H),8.08 (t, 1 H), 7.51 (s, 1 H),7.48 (t, 1 H), 7.37 (d, 1 H),7.18-7.28 (m, 6 H), 7.07(dd, 1 H), 6.77 (s, 1 H),2.77 (t, 2 H), 1.31 (t, 3 H). | pos. mode342 (M + H);neg. mode340 (M − H). | 3-(5-ethyl-2-phenyl-indol-1-yl) benzoic acid |
| 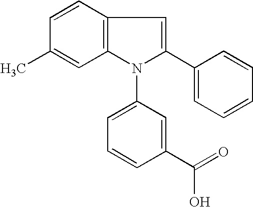 | MeOH-d4; 8.02 (dt, 1 H),7.86-7.92 (m, 1 H), 7.48-7.57 (m, 2 H), 7.36-7.45 (m, 1 H), 7.20-7.30(m, 5 H), 6.98-7.05 (m,2 H), 6.75 (d, 1 H), 2.39 (s,3 H). | pos. mode328 (M + H);neg. mode326 (M − H). | 3-(6-methyl-2-phenyl-indol-1-yl) benzoic acid |
| 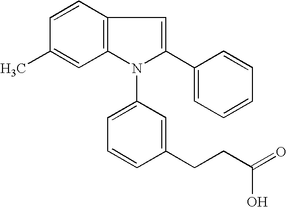 | MeOH-d4; 7.48 (d, 1 H),7.36 (t, 1 H), 7.18-7.29(m, 6 H), 7.11 (t, 1 H), 7.00-7.08 (m, 2 H), 6.95 (ddd,1 H), 6.71 (d, 1 H), 2.90 (t,2 H), 2.51 (t, 2 H), 2.39 (s,3 H). | pos. mode356 (M + H);neg. mode354 (M − H). | 3-[3-(6-methyl-2-phenyl-indol-1-yl)-phenyl]propionic acid |
| 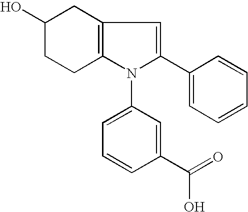 | CDCl3; 8.03 (dt, 1 H),7.97 (t, 1 H), 7.42 (t, 1 H),7.28 (ddd, 1 H), 7.07-7.18 (m, 3 H), 7.01-7.06(m, 2 H), 6.26 (s, 1 H),4.30-4.40 (m 1 H), 3.00(dd, 1 H), 2.50-2.70 (m,3 H), 1.85-2.05 (m, 2 H). | pos. mode334 (M + H);neg. mode332 (M − H). | 3-(5-hydroxy-2-phenyl-4,5,6,7-tetrahydroindol-1-yl) benzoic acid |
| 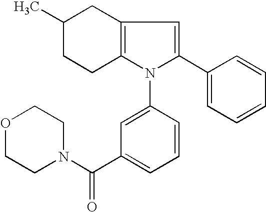 | CDCl3: 7.5 (m, 1 H); 7.4(m, 2 H); 7.0-7.2 (m, 5 H);6.8 (br. s, 1 H); 6.2 (s,1 H); 3.6 (br. s, 4 H); 3.2(br. s, 2 H); 2.8 (br. s, 2 H);2.7 (m, 1 H); 2.5 (m, 2 H);2.2 (m, 1 H); 1.9 (m, 2 H);1.4 (m, 1 H); 1.1 (d, 3 H). | pos. mode401 (M + H). | [3-(5-methyl-2-phenyl-4,5,6,7-tetrahydroindol-1-yl) phenyl] morpholin-4-yl-methanone |
| 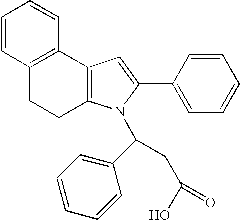 | DMSO-d6; 7.2-8.3 (m,15 H), 5.6 (s, 1 H), 3.8 (dd,2 H), 2.9 (t, 2 H), 2.6 (t,2 H). | neg. mode392 (M − 1). | 3-phenyl-3-(2-phenyl-4,5-dihydrobenzo[e]indol-3-yl)propionic acid |
| 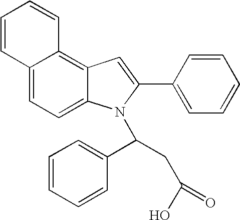 | DMSO-d6; 7.1-8.3 (m,17 H), 6.1 (s, 1 H), 3.8-3.4 (dd, 2 H). | neg. mode390 (M − 1). | 3-phenyl-3-(2-phenylbenzo[e]indol-3-yl)propionic acid |
| 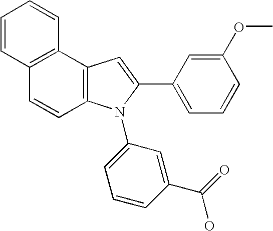 | MeOH-d4; 8.3 (d, 1 H),8.08-8.06 (tt, 1 H), 7.96(d, 1 H), 7.87 (m, 1 H), 7.6-7.2 (m, 9 H), 6.82 (m,2 H), 3.75 (s, 3 H). | pos. mode394 (M + H);neg. mode392 (M − H). | 3-[2-(3-methoxyphenyl)-benzo[e]indol-3-yl]benzoic acid |
| 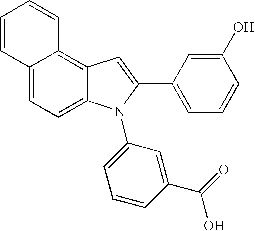 | MeOH-d4; 8.3 (d, 1 H),8.01-7.97 (m, 2 H), 7.88(d, 1 H), 7.58-7.37 (m,6 H), 7.26-7.24 (m, 2 H),7.15-7.11 (t, 1 H), 6.81(t, 1 H), 6.71 (d, 1 H). | pos. mode380 (M + H);neg. mode378 (M − H). | 3-[2-(3-hydroxyphenyl)-benzo[e]indol-3-yl]benzoic acid |
| 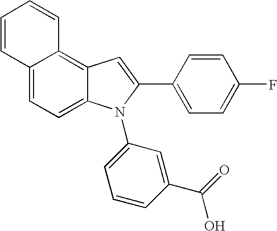 | DMSO-d6; 7.56 (d, 1 H),7.32 (d, 1 H), 7.2 (s, 1 H),7.13 (d, 1 H), 6.8-6.5 (m,10 H), 6.28-6.23 (m, 1 H). | pos. mode382 (M + H);neg. mode380 (M − H). | 3-[2-(4-fluorophenyl)-benzo[e]indol-3-yl]benzoic acid |
| 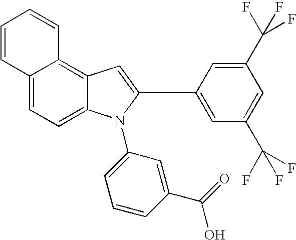 | DMSO-d6; 8.48 (d, 1 H),8.1 (tt, 1 H), 8.08 (s, 1 H),8.04-8.02 (m, 2 H), 7.97-7.93 (m, 3 H), 7.77-7.67 (m, 4 H), 7.55-7.51(m, 1 H), 7.40 (d, 1 H). | pos. mode500 (M + H);neg. mode498 (M − H). | 3-[2-(3,5-bistrifluoromethylphenyl)-benzo[e]indol-3-yl]benzoic acid |
| 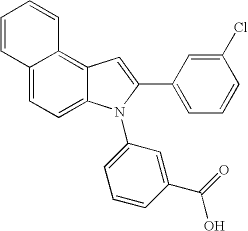 | DMSO-d6; 8.4 (d, 1 H),8.07 (d, 1 H), 8.0 (d, 1 H),7.87 (s, 1 H), 7.72-7.60(m, 5 H), 7.5-7.44 (m,2 H), 7.4-7.33 (d, 3 H),7.2 (s, 1 H). | pos. mode398 (M + H);neg. mode396 (M − H). | 3-[2-(3-chlorophenyl)-benzo[e]indol-3-yl]benzoic acid |
| 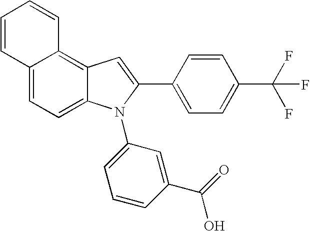 | DMSO-d6; 8.45 (d, 1 H),8.07 (tt, 1 H), 8.0 (d, 1 H),7.9 (t, 1 H), 7.8 (s, 1 H),7.7-7.6 (m, 6 H), 7.54-7.47 (m, 3 H), 7.35 (d,1 H). | pos. mode432 (M + H);neg. mode430 (M − H). | 3-[2-(4-trifluoromethylphenyl)-benzo[e]indol-3-yl]benzoic acid |
| 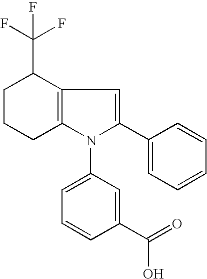 | CDCl3; 8.05 (dt, 1 H),7.98 (br s, 1 H), 7.44 (t,1 H), 7.37 (br d, 1 H), 7.10-7.20 (m, 3 H), 7.05-7.10 (m, 2 H), 6.45 (s,1 H), 3.50 (sext, 1 H), 2.45-2.60 (m, 1 H), 2.35-2.45 (m, 1 H), 1.95-2.15(m, 2 H), 1.85-1.95 (m,1 H), 1.68-1.80 (m, 1 H). | pos. mode386 (M + H);neg. mode384 (M − H). | 3-(2-phenyl-4-trifluoromethyl-4,5,6,7-tetrahydroindol-1-yl)benzoic acid |
| 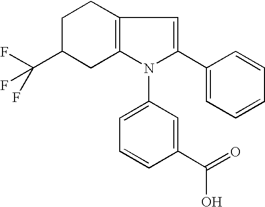 | CDCl3; 8.06 (d, 1 H),7.92 (br s, 1 H), 7.46 (t,1 H), 7.33 (br s, 1 H), 7.08-7.20 (m, 3 H), 7.02-7.08 (m, 2 H), 6.27 (s,1 H), 2.80 (dd, 1 H), 2.40-2.74 (m, 4 H), 2.21 (br d,1 H), 1.75 (qd, 1 H). | pos. mode386 (M + H);neg. mode384 (M − H). | 3-(2-phenyl-6-trifluoromethyl-4,5,6,7-tetrahydroindol-1-yl)benzoic acid |
| 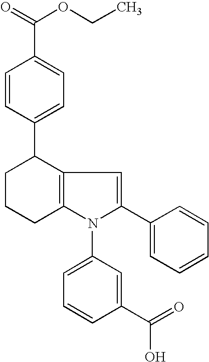 | CDCl3; 7.95-8.10 (m,4 H), 7.45 (t, 1 H), 7.27-7.42 (m, 3 H), 7.05-7.18(m, 3 H), 6.95-7.05 (m,2 H), 6.01 (s, 1 H), 4.38 (q,2 H), 4.07-4.14 (m, 1 H),2.64 (br s, 1 H), 2.45 (brd, 1 H), 2.10-2.23 (m,1 H), 1.90-2.00 (m, 1 H),1.70-1.85 (m, 2 H), 1.39(t, 3 H). | pos. mode466 (M + H);neg. mode464 (M − H). | 3-{4-[4-(ethoxycarbonyl)phenyl]-2-phenyl-4,5,6,7-tetrahydroindol-1-yl}benzoic acid |
| 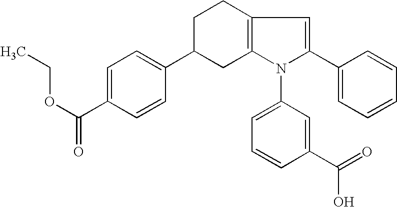 | CDCl3; 7.95-8.05 (m,3 H), 7.92 (s, 1 H), 7.40 (t,1 H), 7.25-7.35 (m, 3 H),7.05-7.20 (m, 5 H), 6.32(s, 1 H), 4.35 (q, 2 H), 3.00-3.13 (m, 1 H), 2.79 (br s,2 H), 2.62 (br s, 2 H), 1.95-2.20 (m, 2 H), 1.37 (t,3 H). | pos. mode466 (M + H);neg. mode464 (M − H). | 3-{6-[4-(ethoxycarbonyl)phenyl]-2-phenyl-4,5,6,7-tetrahydroindol-1-yl}benzoic acid |
| 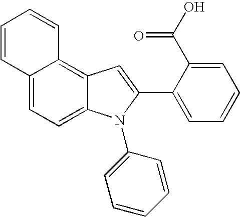 | MeOH-d4; 8.3 (d, 1 H),7.9 (d, 1 H), 7.75 (d, 1 H),7.56-7.26 (d, 12 H), 7.18(s, 1 H). | neg. mode362 (M − H). | 2-(3-phenyl-3H-benzo[e]indol-2-yl)benzoic acid |
| 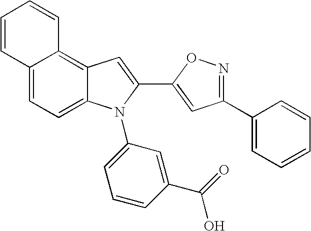 | DMSO-d6; 7.1-8.3 (m,16 H), 6.1 (s, 1 H). | pos. mode431 (M + 1),neg. mode429 (M − 1). | 3-[2-(3-phenylisoxazol-5-yl)-benzo[e]indol-3-yl]benzoic acid |
| 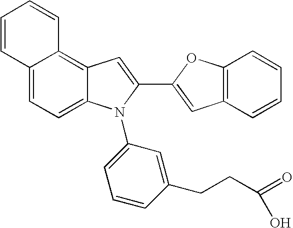 | DMSO-d6; 7.1-8.4 (m,15 H), 6.1 (s, 1 H), 3.1 (t,2 H), 2.7 (t, 2 H). | pos. mode432 (M + 1),neg. mode430 (M − 1). | 3-[3-(2-benzofuran-2-yl-benzo[e]indol-3-yl)-phenyl] propionic acid |
| 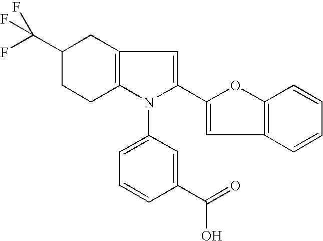 | CDCl3; 8.22 (dt, 1 H),8.07 (t, 1 H), 7.61 (t, 1 H),7.55 (ddd, 1 H), 7.33-7.37 (m, 1 H), 7.28-7.31(m, 1 H), 7.07-7.19 (m,2 H), 6.68 (s, 1 H), 5.60 (s,1 H), 2.93 (dd, 1 H), 2.65-2.75 (m, 1 H), 2.40-2.58(m, 3 H), 2.19 (br d, 1 H),1.76 (qd, 1 H). | neg. mode424 (M − H). | 3-(2-benzofuran-2-yl-5-trifluoromethyl-4,5,6,7-tetrahydroindol-1-yl)benzoic acid |
| 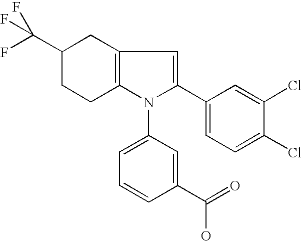 | DMSO-d6; 12.9 (br s,1 H), 7.97 (dt, 1 H), 7.67(br s, 1 H), 7.59 (t, 1 H),7.42 (d, 1 H), 7.40-7.50(m, 1 H), 7.25 (d, 1 H),6.89 (dd, 1 H), 6.48 (s,1 H), 2.81 (dd, 1 H), 2.65-2.78 (m, 1 H), 2.50-2.65(m, 2 H), 2.38 (dd, 1 H),2.10 (br d, 1 H), 1.63 (qd,1 H). | pos. mode454 (M + H);neg. mode452 (M − H). | 3-[2-(3,4-dichlorophenyl)-5-trifluoromethyl-4,5,6,7-tetrahydroindol-1-yl]benzoic acid |
| 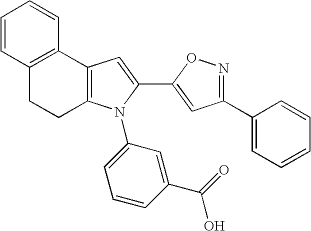 | DMSO-d6; 7.1-8.3 (m,13 H), 6.2 (s, 1 H), 2.9 (t,2 H), 2.6 (t, 2 H). | pos. mode433 (M + 1),neg. mode431 (M − 1). | 3-[2-(3-phenylisoxazol-5-yl)-4,5-dihydrobenzo[e]indol-3-yl]benzoic acid |
| 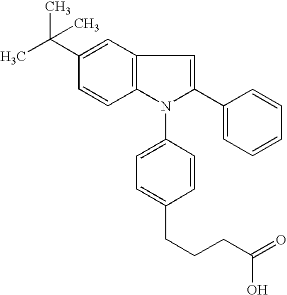 | CDCl3: 7.7 (m, 1 H); 7.1-7.3 (m, 11 H); 6.8 (s, 1 H);2.7 (t, 2 H); 2.4 (t, 2 H); 2.0(m, 2 H); 1.4 (m, 9 H). | pos. mode412 (M + H);neg. mode410 (M − H). | 4-[4-(5-tButyl-2-phenyl-indol-1-yl) phenyl] butyricacid |
| 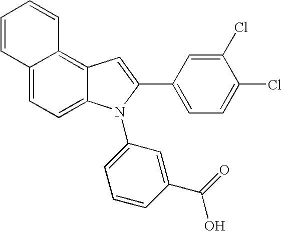 | DMSO-d6; 8.4 (d, 1 H),8.1 (tt, 1 H), 8.0 (d, 1 H),7.9 (t, 1 H), 7.8 (s, 1 H),7.7-7.57 (m, 6 H), 7.50-7.46 (t, 1 H), 7.3 (d, 1 H),7.2 (dd, 1 H). | pos. mode433 (M + H);neg. mode431 (M − H). | 3-[2-(3,4-dichlorophenyl)-benzo[e]indol-3-yl]benzoic acid |
| 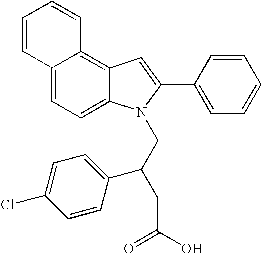 | DMSO-d6; 6.6-8.2 (m,16 H), 4.6 (dd, 2 H), 3.3(dd, 2 H), 2.07 (s, 1 H). | pos. mode430 (M + 1). | 3-(4-chlorophenyl)-4-(2-phenylbenzo[e]indol-3-yl)butyric acid |
| 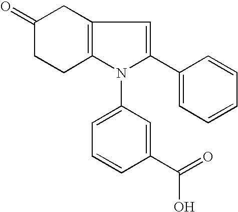 | CDCl3; 8.05 (d, 1 H),7.94 (s, 1 H), 7.45 (t, 1 H),7.31 (d, 1 H), 7.12-7.20(m, 3 H), 7.04-7.10 (m,2 H), 6.27 (s, 1 H), 3.53 (s,2 H), 2.88 (t, 2 H), 2.70 (t,2 H). | 331.8 (M dot) | 3-(5-oxo-2-phenyl-4,5,6,7-tetrahydroindol-1-yl) benzoic acid |
| 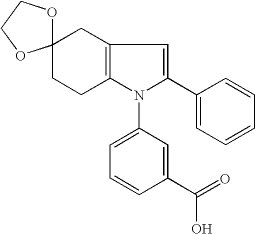 | CDCl3; 8.02 (d, 1 H), 7.99(s, 1 H), 7.40 (t, 1 H), 7.29(t, 1 H), 7.08-7.25 (m,3 H), 7.00-7.06 (m, 2 H),6.24 (s, 1 H), 4.03-4.10(m, 4 H), 2.89 (s, 2 H),2.70-2.60 (m, 2 H), 2.02-1.95 (m, 2 H). | pos. mode376(M + H). | 3-[2′-phenyl-4′,5′,6′,7′-tetrahydrospiro(1,3-dioxolane-2,5′-indol)-1′-yl]benzoic acid |
| 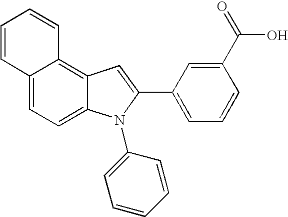 | MeOH-d4; 8.3 (d, 1 H),8.04 (t, 1 H), 7.88 (m, 2 H),7.6-7.3 (m, 12 H). | 363 (M dot). | 3-(3-phenyl-3H-benzo[e]indol-2-yl)benzoic acid |
| 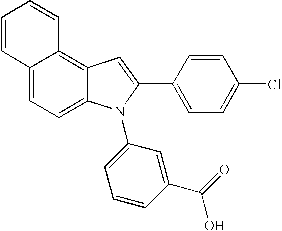 | DMSO-d6; 8.41 (d, 1 H),8.06 (tt, 1 H), 7.97 (d, 1 H),7.86 (t, 1 H), 7.71-7.6(m, 5 H), 7.5-7.46 (m,1 H), 7.43-7.40 (m, 2 H),7.32-7.35 (m, 3 H). | 397 (M dot). | 3-[2-(4-chlorophenyl)-benzo[e]indol-3-yl]benzoic acid |
| 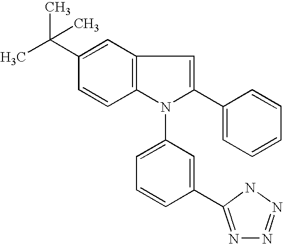 | CDCl3: 8.1 (m, 2 H); 7.7(s, 1 H); 7.5 (t, 1 H); 7.2-7.3 (m, 9 H); 6.8 (s, 1 H);0.9 (m, 9 H). | pos. mode394 (M + H). | 5-tButyl-2-phenyl-1-[3-(1H-tetrazol-5-yl) phenyl]-1H-indole |
| 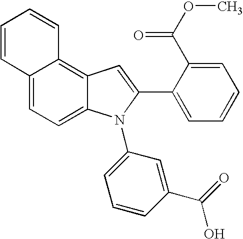 | DMSO-d6; 8.4 (d, 1 H),8.0-7.91 (m, 2 H), 7.78(s, 1 H), 7.68-7.38 (m,11 H), 3.37 (s, 3 H). | pos. mode422 (M + H). | 3-[2-(2-carbomethoxyphenyl)-benzo[e]indol-3-yl]benzoic acid |
| 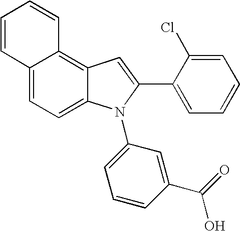 | DMSO-d6; 8.3 (d, 1 H),8.1 (m, 1 H), 8.04 (m, 1 H),7.92 (m, 1 H), 7.6 (m, 2 H),7.46 (m, 5 H), 7.3 (m, 3 H),6.8 (s, 1 H). | neg. mode396 (M − H). | 3-[2-(2-chlorophenyl)-benzo[e]indol-3-yl]benzoic acid |
| 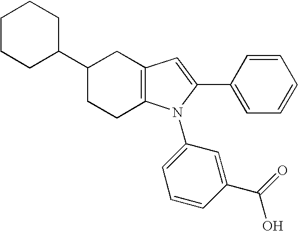 | CDCl3; 8.01 (dt, 1 H),7.96 (br s, 1 H), 7.40 (t,1 H), 7.27 (d, 1 H), 7.12-7.18 (m, 2 H), 7.03-7.12(m, 3 H), 6.26 (s, 1 H),2.67 (dd, 1 H), 2.50-2.62(m, 1 H), 2.32 -2.46 (m,2 H), 1.96 (br d, 1 H), 1.72-1.84 (m, 4 H), 1.68 (br d,1 H), 1.55-1.63 (m, 1 H),1.45 (quint., d, 1 H), 1.00-1.36 (m, 6 H). | 399 (M Dot) | 3-(5-cyclohexyl-2-phenyl-4,5,6,7-tetrahydroindol-1-yl) benzoic acid |
| 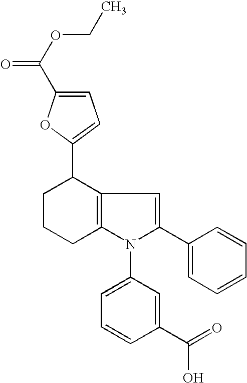 | CDCl3; 8.05 (dt, 1 H),8.00 (br s, 1 H), 7.44 (t,1 H), 7.32 (br d, 1 H), 7.00-7.17 (m, 6 H), 6.31 (s,1 H), 6.21 (dd, 1 H), 4.37(qd, 2 H), 4.22 (br t, 1 H),2.50-2.60 (m, 1 H), 2.37-2.48 (m, 1 H), 2.15-2.27 (m, 1 H), 1.96-2.07(m, 1 H), 1.85-1.95 (m,1 H), 1.72-1.85 (m, 1 H),1.38 (t, 3 H). | pos. mode456 (M + H). | 5-[1-(3-carboxyphenyl)-2-phenyl-4,5,6,7-tetrahydro-1 H-indol-4-yl]-furan-2-carboxylic acid ethyl ester |
| 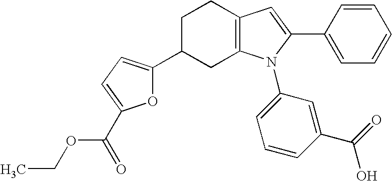 | CDCl3; 8.03 (dt, 1 H),7.93 (br s, 1 H), 7.43 (t,1 H), 7.32 (br d, 1 H), 7.03-7.20 (m, 6 H), 6.28 (s,1 H), 6.14 (dd, 1 H), 4.33(q, 2 H), 3.15-3.27 (m,1 H), 2.63-2.85 (m, 4 H),2.28-2.35 (m, 1 H), 1.88-2.03 (m, 1 H), 1.35 (t,3 H). | pos. mode456 (M + H). | 5-[1-(3-carboxyphenyl)-2-phenyl-4,5,6,7-tetrahydro-1H-indol-6-yl]-furan-2-carboxylic acid ethyl ester |
| 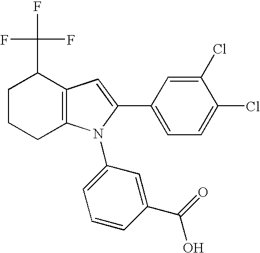 | CDCl3; 8.10 (dt, 1 H),7.94 (br s, 1 H), 7.50 (t,1 H), 7.27-7.37 (m, 1 H),7.22 (d, 1 H), 7.18 (d, 1 H),6.76 (dd, 1 H), 6.47 (s,1 H), 3.40-3.60 (m, 1 H),2.43-2.60 (m, 1 H), 2.30-2.43 (m, 1 H), 1.95-2.15 (m, 2 H), 1.82-1.95(m, 1 H), 1.65-1.80 (m,1 H). | pos. mode454 (M + H). | 3-[2-(3,4-dichlorophenyl)-4-trifluoromethyl-4,5,6,7-tetrahydroindol-1-yl]benzoic acid |
| 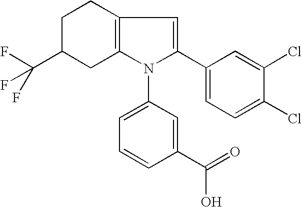 | CDCl3; 8.11 (dt, 1 H),7.88 (br s, 1 H), 7.52 (t,1 H), 7.33 (br s, 1 H), 7.15(d, 1 H), 7.17 (d, 1 H), 6.59(dd, 1 H), 6.29 (s, 1 H),2.78 (dd, 1 H), 2.40-2.70(m, 4 H), 2.15-2.25 (m,1 H), 1.73 (qd, 1 H). | pos. mode454 (M + H). | 3-[2-(3,4-dichlorophenyl)-6-trifluoromethyl-4,5,6,7-tetrahydroindol-1-yl]benzoic acid |
| 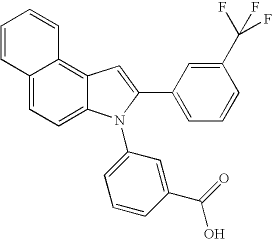 | DMSO-d6; 8.45 (d, 1 H),8.1-7.9 (m, 8 H), 7.74-7.63 (m, 4 H), 7.5 (t, 1 H),7.37 (d, 1 H). | pos. mode432 (M + H). | 3-[2-(3-trifluoromethylphenyl)-benzo[e]indol-3-yl]benzoic acid |
| 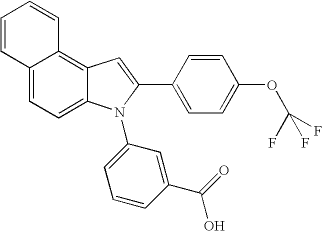 | DMSO-d6; 8.5 (d, 1 H),8.1 (tt, 1 H), 8.0 (d, 1 H),7.9 (t, 1 H), 7.74-7.64(m, 5 H), 7.53-7.46 (m,3 H), 7.3 (m, 3 H). | pos. mode448 (M + H). | 3-[2-(4-trifluoromethoxyphenyl)-benzo[e]indol-3-yl]benzoic acid |
| 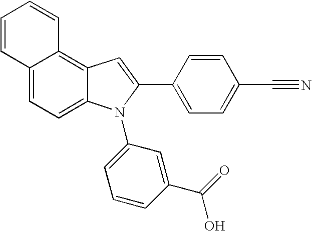 | DMSO-d6; 8.45 (d, 1 H),8.10 (tt, 1 H), 8.01 (d, 1 H),7.92 (t, 1 H), 7.86-7.83(m, 3 H), 7.76-7.72 (m,2 H), 7.70-7.65 (m, 2 H),7.54-7.5 (m, 3 H), 7.38(d, 1 H). | pos. mode389 (M + H). | 3-[2-(4-cyanophenyl)-benzo[e]indol-3-yl]benzoic acid |
| 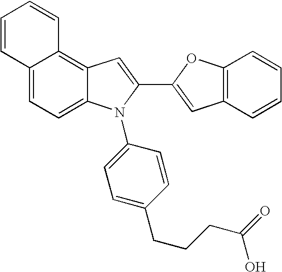 | DMSO-d6; 7.1-8.4 (m,15 H), 5.8 (s, 1 H), 2.7 (t,2 H), 2.06 (t, 2 H), 1.8 (t,2 H). | pos. mode446 (M + 1),neg. mode444 (M − 1). | 4-[4-(2-benzofuran-2-ylbenzo[e]indol-3-yl)-phenyl] butyric acid |
| 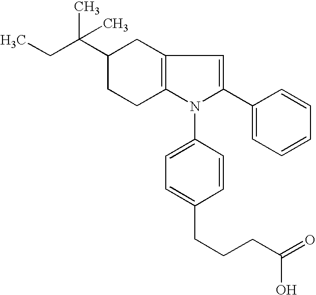 | CDCl3; 7.10-7.17 (m, 4H), 7.01-7.09 (m, 5 H),6.24 (s, 1 H), 2.58-2.72(m, 3 H), 2.47-2.58 (m,1 H), 2.30-2.46 (m, 4 H),1.90-2.05 (m, 3 H), 1.62(td, 1 H), 1.23-1.45 (m,3 H), 0.89 (s, 3 H), 0.88 (s,3 H), 0.84 (t, 3 H). | neg. mode428 (M − H). | 4-{4-[5-(1,1-dimethylpropyl)-2-phenyl-4,5,6,7,-tetrahydroindol-1-yl]-phenyl} butyric acid |
| 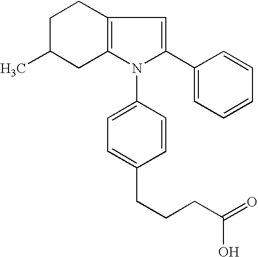 | CDCl3: 7.0-7.2 (m, 9 H);6.2 (s, 1 H); 2.7 (m, 3 H);2.4 (m, 3 H); 2.1 (m, 1 H);2.0 (m, 2 H); 1.9 (m, 2 H);1.4 (m, 2 H); 1.0 (d, 3 H). | pos. mode374 (M + H);neg. mode3.72 (M − H). | 4-[4-(6-methyl-2-phenyl-4,5,6,7-tetrahydroindol-1-yl) phenyl] butyric acid |
| 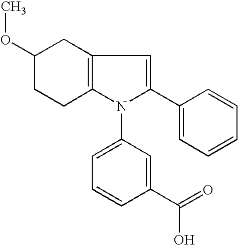 | CDCl3; 8.02 (dt, 1 H),7.97 (t, 1 H), 7.40 (t, 1 H),7.28 (ddd, 1 H), 7.07-7.18 (m, 3 H), 7.01-7.06(m, 2 H), 6.26 (s, 1 H),3.45-3.80 (m, 1 H), 3.46(s, 3 H), 2.99 (dd, 1 H),2.65 (dd, 1 H), 2.40-2.65(m, 2 H), 2.00-2.15 (m,1 H), 1.80--2.00 (m, 1 H). | pos. mode348 (M + H);neg. mode346 (M − H). | 3-(5-methoxy-2-phenyl-4,5,6,7-tetrahydroindol-1-yl) benzoic acid |
| 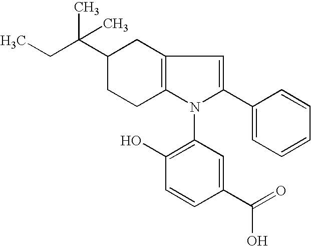 | 2 rotamers, 1:1, MeOH-d4; 7.87 (dd, 0.5 H), 7.85(dd, 0.5 H), 7.55 (d, 0.5 H),7.44 (d, 0.5 H), 7.10 (s,2 H), 7.09 (s, 2 H), 7.00-7.03 (m, 1 H), 6.98 (d,0.5 H), 6.95 (d, 0.5 H),6.18 (s, 0.5 H), 6.17 (s,0.5 H), 2.59 (dt, 1 H), 2.20-2.50 (m, 3 H), 1.85-2.00 (m, 1 H), 1.45-1.62(m, 1 H), 1.25-1.45 (m,3 H), 0.92 (s, 3 H), 0.91 (s,3 H), 0.87 (t, 3 H). | pos. mode404 (M + H);neg. mode402 (M − H). | 3-[5-(1,1-dimethylpropyl)-2-phenyl-4,5,6,7-tetrahydroindol-1-yl] 4-hydroxybenzoic acid |
| 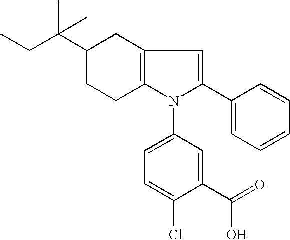 | CDCl3; 7.87 (d, 1 H),7.39 (d, 1 H), 7.16-7.23(m, 2 H), 7.09-7.16 (m,2 H), 7.04-7.09 (m, 2 H),6.26 (s, 1 H), 2.63 (dd,1 H), 2.55 (br d, 1 H), 2.32-2.48 (m, 2 H), 1.99 (br d,1 H), 1.63 (td, 1 H), 1.26-1.46 (m, 3 H), 0.90 (s,3 H), 0.89 (s, 3 H), 0.86 (t,3 H) | pos. mode422 (M + H);neg. mode420 (M − H). | 2-chloro-5-[5-(1,1-dimethylpropyl)-2-phenyl-4,5,6,7-tetrahydroindol-1-yl] benzoic acid |
| 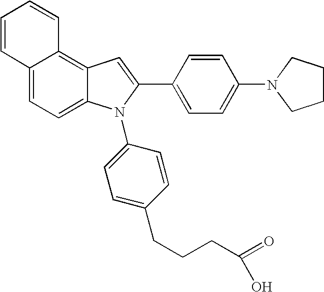 | DMSO-d6; 7.0-8.3 (m,14 H), 6.4 (s, 1 H), 3.1 (q,4 H,) 2.6 (t, 2 H), 2.04 (t,2 H), 1.9 (q, 4 H), 1.8 (t,2 H). | pos. mode475 (M + 1),neg. mode473 (M − 1). | 4-{4-[2-(4-pyrrolidin-1-ylphenyl)-benzo[e]indol-3-yl]-phenyl} butyric acid |
| 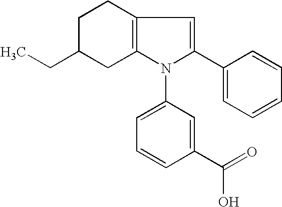 | CDCl3; 8.02 (dt, 1 H),7.94 (br s, 1 H), 7.42 (t,1 H), 7.30 (br d, 1 H), 7.11-7.18 (m, 2 H), 7.02-7.11 (m, 3 H), 6.26 (s,1 H), 2.54-2.74 (m, 2 H),2.44 (dd, 1 H), 2.10-2.20(m, 1 H), 1.94 (br d, 1 H),1.65 (br s, 1 H), 1.30-1.50 (m, 3 H), 0.91 (t, 3 H). | pos. mode346 (M + H);neg. mode344 (M − H). | 3-(6-ethyl-2-phenyl-4,5,6,7-tetrahydroindol-1-yl) benzoic acid |
| 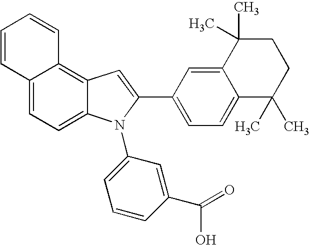 | DMSO-d6; 7.1-8.4 (m,13 H), 7.05 (s, 1 H), 1.5(dd, 4 H), 1.21 (s, 6 H),0.93 (s, 6 H). | pos. mode474 (M + 1). | 3-[2-(5,5,8,8-tetramethyl-5,6,7,8-tetrahydronaphthalen-2-yl)-benzo[e]indol-3-yl]benzoic acid |
| 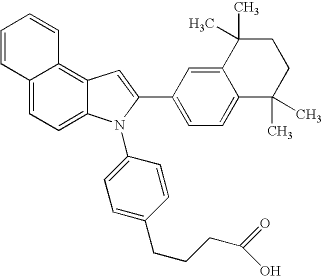 | DMSO-d6; 7.2-8.3 (m,13 H), 6.9 (s, 1 H), 2.6 (t,2 H), 1.9 (t, 2 H), 1.7 (t,2 H), 1.5 (q, 4 H), 1.21 (s,6 H), 0.9 (s, 6 H). | pos. mode516 (M + 1). | 4-{4-[2-(5,5,8,8-tetramethyl-5,6,7,8-tetrahydronaphthalen-2-yl)-benzo[e]indol-3-yl]-phenyl} butyric acid |
| 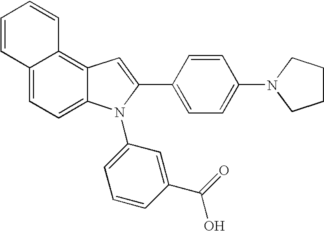 | DMSO-d6; 7.1-8.3 (m,14 H), 6.4 (s, 1 H), 3.2 (q,4 H), 1.9 (q, 4 H). | pos. mode433 (M + 1),neg. mode431 (M − 1). | 3-[2-(4-pyrrolidin-1-ylphenyl)-benzo[e]indol-3-yl] benzoic acid |
| 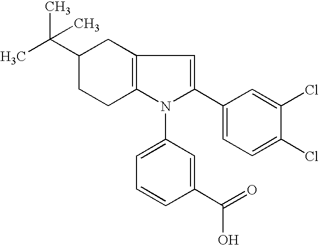 | CDCl3: 8.0 (d, 1 H); 7.9(br. s, 1 H); 7.4 (t, 1 H); 7.2(m, 2 H); 7.1 (d, 1 H); 6.7(dd, 1 H); 6.2 (s, 1 H); 2.7(m, 1 H); 2.5 (m, 1 H); 2.3-2.4 (m, 2 H); 2.0 (m, 1 H);1.4 (m, 1 H); 1.3 (m, 1 H);1.0 (s, 9 H). | pos. mode422 (M + H);neg. mode440 (M − H). | 3-[5-tButyl-2-(3,4-dichlorophenyl)-4,5,6,7-tetrahydroindol-1-yl]benzoic acid |
| 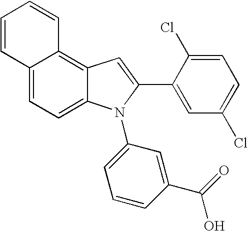 | DMSO-d6; 8.38 (d, 1 H),7.98-7.92 (m, 2 H), 7.84(m, 1 H), 7.7-7.66 (m,2 H), 7.62-7.56 (m, 3 H),7.51 (s, 1 H), 7.48-7.4(m, 4 H). | neg. mode430 (M − H). | 3-[2-(2,5-dichlorophenyl)-benzo[e]indol-3-yl]benzoic acid |
| 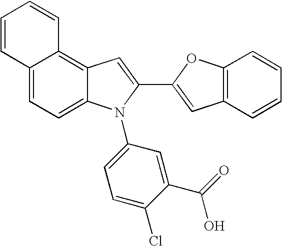 | DMSO-d6; 7.1-8.4 (m,14 H), 6.09 (s, 1 H). | pos. mode438 (M + 1). | 5-(2-benzofuran-2-ylbenzo[e]indol-3-yl) 2-chlorobenzoic acid |
| 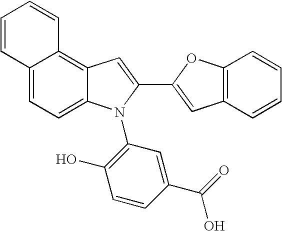 | DMSO-d6; 7.1-8.4 (m,14 H), 5.9 (s, 1 H). | pos. mode420 (M + 1). | 3-(2-benzofuran-2-ylbenzo[e]indol-3-yl) 4-hydroxybenzoic acid |
| 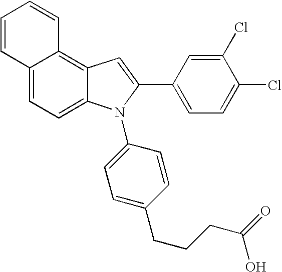 | DMSO-d6; 8.4 (d, 1 H),7.96 (d, 1 H), 7.73 (s, 1 H),7.66-7.56 (m, 3 H), 7.5-7.44 (m, 2 H), 7.4 (d, 2 H),7.34-7.28 (m, 3 H), 7.26(dd, 1 H), 2.65 (t, 2 H), 2.3(t, 2 H), 1.9 (t, 2 H). | pos. mode474 (M + H). | 4-{4-[2-(3,4-dichlorophenyl)-benzo[e]indol-3-yl]-phenyl} butyric acid |
| 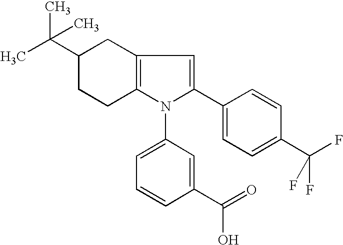 | CDCl3: 8.0 (d, 1 H); 7.9(br. s, 1 H); 7.4 (m, 3 H);7.2 (m, 1 H); 7.1 (d, 2 H);6.4 (s, 1 H); 2.7 (m, 1 H);2.5 (m, 1 H); 2.3-2.4 (m,2 H); 2.0 (m, 1 H); 1.5 (m,1 H); 1.4 (m, 1 H); 1.0 (s,9 H). | TOF pos. mode442 (M + H),EM 442.1994. | 3-[5-tButyl-2-(4-trifluoromethylphenyl)-4,5,6,7-tetrahydroindol-1-yl] benzoic acid |
| 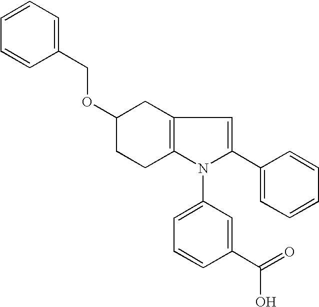 | CDCl3; 8.02 (d, 1 H),7.96 (s, 1 H), 7.33-7.45(m, 5 H), 7.25-7.32 (m,2 H), 7.08-7.18 (m, 3 H),7.00-7.08 (m, 2 H), 6.25(s, 1 H), 4.67 (s, 2 H), 3.86-3.92 (m, 1 H), 3.02 (dd,1 H), 2.71 (dd, 1 H), 2.48-2.58 (m, 2 H), 2.05-2.18(m, 1 H), 1.88-2.04 (m,1 H). | pos. mode424 (M + H);neg. mode422 (M − H). | 3-(5-benzyloxy-2-phenyl-4,5,6,7-tetrahydroindol-1-yl) benzoic acid |
| 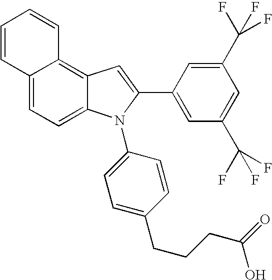 | DMSO-d6; 8.43 (d, 1 H),7.99-7.95 (m, 2 H), 7.87(s, 2 H), 7.69 (d, 1 H), 7.64(t, 1 H), 7.49 (t, 1 H), 7.43-7.34 (m, 6 H), 2.7 (t, 2 H),2.25 (t, 2 H), 1.84 (t, 2 H). | pos. mode542 (M + H);neg. mode540 (M − H). | 4-{4-[2-(3,5-bistrifluoromethylphenyl)-benzo[e]indol-3-yl]-phenyl} butyric acid |
| 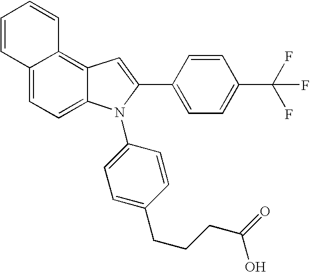 | CDCl3; 8.32 (d, 1 H), 7.91(d, 1 H), 7.6-7.2 (m,13 H), 2.8 (t, 2 H); 2.4 (t,2 H); 2.0 (t, 2 H). | pos. mode474 (M + H);neg. mode472 (M − H). | 4-{4-[2-(4-trifluoromethylphenyl)-benzo[e]indol-3-yl]-phenyl} butyric acid |
| 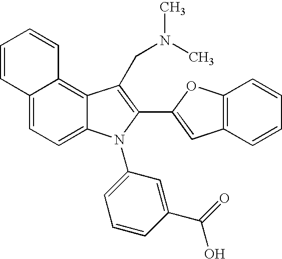 | DMSO-d6; 6.8-8.7 (m,15 H), 3.9 (s, 2 H), 2.27 (s,6 H). | neg. mode459 (M − 1). | 3-(2-benzofuran-2-yl-1-dimethylaminomethyl-benzo[e]indol-3-yl) benzoicacid |
| 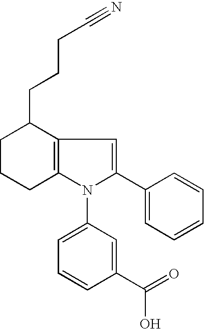 | CDCl3; 8.03 (d, 1 H),7.96 (s, 1 H), 7.42 (t, 1 H),7.30 (d, 1 H), 7.00-7.20(m, 5 H), 6.31 (s, 1 H),2.70-2.85 (m, 1 H), 2.30-2.58 (m, 4 H), 1.75-2.05 (m, 5 H), 1.60-1.85(m, 2 H), 1.40-1.50 (m,1 H). | pos. mode385 (M + H). | 3-[4-(3-cyanopropyl)-2-phenyl-4,5,6,7-tetrahydroindol-1-yl]benzoic acid |
| 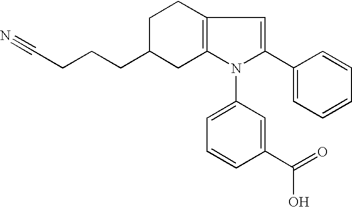 | CDCl3; 8.04 (dt, 1 H),7.94 (br s, 1 H), 7.43 (t,1 H), 7.29 (br d, 1 H), 7.08-7.20 (m, 3 H), 7.02-7.08 (m, 2 H), 6.26 (s,1 H), 2.56-2.74 (m, 2 H),2.45 (dd, 1 H), 2.33 (t,2 H), 2.12-2.23 (m, 1 H),1.90-1.98 (m, 1 H), 1.75-1.85 (m, 1 H), 1.62-1.75 (m, 2 H), 1.40-1.61(m, 3 H) | pos. mode385 (M + H). | 3-[6-[3-cyanopropyl)-2-phenyl-4,5,6,7-tetrahydroindol-1-yl]-benzoic acid |
| 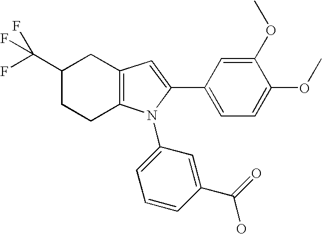 | DMSO-d6; 7.8 (d, 1 H),7.56 (s, 1 H), 7.42 (t, 1 H),7.24 (d, 1 H), 7.09 (d, 1 H),6.46 (d, 1 H), 6.32 (s, 1 H),5.99 (s, 1 H), 3.7 (s, 3 H),3.3 (s, 3 H), 2.8-2.55 (m,4 H), 2.39 (d, 1 H), 2.10 (d,1 H), 1.66-1.61 (m, 1 H). | pos. mode466 (M + H);neg. mode444 (M − H). | 3-[2-(3,4-dimethoxyphenyl)-5-trifluoromethyl-4,5,6,7-tetrahydroindol-1-yl]-benzoic acid |
| 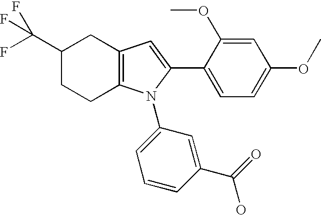 | DMSO-d6; 7.91 (d, 1 H),7.63 (s, 1 H), 7.56 (t, 1 H),7.44 (d, 1 H), 6.78 (d, 1 H),6.57 (d, 1 H), 6.51 (s, 1 H),6.24 (s, 1 H), 3.67 (s, 3 H),3.35 (s, 3 H), 2.81-2.55(m, 4 H), 2.36 (d, 1 H),2.10 (m, 1 H), 1.66-1.61(m, 1 H). | pos. mode466 (M + H);neg. mode444 (M − H). | 3-[2-(2,4-dimethoxyphenyl)-5-trifluoromethyl-4,5,6,7-tetrahydroindol-1-yl]benzoic acid |
| 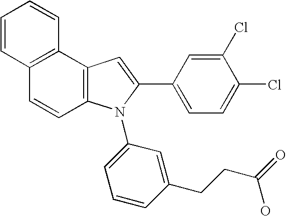 | DMSO-d6; 8.38 (d, 1 H),7.96 (d, 1 H), 7.64-7.3(m, 12 H), 7.1-7.07 (tt,1 H), 2.89 (t, 2 H), 2.55 (t,2 H). | pos. mode426 (M + H);neg. mode424 (M − H). | 3-{3-[2-(4-chlorophenyl)-benzo[e]indol-3-yl]-phenyl} propionic acid |
| 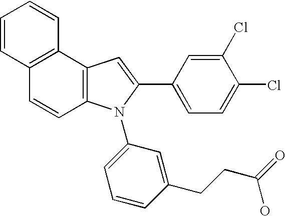 | DMSO-d6; 8.39 (d, 1 H),7.97 (d, 1 H), 7.73 (s, 1 H),7.67-7.33 (m, 9 H); 7.19-7.12 (m, 2 H); 2.9 (t, 2 H);2.55 (t, 2 H). | pos. mode460 (M + H). | 3-{3-[2-(3,4-dichlorophenyl)-benzo[e]indol-3-yl]-phenyl} propionic acid |
| 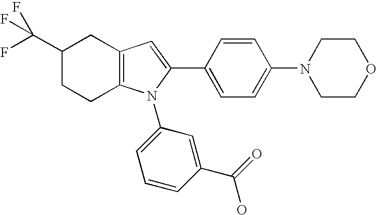 | DMSO-d6; 7.92-7.89 (tt,1 H), 7.62 (s, 1 H), 7.54 (t,1 H), 7.4 (d, 1 H), 6.87 (d,2 H), 6.75 (d, 2 H), 6.16 (s,1 H), 3.68 (t, 4 H), 3.34 (s,2 H), 3.01 (t, 4 H), 2.8-2.76 (dd, 1 H), 2.6 (dd,1 H), 2.34 (m, 1 H), 2.1 (m,1 H), 1.63 (m, 1 H). | pos. mode471 (M + H);neg. mode469 (M − H). | 3-[2-(4-morpholin-4-yl-phenyl)-5-trifluoromethyl-4,5,6,7-tetrahydroindol-1-yl] benzoic acid |
| 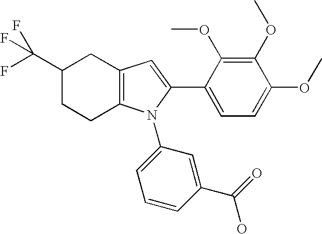 | CDCl3; 7.95-7.88 (m,2 H), 7.34 (t, 1 H), 7.22 (d,1 H), 6.87 (d, 1 H), 6.56 (d,1 H), 6.15 (s, 1 H); 3.83 (s,3 H) 3.65 (s, 3 H); 3.51 (s,3 H); 2.93-2.88 (dd, 1 H);2.71 (t, 2 H); 2.54-2.51(m, 2 H); 2.21 (m, 1 H)1.77-1.72 (m, 1 H). | pos. mode476 (M + H);neg. mode474 (M − H). | 3-[5-trifluoromethyl-2-(2,3,4-trimethoxyphenyl)-4,5,6,7-tetrahydroindol-1-yl] benzoic acid |
| 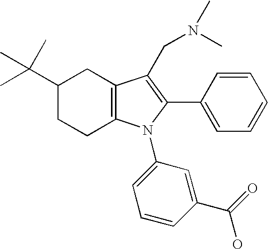 | DMSO-d6; 6.9-7.8 (m,9 H), 3.4 (s, 2 H), 2.2 (s,3 H), 1.9 (s, 3 H), 1.4-1.5(m, 2 H), 0.9 (s, 9 H). | neg. mode429 (M − 1). | 3-(5-tButyl-3-dimethylaminomethyl-2-phenyl-4,5,6,7-tetrahydroindol-1-yl)benzoic acid |
| 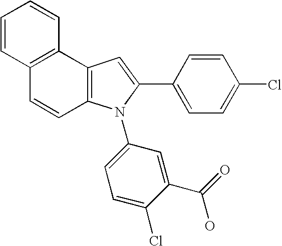 | DMSO-d6; 8.4 (d, 1 H);7.97 (d, 1 H); 7.85 (d, 1 H);7.76-7.46 (m, 9 H); 7.39-7.33 (dd, 1 H); 7.2-7.15(td, 1 H). | pos. mode432 (M + H). | 2-chloro-5-[2-(4-chlorophenyl)-benzo[e]indol-3-yl]benzoic acid |
| 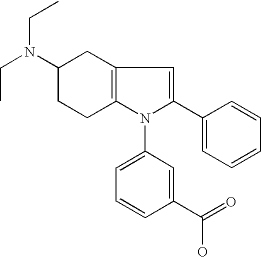 | MeOH-d4; 7.99 (d, 1 H),7.77 (s, 1 H), 7.48 (t, 1 H),7.28-7.33 (m, 1 H), 7.10-7.20 (m, 3 H), 7.00-7.10 (m, 2 H), 6.28 (s,1 H), 3.75-3.88 (m, 1 H),3.37-3.50 (m, 2 H), 3.23-3.35 (m, 2 H), 3.07 (dd,1 H), 2.93 (dd, 1 H), 2.65-2.82 (m, 1 H), 2.59 (br d,1 H), 2.27 (br d, 1 H), 2.03(qd, 1 H), 1.40 (t, 6 H). | pos. mode389 (M + H);neg. mode387 (M − H) | 3-(5-diethylamino-2-phenyl-4,5,6,7-tetrahydroindol-1-yl)benzoic acid |
| 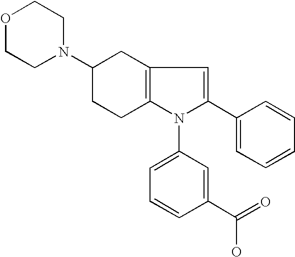 | MeOH-d4; 7.99 (dt, 1 H),7.77 (br s, 1 H), 7.48 (t,1 H), 7.31 (br d, 1 H), 7.08-7.20 (m, 3 H), 7.00-7.06 (m, 2 H), 6.27 (s,1 H), 3.75-4.12 (m, 4 H),3.58-3.72 (m, 1 H), 3.20-3.57 (m, 4 H), 3.10-3.20 (m, 1 H), 2.82-2.95(m, 1 H), 2.68-2.79 (m,1 H), 2.61 (br d, 1 H), 2.40(br d, 1 H), 1.98 (qd, 1 H). | pos. mode403 (M + H);neg. mode401 (M − H). | 3-(5-morpholin-4-yl-2-phenyl-4,5,6,7-tetrahydroindol-1-yl)benzoic acid |
| 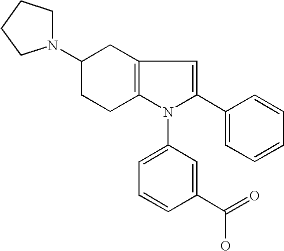 | DMSO-d6; 7.87 (d, 1 H),7.59 (br s, 1 H), 7.47 (t,1 H), 7.32 (br d, 1 H), 7.11-7.20 (m, 2 H), 7.03-7.10 (m, 1 H), 6.94-7.02(m, 2 H), 6.23 (s, 1 H),2.55-2.90 (m, 6 H), 2.30-2.40 (m, 2 H), 2.05-2.18 (m, 2 H), 1.75 (br s,4 H), 1.57-1.70 (m, 1 H). | pos. mode387 (M + H);neg. mode385 (M − H). | 3-(2-phenyl-5-pyrrolidin-1-yl-4,5,6,7-tetrahydroindol-1-yl)benzoic acid |
| 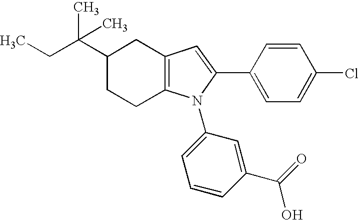 | DMSO-d6; 7.91 (d, 1 H),7.60-7.41 (m, 3 H); 7.25-7.21 (m, 2 H); 7.0-6.96(m, 2 H); 6.27 (s, 1 H);2.54-2.44 (m, 1 H), 2.31-2.25 (m, 2 H); 1.91 (m,1 H); 1.56-1.50 (m, 1 H)1.37-1.24 (m, 4 H); 0.86-0.80 (m, 9 H). | pos. mode422 (M + H),neg. mode420 (M − 1). | 3-[2-(4-chlorophenyl)-5-(1,1-dimethylpropyl)-4,5,6,7-tetrahydroindol-1-yl] benzoic acid |
| 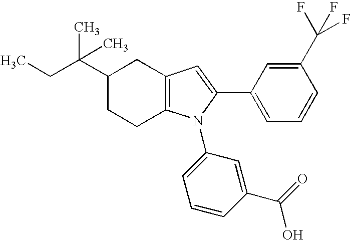 | DMSO-d6; 7.94-7.92 (dt,1 H), 7.65-7.38 (m, 5 H);7.17 (d, 2 H); 6.42 (s, 1 H);2.51-2.48 (m, 1 H), 2.31-2.21 (m, 2 H); 1.95-1.90(m, 1 H); 1.59-1.51 (m,1 H) 1.39-1.23 (m, 4 H);0.87-0.81 (m, 9 H). | pos. mode456 (M + H),neg. mode454 (M − 1). | 3-[5-(1,1-dimethylpropyl)-2-(3-trifluoromethylphenyl)-4,5,6,7-tetrahydro-indol-1-yl] benzoic acid |
| 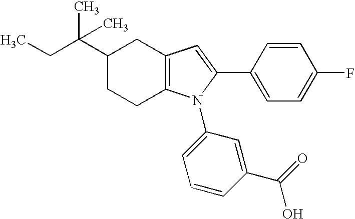 | CDCl3; 8.02-7.93 (m,2 H), 7.41 (t, 1 H); 7.23 (m,1 H); 7.03-6.99 (m, 2 H),6.87-6.83 (m, 2 H) 6.22 (s,1 H); 2.66-2.53 (m, 2 H),2.42-2.35 (m, 2 H); 1.99-1.96 (m, 1 H); 1.66-1.60(m, 1 H) 1.43-1.33 (m,3 H); 0.90-0.83 (m, 9 H). | pos. mode406 (M + H),neg. mode404 (M − 1). | 3-[5-(1,1-dimethylpropyl)-2-(4-fluorophenyl)-4,5,6,7-tetrahydroindol-1-yl]benzoic acid |
| 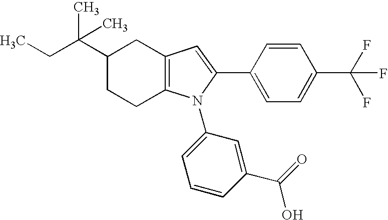 | CDCl₃; 8.05-7.95 (m, 2 H),7.44 (t, 1 H); 7.33-7.13(m, 5 H), 6.34 (s, 1 H);2.67-2.53 (m, 2 H), 2.45-2.35 (m, 2 H); 2.0-1.97(m, 1 H); 1.66-1.59 (m,1 H) 1.43-1.35 (m, 3 H);0.90-0.84 (m, 9 H). | pos. mode456 (M + H),neg. mode454 (M − 1). | 3-[5-(1,1-dimethylpropyl)-2-(4-trifluoromethyl-phenyl)-4,5,6,7-tetrahydro-indol-1-yl]benzoic acid |
| 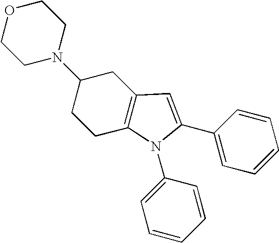 | CDCl3; 7.27-7.38 (m,3 H), 7.01-7.18 (m, 7 H),6.25 (s, 1 H), 3.72-3.80(m, 4 H), 2.75-2.90 (m,2 H), 2.46-2.74 (m, 7 H),2.07-2.17 (m, 1 H), 1.70(qd, 1 H). | pos. mode359 (M + H). | 5-morpholin-4-yl-1,2-diphenyl-4,5,6,7-tetrahyrdro-1H-indole |
| 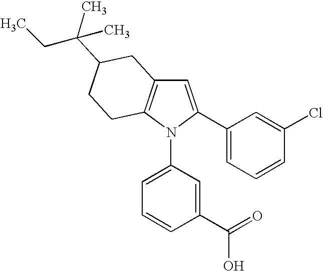 | CDCl3; 8.04 (dd, 1 H),7.94 (br s, 1 H), 7.44 (t,1 H), 7.25-7.30 (m, 1 H),7.08-7.15 (m, 1 H), 6.98-7.06 (m, 2 H), 6.81 (dt,1 H), 6.29 (s, 1 H), 2.63(dd, 1 H), 2.48-2.58 (m,1 H), 2.30-2.46 (m, 2 H),1.98-2.20 (m, 1 H), 1.56-1.68 (m, 1 H), 1.30-1.44 (m, 3 H), 0.90 (s,3 H), 0.89 (s, 3 H), 0.85 (t,3 H). | pos. mode422 (M + H). | 3-[2-(3-chlorophenyl)-5-(1,1-dimethylpropyl)-4,5,6,7-tetrahydroindol-1-yl] benzoic acid |
| 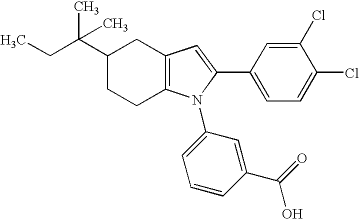 | CDCl₃; 8.08-8.05; (dt,1 H); 7.94 (s, 1 H), 7.46 (t,1 H); 7.29-7.27 (m, 1 H),7.21-7.15 (m, 2 H); 6.76-6.73 (dd, 1 H), 6.29 (s,1 H), 2.65-2.60 (m, 1 H),2.44-2.32 (m, 2 H), 1.98(m, 1 H), 1.66-1.56 (m,1 H), 1.42-1.33 (m, 4 H),0.89-0.83 (m, 9 H). | pos. mode457 (M + H). | 3-[2-(3,4-dichlorophenyl)-5-(1,1-dimethylpropyl)-4,5,6,7-tetrahydroindol-1-yl] benzoic acid |
| 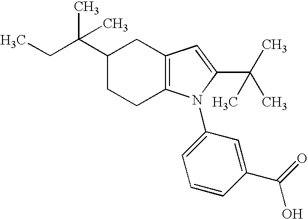 | CDCl3; 8.16-8.14; (m,1 H); 8.05 (m, 1 H), 7.55-7.52 (m, 2 H); 5.90 (s,1 H); 2.43-2.39 (m, 2 H);2.06-2.01 (m, 1 H); 1.59-1.43 (m, 2 H); 1.34-1.29(m, 5 H); 1.13 (s, 9 H);0.87-0.79 (m, 9 H). | pos. mode368 (M + H). | 3-[2-tert-butyl-5-(1,1-dimethylpropyl)-4,5,6,7-tetrahydroindol-1-yl]benzoic acid |
| | CDCl3; 8.03-8.09 (m,2 H), 7.42-7.52 (m, 3 H),7.30-7.42 (m, 4 H), 7.17-7.29 (m, 7 H), 6.94 (dd,1 H), 6.74 (d, 1 H), 5.15 (s,2 H). | pos. mode420 (M + H) | 3-(5-benzyloxy-2-phenylindol-1-yl) benzoicacid |
| | CDCl3; 8.11 (d, 1 H),8.01 (d, 2 H), 7.93 (br s,1 H), 7.52 (t, 1 H), 7.34 (brs, 1 H), 7.14 (d, 2 H), 6.48(s, 1 H), 2.92 (dd, 1 H),2.40-2.78 (m, 4 H), 2.16-2.24 (m, 1 H), 1.64-1.82 (m, 1 H). | pos mode431 (M + H) | 3-[2-(4-nitrophenyl)-5-trifluoromethyl-4,5,6,7-tetrahydroindol-1-yl]benzoic acid |
| 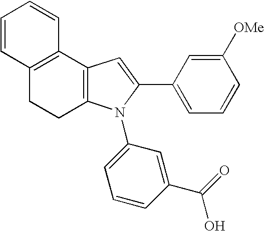 | 7.95 (tt, 1 H), 7.7 (t, 1 H),7.62 (t, 1 H), 7.58-7.46(m, 2 H), 7.22 (d, 2 H),7.08-7.04 (m, 3 H), 6.86-6.82 (m, 2 H), 6.75 (s,1 H), 3.70 (s, 3 H), 2.93 (t,2 H), 2.62 (t, 2 H). | pos. mode396 (M + H);neg. mode394 (M − H). | 3-[2-(3-methoxyphenyl)-4,5-dihydrobenzo[e]indol-3-yl] benzoic acid |
| 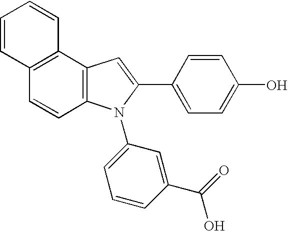 | DMSO-d6; 8.3 (d, 1 H),79 (d, 1 H), 7.75 (d, 1 H),7.56-7.26 (m, 11 H), 7.18(s, 1 H). | pos. mode382 (M + H);neg. mode380 (M − H). | 3-[2-(4-hydroxyphenyl)-benzo[e]indol-3-yl]benzoic acid |
| 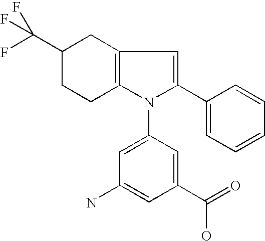 | CDCl3; 7.63 (s, 1 H),7.51 (s, 1 H), 7.03-7.20(m, 5 H), 6.94 (s, 1 H),6.27 (s, 1 H), 2.90 (dd,1 h), 2.38-2.78 (m, 4 H),2.20 (d, 1 H), 1.74 (qd,1 H). | pos. mode401 (M + H) | 3-amino-5-(2-phenyl-5-trifluoromethyl-4,5,6,7-tetrahydroindol-1-yl)benzoic acid |
| 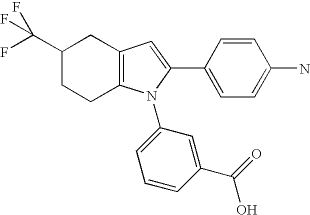 | CDCl3; 8.02 (d, 1 H),7.93 (s, 1 H), 7.41 (t, 1 H),7.25-7.30 (m, 1 H), 6.80-6.90 (m, 2 H), 6.44-6.52 (m, 2 H), 6.16 (s,1 H), 2.89 (dd, 1 H), 2.58-2.76 (m, 2 H), 2.42-2.52(m, 2 H), 2.19 (br d, 1 H),1.64 (qd, 1 H). | pos. mode401 (M + H) | 3-[2-(4-aminophenyl)-5-trifluoromethyl-4,5,6,7-tetrahydroindol-1-yl]benzoic acid |
| 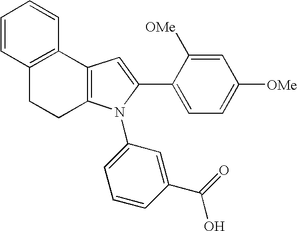 | DMSO-d6; 7.95 (tt, 1 H),7.7 (t, 1 H), 7.59 (t, 1 H),7.51-7.44 (m, 2 H), 7.18(d, 2 H), 7.05-7.0 (m, 3 H),6.8 (m, 1 H), 6.7 (s, 1 H),3.7 (s, 3 H), 3.3 (s, 3 H),2.9 (t, 2 H), 2.6 (t, 2 H). | pos. mode426 (M + H);neg. mode424 (M − H). | 3-[2-(2,4-dimethoxyphenyl)-4,5-dihydrobenzo[e]indol-3-yl]benzoic acid |
| 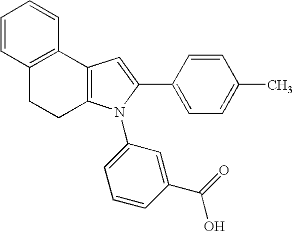 | MeOH-d4 (mixture55%:45%saturated:unsaturated);8.22 (d, 2 H); 7.94 (d, 2 H),7.85-7.83 (d, 2 H), 7.75-7.67 (m, 4 H), 7.60-7.56(m, 4 H), 7.49-7.45 (m,1 H), 7.35-7.28 (m, 4 H),7.20-7.17 (m, 5 H), 7.1-7.07 (m, 2 H), 7.0 (s, 2 H),3.1 (t, 2 H), 2.93 (t, 2 H),2.4 (s, 3 H), 2.33 (s, 3 H). | neg. mode378 (M − H). | 3-(2-p-tolyl-4,5-dihydrobenzo[e]indol-3-yl)benzoic acid |
| 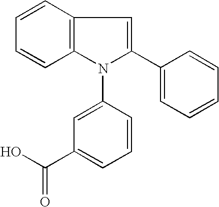 | CDCl3; 8.1 (m, 2 H); 7.7(m, 1 H); 7.5 (t, 1 H); 7.4(m, 1 H); 7.2-7.3 (m, 8 H,ArH); 6.8 (s, 1 H). | pos. mode314 (M + H);neg. mode312 (M − H) | 3-(2-phenylindol-1-yl)benzoic acid |
| 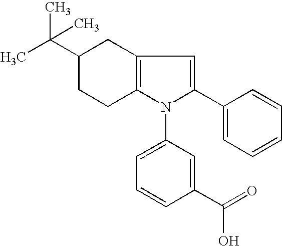 | CDCl3/d3-MeOD; 8.0 (m,2 H); 7.4 (t, 1 H); 7.2 (m,1 H); 7.0-7.2 (m, 5 H,ArH); 6.2 (s, 1 H); 2.7 (m,1 H); 2.5 (s, 1 H); 2.4 (m,2 H); 2.0 (m, 1 H); 1.5 (m,1 H); 1.4 (m, 1 H); 0.9 (s,9 H). | pos. mode374 (M + H);neg. mode372 (M − H) | 3-(5-tert-Butyl-2-phenyl-4,5,6,7-tetrahydroindol-1-yl) benzoic acid |
| 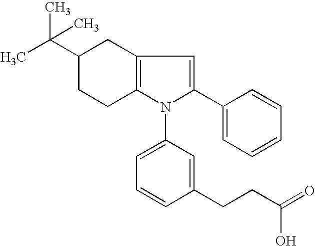 | CDCl3; 7.2 (m, 1 H); 6.9-7.1 (m, 8 H, ArH); 6.2 (s,1 H); 2.9 (t, 2 H); 2.7 (m,1 H); 2.5 (m, 3 H); 2.4 (m,2 H); 2.0 (m, 1 H); 1.5 (m,1 H); 1.4 (m, 1 H); 0.9 (s,9 H). | pos. mode402 (M + H);neg. mode400 (M − H) | 3-[3-(5-tert-Butyl-2-phenyl-4,5,6,7-tetrahydroindol-1-yl)-phenyl] propionic acid |
| 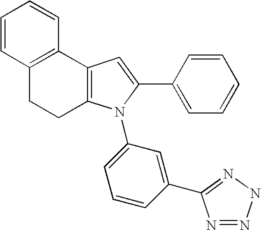 | DMSO-d6; 7.0-8.4 (13 H,ArH); 6.9 (1 H), 2.9 (2 H,CH2), 2.5 (2 H, CH2). | pos. mode342 (M + H);neg. mode340 (M − H) | 2-phenyl-3-[3-(2H-tetrazol-5-yl)-phenyl]-4,5-dihydro-3H-benzo[e]indole |
| 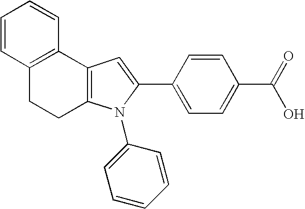 | DMSO-d6; 6.8-7.9 (14 H,ArH), 3.0 (2 H, CH2) 2.7(2 H, CH2). | neg. mode364 (M − 1) | 4-(3-phenyl-4,5-dihydro-3H-benzo[e]indol-2-yl)benzoic acid |
| 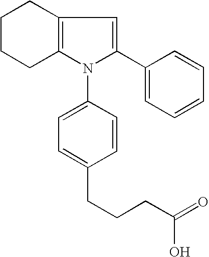 | CDCl3; 7.0-7.2 (m, 9 H,ArH); 6.2 (s, 1 H); 2.6 (m,4 H); 2.4 (m, 4 H); 2.0 (m,3 H); 1.8 (s, 3 H). | neg. mode358 (M − H) | 4-[4-(2-phenyl-4,5,6,7-tetrahydroindol-1-yl)-phenyl] butyric acid |
| 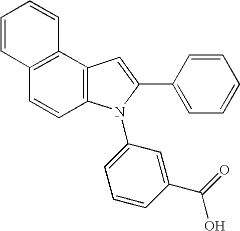 | DMSO-d6; 7.2-8.4(16 H, ArH). | pos. mode364 (M + 1);neg. mode362 (M − 1) | 3-(2-phenylbenzo[e]indol-3-yl) benzoic acid |
| 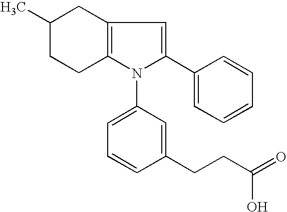 | CDCl3; 7.3 (t, 1 H); 6.9-7.1 (m, 8 H, ArH); 6.2 (s,1 H); 2.9 (t, 2 H); 2.7 (m,1 H); 2.5 (m, 3 H); 2.4 (m,1 H); 2.2 (m, 1 H); 1.9 (m,2 H); 1.4 (m, 1 H); 1.0 (d,3 H). | pos. mode360 (M + H);neg. mode358 (M − H) | 3-[3-(5-methyl-2-phenyl-4,5,6,7-tetrahydroindol-1-yl)-phenyl] propionic acid |
| 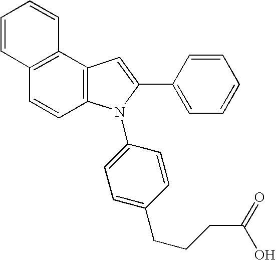 | DMSO-d6; 7.2-8.4(16 H, ArH); 2.7 (2 H,CH2); 2.3 (2 H, CH2); 1.9(2 H, CH2). | pos. mode406 (M + 1);neg. mode404 (M − 1). | 4-[4-(2-phenyl-benzo[e]indol-3-yl)-phenyl] butyric acid |
| 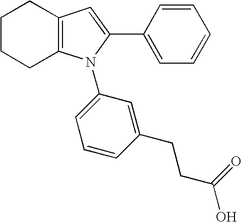 | CDCl3; 7.3 (t, 1 H); 6.9-7.2 (m, 8 H, ArH); 6.2 (s,1 H); 2.9 (t, 2 H); 2.6 (br. s,2 H); 2.5 (t, 2 H); 2.4 (br. s,2 H); 1.8 (br. s, 4 H). | pos. mode346 (M + H) | 3-[3-(2-phenyl-4,5,6,7-tetrahydroindol-1-yl)-phenyl] propionic acid |
| 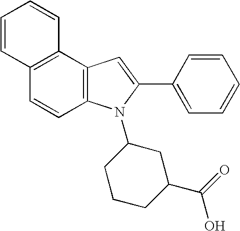 | CDCl3; 7.1-8.4 (11 H,ArH), 6.4 (1 H, ArH), 4.4(1 H, CH) 1.4-2.7 (9 H,CH2). | pos. mode372 (M + 1) | 3-(2-phenylbenzo[e]indol-3-yl)cyclohexanecarboxylicacid |
| 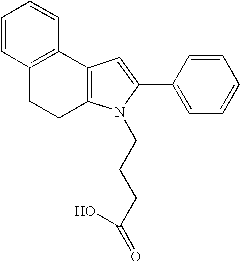 | CD3OD-d4; 7.1-8.2 (10 H,ArH), 4.0 (2 H, CH2), 3.0(2 H, CH2), 2.9 (2 H,CH2), 2.1 (2 H, CH2), 1.9(2 H, CH2). | pos. mode332 (M + 1) | 4-(2-phenyl-4,5-dihydrobenzo[e]indol-3-yl)butyric acid |
| 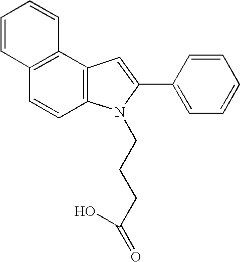 | CD3OD-d4; 7.1-8.2 (12 H,ArH) 4.4 (2 H, CH2) 2.1(2 H, CH2) 1.9 (2 H, CH2). | pos. mode330 (M + 1) | 4-(2-phenyl-benzo[e]indol-3-yl) butyricacid |
| 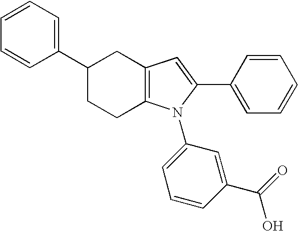 | DMSO-d6; 7.0-7.9 (14 H,ArH), 6.3 (1 H, ArH), 3.0(1 H, CH), 2.8 (1 H, CH2),2.7 (2 H, CH2), 2.4 (1 H,CH2), 1.9 (2 H, CH2). | pos. mode394 (M +1) | 3-(2,5-diphenyl-4,5,6,7-tetrahydroindol-1-yl)benzoic acid |
| 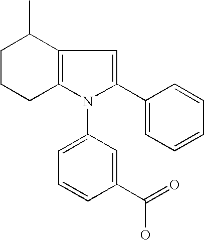 | CDCl3; 8.0 (m, 1 H); 7.9(m, 1 H); 7.4 (t, 1 H); 7.0-7.3 (m, 6 H, ArH); 6.2 (s,1 H); 2.6 (m, 1 H); 2.5 (br.s, 1 H); 2.4 (m, 1 H); 2.1(m, 1 H); 1.9 (m, 2 H); 1.4(m, 1 H); 1.0 (d, 3 H). | pos. mode332 (M + H) | 3-(4-methyl-2-phenyl-4,5,6,7-tetrahydroindol-1-yl) benzoic acid |
| 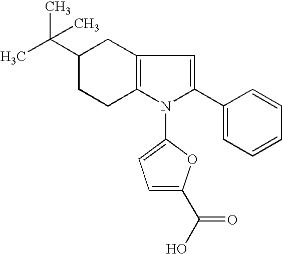 | CDCl3; 7.1-7.3 (m, 6 H,ArH); 6.2 (s, 1 H); 6.0 (d,1 H); 2.6 (m, 2 H); 2.4-2.5(m, 2 H); 2.0 (m, 1 H); 1.5(m, 2 H); 1.0 (s, 9 H). | pos. mode364 (M + H);neg. mode362 (M − H) | 5-(5-tertButyl-2-phenyl-4,5,6,7-tetrahydroindol-1-yl) furan-2-carboxylic acid |
| 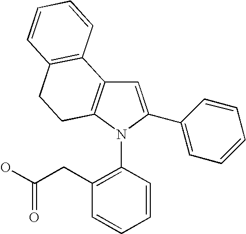 | acetone-d6; 7.5 (m, 5 H);7.2 (m, 7 H); 7.0 (t, 1 H);6.8 (s, 1 H); 3.2 (s, 2 H,CH2); 2.9 (m, 2 H); 2.6(m, 1 H); 2.4 (m, 1 H). | pos. mode380 (M + H) | [2-(2-phenyl-4,5-dihydrobenzo[e]indol-3-yl)-phenyl] acetic acid |
| 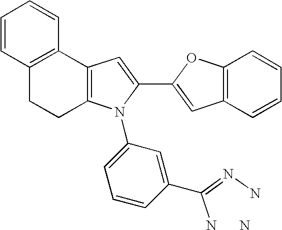 | DMSO-d6; 7.1-8.5(14 H, Ar/NH); 5.8 (1 H);2.9 (2 H, CH2); 2.6 (2 H,CH2). | pos. mode430 (M + 1);neg. mode429 (M − 1). | 2-benzofuran-2-yl-3-[3-(2H-tetrazol-5-yl)-phenyl]-4,5-dihydro-3H-benzo[e]indole |
| 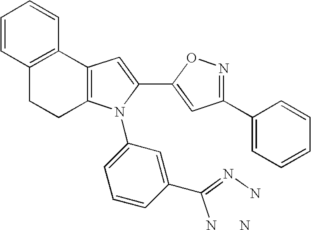 | DMSO-d6; 7.0-8.2(15 H, ArH/NH); 6.3 (1 H);2.9 (2 H, CH2); 2.6 (2 H,CH2). | pos. mode457 (M + 1);neg. mode455 (M − 1). | 2-(3-phenylisoxazol-5-yl)-3-[3-(2H-tetrazol-5-yl)-phenyl]-4,5-dihydro-3H-benzo[e]indole |
| 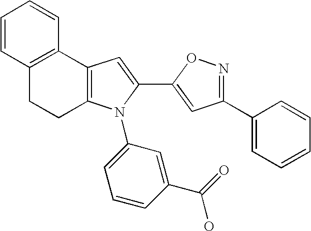 | DMSO-d6; 7.0-8.1 (14 H,ArH); 6.2 (1 H); 2.9 (2 H,CH2); 2.6 (2 H, CH2). | pos. mode433 (M + 1);heg. mode431 (M − 1). | 3-(2-phenylisoxazol-5-yl)-4,5-dihydrobenzo[e]indol-3-yl] benzoic acid |

TABLE 8
Compounds of the Invention and Starting Materials

| product structure | ketone/enamine SM | β-bromo ketone SM |
|---|---|---|
| 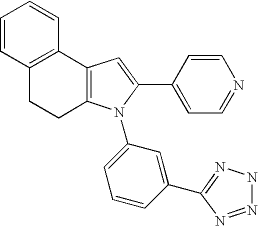 | 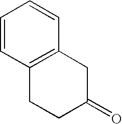 | 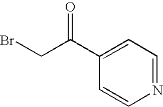 |
| 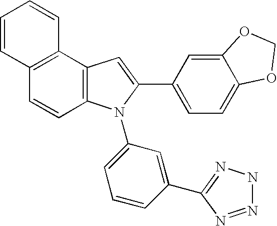 | 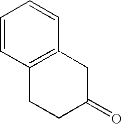 | 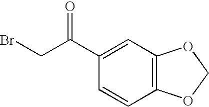 |
| 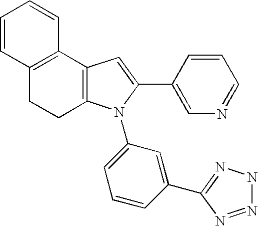 | 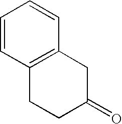 | 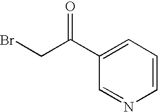 |
| 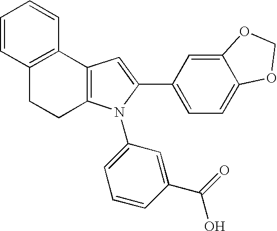 | 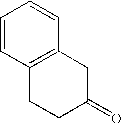 | 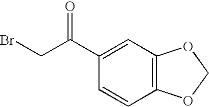 |
| 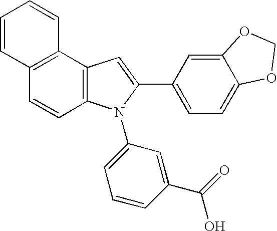 | 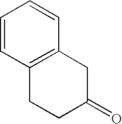 | 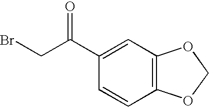 |
| 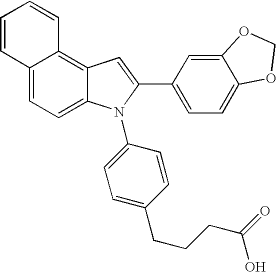 | 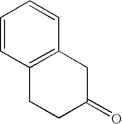 | 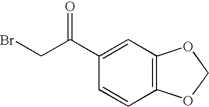 |
| 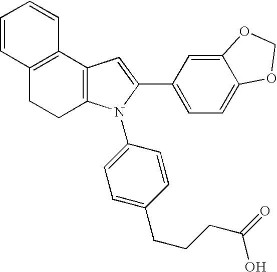 | 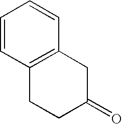 | 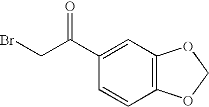 |
| 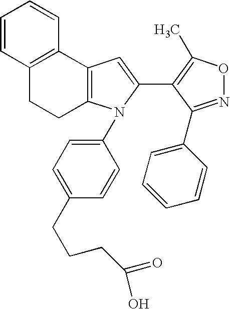 | 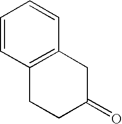 | 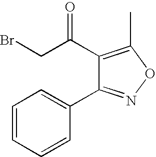 |
| 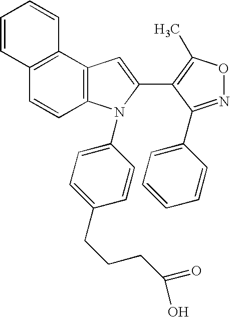 | 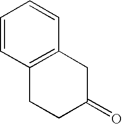 | 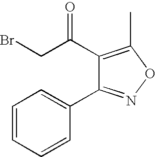 |
| 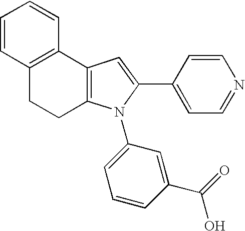 | 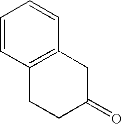 | 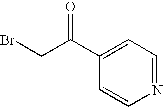 |
| 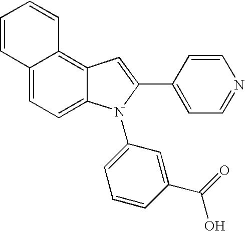 | 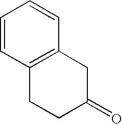 | 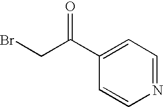 |
| 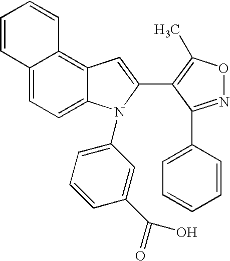 | 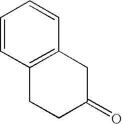 | 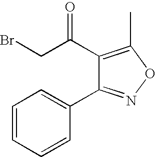 |
| 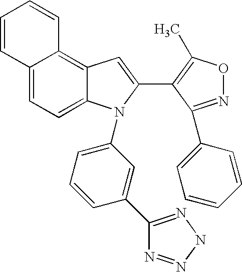 | 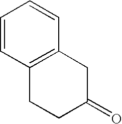 | 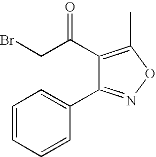 |
| 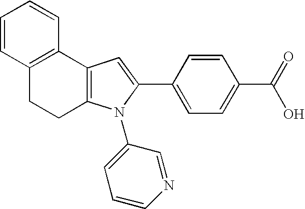 | 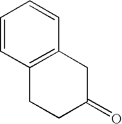 | 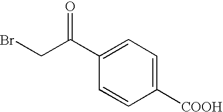 |
| 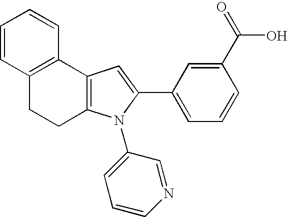 | 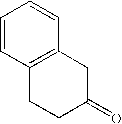 | 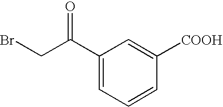 |
| 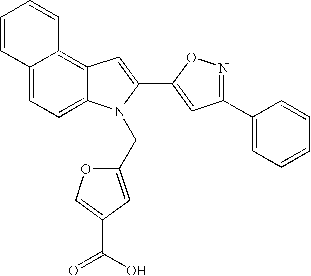 | 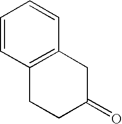 | 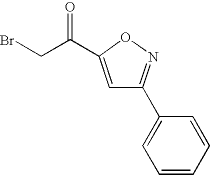 |
| 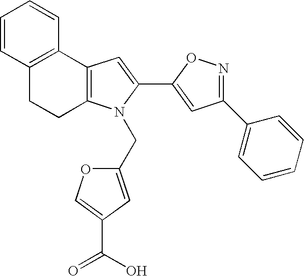 | 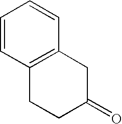 | 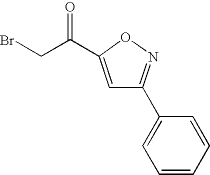 |
| 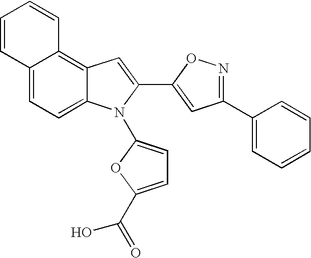 | 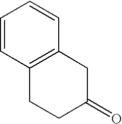 | 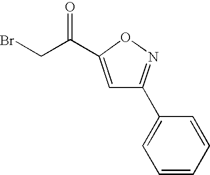 |
| 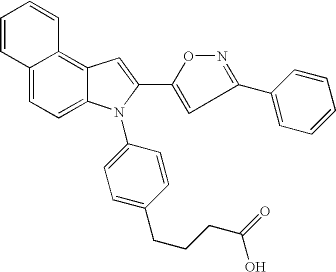 | 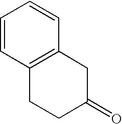 | 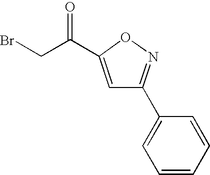 |
| 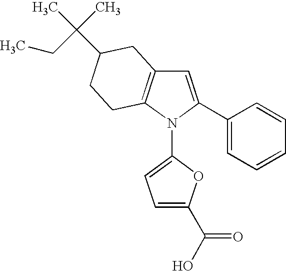 | 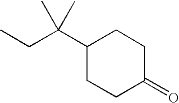 | 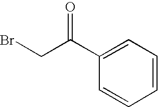 |
| 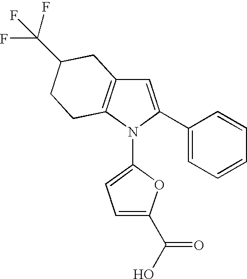 | 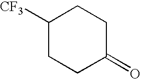 | 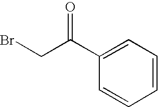 |
| 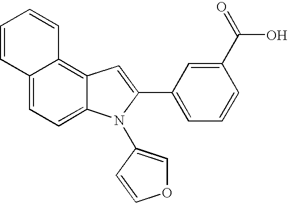 | 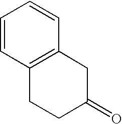 | 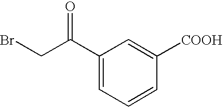 |
| 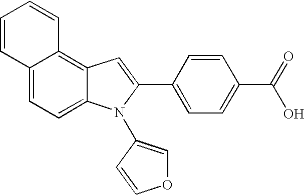 | 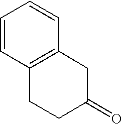 | 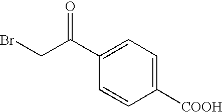 |
| 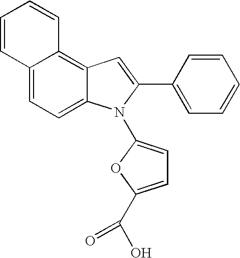 | 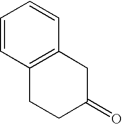 | 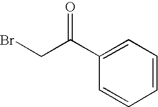 |
| 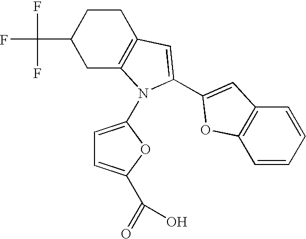 | 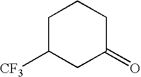 | 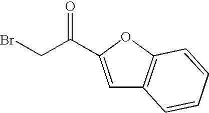 |
| 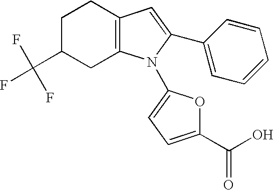 | 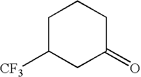 | 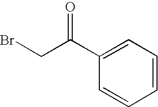 |
| 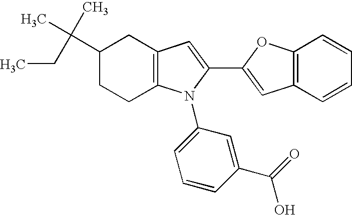 | 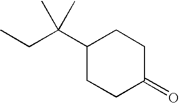 | 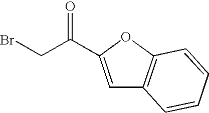 |
| 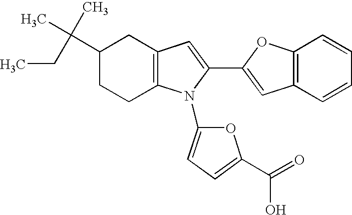 | 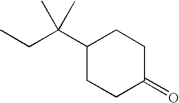 | 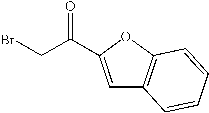 |
| 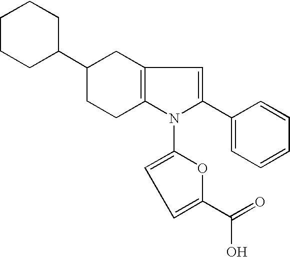 | 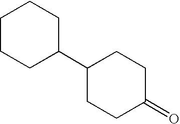 | 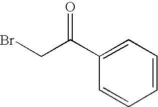 |
| 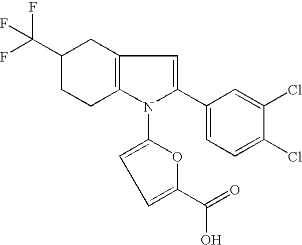 | 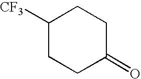 | 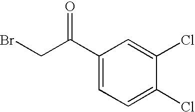 |
| 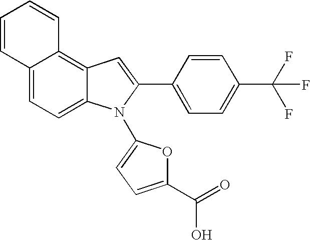 | 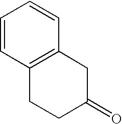 | 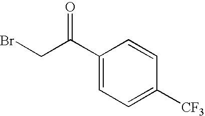 |
| 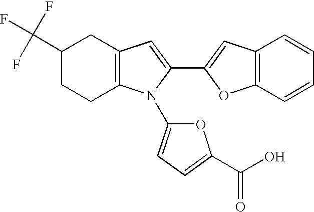 | 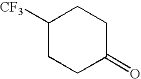 | 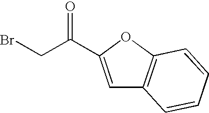 |
| 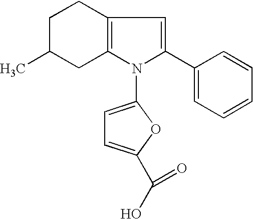 | 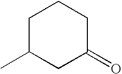 | 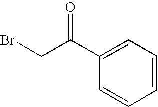 |
| 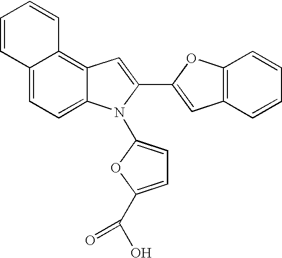 | 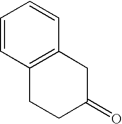 | 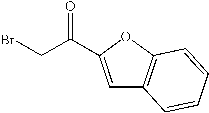 |
| 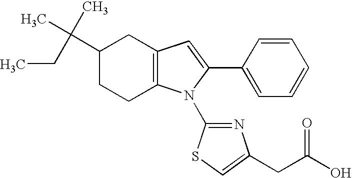 | 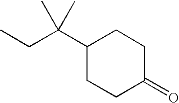 | 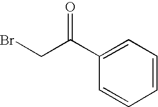 |
| 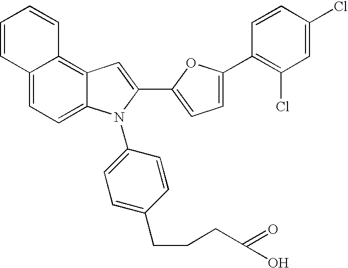 | 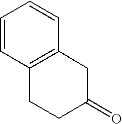 | 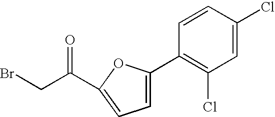 |
| 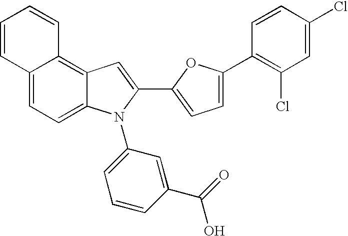 | 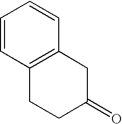 | 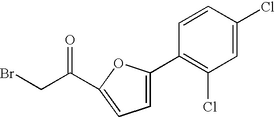 |
| 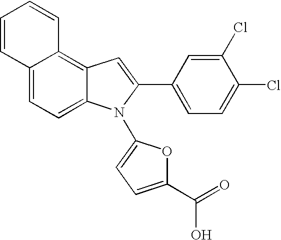 | 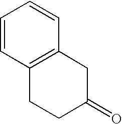 | 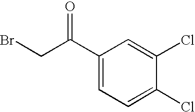 |
| 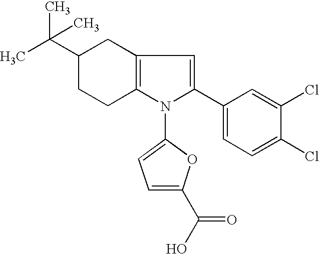 | 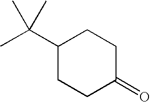 | 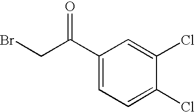 |
| 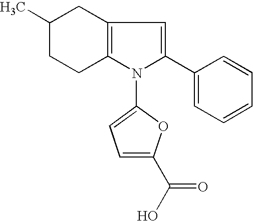 | 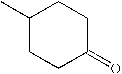 | 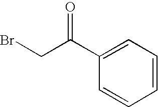 |
| 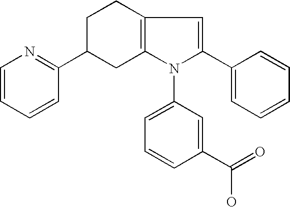 | 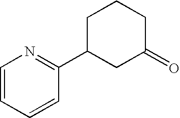 | 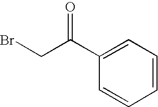 |
| 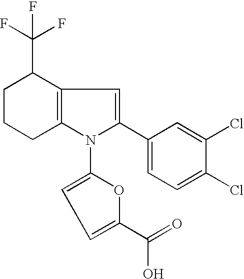 | 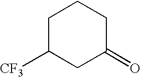 | 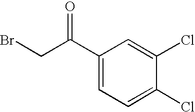 |
| 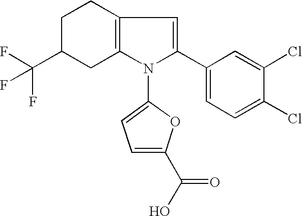 | 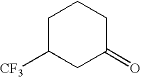 | 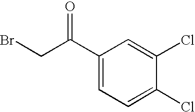 |
| 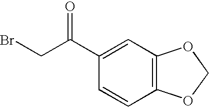 | 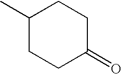 | 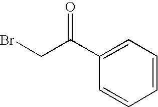 |
| 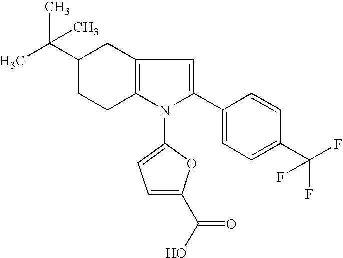 | 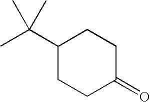 | 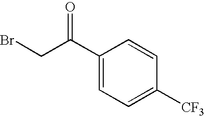 |
| 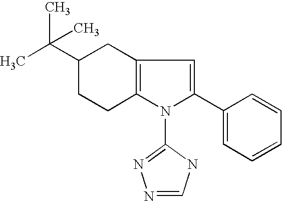 | 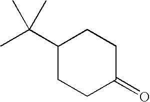 | 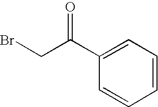 |
| 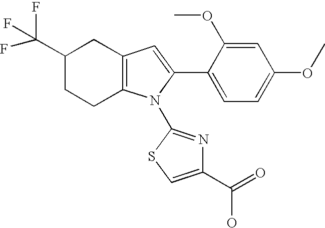 | 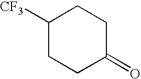 | 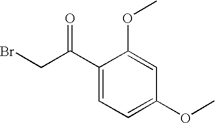 |
| 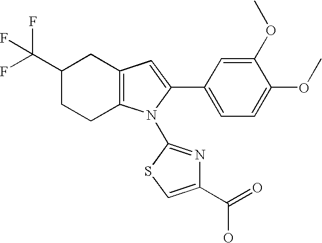 | 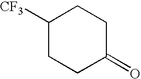 | 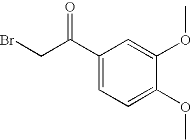 |
| 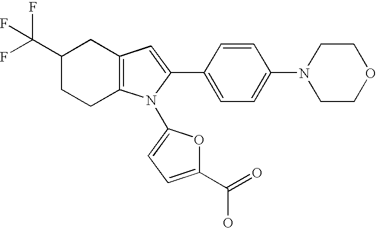 | 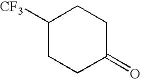 | 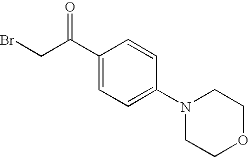 |
| 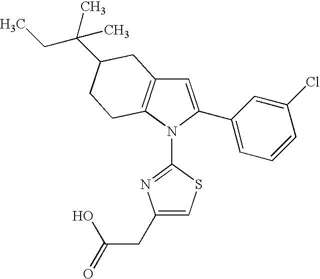 | 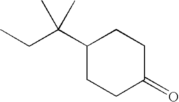 | 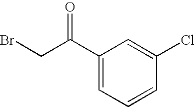 |
| 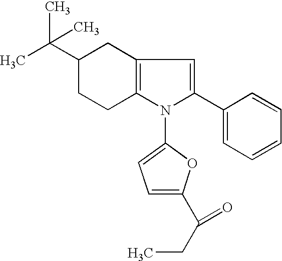 | 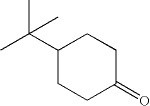 | 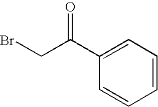 |
| 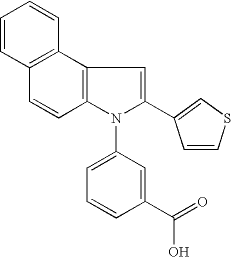 | 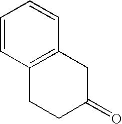 | 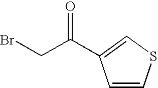 |
| 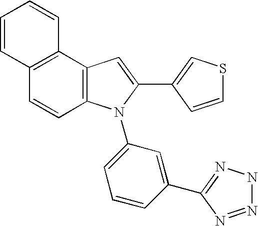 | 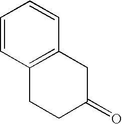 | 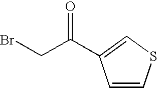 |
| 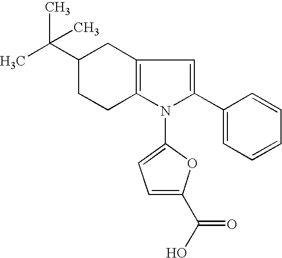 | 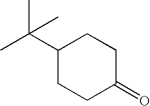 | 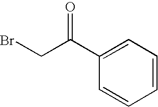 |
| 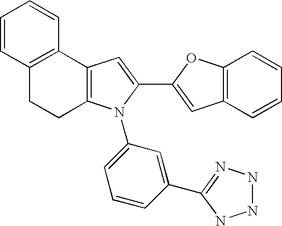 | 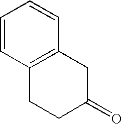 | 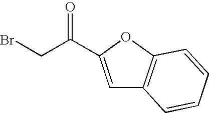 |
| 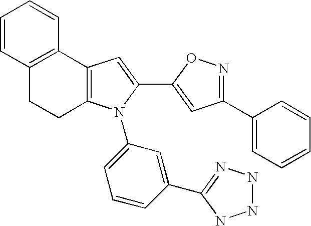 | 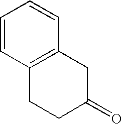 | 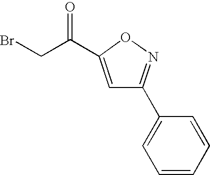 |
| 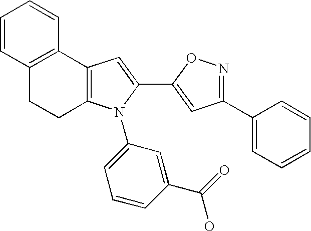 | 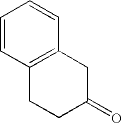 | 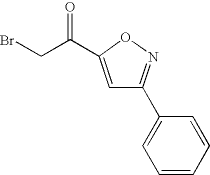 |
| 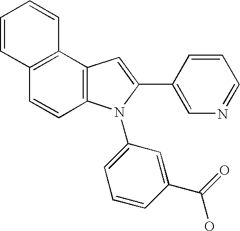 | 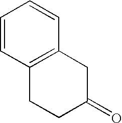 | 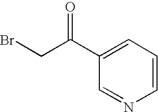 |
| 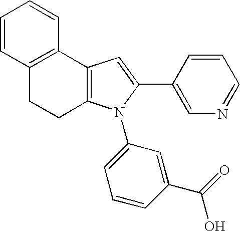 | 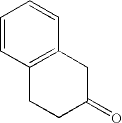 | 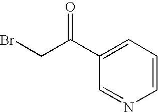 |
| 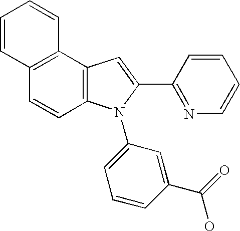 | 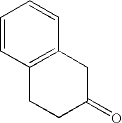 | 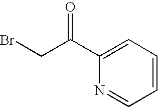 |
| 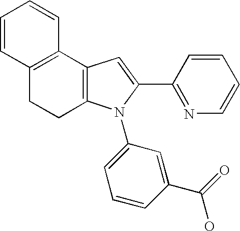 | 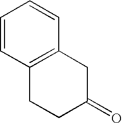 | 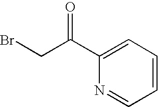 |
| 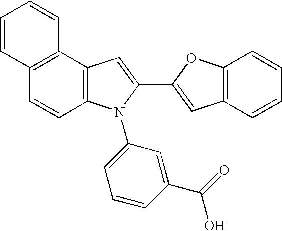 | 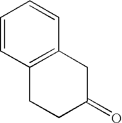 | 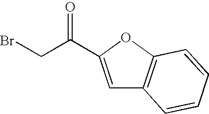 |
| 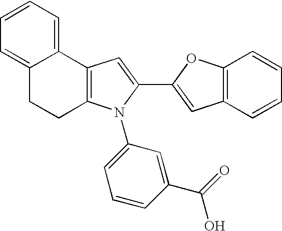 | 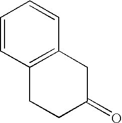 | 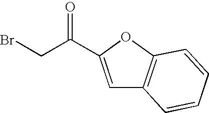 |
| 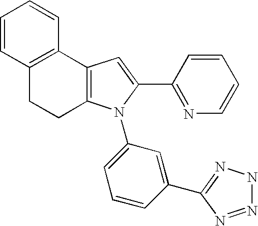 | 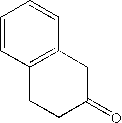 | 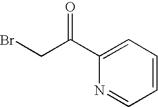 |
| 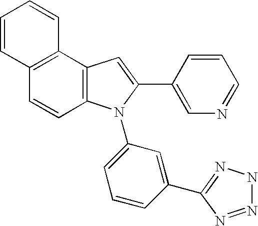 | 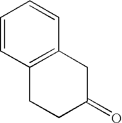 | 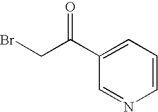 |
| 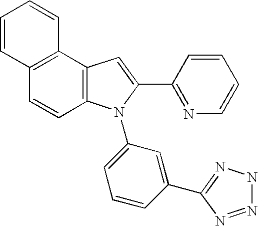 | 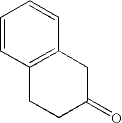 | 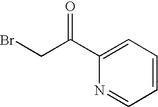 |
| 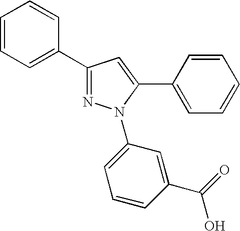 | 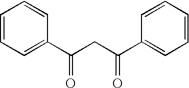 | none |
| 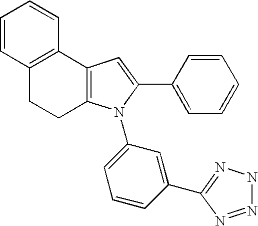 | 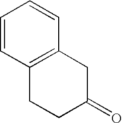 | 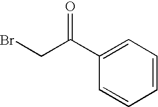 |
| 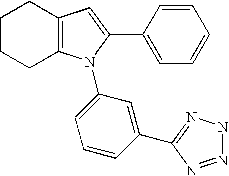 | 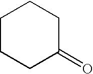 | 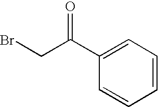 |

| product structure | aniline |
|---|---|
| 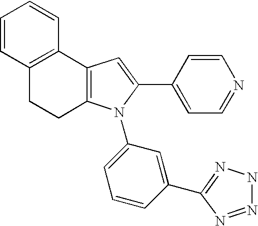 | 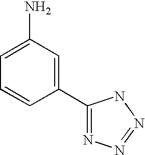 |
| 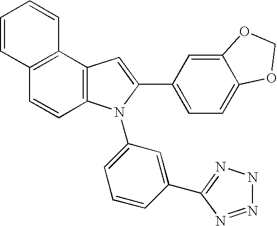 | 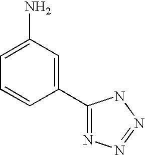 |
| 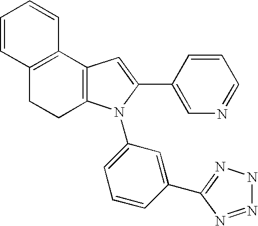 | 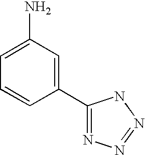 |
| 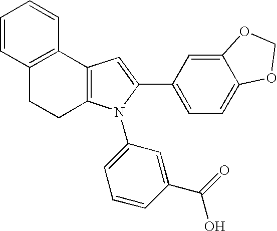 | 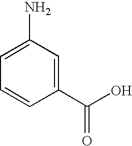 |
| 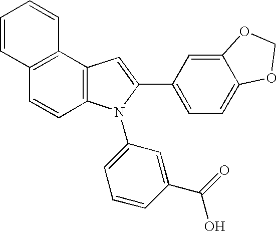 | 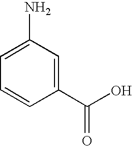 |
| 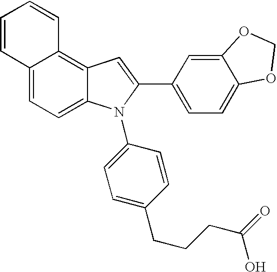 | 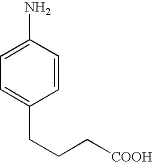 |
| 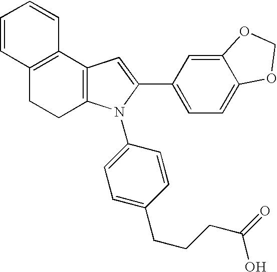 | 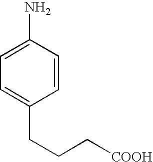 |
| 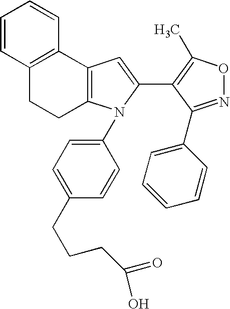 | 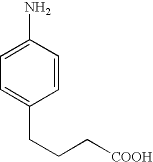 |
| 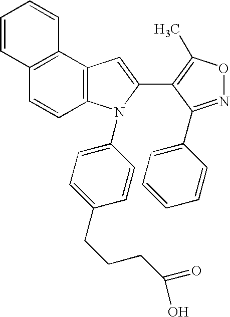 | 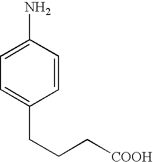 |
| 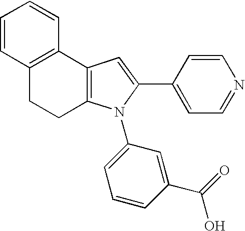 | 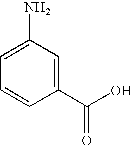 |
| 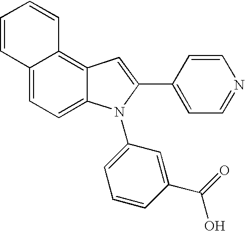 | 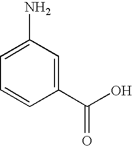 |
| 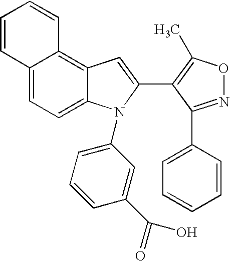 | 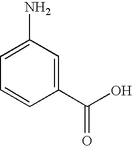 |
| 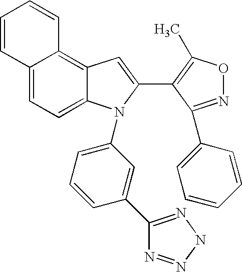 | 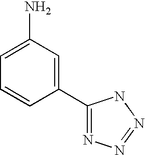 |
| 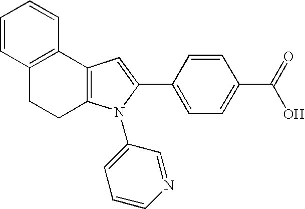 | 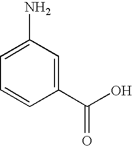 |
| 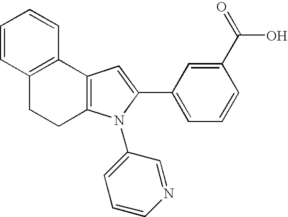 | 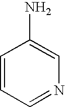 |
| 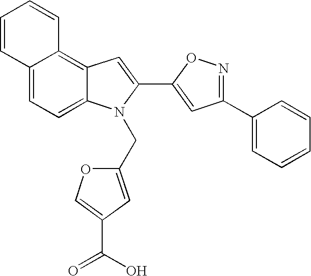 | 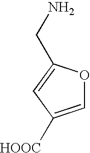 |
| 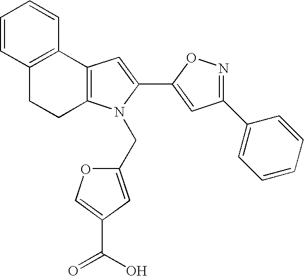 | 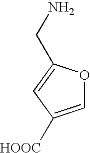 |
| 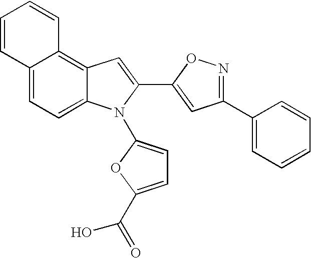 | 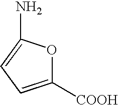 |
| 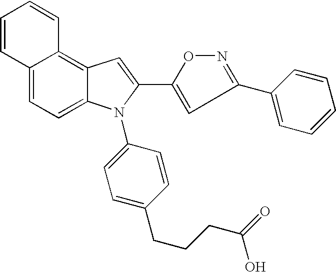 | 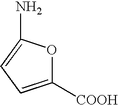 |
| 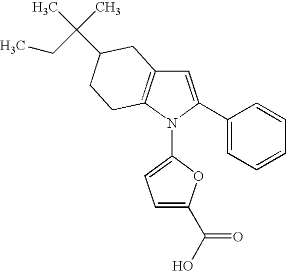 | 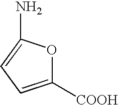 |
| 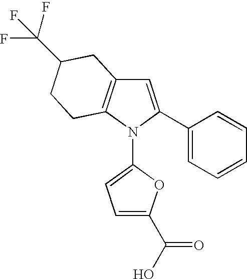 | 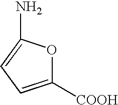 |
| 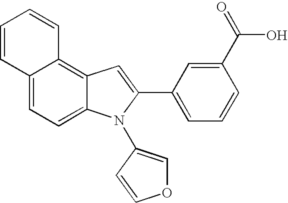 | 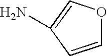 |
| 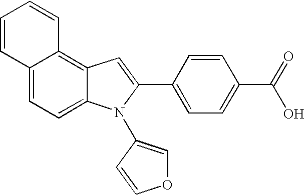 | 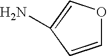 |
| 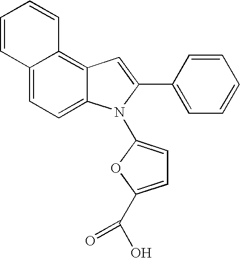 | 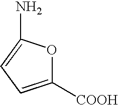 |
| 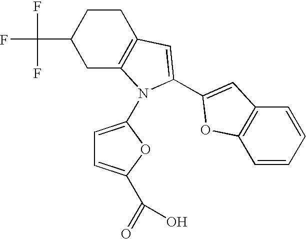 | 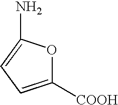 |
| 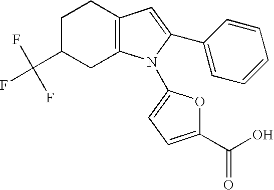 | 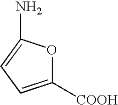 |
| 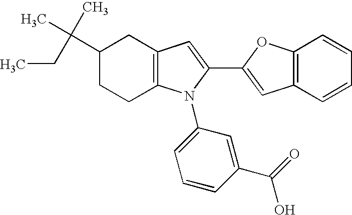 | 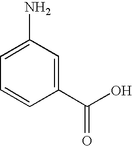 |
| 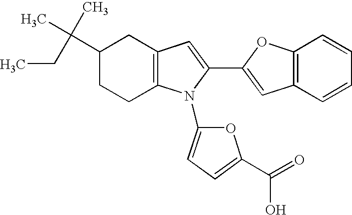 | 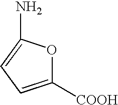 |
| 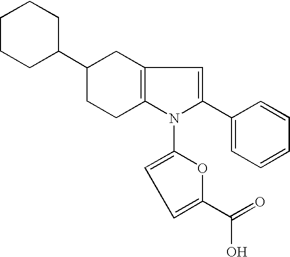 | 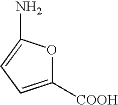 |
| 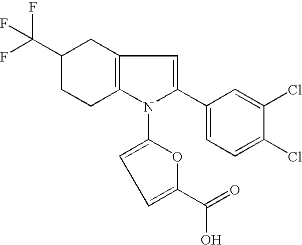 | 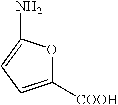 |
| 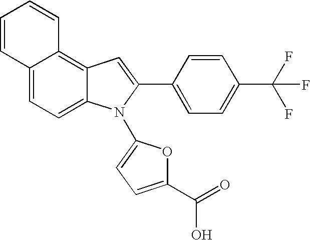 | 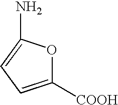 |
| 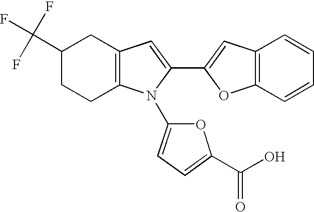 | 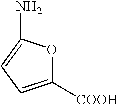 |
| 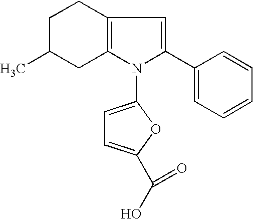 | 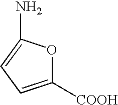 |
| 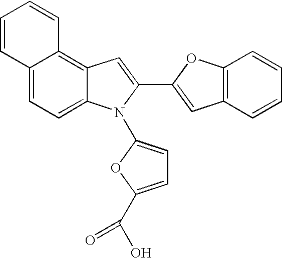 | 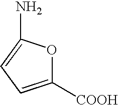 |
| 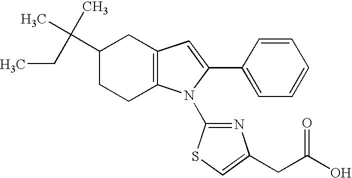 | 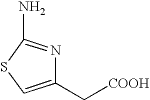 |
| 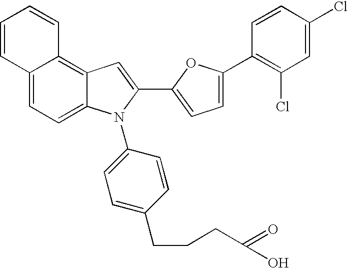 | 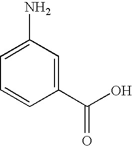 |
| 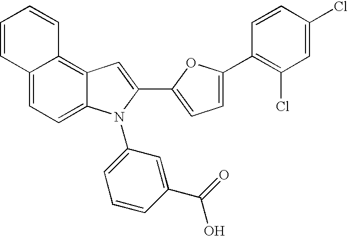 | 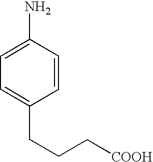 |
| 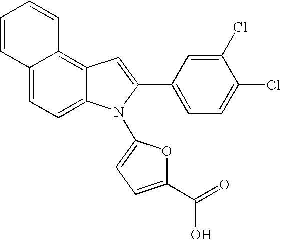 | 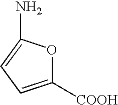 |
| 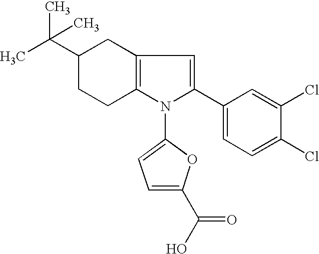 | 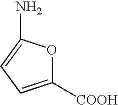 |
| 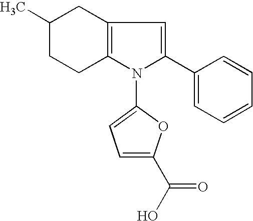 | 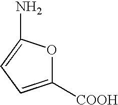 |
| 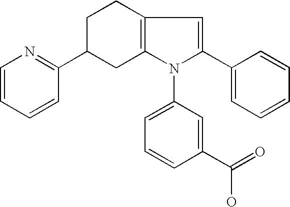 | 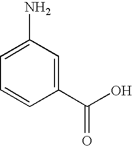 |
| 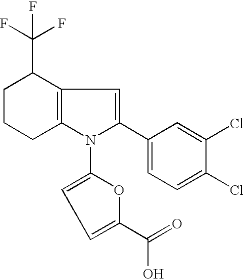 | 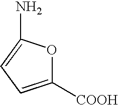 |
| 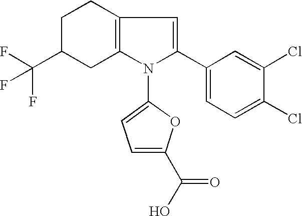 | 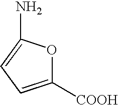 |
| 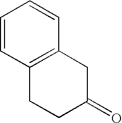 | 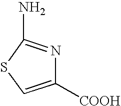 |
| 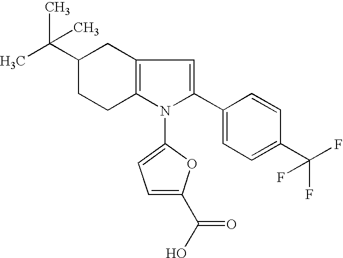 | 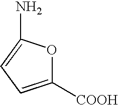 |
| 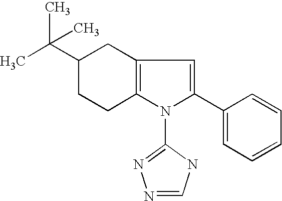 | 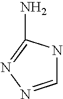 |
| 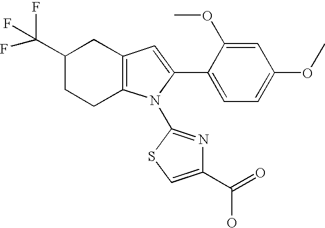 | 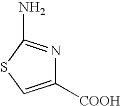 |
| 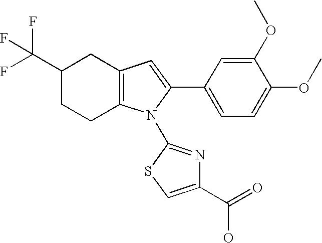 | 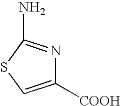 |
| 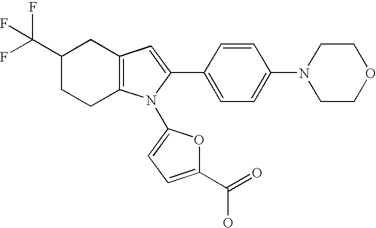 | 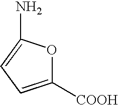 |
| 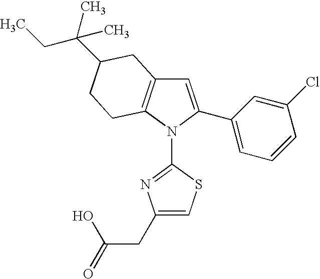 | 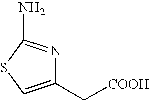 |
| 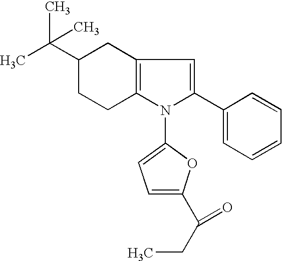 | 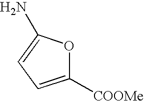 |
| 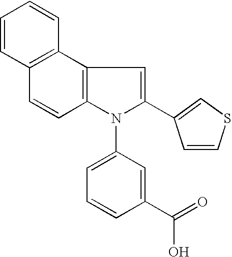 | 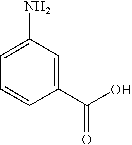 |
| 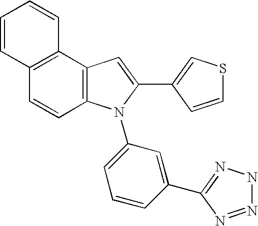 | 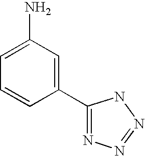 |
| 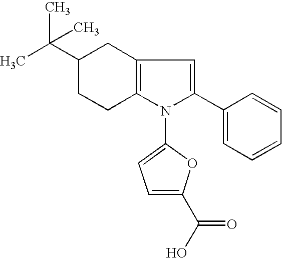 | 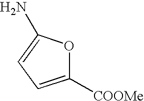 |
| 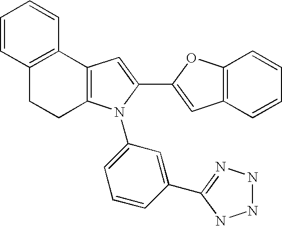 | 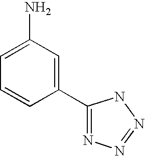 |
| 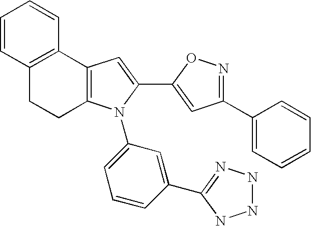 | 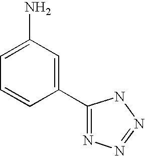 |
| 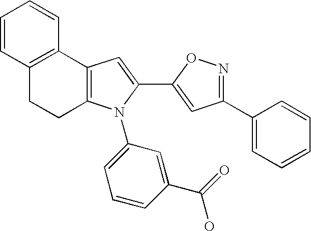 | 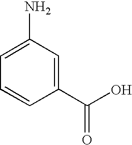 |
| 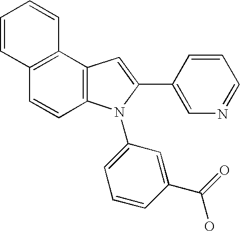 | 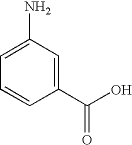 |
| 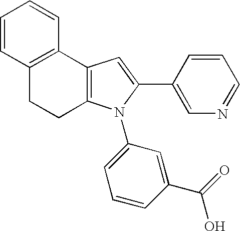 | 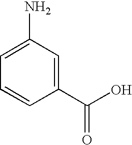 |
| 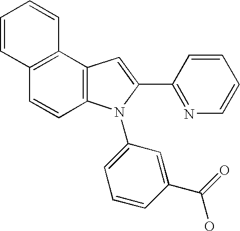 | 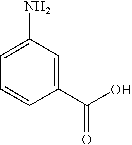 |
| 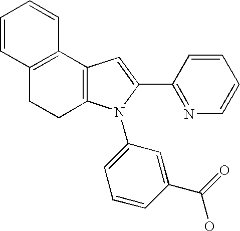 | 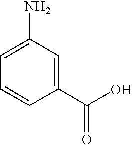 |
| 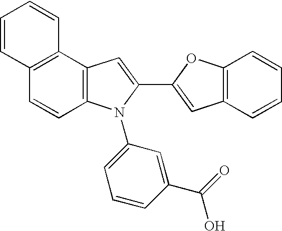 | 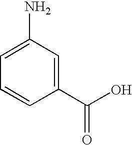 |
| 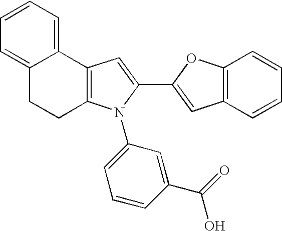 | 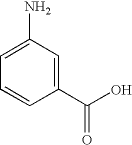 |
| 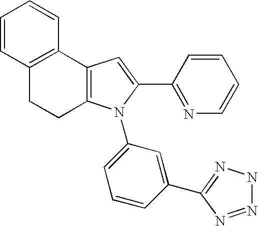 | 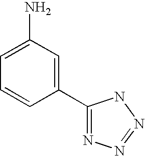 |
| 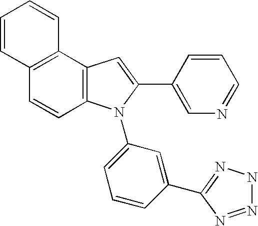 | 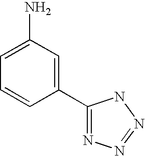 |
| 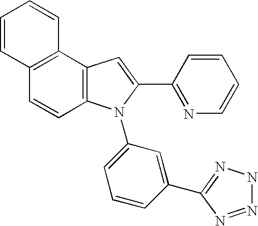 | 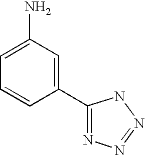 |
| 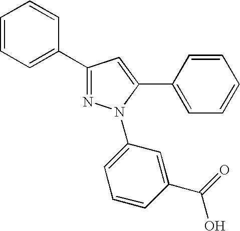 | 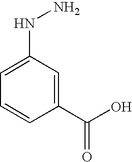 |
| 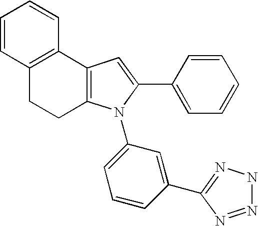 | 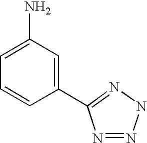 |
| 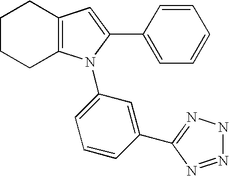 | 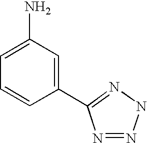 |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A method treating ALS comprising identifying a patient in need of such treatment and administering to said patient a therapeutically effective amount of a compound of Formula I or II, or a pharmaceutically acceptable salts thereof. 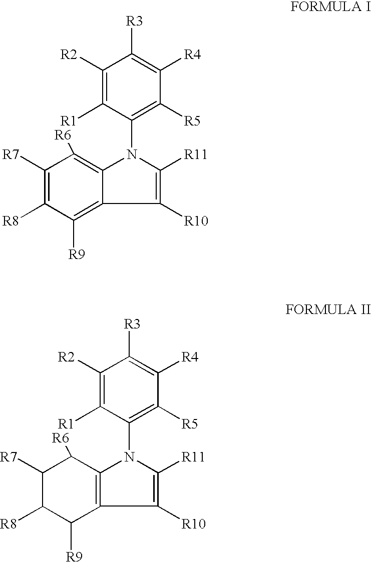
wherein one or more of R1-R5 independently chosen from -L-C(═O)OH, -L-CH═CHC(═O)OH, -L-C(═O)NH₂, -L-C(═O)NH(C₁₋₃ alkyl), -L-C(═O)N(C₁₋₃ alkyl)₂, -L-S(═O)₂(C₁₋₃alkyl), -L-S(═O)₂NH₂, -L-S(═O)₂N(C₁₋₃ alkyl)₂, -L-S(═O)₂NH(C₁₋₃ alkyl), -L-C(═O)NHOH, -L-C(═O)CH₂NH₂, -L-C(═O)CH₂OH, -L-C(═O)CH₂SH, -L-C(═O)NHCN, -L-NHC(═O)ORₒ, -L-C(═O)NHRₒ, -L-NH(C═O)NHRₒ, -L-C(═O)N(Rₒ)₂, -L-NH(C═O)N(Rₒ)₂, -L-sulfo, -L-(2,6 difluorophenol), -L-phosphono, and -L-tetrazolyl, and the others of R1-R5, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;
R6-R10, independent of one another, are chosen from hydro, hydroxyl, halo, alkyl, alkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, —C(═O)—N-morpholino, -cyclohexyl, -morpholino, -pyrrolidinyl, -piperazinyl, —(N-methyl)-piperazinyl, —OCH₂-phenyl, -pyridinyl, methylenedioxy, ethylenedioxy, —C(═O)OCH₂CH₃ substituted furanyl, para-(C(═O)OCH₂CH₃)-phenyl, and —O—Si(CH₃)₂(C(CH₃)₃); two adjacent of R6-R9 can be taken together to form a 4-7 member optionally substituted aryl or cycloalkyl ring;
R11 is an optionally substituted phenyl group or an optionally substituted heterocyclic group;
Rₒ is chosen from alkyl and haloalkyl; and
L can be saturated, partially saturated, or unsaturated, and is chosen from —(CH₂)ₙ—(CH₂)ₙ—, —(CH₂)ₙC(═O)(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, 3, 4, 5, 6, 7, and 8, and wherein each carbon can be optionally substituted with one or more C₁₋₃ alkyl or C₃₋₆ cycloalkyl.
